(19) United States
(12) Patent Application Publication
(10) Pub. No.: US 2022/0119490 A1
(43) Pub. Date: Apr. 21, 2022

(54) MODULATORS OF CELL SURFACE PROTEIN INTERACTIONS AND METHODS AND COMPOSITIONS RELATED TO SAME

(71) Applicant: Genentech, Inc., South San Francisco, CA (US)

(72) Inventors: Nadia MARTINEZ-MARTIN, San Francisco, CA (US); Shannon J. TURLEY, San Anselmo, CA (US); Erik VERSCHUEREN, San Francisco, CA (US)

(21) Appl. No.: 17/489,598

(22) Filed: Sep. 29, 2021

Related U.S. Application Data
(63) Continuation of application No. PCT/US2020/025471, filed on Mar. 27, 2020.
(60) Provisional application No. 63/000,466, filed on Mar. 26, 2020.
(60) Provisional application No. 62/826,904, filed on Mar. 29, 2019.

(51) Int. Cl. C07K 14/705; A61K 39/00; C07K 16/30; C07K 16/28; G01N 33/68

(52) U.S. Cl. CPC C07K 14/70596; A61K 39/0011; C07K 16/3092; C07K 2317/76; G01N 33/6845; C07K 2317/75; C07K 16/2896

(57) ABSTRACT

Provided herein are methods for identifying modulators of cell surface protein interactions and activities, as well as modulators of cell surface protein interactions and activities.

### CROSS-REFERENCE TO RELATED APPLICATIONS

This application is a continuation of International Application No. PCT/US2020/025471, filed on Mar. 27, 2020, which claims benefit of U.S. Application No. 63/000,466, filed Mar. 26, 2020, and U.S. Application No. 62/826,904, filed on Mar. 29, 2019.

### SEQUENCE LISTING

The instant application contains a Sequence Listing which has been submitted electronically in ASCII format and is hereby incorporated by reference in its entirety. Said ASCII copy, created on Sep. 29, 2021, is named 50474-182004_Sequence_Listing_9_29_21_ST25 and is 24,539 bytes in size.

### FIELD OF THE INVENTION

Provided herein are methods for identifying modulators of cell surface protein interactions and activities, as well as modulators of cell surface protein interactions and activities.

### BACKGROUND

Recent annotations predict that over 5,000 genes in the human genome encode plasma membrane-expressed or secreted proteins, i.e., proteins acting at the cell surface. Classes of proteins present at the cell surface include cell surface receptors (e.g., single transmembrane (STM) receptors) and Immunoglobulin Superfamily (IgSF) proteins. Many of these proteins have been linked to cancer and other diseases, and are thus prime targets for development of therapeutic agents; however, the binding partners of many plasma membrane proteins (e.g., STM and IgSF proteins) have remained uncharacterized. This discrepancy is due to the incompatibility of currently used proteomics technologies, e.g., yeast two-hybrid assays and affinity purification/mass spectrometry (AP/MS), for studying plasma membrane-expressed proteins. High-throughput screens for extracellular protein-protein interactions have recently been developed; however, therapeutic agents that modulate the majority of these interactions have not yet been identified.

Thus, there is an unmet need for methods and compositions for the identification of interactions between cell surface proteins, as well as novel modulators of such interactions and methods of using the same.

### SUMMARY OF THE INVENTION

The present invention provides methods for identifying modulators of cell surface protein interactions and activities, as well as modulators of cell surface protein interactions and activities.

In one aspect, the disclosure features a method of identifying an individual having a cancer who may benefit from a treatment comprising a PD-L1 axis binding antagonist, the method comprising determining an expression level of a first member and a second member of at least one of the gene pairs of Table 15 in a sample from the individual, wherein an expression level of the first member of the gene pair that is above a first reference expression level and an expression level of the second member of the gene pair that is above a second reference expression level identifies the individual as one who may benefit from a treatment comprising a PD-L1 axis binding antagonist.

In another aspect, the disclosure features a method of selecting a therapy for an individual having a cancer, the method comprising determining an expression level of a first member and a second member at least one of the gene pairs of Table 15 in a sample from the individual, wherein an expression level of the first member of the gene pair that is above a first reference expression level and an expression level of the second member of the gene pair that is above a second reference expression level identifies the individual as one who may benefit from a treatment comprising a PD-L1 axis binding antagonist.

In some aspects, the individual has an expression level of the first member of the gene pair that is above a first reference expression level and an expression level of the second member of the gene pair that is above a second reference expression level and the method further comprises administering to the individual an effective amount of a PD-L1 axis binding antagonist.

In another aspect, the disclosure features a method of treating an individual having a cancer, the method comprising (a) determining an expression level of a first member and a second member at least one of the gene pairs of Table 15 in a sample from the individual, wherein the expression level of the first member of the gene pair is above a first reference expression level and the expression level of the second member of the gene pair is above a second reference expression level; and (b) administering an effective amount of a PD-L1 axis binding antagonist to the individual.

In another aspect, the disclosure features a method of treating an individual having a cancer, the method comprising administering a PD-L1 axis binding antagonist to an individual who has been determined to have an expression level of a first member of a gene pair of Table 15 that is above a first reference expression level and an expression level of the second member of the gene pair that is above a second reference expression level.

In another aspect, the disclosure features a method of identifying an individual having a cancer who may benefit from a treatment other than or in addition to a PD-L1 axis binding antagonist, the method comprising determining an expression level of a first member and a second member of at least one of the gene pairs of Table 16 in a sample from the individual, wherein an expression level of the first member of the gene pair that is above a first reference expression level and an expression level of the second member of the gene pair that is above a second reference expression level identifies the individual as one who may benefit from a treatment other than or in addition to a PD-L1 axis binding antagonist.

In another aspect, the disclosure features a method of selecting a therapy for an individual having a cancer, the method comprising determining an expression level of a first member and a second member at least one of the gene pairs of Table 16 in a sample from the individual, wherein an expression level of the first member of the gene pair that is above a first reference expression level and an expression level of the second member of the gene pair that is above a second reference expression level identifies the individual as one who may benefit from a treatment other than or in addition to a PD-L1 axis binding antagonist.

In some aspects, the individual has an expression level of the first member of the gene pair that is above a first reference expression level and an expression level of the second member of the gene pair that is above a second reference expression level and the method comprises administering to the individual an effective amount of a treatment other than or in addition to a PD-L1 axis binding antagonist.

In some aspects, the first reference expression level is a pre-assigned expression level and the second reference expression level is a pre-assigned reference expression level.

In some aspects, the sample from the individual is obtained from the individual prior to administration of an anti-cancer therapy. In some aspects, the sample from the individual is obtained from the individual after administration of an anti-cancer therapy.

In some aspects, the sample from the individual is a tumor tissue sample or a tumor fluid sample.

In some aspects, the sample is a formalin-fixed and paraffin-embedded (FFPE) sample, an archival sample, a fresh sample, or a frozen sample. In some aspects, the tumor tissue sample is a FFPE sample.

In some aspects, the expression level of the first member and the second member of the gene pair in the sample is a protein expression level; or the expression level of the first member and the second member of the gene pair in the sample is an mRNA expression level. In some aspects, the expression level of the first member and the second member of the gene pair in the sample is a mRNA expression level of the first member and the second member of the gene pair, respectively.

In some aspects, the mRNA expression level of the first member and the second member of the gene pair is determined by in situ hybridization (ISH), RNA-seq, RT-qPCR, qPCR, multiplex qPCR or RT-qPCR, microarray analysis, SAGE, MassARRAY technique, FISH, or a combination thereof. In some aspects, the first reference expression level is between about 0.25 to about 0.5 counts per million (CPM) and the second reference expression level is between about 0.25 to about 0.5 CPM. In some aspects, the first reference expression level is 0.25 CPM and the second reference expression level is 0.25 CPM.

In some aspects, the first reference expression level and the second reference expression level are expression levels of the first member and the second member of the gene pair, respectively, in a reference population of individuals having a cancer. In some aspects, the cancer is a urinary tract cancer, e.g., a urinary tract carcinoma, e.g., a locally advanced urothelial carcinoma or a metastatic urothelial carcinoma (mUC).

In some aspects, the benefit comprises an extension in the individual's overall survival (OS) as compared to treatment without the PD-L1 axis binding antagonist.

In some aspects, the first member of the gene pair is SIGLEC6 and the second member of the gene pair is NCR1.

In some aspects, the first member of the gene pair is BTN3A1 and the second member of the gene pair is LRRC4B.

In some aspects, the first member of the gene pair is CD80 and the second member of the gene pair is CTLA4.

In some aspects, the first member of the gene pair is BTN3A3 and the second member of the gene pair is LRRC4B.

In some aspects, the first member of the gene pair is EFNB1 and the second member of the gene pair is TRHDE.

In some aspects, the first member of the gene pair is CTLA4 and the second member of the gene pair is PCDHGB4.

In some aspects, the first member of the gene pair is CTLA4 and the second member of the gene pair is FAM200A.

In some aspects, the first member of the gene pair is CA12 and the second member of the gene pair is SIGLEC6.

In some aspects, the first member of the gene pair is ILDR2 and the second member of the gene pair is CLEC12B.

In some aspects, the first member of the gene pair is EFNB1 and the second member of the gene pair is ITLN1.

In some aspects, the first member of the gene pair is CADM1 and the second member of the gene pair is CRTAM.

In some aspects, the first member of the gene pair is CD79B and the second member of the gene pair is CD244.

In some aspects, the first member of the gene pair is DAG1 and the second member of the gene pair is EFNB1.

In some aspects, the first member of the gene pair is EFNB1 and the second member of the gene pair is EVC2.

In some aspects, the first member of the gene pair is GPC4 and the second member of the gene pair is FGFRL1.

In some aspects, the first member of the gene pair is EFNB3 and the second member of the gene pair is EPHB4.

In some aspects, the first member of the gene pair is PTPRD and the second member of the gene pair is LRFN4.

In some aspects, the first member of the gene pair is EFNB1 and the second member of the gene pair is AQPEP.

In some aspects, the first member of the gene pair is EFNB1 and the second member of the gene pair is DSG4.

In some aspects, the first member of the gene pair is LDLR and the second member of the gene pair is LILRB5.

In some aspects, the first member of the gene pair is EFNB3 and the second member of the gene pair is EPHB3.

In some aspects, the first member of the gene pair is PLXNB3 and the second member of the gene pair is SEMA4G.

In some aspects, the first member of the gene pair is EFNB1 and the second member of the gene pair is EPHB6.

In some aspects, the first member of the gene pair is FLT4 and the second member of the gene pair is FLRT2.

In some aspects, the first member of the gene pair is AXL1 and the second member of the gene pair is IL1RL1.

In some aspects, the first member of the gene pair is CD320 and the second member of the gene pair is IGSF5.

In some aspects, the first member of the gene pair is CD59 and the second member of the gene pair is STAB1.

In some aspects, the first member of the gene pair is CNTN3 and the second member of the gene pair is PTPRG.

In some aspects, the first member of the gene pair is EFNB1 and the second member of the gene pair is EPHA3.

In some aspects, the first member of the gene pair is EFNB3 and the second member of the gene pair is EPHB2.

In some aspects, the first member of the gene pair is EGF and the second member of the gene pair is TNFRSF11B.

In some aspects, the first member of the gene pair is ENPEP and the second member of the gene pair is SLITRK1.

In some aspects, the first member of the gene pair is FCGR3B and the second member of the gene pair is EDA2R.

In some aspects, the first member of the gene pair is IL20RA and the second member of the gene pair is CLEC14A.

In some aspects, the first member of the gene pair is IL6R and the second member of the gene pair is BTNL9.

In some aspects, the first member of the gene pair is IZUMO1 and the second member of the gene pair is LILRA5.

In some aspects, the first member of the gene pair is NGFR and the second member of the gene pair is LRRTM3.

In some aspects, the first member of the gene pair is NTM and the second member of the gene pair is AMIGO2.

In some aspects, the first member of the gene pair is PCDHB3 and the second member of the gene pair is IGSF11.

In some aspects, the first member of the gene pair is PTGFRN and the second member of the gene pair is TMEM59L.

In some aspects, the first member of the gene pair is TREM1 and the second member of the gene pair is VSIG8.

In some aspects, the PD-L1 axis binding antagonist is selected from the group consisting of a PD-L1 binding antagonist, a PD-1 binding antagonist, and a PD-L2 binding antagonist.

In some aspects, the PD-L1 axis binding antagonist is a PD-L1 binding antagonist.

In some aspects, the PD-L1 binding antagonist inhibits the binding of PD-L1 to one or more of its ligand binding partners.

In some aspects, the PD-L1 binding antagonist inhibits the binding of PD-L1 to PD-1.

In some aspects, the PD-L1 binding antagonist inhibits the binding of PD-L1 to B7-1.

In some aspects, the PD-L1 binding antagonist inhibits the binding of PD-L1 to both PD-1 and B7-1.

In some aspects, the PD-L1 binding antagonist is an antibody or antigen-binding fragment thereof.

In some aspects, the antibody is selected from the group consisting of atezolizumab, MDX-1105, MEDI4736 (durvalumab), and MSB0010718C (avelumab).

In some aspects, the anti-PD-L1 antibody comprises the following hypervariable regions: (a) an HVR-H1 sequence of GFTFSDSWIH (SEQ ID NO: 19); (b) an HVR-H2 sequence of AWISPYGGSTYYADSVKG (SEQ ID NO: 20); (c) an HVR-H3 sequence of RHWPGGFDY (SEQ ID NO: 21); (d) an HVR-L1 sequence of RASQDVSTAVA (SEQ ID NO: 22); (e) an HVR-L2 sequence of SASFLYS (SEQ ID NO: 23); and (f) an HVR-L3 sequence of QQYLYHPAT (SEQ ID NO: 24).

In some aspects, the anti-PD-L1 antibody comprises: (a) a heavy chain variable (VH) domain comprising an amino acid sequence having at least 90% sequence identity to the amino acid sequence of SEQ ID NO: 3; (b) a light chain variable (VL) domain comprising an amino acid sequence having at least 90% sequence identity to the amino acid sequence of SEQ ID NO: 4; or (c) a VH domain as in (a) and a VL domain as in (b).

In some aspects, the anti-PD-L1 antibody comprises (a) a heavy chain variable (VH) domain comprising an amino acid sequence having at least 95% sequence identity to the amino acid sequence of SEQ ID NO: 3; (b) a light chain variable (VL) domain comprising an amino acid sequence having at least 95% sequence identity to the amino acid sequence of SEQ ID NO: 4; or (c) a VH domain as in (a) and a VL domain as in (b).

In some aspects, the anti-PD-L1 antibody comprises (a) a heavy chain variable (VH) domain comprising an amino acid sequence having at least 96% sequence identity to the amino acid sequence of SEQ ID NO: 3; (b) a light chain variable (VL) domain comprising an amino acid sequence having at least 96% sequence identity to the amino acid sequence of SEQ ID NO: 4; or (c) a VH domain as in (a) and a VL domain as in (b).

In some aspects, the anti-PD-L1 antibody comprises (a) a heavy chain variable (VH) domain comprising an amino acid sequence having at least 97% sequence identity to the amino acid sequence of SEQ ID NO: 3; (b) a light chain variable (VL) domain comprising an amino acid sequence having at least 97% sequence identity to the amino acid sequence of SEQ ID NO: 4; or (c) a VH domain as in (a) and a VL domain as in (b).

In some aspects, the anti-PD-L1 antibody comprises (a) a heavy chain variable (VH) domain comprising an amino acid sequence having at least 98% sequence identity to the amino acid sequence of SEQ ID NO: 3; (b) a light chain variable (VL) domain comprising an amino acid sequence having at least 98% sequence identity to the amino acid sequence of SEQ ID NO: 4; or (c) a VH domain as in (a) and a VL domain as in (b).

In some aspects, the anti-PD-L1 antibody comprises (a) a heavy chain variable (VH) domain comprising an amino acid sequence having at least 99% sequence identity to the amino acid sequence of SEQ ID NO: 3; (b) a light chain variable (VL) domain comprising an amino acid sequence having at least 99% sequence identity to the amino acid sequence of SEQ ID NO: 4; or (c) a VH domain as in (a) and a VL domain as in (b).

In some aspects, the anti-PD-L1 antibody comprises (a) a VH domain comprising the amino acid sequence of SEQ ID NO: 3; and (b) a VL domain comprising the amino acid sequence of SEQ ID NO: 4.

In some aspects, the anti-PD-L1 antibody is atezolizumab (MPDL3280A).

In some aspects, the PD-L1 axis binding antagonist is a PD-1 binding antagonist. In some aspects, the PD-1 binding antagonist inhibits the binding of PD-1 to one or more of its ligand binding partners.

In some aspects, the PD-1 binding antagonist inhibits the binding of PD-1 to PD-L1.

In some aspects, the PD-1 binding antagonist inhibits the binding of PD-1 to PD-L2.

In some aspects, the PD-1 binding antagonist inhibits the binding of PD-1 to both PD-L1 and PD-L2.

In some aspects, the PD-1 binding antagonist is an antibody or antigen-binding fragment thereof.

In some aspects, the antibody is selected from the group consisting of: MDX-1106 (nivolumab), MK-3475 (pembrolizumab), MEDI-0680 (AMP-514), PDR001, REGN2810, and BGB-108.

In some aspects, the PD-1 binding antagonist is an Fc-fusion protein.

In another aspect, the disclosure features a method of treating an individual having a cancer comprising administering to the individual an effective amount of an agonist of CD177 activity.

In another aspect, the disclosure features a method of identifying an individual having a cancer who may benefit from a treatment comprising an agonist of CD177 activity, the method comprising determining an expression level of podoplanin (PDPN) in a sample from the individual, wherein an expression level of PDPN in the sample that is above a reference PDPN expression level identifies the individual as one who may benefit from a treatment comprising an agonist of CD177 activity.

In another aspect, the disclosure features a method of selecting a therapy for an individual having a cancer, the method comprising determining an expression level of PDPN in a sample from the individual, wherein an expression level of PDPN in the sample that is above a reference PDPN expression level identifies the individual as one who may benefit from a treatment comprising an agonist of CD177 activity.

In some aspects, the individual has an expression level of PDPN in the sample that is above a reference PDPN expression level and the method further comprises administering to the individual an effective amount of an agonist of CD177 activity.

In another aspect, the disclosure features a method of treating an individual having a cancer, the method comprising (a) determining an expression level of PDPN in a sample from the individual, wherein the expression level of PDPN in the sample is above a reference PDPN expression level; and (b) administering to the individual an effective amount of an agonist of CD177 activity.

In another aspect, the disclosure features a method of treating an individual having a cancer, the method comprising administering to the individual an effective amount of an agonist of CD177 activity, wherein the expression level of PDPN in a sample from the individual has been determined to be above a reference PDPN expression level.

In some aspects, the CD177 activity is inhibition of PDPN.

In some aspects, the sample from the individual is a tumor tissue sample or a tumor fluid sample. In some aspects, the tumor tissue sample is a formalin-fixed and paraffin-embedded (FFPE) sample, an archival sample, a fresh sample, or a frozen sample.

In some aspects, the expression level of PDPN in the sample is a protein expression level of PDPN or an RNA expression level of PDPN. In some aspects, the expression level of PDPN in the sample is an RNA expression level of PDPN. In some aspects, the RNA expression level of PDPN is determined by in situ hybridization (ISH), RNA-seq, RT-qPCR, qPCR, multiplex qPCR or RT-qPCR, microarray analysis, SAGE, MassARRAY technique, FISH, or a combination thereof.

In some aspects, the reference PDPN expression level is an expression level of PDPN in a population of individuals having a cancer. In some aspects, the cancer is a colorectal cancer (CRC), a squamous cell carcinoma of the head and neck, or a glioma.

In some aspects, the reference PDPN expression level is the 50^{th} percentile of expression levels in the population.

In some aspects, the reference PDPN expression level is the 66^{th} percentile of expression levels in the population.

In some aspects, the reference PDPN expression level is a pre-assigned PDPN expression level.

In some aspects, the cancer is a CRC, a squamous cell carcinoma of the head and neck, or a glioma. In some aspects, the cancer is a CRC, e.g., a stage II CRC or a stage IV CRC.

In some aspects, the benefit comprises an extension in the individual's recurrence-free survival (RFS) as compared to treatment without the agonist of CD177 activity.

In some aspects, the agonist of CD177 activity results in an increase in the binding of PDPN and CD177 relative to binding of the two proteins in the absence of the agonist.

In some aspects, the agonist of CD177 activity results in a change in a downstream activity of PDPN relative to the downstream activity in the absence of the agonist of CD177 activity.

In some aspects, the change in the downstream activity is a decrease in tumor growth.

In some aspects, the change in the downstream activity is a decrease in cancer-associated fibroblast (CAF) contractility.

In some aspects, the agonist of CD177 activity is a small molecule, an antibody or antigen-binding fragment thereof, a peptide, or a mimic.

In some aspects, the agonist of CD177 activity is a peptide. In some aspects, the peptide is a CD177 peptide. In some aspects, the CD177 peptide is an extracellular domain of CD177. In some aspects, the peptide is multimerized, e.g., tetramerized, e.g., tetramerized using streptavidin.

In some aspects, the agonist of CD177 activity is an antibody or antigen-binding fragment thereof.

In some aspects, the antibody or antigen-binding fragment thereof binds PDPN. In some aspects, the antibody or antigen-binding fragment thereof is an antagonist antibody or antigen-binding fragment thereof.

In some aspects, the antibody or antigen-binding fragment thereof binds CD177. In some aspects, the antibody or antigen-binding fragment thereof is an agonist antibody or antigen-binding fragment thereof.

In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the individual is a human.

In another aspect, the disclosure features a collection of polypeptides, wherein each polypeptide comprises an extracellular domain, a tag, and an anchor, and wherein the collection of polypeptides comprises the extracellular domains of at least 81% of the proteins of Table 7.

In some aspects, the collection of polypeptides comprises the extracellular domains of at least 85% of the proteins of Table 7. In some aspects, the collection of polypeptides comprises at least 90% of the proteins of Table 7. In some aspects, the collection of polypeptides comprises the extracellular domains of at least 95% of the proteins of Table 7. In some aspects, the collection of polypeptides comprises the extracellular domains of all of the proteins of Table 7.

In some aspects, the anchor is capable of tethering the extracellular domain to the surface of a plasma membrane of a cell. In some aspects, the anchor is a glycosylphosphatidyl-inositol (GPI) polypeptide.

In some aspects, the tag can be directly or indirectly visualized. In some aspects, the tag comprises a moiety that can be detected using an antibody or an antibody fragment. In some aspects, the tag is a glycoprotein D (gD) polypeptide. In some aspects, the tag comprises a fluorescent protein.

In some aspects, the extracellular domain has a native conformation. In some aspects, the extracellular domain comprises a native post-translational modification.

In some aspects, the cell is a mammalian cell. In some aspects, the cell is a COS7 cell.

In some aspects, the cell has been transiently transfected with a plasmid encoding the polypeptide.

In another aspect, the disclosure features a collection of vectors encoding the collection of polypeptides of any one of the above aspects.

In another aspect, the disclosure features a collection of cells comprising the collection of vectors vectors of the above aspect. In some aspects, a plurality of the cells are capable of expressing at least one polypeptide of any one of the above aspects, optionally wherein different cells express different polypeptides.

In some aspects, each of the one or more of said polypeptides is immobilized to a distinct location on one or more solid surfaces.

In another aspect, the disclosure features a method for identifying a protein-protein interaction, the method comprising providing the collection of polypeptides of any one of the above aspects, optionally wherein said polypeptides are immobilized on one or more solid surfaces; contacting the collection of step (a) with a multimerized query protein under conditions permitting the binding of the multimerized query protein and at least one of the extracellular domains of the polypeptides; and detecting an interaction between the multimerized query protein and the at least one extracellular domain, thereby identifying a protein-protein interaction. In some aspects, one or more of said polypeptides each is immobilized to a distinct location on said one or more solid surfaces. In some aspects, the distinct location comprises a cell that displays the polypeptide.

In some aspects, the cells are mammalian cells.

In some aspects, the contacting step is semi-automated.

In some aspects, detecting an interaction comprises detecting a signal, optionally a fluorescent signal, at a location on the solid surface that is above a threshold level. In some aspects, the detecting is automated.

In some aspects, the interaction is a transient interaction.

In some aspects, the interaction is a low-affinity interaction. In some aspects, the low-affinity interaction is a micromolar-affinity interaction.

In some aspects, the multimerized query protein is a dimerized, trimerized, tetramerized, or pentamerized query protein. In some aspects, the multimerized query protein is a tetramerized query protein. In some aspects, the multimerized query protein comprises an isolated extracellular domain of the query protein. In some aspects, the isolated extracellular domain of the query protein has been biotinylated and conjugated to a fluorescent streptavidin to tetramerize the query protein.

In another aspect, the disclosure features a method of identifying a modulator of the interaction between a protein of Table 1 and a protein of Table 2, the method comprising (a) providing a candidate modulator; (b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

In another aspect, the disclosure features a method of identifying a modulator of a downstream activity of a protein of Table 1, the method comprising (a) providing a candidate modulator; (b) contacting the protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring a downstream activity of the protein of Table 1, wherein a change in the downstream activity in the presence of the candidate modulator relative to the downstream activity in the absence of the candidate modulator identifies the candidate modulator as a modulator of the downstream activity of the protein of Table 1.

In another aspect, the disclosure features a method of identifying a modulator of a downstream activity of a protein of Table 2, the method comprising (a) providing a candidate modulator; (b) contacting the protein of Table 2 with a protein of Table 1 in the presence or absence of the candidate modulator under conditions permitting the binding of the protein of Table 2 to the protein of Table 1, wherein the protein of Table 1 and the protein of Table 2 are reported to interact in Table 3; and (c) measuring a downstream activity of the protein of Table 2, wherein a change in the downstream activity in the presence of the candidate modulator relative to the downstream activity in the absence of the candidate modulator identifies the candidate modulator as a modulator of the downstream activity of the protein of Table 2.

In some aspects, the increase or decrease in binding is at least 70%, as measured by surface plasmon resonance, biolayer interferometry, or an enzyme-linked immunosorbent assay (ELISA).

In some aspects, the modulator is an inhibitor of the downstream activity of the protein of Table 1 or Table 2. In some aspects, the modulator is an activator of the downstream activity of the protein of Table 1 or Table 2.

In some aspects, the change in the downstream activity is a decrease in the amount, strength, or duration of the downstream activity. In some aspects, the change in the downstream activity is an increase in the amount, strength, or duration of the downstream activity.

In some aspects, the modulator is a small molecule, an antibody or antigen-binding fragment thereof, a peptide, a mimic, an antisense oligonucleotide, or a small interfering RNA (siRNA). In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain. In some aspects, the antibody or antigen-binding fragment thereof binds the protein of Table 1. In some aspects, the antibody or antigen-binding fragment thereof binds the protein of Table 2.

In some aspects, the protein of Table 1 is podoplanin (PDPN). In some aspects, the protein of Table 2 is CD177.

In some aspects, the downstream activity is cancer-associated fibroblast (CAF) contractility. In some aspects, CAF contractility is decreased in the presence of the modulator. In some aspects, CAF contractility is decreased by at least 20%, as measured in a gel contraction assay. In some aspects, CAF contractility is decreased by at least 20%, as measured in a 3D gel elongation assay.

In some aspects, the downstream activity is tumor growth. In some aspects, tumor growth is decreased in the presence of the modulator. In some aspects, tumor growth is decreased by at least 20%, as measured in a tumor growth assay.

In some aspects, the modulator is an antibody or antigen-binding fragment thereof targeting PDPN. In some aspects, the modulator is an antibody or antigen-binding fragment thereof targeting CD177.

In some aspects, the protein of Table 1 is PD-L1 (CD274). In some aspects, the protein of Table 2 is EPHA3.

In some aspects, the protein of Table 1 is PD-L2 (PDCD1LG2). In some aspects, the protein of Table 2 is CEACAM4, ICAM5, NECTIN3, PSG9, or TNFRSF11A. In some aspects, the protein of Table 2 is CEACAM4.

In some aspects, the downstream activity is immune checkpoint inhibition. In some aspects, immune checkpoint inhibition is decreased in the presence of the modulator. In some aspects, immune checkpoint inhibition is decreased by at least 30%, as measured in a cell-based assay.

In some aspects, the protein of Table 1 is PTPRD. In some aspects, the PTPRD comprises a G203E and K204E; R232C and R233C; P249L; G285E; E406K; S431L; R561Q; P666S; E755K; V892I; S912F; R995C; or R1088C amino acid substitution mutation or a ΔG61 ΔE106 amino acid deletion mutation. In some aspects, the protein of Table 2 is BMP5, CEACAM3, IL1RAP, IL1RAPL2, LECT1, LRFN5, SIRPG, SLITRK3, SLITRK4, SLITRK6, or TGFA.

In some aspects, the downstream activity is suppression of cell proliferation. In some aspects, suppression of cell proliferation is increased in the presence of the modulator. In some aspects, suppression of cell proliferation is increased by at least 30%, as measured in a colony formation assay. In some aspects, the downstream activity is suppression of STAT3 phosphorylation. In some aspects, suppression of STAT3 phosphorylation is increased in the presence of the modulator. In some aspects, suppression of STAT3 phosphorylation is increased by at least 30%, as measured in a Western blot for phosphorylated STAT3.

In some aspects, the protein of Table 1 is PTPRS. In some aspects, the protein of Table 2 is C6orf25, IL1RAP, IL1RAPL1, IL1RAPL2, LRFN1, LRFN5, LRRC4B, NCAM1, SLITRK1, SLITRK2, SLITRK3, SLITRK4, or SLITRK6.

In some aspects, the protein of Table 1 is PTPRF. In some aspects, the protein of Table 2 is CD177, IL1RAP, or LRFN5.

In some aspects, the downstream activity is inhibition of cell migration. In some aspects, inhibition of cell migration is increased in the presence of the modulator. In some aspects, inhibition of cell migration is increased by at least 20%.

In some aspects, the downstream activity is phosphorylation of EGFR. In some aspects, phosphorylation of EGFR is decreased in the presence of the modulator. In some aspects, phosphorylation of EGFR is decreased by at least 30%, as measured in an assay for phosphorylation of EGFR.

In some aspects, the protein of Table 1 is CHL1. In some aspects, the protein of Table 2 is CNTN1, CNTN5, SIRPA, L1CAM, or TMEM132A.

In some aspects, the downstream activity is suppression of tumor formation. In some aspects, suppression of tumor formation is increased in the presence of the modulator. In some aspects, suppression of tumor formation is increased by at least 20%.

In some aspects, the protein of Table 1 is CNTN1. In some aspects, the protein of Table 2 is CDH6, CHL1, FCGRT, PCDHB7, or SGCG.

In some aspects, the downstream activity is cell proliferation or cell invasion. In some aspects, cell proliferation or cell invasion is decreased in the presence of the modulator. In some aspects, cell proliferation is decreased by at least 20%, as measured in a colony formation assay. In some aspects, cell invasion is decreased by at least 20%, as measured in a gel invasion assay.

In some aspects, the protein of Table 1 is LILRB1. In some aspects, the protein of Table 2 is CLEC6A, CXADR, EDAR, FLT4, IL6R, ILDR1, or LILRA5.

In some aspects, the downstream activity is suppression of phagocytosis. In some aspects, suppression of phagocytosis is decreased in the presence of the modulator. In some aspects, suppression of phagocytosis is decreased by at least 20%.

In some aspects, the protein of Table 1 is LILRB2. In some aspects, the protein of Table 2 is IGSF8 or MOG.

In some aspects, the protein of Table 1 is LILRB3. In some aspects, the protein of Table 2 is LRRC15 or LY6G6F.

In some aspects, the protein of Table 1 is LILRB4. In some aspects, the protein of Table 2 is CNTFR.

In some aspects, the protein of Table 1 is LILRB5. In some aspects, the protein of Table 2 is APLP2, CD177, CLEC10A, CLECSF13, LDLR, PILRA, or UNC5C. In some aspects, the protein of Table 2 is LDLR.

In some aspects, the downstream activity is osteoclast differentiation. In some aspects, osteoclast differentiation is decreased by at least 20% in the presence of the modulator. In some aspects, osteoclast differentiation is measured in an assay for TRAP+ multinucleated cells.

In some aspects, the protein of Table 1 is AXL. In some aspects, the protein of Table 2 is IL1RL1 or VSIG10L.

In some aspects, the downstream activity is activation of the RAS/RAF/MAPK/ERK1/2 pathway, activation of the JAK/STAT pathway, activation of the PI3K signaling pathway, cell migration, formation of filopodia, or phosphorylation of AXL. In some aspects, cell migration is decreased by at least 20% as measured in a gel invasion assay.

In some aspects, the protein of Table 1 is LRRC4B. In some aspects, the protein of Table 2 is BTN3A1 or BTN3A3

In some aspects, expression of the protein of Table 1 or the protein of Table 2 is upregulated or downregulated in tumor tissue relative to healthy tissue.

In another aspect, the disclosure features an isolated modulator of the interaction between a protein of Table 1 and a protein of Table 2, wherein (a) the protein of Table 1 and the protein of Table 2 are reported to interact in Table 3; and (b) the modulator causes an increase or decrease in the binding of the protein of Table 1 to the protein of Table 2 relative to binding in the absence of the modulator.

In another aspect, the disclosure features an isolated modulator of the downstream activity of a protein of Table 1 or a protein of Table 2, wherein (a) the protein of Table 1 and the protein of Table 2 are reported to interact in Table 3; and (b) the modulator causes a change in the downstream activity of the protein of Table 1 or the protein of Table 2 relative to downstream activity in the absence of the modulator.

In some aspects, the increase or decrease in binding is at least 70%, as measured by surface plasmon resonance, biolayer interferometry, or an enzyme-linked immunosorbent assay (ELISA).

In some aspects, the modulator is an inhibitor of the downstream activity of the protein of Table 1 or Table 2. In some aspects, the modulator is an activator of the downstream activity of the protein of Table 1 or Table 2.

In some aspects, the change in the downstream activity is a decrease in the amount, strength, or duration of the downstream activity. In some aspects, the change in the downstream activity is an increase in the amount, strength, or duration of the downstream activity.

In some aspects, the modulator is a small molecule, an antibody or antigen-binding fragment thereof, a peptide, or a mimic. In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain. In some aspects, the antibody or antigen-binding fragment thereof binds the protein of Table 1. In some aspects, the antibody or antigen-binding fragment thereof binds the protein of Table 2.

### BRIEF DESCRIPTION OF THE DRAWINGS

FIG. 1A is a pair of graphs showing the occurrence of main protein domains and motifs according to UniProt annotations in the set of immunoglobulin superfamily (IgSF) members and other non-IgSF proteins that were tested for pairwise interaction in the study. EGF=epidermal growth factor, TNF=tumor necrosis factor.

FIG. 1B is a schematic diagram depicting the automated high-throughput technology for extracellular interactome discovery (extracellular interaction screen). (I) depicts a library of STM receptors (prey proteins) consisting of 1,364 human proteins expressed as receptor extracellular domain (ECD)-Fc fusion proteins for expression in the conditioned media of transfected cells. (II) depicts a library of IgSF priority members (query proteins) to be tested for binding against the collection of STM receptors. IgSF proteins were expressed as pentameric constructs fused to a beta lactamase enzyme for increased binding avidity and sensitive detection of binding partners. (III) depicts the automated extracellular interaction screen. Each IgSF query protein was screened for binding to all STM receptors and processed through a computational pipeline to minimize false positive, incorporate gene expression data, and enable further analyses. (IV) depicts the IgSF interactome, which comprises more than 800 high confidence protein-protein interactions. (V) depicts a method for validation of selected interactions. Validation methods include surface plasmon resonance and immunofluorescence for detection of binding partners on the cell surface.

FIG. 1C is a scatter plot showing two independent replicates of the extracellular interaction screen for each of PD-1 (PDCD1) and PD-L2 (PDCD1LG2). Binding partners known from the literature are depicted in blue; these binding partners are PDCD1LG2 and CD274 for PD-1 and PDCD1 for PD-L2.

FIG. 2A is a network representation of all predicted receptor interaction pairs identified by the extracellular interaction screen (the “IgSF interactome”). Nodes represent the IGSF query and STM prey proteins, and edges represent the interactions between them. The node size corresponds to the number of network neighbors (node degree) to indicate network hubs. Edges representing known interactions (e.g., interactions previously predicted in the Bioplex, Biogrid, or STRING databases) are depicted in red. Recapitulated sub-networks mapping to well-studied immunoglobulin (Ig) families are marked as follows: (1) the Semaphorin-Plexin sub-network; (2) The ephrin receptor tyrosine kinase sub-network; (3) the PD-1/PD-L1 immune regulatory axis; and (4) the PVR/TIGIT sub-network.

FIG. 2B is a ridge plot showing the separation in Specificity Score distributions between the Non-specific, Positive, and Negative classes in the training set.

FIG. 2C is a regression plot depicting that the topological coefficients within the IgSF interactome network follow a power law, a hallmark of scale-free networks.

FIG. 2D is a Venn diagram showing the overlap between the interactions identified in the extracellular interaction screen and known interactions in the Bioplex, Biogrid, and STRING databases.

FIG. 2E is a bar plot showing that the percentage of interactions identified between two extracellular proteins (Bioplex; 1%), according the Human Protein Atlas cellular localization associations, is substantially under-represented relative to the estimated percentage (18%) of extra-cellular proteins in the human proteome.

FIG. 2F is a bar plot showing the number of reported unique interaction pairs (577) broken down by directionality subsets: 114 pairs for which the prey in the STM library was included in the query set and the reported interaction was reciprocally confirmed (red); 124 pairs for which the prey in the STM library was included in the query set and the reported interaction was not reciprocally confirmed (orange); and 463 pairs for which the prey in the STM library was not included in the query set.

FIG. 3A is a network representation showing the IgSF interactome dissected by Markov clustering (MCL) clusters based on network connectivity and healthy tissue gene expression profiles from Genotype-Tissue Expression (GTEx). Edges connecting nodes in a single cluster are annotated in black; edges connecting nodes in different clusters are colored in light grey. All clusters are annotated with their most common statistically significant enriched Biological Process gene ontology (GO) term, corresponding to the numbered legend below. Network nodes with prior annotation of the enriched GO term are colored differentially corresponding to their term. Nodes with an unknown annotation, or different annotation to the cluster assigned term, are shown as diamonds.

FIG. 3B is a set of violin plots showing that the average correlation (Pearson's r) of mRNA expression measured across all GTEx tissues is significantly higher (p<1.2×10⁻²⁰) for all pairs of reported interacting proteins (left plot, yellow) compared to the complement set of all possible non-interacting pairs of network nodes (right plot, blue). The dotted line indicates the 95^{th} percentile of the correlation distribution, above which selected novel interactions, validated in this study, are indicated.

FIG. 3C is a scatter plot showing a comparison of normalized mRNA expression (log₂ nRPKM) of the reported binding partners NECTIN1 and NECTIN4 in the esophagus GTEx Tissue subset. The expression pattern was significantly correlated (q<0.05) with the regression model overlaid (red).

FIG. 3D is a scatter plot showing a comparison of normalized mRNA expression (log₂ nRPKM) of the reported binding partners CEACAM5 and CEACAM7 in the colon GTEx Tissue subset. The expression pattern was significantly correlated (q<0.05) with the regression model overlaid (red).

FIG. 3E is a scatter plot showing a comparison of normalized mRNA expression (log₂ nRPKM) of the reported binding partners LILRA5 and LILRB1 in the blood GTEx Tissue subset. The expression pattern was significantly correlated (q<0.05) with the regression model overlaid (red).

FIG. 3F is a scatter plot showing a comparison of normalized mRNA expression (log₂ nRPKM) of the reported binding partners PTPRZ1 and CNTN1 in the brain GTEx Tissue subset.

FIG. 3G is a set of scatter plots showing a comparison of normalized mRNA expression (log₂ nRPKM) of the reported binding partners L1CAM and CHL1 in specific GTEx Tissue subsets (left to right: colon, small intestine, nerve, and stomach) where the expression pattern was significantly correlated (q<0.05) with the regression model overlaid (red).

FIG. 3H is a schematic showing the design of the cell surface interaction assay. Binding partners (BP) were expressed on cells, and binding of the protein of interest, expressed as recombinant purified ECD, to the cell surface of untransfected (UT) and receptor-expressing cells was analyzed using immunofluorescence. The recombinant proteins were biotinylated and multimerized using fluorescent streptavidin for increased binding avidity and detection of transient interactions.

FIG. 3I is a set of micrographs showing results of the cell surface interaction assay for the soluble query NCR1 and the binding partners SIGLEC6, SIGLEC7, and SIGLEC8 relative to control UT cells. “Binding” shows only the query protein in gray. “Merge” shows the query protein in red and nuclei stained with DAPI in blue. Scale bar=50 μm.

FIG. 3J is a set of micrographs showing results of the cell surface interaction assay for the soluble query CHL1 and the binding partner L1CAM relative to control UT cells. “Binding” shows only the query protein in gray. “Merge” shows the query protein in red and nuclei stained with DAPI in blue. Scale bar=50 μm.

FIG. 3K is a set of micrographs showing results of the cell surface interaction assay for the soluble query CNTN1 and the binding partner PTPRZ1 relative to control UT cells. “Binding” shows only the query protein in gray. “Merge” shows the query protein in red and nuclei stained with DAPI in blue. Scale bar=50 μm.

FIG. 3L is a set of immunoblots showing the results of a co-immunoprecipitation (co-IP) assay for NCR1-FLAG in cells co-expressing SIGLEC7-HA or a vector control.

FIG. 3M is a set of immunoblots showing the results of a co-IP assay for NCR1-FLAG in cells co-expressing SIGLEC8-HA or a vector control.

FIG. 3N is a set of immunoblots showing the results of a co-IP assay for NCR1-FLAG in cells co-expressing CD4-HA or a vector control.

FIG. 4A is an IgSF interactome network representation showing the PD-L1 (CD274) and PD-L2 (PDCD1LG2) immune regulatory cluster (shaded in light red). Edges corresponding to the CD274-EPHA3 and CEACAM4-PDCD1LG2 interactions are depicted with thick lines and highlighted in red. Nodes are colored according their primary assigned GO category, as provided in FIG. 3.

FIG. 4B is a sensorgram showing binding of PD-1, PD-L1, and PD-L2 (expressed as ECD-Fc fusion proteins) to CEACAM4 as analyzed by SPR. Recombinant CEACAM4 (expressed as an ECD-Fc fusion protein) was immobilized on a sensor chip and the indicated proteins were injected at 250 nM concentration. An irrelevant Fc-tagged protein was used as a control. Sensorgram shown is representative of at least 3 independent runs.

FIG. 4C is a sensorgram showing binding of PD-L1, EPHA3, and EPHA5 (expressed as ECD-Fc fusion proteins) to LILRA3 as analyzed by SPR. Recombinant LILRA3 (expressed as an ECD-Fc fusion protein) was immobilized on a sensor chip and the indicated proteins were injected at 250 nM concentration. An irrelevant Fc-tagged protein was used as a control. Sensorgram shown is representative of at least 3 independent runs.

FIG. 4D is a graph showing binding of PD-L1 to its partners PD-1 and EPHA3 in the presence of increasing concentrations of the anti-PD-1/PD-L1 antibody atezolizumab. Response units were measured at the end of the injection. Bar plots shows mean±standard deviation. Experiments are representative of 2 independent assays.

FIG. 4E is a sensorgram showing binding of PD-1, PD-L1, and PD-L2 (expressed as ECD-Fc fusion proteins) to EPHA3 as analyzed by SPR. Recombinant EPHA3 (expressed as an ECD-Fc fusion protein) was immobilized on a sensor chip and the indicated proteins were injected at 250 nM concentration. An irrelevant Fc-tagged protein was used as a control. Sensorgram shown is representative of at least 3 independent runs.

FIG. 4F is a set of micrographs showing results of the cell surface interaction assay for the soluble queries PD-L1 and PD-L2 and the binding partners PD-1, CD80, EPHA3, EPHB1, PD-L2, PD-L1, CEACAM4, and CEACAM5. The query protein is shown in red, and nuclei stained with DAPI are shown in blue. Scale bar=50 μm.

FIG. 4G is a set of immunoblots showing the results of a co-immunoprecipitation (co-IP) assay for LILRB1-FLAG in cells co-expressing EDAR-HA or a vector control.

FIG. 4H is a set of immunoblots showing the results of a co-immunoprecipitation (co-IP) assay for LILRB1-FLAG in cells co-expressing IL6R-HA or a vector control.

FIG. 4I is a set of immunoblots showing the results of a co-immunoprecipitation (co-IP) assay for LILRB4-FLAG in cells co-expressing CNTFR-HA or a vector control.

FIG. 5A is a simplified network representation of interactions in the neural system related community centered around the PTPR family (FIG. 3A, cluster 1) individual binding partners within the SLITRK, NTRK, LFRN, IL1RAP, and LRRC families. Red edges represent interactions that were validated using the cell surface interaction assay, as shown in FIGS. 5B-5E. Dotted edges represent interactions having weak binding.

FIG. 5B is a set of micrographs showing results of the cell surface interaction assay for the soluble queries SLITRK2 (top) and SLITRK3 (bottom) and the binding partners PTPRD and PTPRS relative to control untransfected (UT) cells. “Binding” shows only the query protein in gray. “Merge” shows the query protein in red and nuclei stained with DAPI in blue. Scale bar=50 μm.

FIG. 5C is a set of micrographs showing results of the cell surface interaction assay for the soluble query LFRN5 and the binding partners PTPRD, PTPRS, and PTPRF relative to control UT cells. “Binding” shows only the query protein in gray. “Merge” shows the query protein in red and nuclei stained with DAPI in blue. Scale bar=50 μm.

FIG. 5D is a set of micrographs showing results of the cell surface interaction assay for the soluble query IL1RAP and the binding partners PTPRD, PTPRS, and PTPRF relative to control UT cells. “Binding” shows only the query protein in gray. “Merge” shows the query protein in red and nuclei stained with DAPI in blue. Scale bar=50 μm.

FIG. 5E is a set of micrographs showing results of the cell surface interaction assay for the soluble queries BTN3A1, BTN3A3, and BTN2A2 and the binding partner LRRC4B relative to control UT cells. “Binding” shows only the query protein in gray. “Merge” shows the query protein in red and nuclei stained with DAPI in blue. Scale bar=50 μm.

FIG. 5F is a set of sensorgrams showing binding of ILRAP, ILRAPL1, SLITRK1, SLITRK4, LRFN1, and LRFN4 to PTPRD as analyzed by SPR. The indicated protein (expressed as an ECD-Fc fusion protein) was immobilized on a sensor chip and PTPRD (expressed as an ectodomain fused to an Fc tag) was injected at the indicated concentrations.

FIG. 5G is a set of immunoblots showing the results of a co-immunoprecipitation (co-IP) assay for FAM-FLAG in cells co-expressing BTN2A1-HA or a vector control.

FIG. 5H is a set of immunoblots showing the results of a co-immunoprecipitation (co-IP) assay for LRRC4B-FLAG in cells co-expressing BTN3A2-HA or a vector control.

FIG. 5I is a set of immunoblots showing the results of a co-immunoprecipitation (co-IP) assay for LRRC4B-FLAG in cells co-expressing BTN3A3-HA or a vector control.

FIG. 5J is a set of immunoblots showing the results of a co-immunoprecipitation (co-IP) assay for LRRC4B-FLAG in cells co-expressing EDAR-HA or a vector control.

FIG. 5K is a schematic showing cancer-relevant amino acid substitution mutations in PTPRD.

FIG. 5L is a heat map showing the row-clustered log 2 ratio of normalized absorbance per binding partner, color-coded from white (loss of binding) to red (conserved binding).

FIG. 5M is a network representation showing interactions between LILR family members and their previously uncharacterized binding partners showing concerted up-regulation of LILR proteins in kidney renal clear cell carcinoma (KIRC).

FIG. 5N is a set of micrographs showing results of the cell surface interaction assay for the soluble queries EDAR, LDLR, LILRA5, and CNTFR and the binding partners LILRB1, LILRB2, LILRB3, LILRB4, and LILRB5 relative to control UT cells. The query protein is shown in red, and nuclei stained with DAPI are shown in blue.

FIG. 6A is a network diagram showing the IgSF interactome represented as network communities, augmented with the results of a differential expression analysis between tumor and adjacent normal tissue per TCGA indication. Node color and size are indicative for the number of TCGA indications where the gene was found to be significantly dysregulated (|log 2 fold-change|>1 and p<0.05).

FIG. 6B is a bar plot showing the number of edges connecting significantly deregulated nodes per TCGA indication, in descending order. LUSC=lung squamous cell carcinoma, KIRC=kidney renal clear cell carcinoma, COAD=colon adenocarcinoma, KICH=kidney chromophobe, LUAD=lung adenocarcinoma, UCEC=uterine corpus endometrial carcinoma, KIRP=kidney renal papillary cell carcinoma, BLCA=bladder urothelial carcinoma, LIHC=liver hepatocellular carcinoma, BRCA=breast invasive carcinoma, THCA=thyroid carcinoma, ESCA=esophageal carcinoma, STAD=stomach adenocarcinoma, HNSC=head and neck squamous cell carcinoma, PRAD=prostate adenocarcinoma. Red bars represent the IgSF interactome. Light red bars represent a reference set of unrelated gene pairs reported in TCGA.

FIG. 6C is a network diagram showing up- or down-regulated genes in the immune regulatory community (as defined in FIG. 3B) in kidney renal clear cell carcinoma (KIRC).

FIG. 6D is a network diagram showing up- or down-regulated genes in the immune regulatory community (as defined in FIG. 3B) in head and neck squamous cell carcinoma (HNSC).

FIG. 6E is a set of violin plots showing that the average correlation (Pearson's r) of mRNA expression measured across all Cancer Cell Line Encyclopedia (CCLE) cell lines is significantly higher (p<4×10⁻²) for all pairs of reported interacting proteins (left plot, yellow) compared to the complement set of all possible non-interacting pairs of network nodes (right plot, blue). The dotted line indicates the 95^{th} percentile of the correlation distribution, above which selected novel interactions, validated in this study, are indicated.

FIG. 6F is a scatter plot showing a comparison of relative protein expression of the reported binding partners CEACAM5 and CEACAM6 in the large intestine CCLE Tissue subset. The expression pattern was significantly correlated (q<0.2) with the regression model overlaid (red).

FIG. 6G is a scatter plot showing a comparison of relative protein expression of the reported binding partners CNTN1 and NRCAM in the lung CCLE Tissue subset. The expression pattern was significantly correlated (q<0.2) with the regression model overlaid (red).

FIG. 6H is a scatter plot showing a comparison of relative protein expression of the reported binding partners BTN3A1 and LRRC4B in the large intestine CCLE Tissue subset. The expression pattern was significantly correlated (q<0.2) with the regression model overlaid (red).

FIG. 6I is a scatter plot showing a comparison of relative protein expression of the reported binding partners FLRT3 and UNC5C in the breast CCLE Tissue subset. The expression pattern was significantly correlated (q<0.2) with the regression model overlaid (red).

FIG. 6J is a scatter plot showing a comparison of relative protein expression of the reported binding partners CNTN1 and PTPRZ1 in the hematopoietic and lymphoid tissue CCLE Tissue subset. The expression pattern was significantly correlated (q<0.2) with the regression model overlaid (red).

FIG. 6K is a scatter plot showing a comparison of relative protein expression of the reported binding partners IGSF3 and PTGFRN in the large intestine CCLE Tissue subset. The expression pattern was significantly correlated (q<0.2) with the regression model overlaid (red).

FIG. 6L is a scatter plot showing a comparison of relative protein expression of the reported binding partners IGSF3 and PTGFRN in the breast CCLE Tissue subset. The expression pattern was significantly correlated (q<0.2) with the regression model overlaid (red).

FIG. 6M is a scatter plot showing a comparison of relative protein expression of the reported binding partners AXL and VSIG10L in the hematopoietic and lymphoid tissue CCLE Tissue subset. The expression pattern was significantly correlated (q<0.2) with the regression model overlaid (red).

FIG. 6N is a set of immunoblots showing the results of a co-immunoprecipitation (co-IP) assay for IL1RL1-FLAG and VSIG10L-FLAG in cells co-expressing AXL-HA or a vector control.

FIG. 6O is a set of sensorgrams showing binding of AXL and TYRO3 to IL1RL1 or GAS6 as analyzed using biolayer interferometry.

FIG. 6P is a set of sensorgrams showing binding of AXL and MER to IL1RL1 or GAS6 as analyzed using biolayer interferometry.

FIG. 6Q is a set of micrographs showing results of the cell surface interaction assay for the soluble query AXL and the binding partner IL1RI1 relative to control UT cells. “Binding” shows only the query protein in gray. “Merge” shows the query protein in red and nuclei stained with DAPI in blue. Scale bar=50 μm.

FIG. 7A is a graph showing the distribution of relevant protein domains and motifs according to UniProt annotations for the set of 1,129 unique single-pass transmembrane proteins (“prey constructs”) in the receptor library. ITIM/ITAM=immunoreceptor tyrosine-based inhibition motif/immunoreceptor tyrosine-based activation motif, TNFR=tumor necrosis factor receptor, TLR/ILR=Toll-like receptor/interleukin receptor, Ig-like=immunoglobulin-like, EGF=epidermal growth factor.

FIG. 7B is a Venn diagram showing the overlap between the unique protein entries present in the Biogrid, Bioplex, and STRING databases (three comprehensive protein-protein interaction resources) and the present study.

FIG. 7C is a schematic representation of a set of 384-well plates showing two independent replicates of an automated cell surface interaction assay for PD-1 (PDCD1) tested against the single-pass transmembrane protein (STM) receptor library.

FIG. 7D is a schematic representation of a set of 384-well plates showing two independent replicates of an automated cell surface interaction assay for PD-L2 (PDCD1LG2) tested against the STM receptor library.

FIG. 7E is a box plot showing, from top to bottom: the distribution of the raw enzymatic absorbance background estimate per plate; the maximal enzymatic absorbance controls per plate; the unscaled enzymatic absorbance values across wells; the enzymatic absorbance values corrected by the background estimate per plate; and the subsequent ‘normalized’ absorbance values to the estimated maximal absorbance per plate.

FIG. 8A is a heatmap of clustered Normalized Absorbance values for the 1,364 prey proteins (STM ECDs) by the 445 screened IgSF query proteins.

FIG. 8B is a set of ridge plots depicting the discriminating potential of predictive features between Non-specific, True Positive, and True Negative interactions in the training set. From top to bottom: Normalized Absorbance; Query Z-score for one query screened against the entire STM library; Prey Z-score for a single prey in the STM library across all query proteins that were screened; and custom Specificity Score.

FIG. 8C is a plot showing a principal component analysis (PCA) for the training set negative, training set non-specific, training set positive, predicted non-specific/negative, and predicted positive interactions. Mapping the four-dimensional feature matrix to the first and second principal components shows that predicted true positive interactions (dark blue) align well with the true positive interactions from our training set (light blue) and are well separated from the known non-specific interactions (orange) and sampled true negative interactions (red).

FIG. 8D is a dot plot showing normalized intensity colored by predicted class (red: negative; green: positive/specific: blue: non-specific) for all reciprocally observed interactions of CD274, PDCD1, and PDCD1LG2 showing excellent reciprocal reproducibility and specificity across multiple query clones.

FIG. 8E is a dot plot showing normalized intensity colored by predicted class (red: negative; green: positive/specific: blue: non-specific) for all reciprocally observed interactions of CD274, PDCD1, and PDCD1LG2 showing excellent reciprocal reproducibility and specificity across multiple query clones.

FIG. 8F is a bar plot showing the total number of extracellular interactions in Bioplex (“Bioplex interactions extra-cellular proteins (Human Protein Atlas)”; 627), the number of Bioplex interactions between two proteins we screened for interaction (“Bioplex interactions proteins in this study”; 350), and the total number of reported extracellular interactions in this study (“all interactions in this study”; 577).

FIG. 8G is a histogram showing that the shortest-path distribution in the IgSF interactome network is centered around 6.

FIG. 9A is a clustered heatmap of the row-scaled (Z-transformed), log₂ rpkm counts for all IgSF interactome genes across GTEx Tissue detail categories.

FIG. 9B is a violin plot showing that the tissue expression correlation (Pearson's r in GTEx) distribution mean for all pairs of reported interacting proteins is significantly higher (p<1.2 e⁻²⁰) compared to the distribution of all possible non-interacting pairs (screening library complement). In the clustered network, the intra-cluster correlation is also significantly higher (p<0.05) compared to the distribution of all inter-cluster interactions.

FIG. 9C is a scatter plot of the binding pair CEACAM5 and CEACAM7, for which mRNA expression across all GTEx tissues was highly correlated.

FIG. 9D is a scatter plot of the binding pair LILRB1 and LILRA5, for which mRNA expression across all GTEx tissues was highly correlated.

FIG. 9E is a scatter plot of the binding pair SIGLEC7 and NCR1, for which mRNA expression across all GTEx tissues was highly correlated.

FIG. 9F is a set of faceted scatter plots showing L1CAM and CHL1 mRNA expression across different brain regions, showing constitutively high expression of CHL1 in the cerebellar regions and strongly correlated expression patterns for all other regions.

FIG. 9G is a set of micrographs showing results of a cell surface interaction assay for the soluble query CHL1 and the cell-surface-expressed binding partners BTLA and CNTN5 relative to control untransfected (UT) cells. Query protein binding to the cell surface is shown in red in the merged image, and nuclei stained with DAPI are shown in blue. Scale bar=50 μm.

FIG. 9H is a network diagram showing the new interactions identified using independent assays for the CNTN1 and CHL1 cluster.

FIG. 9I is a set of graphs showing analysis of the binding of NRCAM, NFASC, MCAM, CHL1, and a control protein to CNTN1 using SPR. Recombinant NRCAM, NFASC, MCAM, CHL1, and a control protein were immobilized on a sensor chip and CNTN1 (expressed as a recombinant ECD-Fc) was injected at 250 nM concentration.

FIG. 9J is a clustered heatmap of the row-scaled expression values for a representative network cluster (cluster 1). Network clusters often comprise distinct tissue expression sub-groups, exemplified by the Brain versus Whole Blood, Spleen, Lung and Small Intestine sub-groups.

FIG. 9K is a clustered heatmap showing the representation of simplified GO categories (rows) for all network clusters with >2 members (columns). Cell values are colored according each category's OddsRatio (capped at OddsRatio>50). Network clusters recurrently comprise genes with multiple biological annotations.

FIG. 10A is a clustered tissue expression heatmap for members of the PD-L1/CD274 and PDCD1LG2/PD-L2 immune regulatory cluster (green) along with the Ephrin (purple) and CEACAM (olive) family members, highlighting evidence for co-expression of CEACAM4 with PD-L2 and divergence in co-expression between EPHA3 and the Ephrin cluster.

FIG. 10B is a set of box plots showing expression of PD-L1 (CD274) and EPHA3 in normal tissues based on GTEx data.

FIG. 10C is a set of box plots showing expression of CEACAM4 and PD-L2 in normal tissues based on GTEx data.

FIG. 10D is a sensorgram showing a representative SPR experiment showing EPHA3 binding to PD-L1. PD-L1 was immobilized on sensor chips, and EPHA3, expressed as a recombinant His-tagged ECD, was injected at 0, 50, 10, 20, 50, and 100 nM concentrations. Binding kinetics were calculated in equilibrium.

FIG. 10E is a sensorgram showing a representative SPR experiment showing CEACAM4 binding to PD-L2. PD-L2 was immobilized on sensor chips, and CEACAM4, expressed as a recombinant His-tagged ECD, was injected at 0, 10, 20, 50, 100, and 200 nM concentrations. Binding kinetics were calculated in equilibrium.

FIG. 10F is a set of micrographs showing results of a cell surface interaction assay for the soluble query MDGA1 and the cell-surface-expressed binding partners NLGN3 and NLGN4X relative to control untransfected (UT) cells. Query protein binding to the cell surface is shown in red in the merged image, and nuclei stained with DAPI are shown in blue. Scale bar=50 μm.

FIG. 10G is a set of micrographs showing results of a cell surface interaction assay for the soluble query TREML2 and the cell-surface-expressed binding partner ANTRX1 relative to control untransfected (UT) cells. Query protein binding to the cell surface is shown in red in the merged image, and nuclei stained with DAPI are shown in blue. Scale bar=50 μm.

FIG. 10H is a set of immunoblots showing the results of a co-immunoprecipitation (co-IP) assay for IGSF5-FLAG in cells co-expressing CD300A-HA or a vector control.

FIG. 10I is a set of immunoblots showing the results of a co-immunoprecipitation (co-IP) assay for IGSF5-FLAG in cells co-expressing CD300LF-HA or a vector control.

FIG. 10J is a set of micrographs showing results of a cell surface interaction assay for the soluble queries FLRT1, FLRT2, and FLRT3 and the cell-surface-expressed binding partners UNC5A, UNC5C, and UNC5D relative to control untransfected (UT) cells. Query protein binding to the cell surface is shown in red in the merged image, and nuclei stained with DAPI are shown in blue. Scale bar=50 μm.

FIG. 11A is a clustered heatmap of network nodes by TCGA indications. Cell values represent the log₂ mean change between Tumor and adjacent Normal gene expression levels.

FIG. 11B is a set of micrographs showing results of a cell surface interaction assay for the soluble query CHL1 and the cell-surface-expressed binding partners L1CAM, BTLA, and CNTN5 relative to control untransfected (UT) cells. Query protein binding to the cell surface is shown in red in the merged image, and nuclei stained with DAPI are shown in blue. Scale bar=50 μm.

FIG. 11C is a subnetwork diagram of the IgSF interactome showing putative interactions identified for the CNTN1 and CHL1 IgSF proteins. Edges represented in yellow indicate interactions that were validated by independent technologies.

FIG. 12A is a set of micrographs showing results of a cell surface interaction assay for the soluble query LFRN5 and the binding partners PTPRZ1, PTPRG, PTPRT, PTPRS, PTPRO, PTPRM, PTPRF, and PTPRD relative to control untransfected (UT) cells. “Binding” shows only the query protein in gray. Experiments shown are representative of two independent assays. “Merge” shows the query protein in red and nuclei stained with DAPI in blue. Scale bar=50 μm.

FIG. 12B is a set of micrographs showing results of a cell surface interaction assay for the soluble query SLITKR2 and the binding partners PTPRZ1, PTPRG, PTPRT, PTPRS, PTPRO, PTPRM, PTPRF, and PTPRD relative to control untransfected (UT) cells. “Binding” shows only the query protein in gray. Experiments shown are representative of two independent assays. “Merge” shows the query protein in red and nuclei stained with DAPI in blue. Scale bar=50 μm.

FIG. 12C is a set of micrographs showing results of a cell surface interaction assay for the soluble query IL1RAP and the binding partners PTPRZ1, PTPRG, PTPRT, PTPRS, PTPRO, PTPRM, PTPRF, and PTPRD relative to control untransfected (UT) cells. “Binding” shows only the query protein in gray. Experiments shown are representative of two independent assays. “Merge” shows the query protein in red and nuclei stained with DAPI in blue. Scale bar=50 μm.

FIG. 12D is a set of micrographs showing results of a cell surface interaction assay for the soluble query CNTN1 and the binding partners PTPRZ1, PTPRG, PTPRT, PTPRS, PTPRO, PTPRM, PTPRF, and PTPRD relative to control untransfected (UT) cells. “Binding” shows only the query protein in gray. Experiments shown are representative of two independent assays. “Merge” shows the query protein in red and nuclei stained with DAPI in blue. Scale bar=50 μm.

FIG. 12E is a set of micrographs showing results of a cell surface interaction assay for the soluble query BTN2A2 and the binding partner LRRC4B relative to control untransfected (UT) cells. “Binding” shows only the query protein in gray. Experiments shown are representative of two independent assays. “Merge” shows the query protein in red and nuclei stained with DAPI in blue. Scale bar=50 μm.

FIG. 12F is a set of immunoblots showing the results of a co-immunoprecipitation (co-IP) assay for LRRC4B-FLAG, LRRC4C-FLAG, TGOLN2-FLAG, VSIG8-FLAG, CDH9-FLAG, and ST14-FLAG in cells co-expressing BTN3A1-HA or a vector control.

FIG. 12G is a bar plot showing the binding specificities of the indicated PTPRD variants to the indicated binding partners, relative to wild-type PTPRD.

FIG. 13A is a set of Kaplan-Meier curves and a table showing the survival probability of patients with stage II disease split into tertiles by levels of podoplanin (PDPN) expression (T1, T2, and T3) in the publicly available CRC (stage II) microarray gene expression dataset GSE33113. Log rank p-values (Log rank p) are associated with Kaplan-Meier curves. Cox proportional hazard (CoxPH) p-values are associated with the univariate models detailed in Table 10.

FIG. 13B is a set of Kaplan-Meier curves and a table showing the survival probability of patients with any stage of disease split into tertiles by levels of PDPN expression in the publicly available CRC (all stages) microarray gene expression dataset GSE39582. Log rank p-values are associated with Kaplan-Meier curves. Cox proportional hazard p-values are associated with the univariate models detailed in Table 10.

FIG. 13C is a plot showing the correlation between PDPN expression and tumor content (percentage of cancer cells) in The Cancer Genome Atlas (TCGA).

FIG. 13D is a plot showing the correlation between PDPN and an activated fibroblast signature (fibroblast score) in TCGA.

FIG. 13E is a set of Kaplan-Meier curves and a table showing the survival probability of patients with stage II CRC split into tertiles by expression levels of the activated fibroblast signature (Act. Fib tertiles) in the GSE33113 and GSE39582 datasets. Log rank p-values are associated with Kaplan-Meier curves. Cox proportional hazard p-values are associated with the univariate models detailed in Table 10.

FIG. 13F is a set of Kaplan-Meier curves and a table showing the survival probability of patients with any stage of CRC split into tertiles by expression levels of the activated fibroblast signature (Act. Fib tertiles) in the GSE33113 and GSE39582 datasets. Log rank p-values are associated with Kaplan-Meier curves. Cox proportional hazard p-values are associated with the univariate models detailed in Table 10.

FIG. 14A is a diagram and a set of photomicrographs showing binding of monomeric or tetrameric PD-L1 to PD-1 or PD-L2. The diagram (top) shows normalized fluorescence intensity detected for cells expressing PD-1 or PD-L2 on the cell surface that have been contacted with 5 nM, 10 nM, 50 nM, 200 nM, or 500 nM monomeric or tetrameric PD-L1. The photomicrographs (bottom) are representative images of fluorescence for cells expressing PD-1 or PD-L2 on the cell surface that have been contacted with 500 nM monomeric or tetrameric PD-L1.

FIG. 14B is a diagram and a set of photomicrographs showing binding of monomeric or tetrameric poliovirus receptor (PVR) to CD96, CD226, or TIGIT. The diagram (top) shows normalized fluorescence intensity of fluorescent streptavidin detected for cells expressing CD96, CD226, or TIGIT on the cell surface that have been contacted with 5 nM, 10 nM, 50 nM, 200 nM, or 500 nM monomeric or tetrameric PVR. The photomicrographs (bottom) are representative images of fluorescence for cells expressing CD96, CD226, or TIGIT on the cell surface that have been contacted with 500 nM monomeric or tetrameric PVR.

FIG. 14C is a schematic diagram showing the design of members of the ectodomain-gD-GPI library. Left: an untagged, full-length single-pass transmembrane (STM) protein. Right: an ectodomain-gD-GPI protein comprising the ectodomain of the STM protein, a glycoprotein D (gD) tag, and a glycosylphosphatidyl-inositol (GPI) linker.

FIG. 14D is a graph showing a quantification of surface expression (as fluorescence intensity per cell) of members of the ectodomain-gD-GPI library, as measured using an anti-gD antibody. Representative images of surface staining for not detectable (no), low, medium, and high expressers are shown. Dotted lines indicate arbitrary cut-offs for the different expression levels. Expression is representative of two independent assays.

FIG. 14E is a schematic representation of an automated cell-based platform for receptor discovery (cell surface interaction screen). (1) depicts a library consisting of about 1200 unique single transmembrane (STM) receptors expressed as the STM extracellular domain (ECD) fused to a gD-GPI tag. (2) depicts tetramerization of the query protein podoplanin (PDPN). PDPN was expressed as the PDPN extracellular domain (ECD) fused to an Avidity AVITAG™ (Avi tag) for site-directed biotinylation, was biotinylated, and was conjugated to fluorescent streptavidin (SA) to form a high avidity tetramer. (3) depicts automated receptor-ligand interaction screens (cell surface interaction screens). Individual single transmembrane (STM) receptors were transfected into mammalian cells and were cultured in individual wells on a plate. The high avidity PDPN tetramer was incubated with the cells at 4° C. When PDPN interacted with the STM receptor, the fluorescent SA tag was retained at the cell surface. (4) depicts high content fluorescent imaging of individual wells of the assays of (3). (5) depicts a representation of fluorescent signal intensity. Background is calculated by averaging signal intensity for interaction with all receptors within the library. (6) depicts a representative surface plasmon resonance (SPR) plot. SPR is used as an orthogonal technique to further validate interactions.

FIG. 14F is an intersection plot showing the results of an automated cell surface interaction screen in which the immune receptor B7-H3 (CD276) was tested for interaction with a library of ectodomain-gD-GPI STM proteins. Each circle represents a binding interaction between B7-H3 and an STM receptor. Unique high-scoring hits are shown in red circles. Hits shown in gray circles are empirically determined non-specific binders. The interleukin-20 receptor subunit alpha (IL20-RA) was identified as an interacting partner.

FIG. 14G is an intersection plot showing the results of an automated cell surface interaction screen in which the immune receptor B7-H3 (CD276) was tested for interaction with a library of full-length STM proteins not comprising tags. Each circle represents a binding interaction between B7-H3 and an STM receptor. Unique high-scoring hits are shown in red circles. Hits shown in gray circles are empirically determined non-specific binders. The interleukin-20 receptor subunit alpha (IL20-RA) was identified as an interacting partner.

FIG. 14H is an intersection plot showing the results of an automated cell surface interaction screen in which podoplanin (PDPN) was tested for interaction with a library of STM proteins. Hits shown in gray circles are empirically determined non-specific binders. Each interaction was tested in duplicate. Each circle represents a binding interaction between PDPN and an STM receptor. CD177 was identified as a novel interacting partner.

FIG. 14I is a sensorgram showing binding of PDPN and CD177 as analyzed by surface plasmon resonance (SPR). Recombinant CD177 (expressed as an ECD) was immobilized on a sensor chip and recombinant PDPN (expressed as an Fc-tagged ECD) and a control protein were injected at the concentrations indicated. Dissociation constant (K_{D}) values for each interaction, measured using recombinant PDPN expressed as a monomeric ectodomain, are indicated.

FIG. 14J is a sensorgram showing the absence of binding between a control ECD and PDPN as analyzed by SPR. The control ECD was immobilized on a sensor chip and recombinant PDPN (expressed as an Fc-tagged ECD) and a control protein were injected at the concentrations indicated. Dissociation constant (K_{D}) values for each interaction, measured using recombinant PDPN expressed as a monomeric ectodomain, are indicated.

FIG. 15A is a pair of graphs showing CD177 expression on neutrophils. The left panel is a representative histogram showing CD177 expression on neutrophils in healthy blood from three different donors and an isotype control. The right panel is a graph depicting the percentage of neutrophils that were CD177+ in donor blood samples (n=31).

FIG. 15B is a set of plots showing the gating strategy for identification of neutrophils in healthy volunteer blood, CRC patient blood, adjacent normal colon tissue (adjacent colon), and cancerous colon tissue (CRC colon) using flow cytometry. Blood plots represent data from blood samples in which red blood cells (RBCs) were lysed. Colon tissue plots represent data from single-cell suspensions of colon tissue samples in which red blood cells were lysed and the suspension was strained with a 70-micron mesh. All samples were stained with 7-aminoactinomycin D (7-AAD) to gate out dead cells and incubated with the indicated antibodies. Shaded regions represent the data selected for analysis (“gate”). Gates were chosen to include events that were live (as indicated by absence of 7-AAD staining), were singlets, and were CD45⁺. Data are representative of 4-7 independent donors.

FIG. 15C is a graph showing the percentage of cells that were neutrophils in samples from 4-7 independent donors for healthy volunteer blood (norm blood), CRC patient blood (pt blood), adjacent normal colon tissue (adj colon), and cancerous colon tissue (CRC colon). ***p<0.001, Kruskal-Wallis with Dunn's multiple comparisons test.

FIG. 15D is a graph showing the percentage of neutrophils that were CD177⁺ in samples from 4-7 independent donors for healthy volunteer blood, CRC patient blood, adjacent normal colon tissue, and cancerous colon tissue.

FIG. 15E is a representative histogram showing CD177 expression levels in neutrophils in healthy blood, patient blood, adjacent normal (adj) colon tissue, and cancerous (CRC) colon tissue compared to isotype controls. Data are representative of 4-7 independent donors.

FIG. 15F is a set of plots showing the gating strategy for identification of stromal cells in adjacent normal colon tissue, cancerous (CRC) colon tissue, and diverticulitis (Div) colon tissue using flow cytometry. Plots represent data from single cell suspensions of tissue samples. Samples were stained as indicated. Gates were chosen to include events that were live (as indicated by absence of 7-AAD staining), were singlets, were CD45⁻ (i.e., were not immune cells), and were EpCAM⁻ (i.e., were not tumor cells) for examination of stromal cells. DN=double negative T cell; BEC=blood endothelial cells. Data are representative of 3-7 independent samples.

FIG. 15G is a graph showing the percentage of cells that are PDPN⁺ in CRC colon cells that are cancer-associated fibroblast (CAF) cells (EpCAM⁻, CD45⁻, CD31⁻); endothelial cells (EC) (EpCAM⁻, CD45⁻, CD31⁺); tumor cells (TC) (EpCAM⁺), myeloid cells (CD45⁺, CD11B⁺), CD4 T cells (CD45⁺, CD3⁺, CD4⁺); or CD8 T cells (CD45⁺, CD3⁺, CD8⁺). Data are representative of 3-7 independent samples. **p<0.01, ***p<0.001, Kruskal-Wallis with Dunn's multiple comparisons test.

FIG. 15H is a graph showing the mean fluorescent intensity (MFI) of PDPN in CRC colon cells that are cancer-associated fibroblast (CAF) cells (EpCAM⁻, CD45⁻, CD31⁻); endothelial cells (EC) (EpCAM⁻, CD45⁻, CD31⁺); tumor cells (TC) (EpCAM⁺), myeloid cells (CD45⁺, CD11B⁺), CD4 T cells (CD45⁺, CD3⁺, CD4⁺); or CD8 T cells (CD45⁺, CD3⁺, CD8⁺). Data are representative of 3-7 independent samples. **p<0.01, ****p<0.0001, Kruskal-Wallis with Dunn's multiple comparisons test.

FIG. 15I is a graph showing the percentage of fibroblasts that are PDPN⁺ in adjacent normal (adj) colon tissue, cancerous (CRC) colon tissue, and diverticulitis (div) colon tissue. Data are representative of 3-7 independent samples.

FIG. 15J is a graph showing the MFI of PDPN fibroblasts in adjacent normal (adj) colon tissue, cancerous (CRC) colon tissue, and diverticulitis (div) colon tissue. Data are representative of 3-7 independent samples.

FIG. 16A is a pair of micrographs from a tissue microarray showing adjacent normal colon tissue stained for CD177 (blue) and PDPN (pink). PDPN staining was largely absent on fibroblasts and marked the lymphatics. CD177 staining was rarely observed. Lower panels show magnified images.

FIG. 16B is a pair of micrographs from a tissue microarray showing cancerous (CRC) colon tissue stained for CD177 (blue) and PDPN (pink). PDPN staining was largely absent on fibroblasts and marked the lymphatics. CD177 staining was rarely observed. This tumor showed strong PDPN staining in the stroma surrounding the tumor beds, but not in the epithelial cells themselves. Lower panels show magnified images.

FIG. 16C is a set of micrographs showing representative images of dual immunofluorescence staining for PDPN and CD177 in cancerous (CRC) colon cells. The first panel shows an overview of the tissue with PDPN (green) and CD177 (red) staining. The second, third, and fourth panels show an inset of the micrograph of the first panel, as indicated by the box in the first panel. The second panel shows PDPN and CD77 staining. The third panel shows only CD177 staining. The fourth panel shows only PDPN staining. Scale bars 50 μm.

FIG. 16D is a set of micrographs showing representative images of dual immunofluorescence staining for myeloperoxidase (MPO; a marker of neutrophils) and CD177 in CRC cells. The first panel shows an overview of the tissue with MPO (green) and CD177 (red) staining. The second, third, and fourth panels show an inset of the micrograph of the first panel, as indicated by the box in the first panel. The second panel shows MPO and CD77 staining. The third panel shows only CD177 staining. The fourth panel shows only MPO staining. Scale bars 50 μm.

FIG. 16E is a bar graph showing the percentage of normal adjacent colon (normal) and CRC cancer (tumor) cells that were negative (−) or positive (+) for staining for PDPN or CD177.

FIG. 17A is a bar graph showing the morphology index of wild-type cancer-associated fibroblasts (CAFs) seeded into 3D gels and treated with an isotype control, recombinant human CLEC-2 (rCLEC-2), or recombinant human CD177 (r-CD177). Dots represent single wells containing >50 cells and 6 independent experiments. Data are plotted relative to the isotype control for each experiment. **p<0.01, Kruskal-Wallis with Dunn's multiple comparisons test.

FIG. 17B is a set of micrographs showing representative images of cancer-associated fibroblasts in 3D gels stained for actin and DAPI. The left panel shows a cell treated with the isotype control. The center panel shows a cell treated with recombinant human CLEC-2 (rCLEC-2). The right panel shows a cell treated with recombinant human CD177 (rCD177). Scale bar: 20 μm.

FIG. 17C is a bar graph showing the morphology index of wild-type cancer-associated fibroblasts (CAFs) seeded into 3D gels either alone (−) or with a 5:1 ratio of primary neutrophils (neut) or T cells isolated from blood. Dots represent single wells containing >50 cells and 2-3 independent experiments representing 5 donors each. **p<0.01, Kruskal-Wallis with Dunn's multiple comparisons test.

FIG. 17D is a bar graph showing the morphology index of primary human fibroblasts from healthy human bladder, colon, or ovary (HOF) tissues seeded into 3D gels and treated with an isotype control, recombinant human CLEC-2 (r-CLEC2), or recombinant human CD177 (r-CD177). Dots represent single wells containing >50 cells and 3 independent experiments. *p<0.05, Kruskal-Wallis with Dunn's multiple comparisons test.

FIG. 17E is a representative histogram showing staining for PDPN expression on fibroblasts from healthy human bladder, colon, and ovary (HOF) tissues and an isotype control.

FIG. 17F is a bar graph showing contraction (in μm) of wild-type cancer-associated fibroblasts (CAFs) treated with an isotype control, recombinant human CLEC-2 (rCLEC-2), or recombinant human CD177 (r-CD177) relative to unstimulated cells (un). Dots represent the average of 2-3 wells per condition. Graph comprises data from 4 independent experiments. *p<0.05, Kruskal-Wallis with Dunn's multiple comparisons test.

FIG. 18A is a schematic representation depicting the workflow of a multiplexed global proteomic and phospho-proteomic experiment in parallel with tandem mass tagging and fractionation for deep coverage. Cancer-associated fibroblasts (CAFs) were treated in duplicate with a DMSO control, CLEC-2, or CD177 for 2 minutes or 30 minutes and then lysed. The protein lysate was reduced and digested with LysC and trypsin, then peptides were normalized. Peptides were labeled with tandem mass tags (TMT). A proportion of peptides (˜0.5 mg) were used for protein profiling. These peptides were fractionated by high pH reversed-phase fractionation (Hi pH RP). Samples were run on an HPLC, and peptides were identified by liquid chromatography-mass spectrometry (LC MS/MS) analysis. The remainder of the peptides (˜6.5 mg) was used for global phosphorylation analysis. Peptides were fractionated by strong cation-exchange chromatography (SCX) and analyzed by liquid chromatography-mass spectrometry (LC MS/MS).

FIG. 18B is a heatmap showing the fold change in protein phosphorylation (compared to an untreated control) for all phosphosites that changed significantly (|Log₂ FC|>1 and p<0.05) in CAFs treated with CLEC-2 or CD177 for 2 minutes (2′) or 30 minutes (30′).

FIG. 18C is a volcano plot showing significant changes in protein phosphorylation in CAFs stimulated with CD177 (circles) or CLEC-2 (triangles) relative to untreated cells after 2 minutes.

FIG. 18D is a volcano plot showing significant changes in protein phosphorylation in CAFs stimulated with CD177 (circles) or CLEC-2 (triangles) relative to untreated cells after 30 minutes.

FIG. 18E is a bar graph showing enriched (q<0.05) gene ontology (GO) pathways represented by the proteins for which phosphosites were significantly altered in the CLEC-2 or CD177 treatment group compared to untreated CAFs. Numbers indicate the phospho-site groups associated with each curated GO term.

FIG. 18F is a table depicting the relative abundance change across treatment conditions (relative to an unstimulated control) for selected phosphorylation sites on proteins with enriched biological process pathways. Grouped phosphorylation sites are indicated with a separating “/”.

FIG. 18G is a Venn diagram indicating the ˜70% overlap (2,912 proteins) between the unique proteins identified in the global proteome (6,309 proteins) and the phospho-proteome (4,122 proteins). Overall, 18,558 unique phosphopeptides with high quality reporter ion intensities were identified, which mapped to 4,122 unique proteins with at least one phosphorylated residue.

FIG. 18H is a Venn diagram summarizing the number of phosphosites that were altered significantly (p<0.05 and |Log 2 Fold Change|>1) by CLEC-2 (190 phosphosites), CD177 (54 phosphosites) or both (32 phosphosites) at 30 min.

FIG. 18I is a ternary plot depicting all phosphopeptides detected in the phosphoproteomic assay. Significantly altered phosphosites are depicted as upward triangles (de-phosphorylated compared to unstimulated) or downward triangles (hyper-phosphorylated compared to unstimulated) and are colored according to the fold change measured between CLEC-2 at 30 min (purple) or CD177 at 30 min (green).

FIG. 19A is a set of Kaplan-Meier curves and a table showing the survival probability of patients with stage II disease split into tertiles by levels of podoplanin (PDPN) expression (T1, T2, and T3) in the publicly available CRC microarray gene expression dataset GSE39582. Log rank p-values (Log rank p) are associated with Kaplan-Meier curves. Cox proportional hazard (CoxPH) p-values are associated with the univariate models detailed in Table 10.

FIG. 19B is a set of Kaplan-Meier curves and a table showing the survival probability of patients with stage IV disease split into tertiles by levels of podoplanin (PDPN) expression (T1, T2, and T3) in the publicly available CRC microarray gene expression dataset GSE39582. Log rank p-values (Log rank p) are associated with Kaplan-Meier curves. Cox proportional hazard (CoxPH) p-values are associated with the univariate models detailed in Table 10.

FIG. 19C is a set of Kaplan-Meier curves and a table showing the survival probability of patients with stage II disease split into tertiles by levels of FAP⁺ fibroblast (FAP⁺ fib) signature expression (T1, T2, and T3) in the publicly available CRC microarray gene expression dataset GSE33113. Log rank p-values (Log rank p) are associated with Kaplan-Meier curves. Cox proportional hazard (CoxPH) p-values are associated with the univariate models detailed in Table 10.

FIG. 19D is a set of Kaplan-Meier curves and a table showing the survival probability of patients with all stages of disease split into tertiles by levels of FAP⁺ fibroblast signature expression (T1, T2, and T3) in the publicly available CRC microarray gene expression dataset GSE39582. Log rank p-values (Log rank p) are associated with Kaplan-Meier curves. Cox proportional hazard (CoxPH) p-values are associated with the univariate models detailed in Table 10.

FIG. 19E is a set of Kaplan-Meier curves and a table showing the survival probability of patients with stage II disease split into tertiles by levels of active (Act.) fibroblast signature expression (T1, T2, and T3) in the publicly available CRC microarray gene expression dataset GSE39582. Log rank p-values (Log rank p) are associated with Kaplan-Meier curves. Cox proportional hazard (CoxPH) p-values are associated with the univariate models detailed in Table 10.

FIG. 19F is a set of Kaplan-Meier curves and a table showing the survival probability of patients with stage II disease split into tertiles by levels of FAP⁺ fibroblast signature expression (T1, T2, and T3) in the publicly available CRC microarray gene expression dataset GSE39582. Log rank p-values (Log rank p) are associated with Kaplan-Meier curves. Cox proportional hazard (CoxPH) p-values are associated with the univariate models detailed in Table 10.

FIG. 20 is an intersection plot showing the results of an automated cell surface interaction screen in which podoplanin (PDPN), expressed as a tetramer using fluorescent streptavidin, was tested in duplicate for interaction with a library of full-length, untagged STM proteins. The newly identified binding partner CD177 was not present in this receptor library. Images from individual wells were acquired using a high content microscope, images were processed using the InCell Developer software, and the amount of PDPN binding to the cell surface was represented as signal over background (S/N ratio). Each circle represents a binding interaction between PDPN and an STM receptor. Green dots represent non-specific binders observed as hits in unrelated screens. CLEC-2 was identified as a novel interacting partner.

FIG. 21 is a sensorgram showing binding of PDPN and CLEC-2 as analyzed by SPR. Recombinant CLEC-2 was immobilized on a sensor chip and purified PDPN (expressed as an Fc-tagged ECD) and an irrelevant FC-tagged ECD control protein were injected at the concentrations indicated. Dissociation constant (K_{D}) values for each interaction, measured using recombinant PDPN expressed as a monomeric ectodomain, are indicated.

FIG. 22A is a box plot showing the level of CD177 RNA expression (log₂ nRPKM) in normal colon tissue and CRC tumors in a The Cancer Genome Atlas (TCGA) dataset.

FIG. 22B is a box plot showing the level of PDPN RNA expression (log₂ nRPKM) in normal colon tissue and CRC tumors in a The Cancer Genome Atlas (TCGA) dataset.

FIG. 22C is a box plot showing the level of PDPN and CD177 RNA expression in CRC tumors in the GSE39582 dataset.

FIG. 22D is a box plot showing the level of PDPN and CD177 RNA expression in CRC tumors in the GSE33113 dataset.

FIG. 23A is a set of micrographs showing serial sections of normal adjacent (adj) colon tissue stained for PDPN (left panel) and CD177 (right panel). PDPN staining was largely absent on fibroblasts and marked the lymphatics. CD177 staining was rarely observed.

FIG. 23B is a set of micrographs showing serial sections of cancerous (CRC) colon tissue stained for PDPN (left panel) and CD177 (right panel). Insets show magnified images. This tumor showed strong PDPN staining in the stroma surrounding the tumor beds, but not in the epithelial cells themselves.

FIG. 23C is a set of micrographs showing representative images of dual immunofluorescence staining for PDPN and CD177 in adjacent normal colon tissue. The first panel shows an overview of the tissue with PDPN and CD177 staining. The second, third, and fourth panels show an inset of the micrograph of the first panel, as indicated by the box in the first panel. The second panel shows PDPN and CD77 staining. The third panel shows only CD177 staining. The fourth panel shows only PDPN staining.

FIG. 23D is a set of micrographs showing representative images of dual immunofluorescence staining for myeloperoxidase (MPO; a marker of neutrophils) and CD177 in adjacent normal colon tissue. The first panel shows an overview of the tissue with MPO and CD177 staining. The second, third, and fourth panels show an inset of the micrograph of the first panel, as indicated by the box in the first panel. The second panel shows PDPN and CD77 staining. The third panel shows only CD177 staining. The fourth panel shows only MPO staining.

FIG. 24A is a set of density plots depicting the changes observed in phosphorylated residues (x-axis) versus total protein abundance changes (y-axis) for proteins that were identified both in the proteomic assay and the phospho-proteomic assay in CAFs that were stimulated with CD177 or CLEC-2 for 2 minutes or 30 minutes.

FIG. 24B is a heat map showing the fold change in protein phosphorylation (compared to an untreated control) for all phosphosites that changed significantly (|Log₂ FC|>1 and p<0.05) in CAFs treated with CLEC-2 or CD177 for 2 minutes (2′) or 30 minutes (30′), as shown in FIG. 18B, including the identity of phosphoproteins for which significant changes were observed.

FIG. 25 is a pair of illustrations showing a model for CD177 and PDPN interactions in the tumor microenvironment. The left panel shows an overview of a tissue. Dense bands of CAFs form between islands of tumor beds. These fibers generally run parallel to tumor beds and contain many immune cells. Specifically, neutrophils and regulatory T cells (Tregs) can be found in these stroma-rich regions. While there are still numerous PDPN⁺ CAFs not in contact with these CD177⁺ immune cells, some CAFs will interact with these cells and receive inhibitory signals downstream of PDPN. The right panel shows a model of the molecular interaction between CD177 and PDPN and downstream outcomes in the CAF. CD177 engagement alters contraction, motility, extracellular matrix (ECM) remodeling, and metabolism in the CAFs.

FIG. 26A is a histogram showing the expression of podoplanin (PDPN) on wild-type (WT) cancer-associated fibroblasts (CAFs) and on Pdpn−/− CAFs compared to an isotype control sample.

FIG. 26B is a graph showing the morphology index (perimeter²/4*π*area) of WT and Pdpn−/− CAFs seeded into 3D gels. Each dot represents an average of one well containing >50 cells, and the plot is representative of 3 independent experiments. *p<0.05, Mann-Whitney U test.

FIG. 26C is a pair of micrographs showing WT (left) and Pdpn−/− (right) CAFs in 3D gels with staining for actin (red) and nuclei (DAPI; blue).

FIG. 26D is a graph showing the relative contraction of Pdpn−/− cells compared with WT cells. Each dot represents the average of 3-4 wells each from 4 independent experiments. *p<0.05, Mann-Whitney U test.

FIG. 26E is a graph showing the percent confluency of WT and Pdpn−/− CAFs over time. Each point represents the mean of 16 different fields of view from 4 wells per condition and the plot is representative of 4-6 independent experiments. ****p<0.0001, ANOVA.

FIG. 27A is a set of photomicrographs showing representative images from single wells of SW480 tumor organoids expressing red fluorescent protein (RFP) in 3D culture at 8 days of growth with no CAFs (tumor only), Pdpn^{-/-} CAFs, or wild-type (WT) CAFs. Wells without tumor cells (WT CAFs only and Pdpn^{-/-} CAFs only) are shown as controls.

FIG. 27B is a graph showing the total area (in μm²) of spheroids from maximum intensity projections of the wells of FIG. 27A at time points between 1 and 8 days in culture. Data are representative of two independent experiments (n=4 wells per condition in each experiment). Dunn's multiple comparison test was performed on all conditions against the tumor only control. *p<0.05, ****p<0.0001.

FIG. 27C is a set of photomicrographs showing representative images from single wells of SW480 tumor organoids expressing RFP in 3D culture at 17 days of growth with no CAFs (tumor only), WT CAFs, or WT CAFs treated with a control, tetrameric CD177, or tetrameric CLEC-2. A well without tumor cells (WT CAFs only) is shown as a control.

FIG. 27D is a graph showing the total area (in μm²) of spheroids from maximum intensity projections of the wells of FIG. 27C at time points between 1 and 17 days in culture. Data are representative of two independent experiments (n=4 wells per condition in each experiment). Dunn's multiple comparison test showed a significant difference of ****p<0.0001 between the tumor WT CAFs group to all other conditions except to the tumor+WT CAFs+control group.

FIG. 28A is a heat map showing all genes in the IgSF Interactome and their association with a CD8+ Teff cell signature, a pan-fibroblast TFGb signature, and gene sets based on the Lund subtyping scheme for immune desert (UroA: urothelial-like A), immune excluded (Inf: infiltrated) or immune inflamed tumors (UroB: urothelial-like B, SCCL: basal/SCC-like).

FIG. 28B is a volcano plot showing protein interactions significantly associated with positive (blue) or negative (red) clinical outcome. Select interactions are highlighted.

FIG. 28C is a scatter plot showing interactions with a high synergistic effect visualized by comparing the hazard ratio computed from joint expression of interacting pairs to the compound hazard ratio of the individual genes. Select interactions with improved predictive power for clinical outcome over single genes are highlighted. Red represents interactions predictive for lack of response; blue shows interactions associated with response.

FIG. 28D is a forest plot showing increased significance for association with lack of response and patient survival for each of the EFNB1 interactions, relative to the individual genes. Improved prediction of poor overall survival of EFNB1/EVC2 interactions (HR: 1.61; 95% CI: 1.24; 2.09, P=3.78e-4) than individual genes of EFNB1 (HR: 1.25; 95% CI: 0.97; 1.62, P=0.087) and EVC2 (HR: 1.32; 95% CI: 1.02; 1.71, P=0.035, n=348 patients).

FIG. 28E is a survival plot showing the probability of survival for individuals having an expression level of EFNB1 that is greater than or less than or equal to a median expression level, an expression level of EVC2 that is greater than or less than or equal to a median expression level, or an expression level of EFNB1 and EVC2 that is greater than or less than or equal to a median expression level.

FIG. 28F is a box-and-whisker plot for the EFNB1/ECV2 interaction and each gene separately. Whisker plots represent minimum and maximum, and black circles are outliers. Y axis: Z score expression. Responder group: complete response (CR) and partial response (PR); non-responder group: progressive disease (PD) and stable disease (SD). (p-values: EFBN1: 0.0008901; EVC2: 0.009046; EFBN1/EVC2: 9.19×10⁻⁵, n=298 patients).

FIG. 29A is a heat map showing the clustered, differential expression between tumor and adjacent normal samples for all IgSF interactome genes (rows) by TCGA tumor indications (columns). Cell values represent the mean change between Tumor and adjacent Normal log₂ rsem values.

FIG. 29B is a network diagram showing jointly up- (red) and down-regulated (blue) genes within the LILR family of proteins in the TCGA tumor indication HNSC (head neck squamous cell carcinoma).

FIG. 29C is a network diagram showing jointly up- (red) and down-regulated (blue) genes within the LILR family of proteins in the TCGA tumor indication KIRC (kidney renal clear cell carcinoma).

FIG. 29D is a clustered heat map showing normalized protein expression values for network genes in the CCLE, partitioned by Tissue.

FIG. 29E is a scatter plot showing a comparison of relative protein expression of the reported binding partners IGSF3 and PTGFRN in the lung CCLE Tissue subset. The expression pattern was significantly correlated (q<0.2) with the regression model overlaid (red).

FIG. 29F is a scatter plot showing a comparison of relative protein expression of the reported binding partners IGSF3 and PTGFRN in the upper aerodigestive tract CCLE Tissue subset. The expression pattern was significantly correlated (q<0.2) with the regression model overlaid (red).

FIG. 29G is a scatter plot showing a comparison of relative protein expression of the reported binding partners IGSF3 and PTGFRN in the esophagus CCLE Tissue subset. The expression pattern was significantly correlated (q<0.2) with the regression model overlaid (red).

FIG. 29H is a scatter plot showing a comparison of relative protein expression of the reported binding partners CEACAM5 and CEACAM6 in the lung CCLE Tissue subset. The expression pattern was significantly correlated (q<0.2) with the regression model overlaid (red).

FIG. 29I is a scatter plot showing a comparison of relative protein expression of the reported binding partners CEACAM5 and CEACAM6 in the lung CCLE Tissue subset. The expression pattern was significantly negatively correlated (q<0.2) with the regression model overlaid (red).

FIG. 29J is a scatter plot showing a comparison of relative protein expression of the reported binding partners ICOSLG and NTM in the lung CCLE Tissue subset. The expression pattern was significantly negatively correlated (q<0.2) with the regression model overlaid (red).

FIG. 30A is a volcano plot showing protein interactions significantly correlated with CD8+ Teff cells. Select interactions are highlighted.

FIG. 30B is a network diagram showing the PD-1/PD-L1 community of immune-related interactions colored by hazard ratio, with a visual indication of the inhibited interaction by atezolizumab.

FIG. 30C is a network diagram showing select binding partners for LRRC4B, colored by hazard ratio.

FIG. 30D is a network diagram showing select interaction within the ephrin family, colored by hazard ratio.

FIG. 30E is a forest plot showing increased significance for association with lack of response and patient survival for each of the CD274 (PD-L1) interactions, relative to the individual genes.

FIG. 30F is a forest plot showing increased significance for association with lack of response and patient survival for each of the LRRC4B interactions, relative to the individual genes

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) aspects that are directed to that value or parameter per se.

The term “single transmembrane receptor,” “single-pass transmembrane receptor,” or “STM receptor,” as used herein, refers to a protein having a single transmembrane domain. In some aspects, the STM receptor is expressed on the cell surface. Exemplary STM receptors are provided in Table 5, Table 7, and Table 8 and in Martinez-Martin et al., Cell, 174(5): 1158-1171, 2018 and Clark et al., Genome Res, 13: 2265-2270, 2003. In some aspects, the STM protein has the UniProt annotation “leucine-rich,” “cysteine-rich,” “ITIM/ITAM” (immunoreceptor tyrosine-based inhibition motif/immunoreceptor tyrosine-based activation motif), “TNFR” (tumor necrosis factor receptor), “TLR/ILR” (Toll-like receptor/interleukin receptor), “semaphorin,” “Kinase-like,” “Ig-like” (immunoglobulin-like), “fibronectin,” “ephrin,” “EGF,” “cytokineR,” or “cadherin”. STM receptors may be identified based on, e.g., the presence of a signal peptide or a predicted transmembrane region in the amino acid sequence. In some aspects, the STM receptor is expressed as an extracellular domain.

The term “immunoglobulin superfamily protein” or “IgSF protein,” as used herein, refers to a protein containing at least one immunoglobulin (Ig) domain or immunoglobulin fold, having the annotation “immunoglobin-like superfamily,” e.g., in the UniProt database, or otherwise indicated to have structural or functional similarity to such a protein. In some aspects, the IgSF protein has the annotation “immunoglobulin-like domain superfamily” in the UniProt database. In some aspects, the IgSF protein is included based on its participation in key biological activities, e.g., leukocyte activation, cell-cell adhesion, cell communication, or signal transduction. In some aspects, the IgSF protein has the UniProt annotation “TFNR” (transcription factor-like nuclear regulator), “TLR/ILR (Toll-like receptor/interleukin receptor), “semaphorin,” “Kinase-like,” “IgSF/Ig-like fold,” “Ig-like fold,” “fibronectin,” “ephrin,” “EGF,” “CytokineR,” or “cadherin”. In some aspects, the IgSF protein is expressed on the cell surface. In other aspects, the IgSF protein is secreted. Exemplary IgSF proteins are provided in Table 4 and in Ozkan et al., Cell, 154(1): 228-239, 2013 and Yap et al., J Mol Biol, 426(4), 945-961. In some aspects, the IgSF superfamily protein is Programmed cell death 1 ligand 1 (PD-L1; CD274), Programmed cell death 1 ligand 2 (PD-L2; CD274; PDCD1LG2), Receptor-type tyrosine-protein phosphatase delta (PTPRD), Receptor-type tyrosine-protein phosphatase S (PTPRS), Receptor-type tyrosine-protein phosphatase S (PTPRF), Neural cell adhesion molecule L1-like protein (“Close homolog of L1”; CHL1), Contactin 1 (CNTN1), Leukocyte immunoglobulin-like receptor subfamily B member 1 (LILRB1), Leukocyte immunoglobulin-like receptor subfamily B member 3 (LILRB3), Leukocyte immunoglobulin-like receptor subfamily B member 4 (LILRB4), Leukocyte immunoglobulin-like receptor subfamily B member 5 (LILRB5), MAM domain-containing glycosylphosphatidylinositol anchor protein 1 (MDGA1), or Tyrosine-protein kinase receptor AXL (UFO). In some aspects, the IgSF protein is expressed as an extracellular domain. In some aspects, the IgSF protein is a secreted protein.

As used herein, the term “immunoglobulin domain” or “Ig domain” refers to a domain of an IgSF protein that is characterized by about 7-9 antiparallel β-strands comprising a two-layer β-sheet sandwich, spanning about 70-125 amino acid residues. In some aspects, the immunoglobulin domain contains a conserved disulfide bond connecting its B and F strands. Exemplary immunoglobulin domains are described in Bork et al., JMB, 242(4): 309-320, 1194 and Yap et al., J Mol Biol, 426(4), 945-961.

As used herein, the term “extracellular domain” or “ECD” refers to a protein domain that is predicted to be localized outside of the outer plasma membrane of the cell. In some instances, the ECD is an ECD of a receptor, e.g., a STM receptor. In some aspects, the ECD is an ECD of an IgSF protein. In some aspects, the ECD is the ECD of PDPN. In some aspects, the boundaries of the extracellular domain may be identified by prediction of domains that indicate that the protein crosses the plasma membrane, e.g., a transmembrane domain (e.g., a transmembrane helix). In some aspects, the presence of an extracellular domain may be predicted by the presence of a domain, sequence, or motif that indicates that the protein is trafficked to the plasma membrane, e.g., a signal sequence or a glycosylphosphatidylinositol (GPI) linkage site. In some aspects, the boundaries of the ECD are determined according to UniProt annotations. In some aspects, the ECD is soluble. In some aspects, the extracellular domain is expressed in the context of a full-length protein. In other aspects, the extracellular domain is expressed as an isolated extracellular domain, e.g., a sequence of amino acid residues comprising only the amino acid residues of a protein that are predicted to be extracellular.

In some aspects, the isolated ECD is included in a fusion protein. In some aspects, inclusion in a fusion protein increases solubility, ease of expression, ease of capture (e.g., on a protein A-coated plate), multimerization, or some other desirable property of the ECD. In some aspects, the ECD or ECD fusion protein is a monomer. In other aspects, the ECD or ECD fusion protein is a multimer, e.g., a tetramer or a pentamer. In some aspects, the ECD is fused to a human IgG. In some aspects, the ECD is fused to a human Fc tag. In some aspects, the ECD is fused to an Avidity AVITAG™ (Avi tag). In some aspects, the ECD is fused to a polyhistidine (His) tag. In some aspects, the ECD is fused to a glycoprotein D (gD) tag and a glycosylphosphatidylinositol (GPI) linker, e.g., a gD-GPI tag. In other aspects, the ECD is fused to the pentamerization domain of rat cartilaginous oligomeric matrix protein (COMP) and the β-lactamase protein, e.g., as described in Bushell et al., Genome Res, 18: 622-630, 2008. In some aspects, the ECD fusion protein further includes a cleavage sequence, e.g., a TEV cleavage sequence, to allow removal of one or more domains. In some instances, an ECD fusion protein having an Avi tag and an Fc tag cleavable at a cleavage sequence is further processed to remove the Fc tag, to biotinylate the Avi tag, and to fuse the biotinylated ECD fusion protein to a fluorescent streptavidin (SA), e.g., to form a tetramerized ECD fusion protein. In some instances, the isolated ECD or ECD fusion protein is purified.

As used herein, a “modulator” is an agent that modulates (e.g., increases, decreases, activates, or inhibits) a given biological activity, e.g., an interaction or a downstream activity resulting from an interaction. A modulator or candidate modulator may be, e.g., a small molecule, an antibody, an antigen-binding fragment (e.g., a bis-Fab, an Fv, a Fab, a Fab′-SH, a F(ab′)₂, a diabody, a linear antibody, an scFv, an ScFab, a VH domain, or a VHH domain), a peptide, a mimic, an antisense oligonucleotide, or a small interfering RNA (siRNA).

By “increase” or “activate” is meant the ability to cause an overall increase, for example, of 20% or greater, of 50% or greater, or of 75%, 85%, 90%, or 95% or greater. In certain aspects, increase or activate can refer to a downstream activity of a protein-protein interaction.

By “reduce” or “inhibit” is meant the ability to cause an overall decrease, for example, of 20% or greater, of 50% or greater, or of 75%, 85%, 90%, or 95% or greater. In certain aspects, reduce or inhibit can refer to a downstream activity of a protein-protein interaction.

“Affinity” refers to the strength of the sum total of noncovalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and ligand). The affinity of a molecule X for its partner Y can generally be represented by the dissociation constant (K_{D}). Affinity can be measured by common methods known in the art, including those described herein.

“Complex” or “complexed” as used herein refers to the association of two or more molecules that interact with each other through bonds and/or forces (e.g., Van der Waals, hydrophobic, hydrophilic forces) that are not peptide bonds. In one aspect, a complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

A “disorder” is any condition that would benefit from treatment including, but not limited to, chronic and acute disorders or diseases including those pathological conditions which predispose the mammal to the disorder in question. In one aspect, the disorder is a cancer.

The terms “cancer” and “cancerous” refer to or describe the physiological condition in mammals that is typically characterized by unregulated cell growth/proliferation. Aspects of cancer include solid tumor cancers and non-solid tumor cancers. Solid cancer tumors include, but are not limited to a colorectal cancer, a head and neck cancer (e.g., a squamous cell carcinoma of the head and neck), a glioma, a melanoma, a breast cancer, a lung cancer, a bladder cancer, a kidney cancer, an ovarian cancer, a pancreatic cancer, or a prostate cancer, or metastatic forms thereof. In some aspects, the cancer is a colorectal cancer (CRC). In some aspects, the cancer is a head and neck cancer. Further aspects of head and neck cancer include a squamous cell carcinoma of the head & neck (SCCHN). In some aspects, the cancer is a breast cancer. Further aspects of breast cancer include a hormone receptor-positive (HR+) breast cancer, e.g., an estrogen receptor-positive (ER+) breast cancer, a progesterone receptor-positive (PR+) breast cancer, or an ER+/PR+ breast cancer. Other aspects of breast cancer include a HER2-positive (HER2+) breast cancer. Yet other aspects of breast cancer include a triple-negative breast cancer (TNBC). In some aspects, the breast cancer is an early breast cancer. In some aspects, the cancer is a lung cancer. Further aspects of lung cancer include an epidermal growth factor receptor-positive (EGFR+) lung cancer. Other aspects of lung cancer include an epidermal growth factor receptor-negative (EGFR−) lung cancer. Yet other aspects of lung cancer include a non-small cell lung cancer, e.g., a squamous lung cancer or a non-squamous lung cancer. Other aspects of lung cancer include a small cell lung cancer. In some aspects, the cancer is a urinary tract cancer. Urinary tract cancers include urothelial carcinomas (UC), non-urothelial carcinomas of the urinary tract, and carcinomas of the urinary tract having mixed histology. Non-urothelial carcinomas of the urinary tract include all subtypes listed in the World Health Organization classification, e.g., a squamous cell carcinoma, a verrucous carcinoma, an adenocarcinoma, a glandular carcinoma, a carcinoma of the Bellini collecting duct, a neuroendocrine carcinoma, or a small cell carcinoma. The adenocarcinoma may be an enteric adenocarcinoma, a mucinous adenocarcinoma, a signet-ring cell adenocarcinoma, or a clear cell adenocarcinoma. Urinary tract cancers may be located in the bladder, the renal pelvis, the ureter, or the urethra. In some aspects, the urinary tract cancer (e.g., urothelial carcinoma, non-urothelial carcinoma, or carcinoma of the urinary tract having mixed histology) is locally advanced, e.g., stage T4b N_{any} or T_{any} N2-3, according to the TNM classification, at the onset of treatment. In some aspects, the urinary tract cancer is a metastatic urothelial carcinoma (mUC), a metastatic form of a non-urothelial carcinoma of the urinary tract, or a metastatic form of a carcinoma of the urinary tract having mixed histology. In some aspects, the urinary tract cancer is TNM stage M1, according to the TNM classification, at the onset of treatment. In some aspects, the cancer is a bladder cancer. Further aspects of bladder cancer include a urothelial bladder cancer (UBC), a muscle invasive bladder cancer (MIBC), or a non-muscle invasive bladder cancer (NMIBC). In some aspects, the cancer is a kidney cancer. Further aspects of kidney cancer include a renal cell carcinoma (RCC). In some aspects, the cancer is a liver cancer. Further aspects of liver cancer include a hepatocellular carcinoma. In some aspects, the cancer is a prostate cancer. Further aspects of prostate cancer include a castration-resistant prostate cancer (CRPC). In some aspects, the cancer is a metastatic form of a solid tumor. In some aspects, the metastatic form of a solid tumor is a metastatic form of a melanoma, a breast cancer, a colorectal cancer, a lung cancer, a head and neck cancer, a bladder cancer, a kidney cancer, an ovarian cancer, a pancreatic cancer, or a prostate cancer. In some aspects, the cancer is a non-solid tumor cancer. Non-solid tumor cancers include, but are not limited to, a B-cell lymphoma. Further aspects of B-cell lymphoma include, e.g., a chronic lymphocytic leukemia (CLL), a diffuse large B-cell lymphoma (DLBCL), a follicular lymphoma, myelodysplastic syndrome (MDS), a non-Hodgkin lymphoma (NHL), an acute lymphoblastic leukemia (ALL), a multiple myeloma, an acute myeloid leukemia (AML), or a mycosis fungoides (MF).

The terms “host cell,” “host cell line,” and “host cell culture” are used interchangeably and refer to cells into which exogenous nucleic acid has been introduced, including the progeny of such cells. Host cells include “transfected cells,” “transformed cells,” and “transformants,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In some aspects, the host cell is stably transformed with the exogenous nucleic acid. In other aspects, the host cell is transiently transformed with the exogenous nucleic acid.

The term “cancer-associated fibroblast” (“CAF”) or “tumor-associated fibroblast,” as used herein, refers to a fibroblast cell (e.g., a mammalian stromal cell) present in or associated with the tumor microenvironment (TME), e.g., the stroma. In some aspects, the CAFs are active fibroblasts. CAFs may regulate the structure and/or function of the TME, e.g., via extracellular matrix (ECM) remodeling and/or secretion of soluble factors, e.g., growth factors and/or inflammatory factors. CAFs may contribute to tumorigenesis, tumor growth, tumor invasion, angiogenesis or metastasis. CAFs may impair anti-tumor immunity. In some aspects, CAFs express podoplanin (PDPN). In some aspects, CAFs are characterized by actomyosin contractility, a property that affects tissue stiffness. In some aspects, CAFs are associated with an activated fibroblast signature and/or an FAP⁺ fibroblast signature, e.g., express genes provided in Table 11 and/or Table 12.

The term “podoplanin” or “PDPN,” as used herein, broadly refers to any native PDPN from any mammalian source, including primates (e.g. humans) and rodents (e.g., mice and rats), unless otherwise indicated. PDPN is also called gp38, Aggrus, and T1α. The term encompasses full-length PDPN and isolated regions or domains of PDPN, e.g., the PDPN extracellular domain (ECD). The term also encompasses naturally occurring variants of PDPN, e.g., splice variants or allelic variants. The amino acid sequence of an exemplary human PDPN is shown under UniProt Accession No. Q86YL7. Minor sequence variations, especially conservative amino acid substitutions of PDPN that do not affect PDPN function and/or activity, are also contemplated by the invention.

The term “cluster of differentiation 177” or “CD177,” as used herein, broadly refers to any native CD177 from any mammalian source, including primates (e.g. humans) and rodents (e.g., mice and rats), unless otherwise indicated. The term encompasses full-length CD177 and isolated regions or domains of CD177, e.g., the CD177 ECD. The term also encompasses naturally occurring variants of CD177, e.g., splice variants or allelic variants. The amino acid sequence of an exemplary human CD177 is shown under UniProt Accession No. Q8N6Q3. Minor sequence variations, especially conservative amino acid substitutions of CD177 that do not affect CD177 function and/or activity, are also contemplated by the invention.

The term “agonist of CD177 activity” or “CD177 agonist” refers to a molecule that increases signal transduction resulting from the interaction of CD177 with one or more of its binding partners, e.g., PDPN. The agonist of CD177 activity may result in an increase in the binding of CD177 to one or more of its binding partners (e.g., PDPN) relative to binding of the two proteins in the absence of the agonist. Agonists of CD177 activity may include antibodies, antigen binding fragments thereof, immunoadhesins, fusion proteins, peptides (e.g., multimerized peptides, e.g., multimerized CD177 polypeptides), oligopeptides, and other molecules that increase signal transduction resulting from the interaction of CD177 with one or more of its binding partners, e.g., PDPN.

The term “Programmed cell death 1 ligand 1” or “PD-L1,” as used herein, broadly refers to any native PD-L1 from any mammalian source, including primates (e.g. humans) and rodents (e.g., mice and rats), unless otherwise indicated. PD-L1 is also called CD274. The term encompasses full-length PD-L1 and isolated regions or domains of PD-L1, e.g., the PD-L1 ECD. The term also encompasses naturally occurring variants of PD-L1, e.g., splice variants or allelic variants. The amino acid sequence of an exemplary human PD-L1 is shown under UniProt Accession No. Q9NZQ7. Minor sequence variations, especially conservative amino acid substitutions of PD-L1 that do not affect PD-L1 function and/or activity, are also contemplated by the invention.

The term “ephrin type-A receptor 3” or “EPHA3,” as used herein, broadly refers to any native EPHA3 from any mammalian source, including primates (e.g. humans) and rodents (e.g., mice and rats), unless otherwise indicated. The term encompasses full-length EPHA3 and isolated regions or domains of EPHA3, e.g., the EPHA3 ECD. The term also encompasses naturally occurring variants of EPHA3, e.g., splice variants or allelic variants. The amino acid sequence of an exemplary human EPHA3 is shown under UniProt Accession No. P29320. Minor sequence variations, especially conservative amino acid substitutions of EPHA3 that do not affect EPHA3 function and/or activity, are also contemplated by the invention.

The term “Programmed cell death 1 ligand 2” or “PD-L2,” as used herein, broadly refers to any native PD-L2 from any mammalian source, including primates (e.g. humans) and rodents (e.g., mice and rats), unless otherwise indicated. PD-L2 is also called PDCD1LG2. The term encompasses full-length PD-L2 and isolated regions or domains of PD-L2, e.g., the PD-L2 ECD. The term also encompasses naturally occurring variants of PD-L2, e.g., splice variants or allelic variants. The amino acid sequence of an exemplary human PD-L2 is shown under UniProt Accession No. Q9BQ51. Minor sequence variations, especially conservative amino acid substitutions of PD-L2 that do not affect PD-L2 function and/or activity, are also contemplated by the invention.

The term “CEACAM4,” as used herein, broadly refers to any native CEACAM4 from any mammalian source, including primates (e.g. humans) and rodents (e.g., mice and rats), unless otherwise indicated. The term encompasses full-length CEACAM4 and isolated regions or domains of CEACAM4, e.g., the CEACAM4 ECD. The term also encompasses naturally occurring variants of CEACAM4, e.g., splice variants or allelic variants. The amino acid sequence of an exemplary human CEACAM4 is shown under UniProt Accession No. O75871. Minor sequence variations, especially conservative amino acid substitutions of CEACAM4 that do not affect CEACAM4 function and/or activity, are also contemplated by the invention.

The term “Receptor-type tyrosine-protein phosphatase delta” or “PTPRD,” as used herein, broadly refers to any native PTPRD from any mammalian source, including primates (e.g. humans) and rodents (e.g., mice and rats), unless otherwise indicated. The term encompasses full-length PTPRD and isolated regions or domains of PTPRD, e.g., the PTPRD ECD. The term also encompasses naturally occurring variants of PTPRD, e.g., splice variants or allelic variants. The amino acid sequence of an exemplary human PTPRD is shown under UniProt Accession No. P23468. Minor sequence variations, especially conservative amino acid substitutions of PTPRD that do not affect PTPRD function and/or activity, are also contemplated by the invention.

The term “Receptor-type tyrosine-protein phosphatase F” or “PTPRF,” as used herein, broadly refers to any native PTPRF from any mammalian source, including primates (e.g. humans) and rodents (e.g., mice and rats), unless otherwise indicated. The term encompasses full-length PTPRF and isolated regions or domains of PTPRF, e.g., the PTPRF ECD. The term also encompasses naturally occurring variants of PTPRF, e.g., splice variants or allelic variants. The amino acid sequence of an exemplary human PTPRF is shown under UniProt Accession No. P10586. Minor sequence variations, especially conservative amino acid substitutions of PTPRF that do not affect PTPRF function and/or activity, are also contemplated by the invention.

The term “Receptor-type tyrosine-protein phosphatase S” or “PTPRS,” as used herein, broadly refers to any native PTPRS from any mammalian source, including primates (e.g. humans) and rodents (e.g., mice and rats), unless otherwise indicated. The term encompasses full-length PTPRS and isolated regions or domains of PTPRS, e.g., the PTPRS ECD. The term also encompasses naturally occurring variants of PTPRS, e.g., splice variants or allelic variants. The amino acid sequence of an exemplary human PTPRS is shown under UniProt Accession No. Q13332. Minor sequence variations, especially conservative amino acid substitutions of PTPRS that do not affect PTPRS function and/or activity, are also contemplated by the invention.

The term “CHL1,” as used herein, broadly refers to any native CHL1 from any mammalian source, including primates (e.g. humans) and rodents (e.g., mice and rats), unless otherwise indicated. The term encompasses full-length CHL1 and isolated regions or domains of CHL1, e.g., the CHL1 ECD. The term also encompasses naturally occurring variants of CHL1, e.g., splice variants or allelic variants. The amino acid sequence of an exemplary human CHL1 is shown under UniProt Accession No. O00533. Minor sequence variations, especially conservative amino acid substitutions of CHL1 that do not affect CHL1 function and/or activity, are also contemplated by the invention.

The term “Contactin 1” or “CNTN1,” as used herein, broadly refers to any native CNTN1 from any mammalian source, including primates (e.g. humans) and rodents (e.g., mice and rats), unless otherwise indicated. The term encompasses full-length CNTN1 and isolated regions or domains of CNTN1, e.g., the CNTN1 ECD. The term also encompasses naturally occurring variants of CNTN1, e.g., splice variants or allelic variants. The amino acid sequence of an exemplary human CNTN1 is shown under UniProt Accession No. Q12860. Minor sequence variations, especially conservative amino acid substitutions of CNTN1 that do not affect CNTN1 function and/or activity, are also contemplated by the invention.

The term “Leukocyte immunoglobulin-like receptor subfamily B member 1” or “LILRB1,” as used herein, broadly refers to any native LILRB1 from any mammalian source, including primates (e.g. humans) and rodents (e.g., mice and rats), unless otherwise indicated. The term encompasses full-length LILRB1 and isolated regions or domains of LILRB1, e.g., the LILRB1 ECD. The term also encompasses naturally occurring variants of LILRB1, e.g., splice variants or allelic variants. The amino acid sequence of an exemplary human LILRB1 is shown under UniProt Accession No. Q8NHL6. Minor sequence variations, especially conservative amino acid substitutions of LILRB1 that do not affect LILRB1 function and/or activity, are also contemplated by the invention.

The term “Leukocyte immunoglobulin-like receptor subfamily B member 2” or “LILRB2,” as used herein, broadly refers to any native LILRB2 from any mammalian source, including primates (e.g. humans) and rodents (e.g., mice and rats), unless otherwise indicated. The term encompasses full-length LILRB2 and isolated regions or domains of LILRB2, e.g., the LILRB2 ECD. The term also encompasses naturally occurring variants of LILRB2, e.g., splice variants or allelic variants. The amino acid sequence of an exemplary human LILRB2 is shown under UniProt Accession No. Q8N423. Minor sequence variations, especially conservative amino acid substitutions of LILRB2 that do not affect LILRB2 function and/or activity, are also contemplated by the invention.

The term “Leukocyte immunoglobulin-like receptor subfamily B member 3” or “LILRB3,” as used herein, broadly refers to any native LILRB3 from any mammalian source, including primates (e.g. humans) and rodents (e.g., mice and rats), unless otherwise indicated. The term encompasses full-length LILRB3 and isolated regions or domains of LILRB3, e.g., the LILRB3 ECD. The term also encompasses naturally occurring variants of LILRB3, e.g., splice variants or allelic variants. The amino acid sequence of an exemplary human LILRB3 is shown under UniProt Accession No. O75022. Minor sequence variations, especially conservative amino acid substitutions of LILRB3 that do not affect LILRB3 function and/or activity, are also contemplated by the invention.

The term “Leukocyte immunoglobulin-like receptor subfamily B member 4” or “LILRB4,” as used herein, broadly refers to any native LILRB4 from any mammalian source, including primates (e.g. humans) and rodents (e.g., mice and rats), unless otherwise indicated. The term encompasses full-length LILRB4 and isolated regions or domains of LILRB4, e.g., the LILRB4 ECD. The term also encompasses naturally occurring variants of LILRB4, e.g., splice variants or allelic variants. The amino acid sequence of an exemplary human LILRB4 is shown under UniProt Accession No. Q8NHJ6. Minor sequence variations, especially conservative amino acid substitutions of LILRB4 that do not affect LILRB4 function and/or activity, are also contemplated by the invention.

The term “Leukocyte immunoglobulin-like receptor subfamily B member 5” or “LILRB5,” as used herein, broadly refers to any native LILRB5 from any mammalian source, including primates (e.g. humans) and rodents (e.g., mice and rats), unless otherwise indicated. The term encompasses full-length LILRB5 and isolated regions or domains of LILRB5, e.g., the LILRB5 ECD. The term also encompasses naturally occurring variants of LILRB5, e.g., splice variants or allelic variants. The amino acid sequence of an exemplary human LILRB5 is shown under UniProt Accession No. O75023. Minor sequence variations, especially conservative amino acid substitutions of LILRB5 that do not affect LILRB5 function and/or activity, are also contemplated by the invention.

The term “AXL,” as used herein, broadly refers to any native AXL from any mammalian source, including primates (e.g. humans) and rodents (e.g., mice and rats), unless otherwise indicated. The term encompasses full-length AXL and isolated regions or domains of AXL, e.g., the AXL ECD. AXL is also known as UFO. The term also encompasses naturally occurring variants of AXL, e.g., splice variants or allelic variants. The amino acid sequence of an exemplary human AXL is shown under UniProt Accession No. P30530. Minor sequence variations, especially conservative amino acid substitutions of AXL that do not affect AXL function and/or activity, are also contemplated by the invention.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein any form of the protein that results from processing in the cell. The term also encompasses naturally occurring variants of the protein, e.g., splice variants or allelic variants, e.g., amino acid substitution mutations or amino acid deletion mutations. The term also includes isolated regions or domains of the protein, e.g., the extracellular domain (ECD).

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “interactome,” as used herein, refers to the set of molecular interactions, e.g., protein-protein interactions, occurring within a set of molecules. In some aspects, the interactome is represented as a network, e.g., a network in which nodes represent a specific molecule and edges connect nodes for which an assay (e.g., a cell surface interaction assay or an extracellular interaction assay) detects interaction between the two nodes.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the term “immune checkpoint inhibitor” refers to a therapeutic agent that targets at least one immune checkpoint protein to alter the regulation of an immune response, e.g., down-modulating, inhibiting, up-modulating, or activating an immune response. The term “immune checkpoint blockade” may be used to refer to a therapy comprising an immune checkpoint inhibitor. Immune checkpoint proteins are known in the art and include, without limitation, cytotoxic T-lymphocyte antigen 4 (CTLA-4), programmed cell death 1 (PD-1), programmed cell death ligand 1 (PD-L1), programmed cell death ligand 2 (PD-L2), V-domain Ig suppressor of T cell activation (VISTA), B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some aspects, an immune checkpoint protein may be expressed on the surface of an activated T cell. Therapeutic agents that can act as immune checkpoint inhibitors useful in the methods of the present invention, include, but are not limited to, therapeutic agents that target one or more of CTLA-4, PD-1, PD-L1, PD-L2, VISTA, B7-H2, B7-H3, B7-H4, B7-H6, 2B4, ICOS, HVEM, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some aspects, an immune checkpoint inhibitor enhances or suppresses the function of one or more targeted immune checkpoint proteins. In some aspects, the immune checkpoint inhibitor is a PD-L1 axis binding antagonist, such as atezolizumab.

The term “PD-L1 axis binding antagonist” refers to a molecule that inhibits the interaction of a PD-1 axis binding partner with either one or more of its binding partner, so as to remove T cell dysfunction resulting from signaling on the PD-1 signaling axis—with a result being to restore or enhance T cell function (e.g., proliferation, cytokine production, target cell killing). As used herein, a PD-L1 axis binding antagonist includes a PD-L1 binding antagonist, a PD-1 binding antagonist, and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, or PD-L2. In some aspects, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one aspect, a PD-1 binding antagonist reduces the negative co-stimulatory signal mediated by or through cell surface proteins expressed on T lymphocytes mediated signaling through PD-1 so as render a dysfunctional T cell less dysfunctional (e.g., enhancing effector responses to antigen recognition). In some aspects, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108.

The term “PD-L1 binding antagonist” refers to a molecule that decreases, blocks, inhibits, abrogates or interferes with signal transduction resulting from the interaction of PD-L1 with either one or more of its binding partners, such as PD-1 and B7-1. In some aspects, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some aspects, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and B7-1. In one aspect, a PD-L1 binding antagonist reduces the negative co-stimulatory signal mediated by or through cell surface proteins expressed on T lymphocytes mediated signaling through PD-L1 so as to render a dysfunctional T cell less dysfunctional (e.g., enhancing effector responses to antigen recognition). In some aspects, a PD-L1 binding antagonist is an anti-PD-L1 antibody. In still another specific aspect, an anti-PD-L1 antibody is MPDL3280A (atezolizumab, marketed as TECENTRIQ™ with a WHO Drug Information (International Nonproprietary Names for Pharmaceutical Substances), Recommended INN: List 74, Vol. 29, No. 3, 2015 (see page 387)). In a specific aspect, an anti-PD-L1 antibody is YW243.55.S70. In another specific aspect, an anti-PD-L1 antibody is MDX-1105. In another specific aspect, an anti PD-L1 antibody is MSB0015718C. In still another specific aspect, an anti-PD-L1 antibody is MEDI4736.

The term “PD-L2 binding antagonist” refers to a molecule that decreases, blocks, inhibits, abrogates or interferes with signal transduction resulting from the interaction of PD-L2 with either one or more of its binding partners, such as PD-1. In some aspects, a PD-L2 binding antagonist is a molecule that inhibits the binding of PD-L2 to one or more of its binding partners. In a specific aspect, the PD-L2 binding antagonist inhibits binding of PD-L2 to PD-1. In some aspects, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T cell less dysfunctional (e.g., enhancing effector responses to antigen recognition). In some aspects, a PD-L2 binding antagonist is an immunoadhesin.

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

An “antigen-binding fragment” or “antibody fragment” refers to a molecule other than an intact antibody that comprises a portion of an intact antibody that binds the antigen to which the intact antibody binds. Examples of antigen-binding fragments include but are not limited to bis-Fabs; Fv; Fab; Fab, Fab′-SH; F(ab′)₂; diabodies; linear antibodies; single-chain antibody molecules (e.g., scFv, ScFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

“Fv” consists of a dimer of one heavy- and one light-chain variable region domain in tight, noncovalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The terms “full-length antibody,” “intact antibody,” and “whole antibody” are used herein interchangeably to refer to an antibody having a structure substantially similar to a native antibody structure or having heavy chains that contain an Fc region as defined herein.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

The term “small molecule” refers to any molecule with a molecular weight of about 2000 daltons or less, e.g., about 1000 daltons or less. In some aspects, the small molecule is a small organic molecule.

The term “mimic” or “molecular mimic,” as used herein, refers to a polypeptide having sufficient similarity in conformation and/or binding ability (e.g., secondary structure, tertiary structure) to a given polypeptide or to a portion of said polypeptide to bind to a binding partner of said polypeptide. The mimic may bind the binding partner with equal, less, or greater affinity than the polypeptide it mimics. A molecular mimic may or may not have obvious amino acid sequence similarity to the polypeptide it mimics. A mimic may be naturally occurring or may be engineered. In some aspects, the mimic is a mimic of the protein of Table 1. In other aspects, the mimic is a mimic of the protein of Table 2. In yet other aspects, the mimic is a mimic of another protein that binds to the protein of Table 1 or the protein of Table 2. In some aspects, the mimic may perform all functions of the mimicked polypeptide. In other aspects, the mimic does not perform all functions of the mimicked polypeptide.

As used herein, the term “conditions permitting the binding” of two or more proteins to each other (e.g., a protein of Table 1 and a protein of Table 2) refers to conditions (e.g., protein concentration, temperature, pH, salt concentration) under which the two or more proteins would interact in the absence of a modulator or a candidate modulator. Conditions permitting binding may differ for individual proteins and may differ between protein-protein interaction assays (e.g., surface plasmon resonance assays, biolayer interferometry assays, enzyme-linked immunosorbent assays (ELISA), extracellular interaction assays, and cell surface interaction assays.

The term “survival” refers to the patient remaining alive, and includes overall survival as well as progression-free survival.

As used herein, “recurrence-free survival” or “RFS” refers to the length of time after primary treatment for a cancer ends that the patient survives without any signs or symptoms of that cancer. RFS may also be referred to as “disease-free survival” or “DFS”. In some aspects, Disease-free survival (RFS) is defined as the time between randomization (e.g., assignment to an adjuvant treatment group) and recurrence of a disease (e.g., a cancer), new occurrence of a disease (e.g., a cancer), or death from any cause.

As used herein, “progression-free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” or “OS” refers to the percentage of individuals in a group who are likely to be alive after a particular duration of time.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, an agent (e.g., a modulator, a PD-L1 axis binding antagonist, or an agonist of CD177 activity) is used to delay development of a disease or to slow the progression of a disease.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human.

As used herein, “administering” is meant a method of giving a dosage of a compound to a subject. In some aspects, the compositions utilized in the methods herein are administered intravenously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravascularly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, and tissue culture medium, tissue extracts such as homogenized tissue, tumor tissue, cellular extracts, and combinations thereof. The sample may be an archival sample, a fresh sample, or a frozen sample. In some aspects, the sample is a formalin-fixed and paraffin-embedded (FFPE) tumor tissue sample.

### II. Methods of Identifying Protein-Protein Interactions

A. Assays for Protein-Protein Interaction

Interactions between two proteins are commonly tested using yeast two-hybrid (Y2H) assays and/or biochemical purification assays (e.g., affinity purification-mass spectrometry (AP/MS)); however, these methods are not well-suited to testing interactions between cell surface proteins. Cell surface proteins are often not soluble in the cytoplasm and/or nucleoplasm (e.g., due to hydrophobic transmembrane regions) and are thus inappropriate for Y2H assays. Further, interactions between cell surface proteins are often low-affinity and/or highly transient, e.g., having a half-life of less than one second, and are thus not compatible with assays involving lengthy purification protocols (e.g., AP/MS) (Bushell et al., Genome Res, 18: 622-630, 2008). The protein-protein interaction assays described herein, e.g., extracellular interaction assays and cell surface interaction assays, are compatible with cell surface proteins and are thus able to identify novel interactions among these proteins.

i. Extracellular Interaction Assays

In some aspects of the invention, the protein-protein interaction assay is an extracellular interaction assay, e.g., an avidity-based extracellular interaction screen (AVEXIS) (Bushell et al., Genome Res, 18: 622-630, 2008; Martinez-Martin et al., J Immunol Res, 2197615, 2017). In this type of assay, one or more prey proteins (e.g., one or more STM receptors) and one or more bait proteins (e.g., one or more IgSF proteins) are expressed as soluble extracellular domain (ECD) fusion proteins, as described below, and are assayed for interaction (e.g., using a colorimetric assay) in conditioned media.

ia. Extracellular Interaction Assay Prey Proteins

In some aspects, the prey protein or prey proteins comprise one or more fusion proteins (e.g., a library of prey fusion proteins) in which the extracellular domain (ECD) of a prey protein of interest (e.g., an STM protein) is conjugated (e.g., fused) to one or more additional moieties (e.g., an IgG or an Fc tag, e.g., a human Fc tag) such that the prey fusion protein is soluble. ECDs may be identified as described in Section 2B(i).

ib. Extracellular Interaction Assay Bait Proteins

In some aspects, the bait protein (query protein) or bait proteins comprise one or more fusion proteins (e.g., a library of bait fusion proteins) in which the ECD of a bait protein of interest is conjugated (e.g., fused) to one or more additional moieties such that the bait fusion protein is soluble. The additional moiety may also increase the avidity of the bait fusion protein for the prey protein, e.g., by multimerizing the bait protein ECD. Increasing avidity may increase the detection of low-affinity interactions. In some aspects, the additional moiety or moieties cause pentamerization of the bait protein ECD. In some aspects, the additional moiety is the pentamerization domain of rat cartilaginous oligomeric maintenance protein (COMP). ECDs may be identified as described in Section 2B(i).

The bait protein may also be conjugated to a moiety that allows detection of the bait protein, e.g., the beta-lactamase (β-lactamase) protein. β-lactamase hydrolyzes the substrate nitrocefin, producing a yellow to red color change, which may be detected in a colorimetric assay (e.g., measurement of absorbance at 485 nm).

In some aspects, the bait fusion protein comprises both a COMP pentamerization domain and a β-lactamase protein.

ic. Expression

The bait fusion protein and/or prey fusion protein may be expressed (e.g., transfected, e.g., transiently transfected) in a cell. The cell may be a human cell, e.g., a HEK293 cell (e.g., an Expi293F cell). In some aspects, the bait fusion protein and/or prey fusion protein is expressed in conditioned media, e.g., the conditioned media of transfected cells, e.g., the conditioned media of transfected human cells. Expression in human cells may increase the likelihood that posttranslational modifications (e.g., disulfide bonds, addition of one or more glycans) occur, thus increasing the likelihood that functionally relevant interactions may be detected.

Cells may be removed from the conditioned media (e.g., by centrifugation) after a period of growth (e.g., 7 days); bait fusion proteins and/or prey fusion proteins are present in the conditioned media from which cells have been removed (e.g. the supernatant of a centrifugation step).

id. Assay for Interaction

The prey fusion protein may be captured from conditioned media, e.g., captured on a protein A-coated substrate based on the affinity between protein A and an Fc tag of the prey fusion protein. The substrate may be a well, e.g., a well in a 384-well plate.

The bait fusion protein may be assayed directly in the conditioned media. The concentration of the bait protein may be normalized before the assay for interaction is performed, e.g., by dilution.

To perform the protein-protein interaction assay, a solution comprising the bait protein may be added to one or more substrates comprising a prey protein (e.g., to one or more wells of a 384-well plate). The assay may then be incubated and washed one or more times to remove non-bound bait protein. In aspects in which the bait fusion protein comprises β-lactamase, interaction between the bait fusion protein and the prey fusion protein may be detected by the addition of the substrate nitrocefin and measurement of nitrocefin hydrolysis, e.g., by measuring absorbance at 485 nm. A relatively high absorbance indicates that the bait fusion protein is retained, i.e., that the bait fusion protein and the prey fusion protein interact.

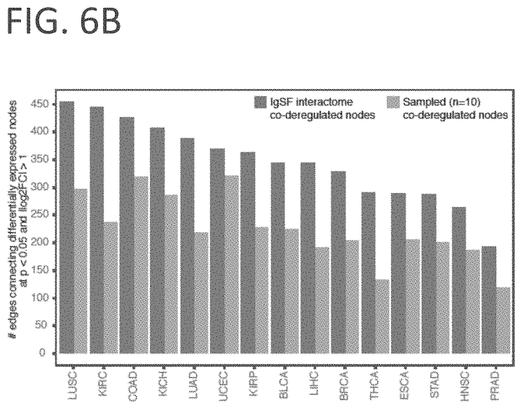

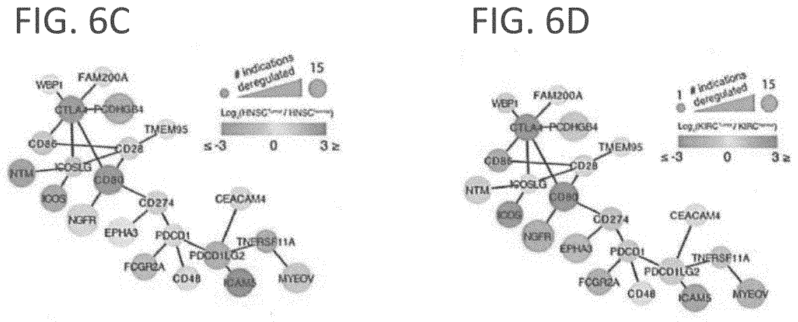

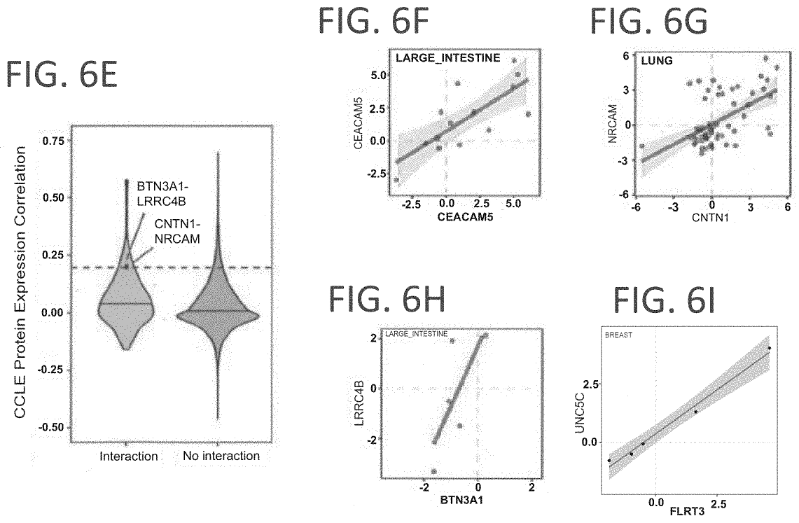

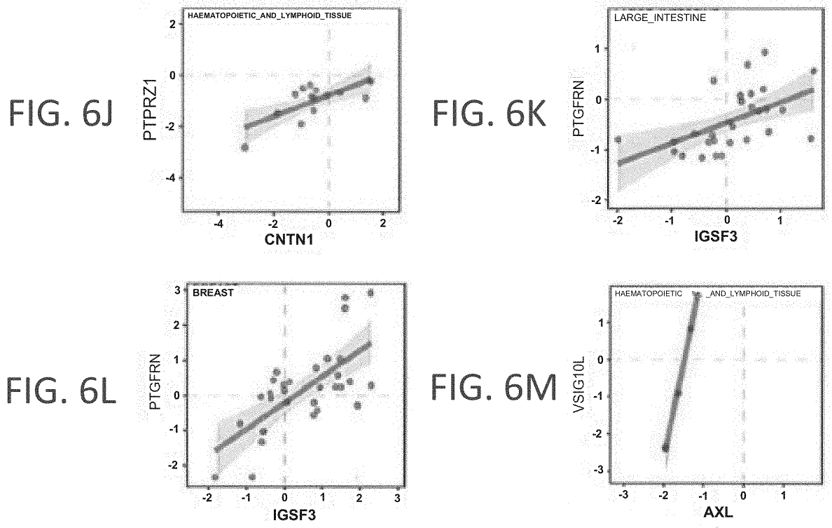

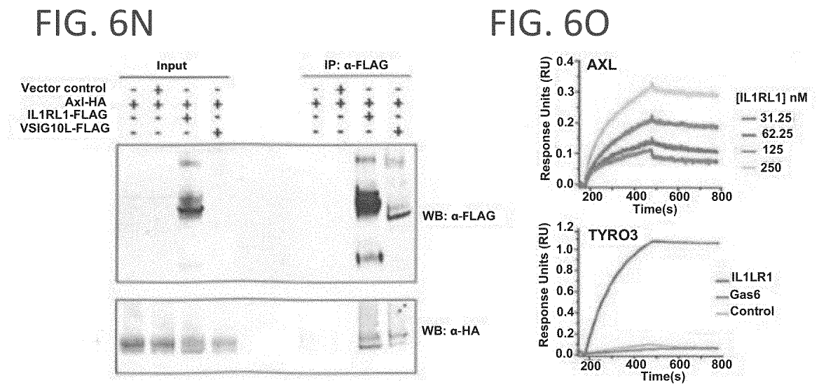

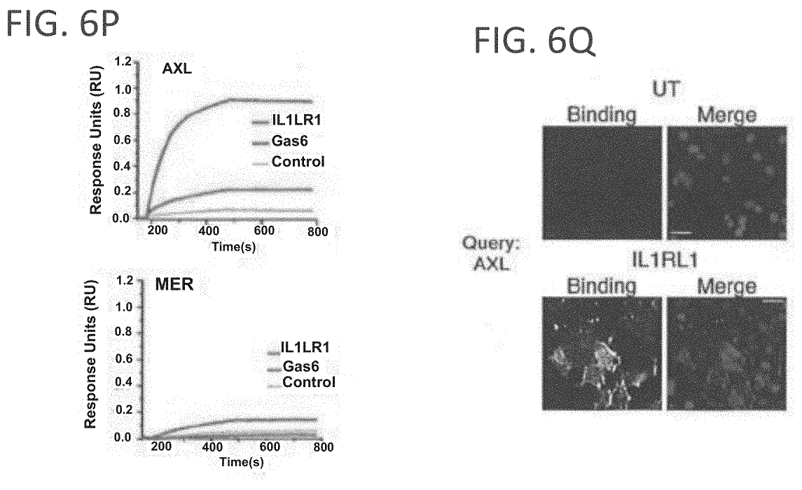

In some aspects, the assay is quantitative and the level of absorbance may be used to measure the relative strength of the interaction (e.g., as in FIG. 6G).

ie. Automated Extracellular Interaction Screen

The extracellular interaction assay may be a high-throughput assay, e.g., a screen. A screen may include between 1 and more than 1500 prey fusion proteins (e.g., 1, more than 1, more than 10, more than 100, more than 250, more than 500, more than 750, more than 1000, more than 1250, or more than 1500 prey fusion proteins) and between 1 and more than 1500 bait fusion proteins (e.g., 1, more than 1, more than 10, more than 100, more than 250, more than 500, more than 750, more than 1000, more than 1250, or more than 1500 bait fusion proteins). In some aspects, the assay uses an integrated robotic system. In some aspects, the assay is performed in one or more 384-well plates. In some aspects, assays are assessed by a computational pipeline, e.g., a supervised classification algorithm, to determine whether interaction has occurred.

ii. Cell Surface Interaction Assay

In some aspects of the invention, the protein-protein interaction assay is a cell surface interaction assay. In this type of assay, one or more prey proteins (e.g., one or more STM receptors) are expressed as extracellular domain (ECD) fusion proteins on the cell surface and are tested for interaction with one or more bait proteins (e.g., an IgSF protein or PDPN) expressed as a soluble extracellular domain using, e.g., a fluorescent assay wherein the bait protein comprises a fluorescent tag.

In some aspects, the invention comprises a method for identifying a protein-protein interaction, the method comprising (a) providing the collection of polypeptides, wherein each polypeptide comprises an extracellular domain, a tag, and an anchor, and wherein the collection of polypeptides comprises the extracellular domains of all or a subset of the proteins of Table 7, optionally wherein said polypeptides are immobilized on (e.g., attached to or fixed to) on one or more solid surfaces, e.g., wells of a plate or a set of plates; (b) contacting the collection of step (a) with a multimerized query protein under conditions permitting the binding of the multimerized query protein and at least one of the extracellular domains of the polypeptides; and (c) detecting an interaction between the multimerized query protein and the at least one extracellular domain, thereby identifying a protein-protein interaction. Each of the polypeptides may be localized to (e.g., immobilized to) a distinct location (e.g., a distinctly interrogatable location, e.g., a location that can be interrogated distinctly by the methods described herein) on the one or more solid surfaces. For example, each distinct location may comprise one or more cells that display the polypeptide. Exemplary collections of polypeptides that may be used in the method are described in Section IIB.

In some aspects, the invention comprises a method for identifying a protein-protein interaction, the method comprising: (a) providing a solid surface or a set of solid surfaces comprising a number of locations, each of the locations comprising a unique polypeptide from a collection of polypeptides, wherein each polypeptide comprises an extracellular domain, a tag, and an anchor, and wherein the collection of polypeptides comprises the extracellular domains of all or a subset of of the proteins of Table 7; (b) contacting the solid surface of step (a) with a multimerized query protein under conditions permitting the binding of the multimerized query protein and the extracellular domains of the polypeptides; and (c) detecting an interaction between the multimerized query protein and at least one polypeptide from the collection of polypeptides, thereby identifying a protein-protein interaction.

In some aspects, the solid surface or set of solid surfaces together comprise at least 965 locations, each of the locations comprising a unique polypeptide from a collection of polypeptides, wherein each polypeptide comprises an extracellular domain, a tag, and an anchor, and wherein the collection of polypeptides comprises the extracellular domains of at least 81% of the proteins of Table 7.

In some aspects, each of the locations comprises a cell, e.g., a mammalian cell, that displays the unique polypeptide. In some aspects, the cell has been transfected, e.g., transiently transfected, with a vector encoding the unique polypeptide. In some aspects, the transient transfection is semi-automated.

In some aspects, the multimerized query protein is a query protein as described in described in Section IIA(iib). The multimerized query protein may be, e.g., a dimerized, trimerized, tetramerized, or pentamerized query protein. In some aspects, the multimerized query protein is a tetramerized query protein. The multimerized query protein may comprise an isolated extracellular domain of the query protein. For example, the isolated extracellular domain may be biotinylated and conjugated to a fluorescent streptavidin to tetramerize the query protein.

In some aspects, protein-protein interactions are identified as described in Section IIA(iid).

iia. Cell Surface Interaction Assay Prey Proteins

In some aspects, the prey protein or prey proteins comprise one or more fusion proteins (e.g., a library of prey fusion proteins) in which the extracellular domain (ECD) of a prey protein of interest (e.g., an STM protein) is conjugated (e.g., fused) to one or more additional moieties (e.g., a glycosylphosphatidylinositol (GPI)-gD (gDGPI) tag) such that the prey fusion protein is expressed on the cell surface. ECDs may be identified as described in Section 2B(i).

In some aspects in which the polypeptide comprises an extracellular domain, a tag, and an anchor, the anchor is capable of tethering the extracellular domain to the surface of a plasma membrane of a cell. In some aspects, the anchor is a glycosylphosphatidyl-inositol (GPI) polypeptide. In some aspects, the anchor is a moiety used in protein lipidation, e.g., a moiety used in cysteine palmitoylation, glycine myristoylation, lysine fatty-acylation, cholesterol esterification, cysteine prenylation, or serine fatty-acylation.

In some aspects, the tag can be directly or indirectly visualized, or otherwise detected. For example, the tag may comprise a moiety that can be detected using an antibody or an antibody fragment, e.g., may be a glycoprotein D (gD) polypeptide. In some aspects, the tag comprises a fluorescent protein.

iib. Cell Surface Interaction Assay Bait Proteins

The bait protein (query protein) or bait proteins may comprise one or more fusion proteins (e.g., a library of bait fusion proteins) in which the ECD of a bait protein of interest is conjugated to one or more additional moieties such that the bait fusion protein is soluble. The additional moiety or moieties may also increase the avidity of the bait fusion protein for the prey protein, e.g., by multimerizing the bait protein ECD. Increasing avidity may increase the detection of low-affinity interactions. In some aspects, the additional moiety causes tetramerization of the bait protein ECD.

In some aspects, the bait fusion protein comprises an Avi tag, a cleavage sequence (e.g., a TEV cleavage sequence), and an Fc tag, such that the Fc tag can be cleaved from the protein upon addition of the enzyme TEV protease. To prepare this protein for a cell surface interaction assay, the Fc tag is cleaved, the Avi tag is biotinylated, and the biotinylated bait fusion protein is conjugated to a fluorescent streptavidin (SA), e.g., a streptavidin conjugated to allophycocyanin (APC), to form a tetramerized bait fusion protein detectable in a fluorescence assay.

iic. Expression

The prey fusion protein may be expressed (e.g., transfected, e.g., transiently transfected) in a cell. The cell may be a human cell, e.g., a COS7 cell. Transfected cells may be placed in a well, e.g., a well in a 384-well plate.

The bait fusion protein may be expressed (e.g., transfected, e.g., transiently transfected) in a cell, e.g., a mammalian cell. Bait fusion proteins may be purified using standard protocols, e.g., as described in Ramani et al., Anal Biochem, 420: 127-138, 2012.

iid. Assay for Interaction

To perform the protein-protein interaction assay, a solution comprising the bait protein (e.g., the purified bait fusion protein conjugated to fluorescent SA) may be added to one or more wells containing cells expressing a prey protein (e.g., to one or more wells of a 384-well plate). The assay may then be incubated and washed one or more times to remove non-bound bait protein. The cells may then be fixed, e.g., with 4% paraformaldehyde, to preserve protein-protein interactions.

In some aspects, detecting an interaction comprises detecting a signal, e.g., a fluorescent signal, at a location on the solid surface that is above a threshold level (e.g., a signal indicating the presence of a query protein at the location, e.g., a signal from a moiety comprised by the bait fusion protein (e.g., multimerized query protein)). The signal may be directly or indirectly visualizable or otherwise detectable. In some aspects, the detecting is semi-automated or automated. The interaction may be a transient interaction and/or a low-affinity interaction, e.g., a micromolar-affinity interaction.

In aspects in which the bait fusion protein (e.g., a multimerized query protein) comprises a fluorescent SA, interaction between the bait fusion protein and the prey fusion protein may be detected by fluorescence microscopy. Relatively high fluorescence indicates that the bait fusion protein is present, i.e., that the bait fusion protein and the prey fusion protein interact.

iie. Automated Cell Surface Interaction Screen

The extracellular interaction assay may be a high-throughput assay, e.g., a screen. A screen may include between 1 and more than 1500 prey fusion proteins (e.g., 1, more than 1, more than 10, more than 100, more than 250, more than 500, more than 750, more than 1000, more than 1250, or more than 1500 prey fusion proteins) and between 1 and more than 1500 bait fusion proteins (e.g., 1, more than 1, more than 10, more than 100, more than 250, more than 500, more than 750, more than 1000, more than 1250, or more than 1500 bait fusion proteins). In some aspects, the assay uses an integrated robotic system. In some aspects, the assay is performed in one or more 384-well plates. In some aspects, assays are assessed by a computational pipeline, e.g., a custom analysis module, to determine whether interaction has occurred.

iii. Surface Plasmon Resonance (SPR) Assays

Protein-protein interactions may also be assayed using a surface plasmon resonance (SPR) assay. In some aspects, SPR assays are used to confirm or validate assays detected in an extracellular interaction assay or a cell surface interaction assay, e.g., a high-throughput extracellular interaction screen or a high-throughput cell surface interaction screen.

In some aspects, a prey protein is expressed as a fusion protein comprising the ECD of the protein conjugated to an additional moiety, e.g., an Fc tag. The prey fusion protein may be purified. The prey protein may be immobilized on a sensor chip, e.g. a GLC or CM5 sensor chip, by amine coupling.

The bait protein may be provided in a soluble form, e.g., as an ECD fused to a soluble tag. The bait fusion protein may be purified.

B. Protein, Vector, and Cell Libraries

Proteins assayed in the invention include cell surface proteins, e.g., STM receptors and IgSF proteins. Proteins may be full-length proteins (e.g., secreted proteins), one or more domains or regions of a full-length protein (e.g., an extracellular domain), or fusion proteins comprising one or more domains of a protein of interest and one or more additional polypeptide sequences. In some aspects, a protein is a fusion protein having an extracellular domain of a protein of interest, e.g., an STM receptor or an IgSF protein, and one or more additional polypeptide sequences, e.g., additional polypeptide sequences that allow the protein to be used in an assay. Proteins may be grouped into “libraries,” i.e., collections of proteins in a particular class (i.e., STM receptors or IgSF proteins) having a shared format, construction, or modification (e.g., fusion proteins comprising the ECD of the protein of interest and the same or similar additional polypeptide sequences). Particular examples of libraries are described below.

i. Extracellular Domains

In some aspects, the protein is expressed as one or more domains of the full-length protein, e.g., an extracellular domain (ECD). An ECD is a domain of a protein that is predicted to be localized outside of the plasma membrane of the cell. This domain of the protein is thus available to interact with the extracellular environment, e.g., interact with soluble proteins and ECDs of other proteins on the cell or on an adjacent cell. In some aspects, the ECD is soluble.

The ECD or ECDs of a protein may be identified by bioinformatics analysis, e.g., by analysis of UniProt annotations. For example, the boundaries of the ECD may be identified relative to the boundary of an adjacent predicted transmembrane region, e.g., a transmembrane helix. In some aspects, the presence of an extracellular domain may be predicted by the presence of a domain, sequence, or motif that indicates that the protein is trafficked to the plasma membrane, e.g., a signal sequence or a glycosylphosphatidylinositol (GPI) linkage site.

In some aspects, the extracellular domain is expressed in the context of a full-length protein. In other aspects, the extracellular domain is expressed as an isolated extracellular domain, e.g., a sequence of amino acid residues comprising only the amino acid residues of a protein that are predicted to be extracellular. In some aspects, the isolated extracellular domain is expressed in a fusion protein.

ia. ECD Fusion Proteins

In some aspects, the isolated ECD is included in a fusion protein, e.g., is expressed as part of a polypeptide chain comprising one or more other polypeptide sequences. The isolated ECD may be fused directly or indirectly to one or more moieties that confer or increase one or more desirable properties, e.g., solubility, ease of expression, ease of capture, or multimerization. In some aspects, the ECD fusion protein may be for use in an assay, e.g., extracellular interaction assay, a cell surface interaction assay, or a surface plasmon resonance assay.

In some aspects, the ECD fusion protein is a monomer. In other aspects, the ECD fusion protein is a multimer, e.g., a tetramer or a pentamer. In some aspects, the ECD is fused to a human IgG. In some aspects, the ECD is fused to a human Fc tag. In some aspects, the ECD is fused to an Avi tag. In some aspects, the ECD is fused to a polyhistidine (His) tag. In some aspects, the ECD is fused to a (GPI)-gD (gDGPI) tag. In other aspects, the ECD is fused to the pentamerization domain of rat cartilaginous oligomeric matrix protein (COMP) and the β-lactamase protein, e.g., as described in Bushell et al., Genome Res, 18: 622-630, 2008.

In some aspects, the ECD fusion protein further includes a cleavage sequence, e.g., a TEV cleavage sequence, to allow selective removal of one or more domains. In some instances, an ECD fusion protein having an Avi tag and an Fc tag cleavable at a cleavage sequence is further processed to remove the Fc tag, to biotinylate the Avi tag, and to fuse the biotinylated ECD fusion protein to a fluorescent streptavidin (SA), e.g., to form a tetramerized ECD fusion protein. In some instances, the isolated ECD or ECD fusion protein is purified.

In some aspects, a fusion protein comprises the full sequence of the protein (e.g., the full sequence of a secreted protein, e.g., a secreted IgSF protein) fused to one or more of the other polypeptide sequences described herein.

ii. STM Receptors

Single transmembrane (STM) receptor proteins are a large category of membrane-bound receptors having a single domain passing through the plasma membrane. Many STM receptors are expressed on the cell surface, and thus may participate in the extracellular interactome. Exemplary STM receptors are provided in Table 5, Table 7, and Table 8 and in Martinez-Martin et al., Cell, 174(5): 1158-1171, 2018 and Clark et al., Genome Res, 13: 2265-2270, 2003.

STM receptors included in the STM library were identified by bioinformatics analysis, e.g., by using algorithms for the prediction of protein features, e.g., protein domains and subcellular localizations. Exemplary algorithms for predicting protein domains and/or subcellular localizations include the TMHMM and SignalP Servers (University of Denmark) and Phobius (Centre Stockholm Bioinformatics).

In some aspects, the STM receptor has the UniProt annotation “leucine-rich,” “cysteine-rich,” “ITIM/ITAM” (immunoreceptor tyrosine-based inhibition motif/immunoreceptor tyrosine-based activation motif), “TNFR” (tumor necrosis factor receptor), “TLR/ILR” (Toll-like receptor/interleukin receptor), “semaphorin,” “Kinase-like,” “Ig-like” (immunoglobulin-like), “fibronectin,” “ephrin,” “EGF,” “cytokineR,” or “cadherin”.

iia. STM Receptor Library for Extracellular Interaction Assays

In some aspects, the invention features an STM receptor library for use in an extracellular interaction assay. Proteins included in this library are provided in Table 5 and are “prey” fusion proteins constructed as described in Section IIA(ia) herein.

iib. STM Receptor Library for Cell Surface Interaction Assays

In some aspects, the invention features an STM receptor library for use in a cell surface interaction assay. Proteins included in this library are provided in Table 7 and are “prey” fusion proteins constructed as described in Section IIA(iia) herein.

Polypeptide Library

In some aspects, the invention features a collection of polypeptides (e.g., a polypeptide library), wherein each polypeptide comprises an extracellular domain, a tag, and an anchor, and wherein the collection of polypeptides comprises the extracellular domains of all or a subset of the proteins of Table 7. In some aspects, the anchor is at N-terminus of the polypeptide and the extracellular domain is at the C-terminus of the polypeptide. In other aspects, the anchor is at the C-terminus of the polypeptide and the extracellular domain is at the N-terminus of the polypeptide. In some aspects, the collection of polypeptides comprises the extracellular domains of at least 81% of the proteins of Table 7.

In some aspects, the collection of polypeptides comprises the extracellular domains of at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the proteins of Table 7.

In some aspects, the collection of polypeptides comprises the extracellular domains of at least 81% to 100% of the proteins of Table 7, e.g., comprises at least 85%, 90%, 95%, or 100% of (e.g., comprise all of) the proteins of Table 7, e.g., comprises the extracellular domains of 81%-85%, 83%-87%, 85%-89%, 87%-91%, 89%-93%, 91%-95%, 93%-97%, 95%-99%, or 100% of the proteins of Table 7.

In some aspects, the collection of polypeptides comprises the extracellular domains of at least 80% to 81% of the proteins of Table 7, e.g., comprises at least 80.1%, 80.2%, 80.3%, 80.4%, 80.5%, 80.6%, 80.7%, 80.75%, 80.8%, or 80.9% of the proteins of Table 7.

In some aspects, the collection of polypeptides comprises the extracellular domains of at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or all 1195 of the proteins of Table 7, e.g., comprise the extracellular domains of 100-150, 150-200, 200-250, 250-300, 300-350, 350-400, 400-450, 450-500, 500-550, 550-600, 600-650, 650-700, 750-800, 800-850, 850-900, 900-950, 950-1000, 1000-1050, 1050-1100, 1100-1150, or all 1195 of the polypeptides of Table 7.

In some aspects, the collection of polypeptides comprises the extracellular domains of at least 965 to at least 970 of the proteins of Table 7, e.g., comprises the extracellular domains of at least 965, 966, 967, 968, 969, or 970 of the proteins of Table 7.

In some aspects, the collection of polypeptides comprises the extracellular domain of at least one of the proteins of Table 17, e.g., comprises the extracellular domains of at least 2, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or all 231 of the proteins of Table 17, e.g., comprise the extracellular domains of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 101-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 214-220, 220-225, 225-230, or all 231 of the polypeptides of Table 17.

In some aspects, the extracellular domain of the prey protein (e.g., STM protein) has a native conformation, e.g., a conformation observed in the wild-type protein. In some aspects, the extracellular domain of the prey protein (e.g., STM protein) comprises a native post-translational modification.

In some aspects, the cell is a mammalian cell, e.g., a COS7 cell.

In some aspects, the cell has been transiently transfected with a plasmid encoding the polypeptide.

Plasmid Library

In some aspects, the invention comprises a collection of vectors (e.g., plasmids), each encoding a polypeptide comprising an extracellular domain, a tag, and an anchor, wherein the collection of polypeptides encoded by the vectors comprises the extracellular domains of all or a subset of the proteins of Table 7, e.g., encoding a collection of polypeptides comprising at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the proteins of Table 7. Exemplary collections of polypeptides that may be encoded by the plasmids are described in Section IIB.

In some aspects, the collection of polypeptides encoded by the vectors comprises at least 81% of the proteins of Table 7, e.g., comprises the extracellular domains of at least 81% to 100% of the proteins of Table 7, e.g., comprises at least 85%, 90%, 95%, or 100% of (e.g., comprises all of) the proteins of Table 7, e.g., comprises the extracellular domains of 81%-85%, 83%-87%, 85%-89%, 87%-91%, 89%-93%, 91%-95%, 93%-97%, 95%-99%, or 100% of the proteins of Table 7.

In some aspects, the collection of polypeptides encoded by the vectors comprises the extracellular domains of at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or all 1195 of the proteins of Table 7.

In some aspects, the collection of polypeptides encoded by the vectors comprises at least 965 of the proteins of Table 7. In some aspects, the collection of polypeptides encoded by the vectors comprises the extracellular domains of at least 965 to at least 970 of the proteins of Table 7, e.g., comprises the extracellular domains of at least 965, 966, 967, 968, 969, or 970 of the proteins of Table 7.

In some aspects, the collection of polypeptides encoded by the vectors comprises the extracellular domain of at least one of the proteins of Table 17, e.g., comprises the extracellular domains of at least 2, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or all 231 of the proteins of Table 17, e.g., comprise the extracellular domains of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 101-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 214-220, 220-225, 225-230, or all 231 of the polypeptides of Table 7.

Cell Library

In some aspects, the invention comprises a collection of cells that have been transformed with the above-described vectors (e.g., plasmids), i.e., have each been transformed with a vector encoding a polypeptide comprising an extracellular domain, a tag, and an anchor, wherein the collection of polypeptides encoded by the vectors comprises the extracellular domains of all or a subset of the proteins of Table 7, e.g., at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the proteins of Table 7. Exemplary collections of polypeptides that may be comprised by the cells are described in Section IIB.

In some aspects, the collection of vectors comprised by the cells encodes the extracellular domains of at least 81% of the proteins of Table 7, e.g., comprises the extracellular domains of at least 81% to 100% of the proteins of Table 7, e.g., comprises at least 85%, 90%, 95%, or 100% of (e.g., comprises all of) the proteins of Table 7, e.g., comprises the extracellular domains of 81%-85%, 83%-87%, 85%-89%, 87%-91%, 89%-93%, 91%-95%, 93%-97%, 95%-99%, or 100% of the proteins of Table 7.

In some aspects, the collection of vectors comprised by the cells encodes the extracellular domains of at least of at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or all 1195 of the proteins of Table 7.

In some aspects, the collection of vectors comprised by the cells encodes at least 965 of the proteins of Table 7.

In some aspects, the collection of vectors comprised by the cells encodes the extracellular domains of at least 965 to at least 970 of the proteins of Table 7, e.g., comprises the extracellular domains of at least 965, 966, 967, 968, 969, or 970 of the proteins of Table 7.

In some aspects, the collection of vectors comprised by the cells encodes the extracellular domain of at least one of the proteins of Table 17, e.g., comprises the extracellular domains of at least 2, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or all 231 of the proteins of Table 17, e.g., comprise the extracellular domains of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 101-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 214-220, 220-225, 225-230, or all 231 of the polypeptides of Table 17.

In some aspects, each cell of the collection of cells is capable of expressing the polypeptide encoded by the vector with which it has been transformed. In some aspects, each of a plurality of cells of the collection of cells is capable of expressing the polypeptide encoded by the vector with which it has been transformed.

iic. PDPN

Podoplanin (PDPN) is an STM receptor. PDPN may be highly expressed on the surface of fibroblasts (e.g., cancer-associated fibroblasts), lymphatic endothelial cells, and type I alveolar cells. PDPN is overexpressed in many tumor tissues, and its expression in tumor tissues is associated with poor prognosis. PDPN is known to act as a master regulator of actomyosin contractility in mouse fibroblasts via interaction with the C-type lectin receptor CLEC-2 (CLEC1B) (Astarita et al., Nat Immunol, 16: 75-84, 2015; Acton et al., Nature, 514: 498-502, 2014). In some aspects, PDPN acts as a regulator of actomyosin contractility in human CAFs.

PDPN for Cell Surface Interaction Assay

In some aspects, the invention features a PDPN fusion protein for use in a cell surface interaction assay. This protein is a “bait” fusion protein constructed as described in Section IIA(iib) herein.

iii. IgSF Proteins

The immunoglobulin superfamily (IgSF) is the largest family of secreted and cell surface-expressed proteins encoded by the human genome and the most highly represented extracellular protein domain in humans. IgSF proteins are known to function through the formation of homophilic and heterophilic complexes that mediate a wide array of functionalities, e.g., modulation of axon guidance, modulation of synaptic plasticity, control of cell migration, control of cell adhesion, and self vs. non-self recognition. As such, these proteins constitute a major focus for drug development efforts. Some IgSF proteins are expressed on the cell surface (e.g., transmembrane proteins); others are secreted. Exemplary IgSF proteins are provided in Table 4 and in Ozkan et al., Cell, 154(1): 228-239, 2013.

The Immunoglobulin Superfamily (IgSF) library comprises proteins having containing at least one immunoglobulin (Ig) domain or immunoglobulin fold, having the annotation “immunoglobin-like superfamily,” e.g., in the SwissProt database, or otherwise indicated to have structural or functional similarity to such a protein. In some aspects, IgSF proteins are identified by the annotation “immunoglobulin-like domain superfamily” for the protein in the SwissProt database. In some aspects, IgSF proteins are identified based on the protein's participation in key biological activities.

In some aspects, the IgSF protein has the UniProt annotation “TFNR” (transcription factor-like nuclear regulator), “TLR/ILR (Toll-like receptor/interleukin receptor), “semaphorin,” “Kinase-like,” “IgSF/Ig-like fold,” “Ig-like fold,” “fibronectin,” “ephrin,” “EGF,” “CytokineR,” or “cadherin”.

In some aspects, the IgSF superfamily protein is Programmed death ligand 1 (PD-L1; CD274), Programmed cell death 1 ligand 2 (PD-L2; CD274; PDCD1LG2), Receptor-type tyrosine-protein phosphatase delta (PTPRD), Receptor-type tyrosine-protein phosphatase S (PTPRS), Receptor-type tyrosine-protein phosphatase F (PTPRF), Neural cell adhesion molecule L1-like protein (“Close homolog of L1”; CHL1), Contactin 1 (CNTN1), Leukocyte immunoglobulin-like receptor subfamily B member 1 (LILRB1), Leukocyte immunoglobulin-like receptor subfamily B member 2 (LILRB2), Leukocyte immunoglobulin-like receptor subfamily B member 3 (LILRB3), Leukocyte immunoglobulin-like receptor subfamily B member 4 (LILRB4), Leukocyte immunoglobulin-like receptor subfamily B member 5 (LILRB5), MAM domain-containing glycosylphosphatidylinositol anchor protein 1 (MDGA1), or Tyrosine-protein kinase receptor AXL.

iiia. IgSF Library for Extracellular Interaction Assay

In some aspects, the invention features an IgSF receptor library for use in an extracellular interaction assay. Proteins included in this library are provided in Table 4 and are “bait” fusion proteins constructed as described in Section IIA(ib) herein.

iiib. IgSF Proteins for Cell Surface Interaction Assay

In some aspects, the invention features IgSF receptors for use in a cell surface interaction assay. Proteins included in this library are provided in Table 4 and are “bait” fusion proteins constructed as described in Section IIA(iib) herein.

iv. Bait Protein and Prey Protein Libraries

In some aspects, the invention features a bait protein library comprising the proteins provided in Table 1. Proteins may be constructed as “bait” fusion proteins described in Section IIA(ib) or Section IIA(iib) herein.

In some aspects, the invention features a prey protein library comprising the proteins provided in Table 2. Proteins may be constructed as “prey” fusion proteins described in Section IIA(ia) or Section IIA(iia) herein.

C. Protein-Protein Interaction Networks

In some aspects, interactions between bait proteins (Table 1) and prey proteins (Table 2) may be tested using extracellular interaction assays (including a high-throughput extracellular interaction screen), cell surface assays (including a high-throughput cell surface interaction screen), and surface plasmon resonance assays as described in Section IIA. In some aspects, the assay includes between 1 and more than 1500 prey proteins (e.g., 1, more than 1, more than 10, more than 100, more than 250, more than 500, more than 750, more than 1000, more than 1250, or more than 1500 prey proteins) and between 1 and more than 1500 bait proteins (e.g., 1, more than 1, more than 10, more than 100, more than 250, more than 500, more than 750, more than 1000, more than 1250, or more than 1500 bait proteins. In some aspects, the assay identifies between 1 and 900 protein-protein interactions (e.g. 1, more than 1, more than 10, more than 100, more than 250, more than 500, more than 750, more than 850, or 900 protein-protein interactions. In some aspects, these analyses identify previously unrecognized communities of functionally related proteins, uncover binding partners for orphan proteins, and reveal receptor-ligand interactions prominently deregulated in cancer.

i. STM Receptor—IgSF Interactome

In some aspects, the IgSF proteins provided in Table 4 and the STM proteins provided in Table 5 are tested for interaction in a high-throughput extracellular interaction screen, as described in Section IIA(ie) herein. In some aspects, the IgSF proteins are constructed as a bait library (as in Section IIB(iiia)), and the STM proteins are constructed as a prey library (as in Section IIB(iia)). In some aspects, the detected interactions are assembled into a network. In some aspects, selected interactions are further tested, e.g., in a cell surface interaction screen (described in Section IIa(ii)) or an SPR assay (described in Section IIa(iii)).

ia. Proteins Interacting with PD-L1 and/or PD-L2

The IgSF proteins PD-L1 (CD274) and PD-L2 (PDCD1LG2) are immune checkpoint proteins and play key roles in immunosuppressive functions that may result in tumor immune escape (Chen and Mellman, Immunity, 39: 1-10, 2013). Accordingly, therapies targeting PD-L1 have been a major focus of research. Antibody blockade of PD-L1 is a preferred immunotherapeutic strategy for the treatment of solid tumors; however, many patients do not respond or do not display durable responses to antibody blockade of PD-L1, indicating that therapies targeting other immunosuppressive pathways are needed.

In some aspects, the assays described herein may identify an interaction between PD-L1 and ephrin type-A receptor 3 (EPHA3). EPHA3 is a receptor tyrosine kinase activated by the binding of an ephrin protein and having roles in signal transduction and the control of multiple cellular processes, e.g., cell growth, migration, and adhesion (Lisabeth et al., Cold Spring Harb Perspect Biol, 5, 2013). EPHA3 has also been identified as one of the most frequently mutated genes in certain tumors (Andretta et al., Sci Rep, 7: 41576, 2017). The downstream effects of the interaction between PD-L1 and EPHA3 may therefore include modulation of immune checkpoint function, e.g., immunosuppression, and/or modulation of EPHA3 kinase functions.

In some aspects, the assays described herein may identify an interaction between PD-L2 and CEACAM4. The CEACAM family has been shown to have a role in regulation of the immune system: CEACAM1 has been identified as a ligand for the inhibitory receptor TIM-3 (Huang et al., Nature, 517: 386-390, 2002). The interaction between CEACAM4 and PD-L2 may contribute to PD-1-independent functions of PD-L2, e.g., functions described in Liu et al., J Exp Med, 197: 1721-1730, 2003, e.g., phagocytosis. The downstream effects of the interaction between PD-L2 and CEACAM4 may therefore include modulation of immune checkpoint function, e.g., immunosuppression (Delgado Tascon et al., J Leukoc Biol, 97: 521-531, 2015; Xiao et al., J Exp Med, 211: 943-959, 2014).

In some aspects, the assay or assays described herein may identify interactions between PD-L2 and CEACAM4; PD-L2 and ICAM5; PD-L2 and NECTIN3; PD-L2 and PSG9; and PD-L2 and TNFRSF11A.

ib. Proteins Interacting with PTPR Proteins

The PTPR proteins PTPRD, PTPRS, and PTPRF are receptor-type tyrosine-protein phosphatases. Tyrosine phosphorylation and dephosphorylation regulate a multitude of cellular processes, and aberrations in tyrosine phosphorylation/dephosphorylation are associated with tumor formation. In particular, PTPRD and PTPRS have been described as key modulators of nervous system processes, e.g., synapse formation and axonal growth, and have also been identified as tumor suppressors having a high mutation rate in neuroblastoma, glioma, colon cancer, and breast cancer (Veeriah et al., Proc Natl Acad Sci USA, 106: 9435-9440, 2009; Wang et al., Hepatology, 62:1201-1214, 2015).

In some aspects, the assays described herein may identify interactions between PTPRS, PTPRD, and/or PTPRF and members of the SLIT and NTRK-like (SLITRK) family (e.g., SLITRK1, SLITRK2, SLITRK3, SLITRK4, and SLITRK6), and may also identify interactions between PTPRS, PTPRF, and/or PTPRD and members of the Leucine-rich repeat and fibronectin type III domain-containing protein (LRFN) family, Interleukin-1 receptor accessory protein (IL1RAP), and the related proteins IL1RAPL1 and IL1RAPL2. SLITRKs, LRFNs, and IL1RAP-like proteins have been implicated in nervous system disorders including tumors. For example, IL1RAP is required for IL-1 signaling as has been reported to be a biomarker for chronic myeloid leukemia stem cells (Zhao et al., Int J Clin Exp Med, 7: 4787-4798, 2014). The downstream effects of the identified interactions may therefore include changes in phosphatase activity, e.g., changes in tyrosine phosphorylation/dephosphorylation, e.g., tyrosine phosphorylation/dephosphorylation of proteins implicated in diseases including cancer.

In some aspects, the assay or assays may identify interactions between PTPRD and BMP5, CEACAM3, IL1RAP, IL1RAPL2, LECT1, LRFN5, SIRPG, SLITRK3, SLITRK4, SLITRK6, and TGFA. PTPRD has been associated with suppression of cell proliferation and STAT3 phosphorylation (Veeriah et al., PNAS, 106(23): 9435-9440, 2009; Peyser et al., PLoS ONE, 10.1371/journal.pone.0135750, 2015).

In some aspects, the assay or assays may identify interactions between PTPRS and C6orf25, IL1RAP, IL1RAPL1, IL1RAPL2, LRFN1, LRFN5, LRRC4B, NCAM1, SLITRK1, SLITRK2, SLITRK3, SLITRK4, and SLITRK6. PTPRS has been associated with inhibition of cell migration (Wang et al., Hepatology, 62(4): 1201-1214, 2015) and with activation of the PI3K signaling pathway (Suarez Pestana et al., Oncogene, 18: 4069-4079, 1999; Morris et al., PNAS, 108(47): 19024-19029, 2011).

In some aspects, the assay or assays may identify interactions between PTPRF and CD177, IL1RAP, and LRFN5. PTPRF has been associated with inhibition of cell migration and phosphorylation of EGFR (Du et al., J Cell Sci, 126: 1440-1453, 2013).

Proteins Interacting with PTPRD Mutants

In some aspects, the assay or assays may identify interactions between forms of PTPRD having one or more disease-relevant (e.g., cancer-relevant) mutations (PTPRD mutants), e.g., an amino acid substitution mutation or an amino acid deletion mutation, and IL1RAP, IL1RAPL1, IL1RAPL2, LRFN4, LRFN5, LRRC4B, NTRK3, SLITRK1, SLITRK3, or SLITRK6. Disease-relevant variations in PTPRD (Table 9) occur in the immunoglobulin (IG) domains IG1, IG2, and IG3 and in the fibronectin (FN) domains FN1-FN8 of the PTPRD ECD (FIG. 6F). Specific amino acid substitution or deletion mutations include ΔG61 ΔE106 (IG1), G203E K204E (IG2), R232C R233C (IG2), P249L (IG3), G285E (IG3), E406K (FN1), S431L (FN2), R561Q (FN3), P666S (FN4), E755K (FN5), V892I (FN6), S912F (FN7), R995C (FN7), and R1088C (FN8). In some aspects, the PTPRD variant is expressed as a bait protein as described in Section IIA(ib) herein and assayed for interaction with a prey protein in an extracellular interaction assay as described in Section IIA(i) herein. In some aspects, the strength of the interaction between a PTPRD variant and a binding partner may be decreased relative to the strength of an interaction between a wild-type PTPRD protein and the binding partner. In other aspects, the strength of the interaction may be similar for the variant and wild-type PTPRD proteins.

ic. Proteins Interacting with CHL1 and/or CNTN1

Neural cell adhesion molecule L1-like protein (CHL1) and Contactin 1 (CNTN1) are IgSF proteins that act as cell adhesion molecules (CAMs). Overexpression of CAMs is associated with poor prognosis in patents with cancers (Yan et al., Cancer Res., 76(6), 1603-1614, 2016).

CHL1 is involved in neural cell adhesion and has roles in central nervous system (CNC) development. CHL1 has been associated with suppression of cell proliferation (Tang et al., Oncogene, doi: 10.1038/s41388-018-0648-7); suppression of tumor formation (Tang et al., Oncogene, doi: 10.1038/s41388-018-0648-7); and suppression of cell invasion (He et al., Biochem Biophys Res Commun, 438: 433-438, 2013).

In some aspects, the assays described herein may identify interactions between CHL1 and contactin 5 (CNTN5), L1 cell adhesion molecule (L1CAM), two proteins involved in cell adhesion, and B- and T-lymphocyte attenuator (BTLA).

Upregulation of CNTN1 is strongly associated with prostate cancer cell invasion. CNTN1 has been associated with suppression of cell proliferation; suppression of cell invasion (Yan et al., Cancer Res, 76(6): 1603-1614, 2016) and activation of the RhoA and AKT signaling pathways (Yan et al, PLoS ONE, 8(5): e65463, 2013; Chen et al., Front Mol Neurosci, 11, Article 422 (2018); Su et al., Cancer Res, 66(5): 2553-2561, 2006).

In some aspects, the assay or assays may identify interactions between CHL1 and SIRPA, CNTN1, CNTN5, L1CAM, and TMEM132A.

In some aspects, the assay or assays may identify an interaction between CNTN1 and CDH6, CHL1, FCGRT, PCDHB7, and SGCG. The downstream effects of the identified interactions may include modulation of cell adhesion, e.g., neural cell adhesion.

id. Proteins Interacting with LILRB Proteins

The leukocyte immunoglobulin-like receptor B proteins (LILRBs) are IgSF proteins characterized by the presence of cytosolic immunoreceptor tyrosine-based activating motifs (ITAM) or immunoreceptor tyrosine-based inhibitory motifs (ITIM) (Brown et al., Tissue Antigens, 64: 215-225, 2004). LILR proteins may be activating or inhibitory receptors, are mainly expressed in myeloid cells and lymphocytes, and have been associated with roles in cancer, autoimmune disease, infectious disease, and macrophage phagocytosis.

In some aspects, the assays described herein may identify interactions between LILRB5 and low-density lipoprotein receptor (LDLR). LDLR mediates the endocytosis of low-density lipoprotein (LDL) and has been implicated in diseases including familial hypercholesterolemia and coronary artery disease.

The assays described herein may also identify interactions between identify interactions between LILRB1 and CLEC6A, CXADR, EDAR, FLT4, IL6R, ILDR1, and LILRA5. LILRB1 is a component of the MHC class 1-LILRB1 signaling axis, which has been shown to protect cells (e.g., tumor cells) from phagocytosis (Barkal et al., Nature Immunol, 19: 76-84, 2017). The downstream effects of the identified interactions may therefore include modulation of phagocytosis (Section IIIB(vii)). Downregulation of interactions involving LILRB1 may lead to increased phagocytosis, e.g., increased phagocytosis of tumor cells. LILRB1 has also been associated with osteoclast differentiation (Mori et al., J Immunol, 181(7): 4742-4751, 2008).

The assays described herein may also identify interactions between LILRB2 and IGSF8 and MOG. LILRB2 has been associated with M2 macrophage polarization (Chen et al., J Clin Invest, 128(12), 5647-5662).

The assays described herein may also identify interactions between LILRB3 and LRRC15 and LY6G6F. LILRB3 has been associated with osteoclast differentiation (Mori et al., J Immunol, 181(7): 4742-4751, 2008).

The assays described herein may also identify interactions between LILRB4 and CNTFR. LILRB4 has been associated with osteoclast differentiation (Mori et al., J Immunol, 181(7): 4742-4751, 2008).

In some aspects, the assay or assays may identify interactions between LILRB5 and APLP2, CD177, CLEC10A, LDLR, PILRA, and UNC5C.

ie. Proteins Interacting with MDGA1

MAM domain containing glycosylphosphatidylinositol anchor 1 (MDGA1) is in IgSF protein that is expressed in the nervous system. In some aspects, the assay or assays described herein may identify interactions between MDGA1 and NLGN3 and NLGN4X.

if. Proteins Interacting with AXL

The tyrosine-protein kinase receptor AXL is an IgSF protein that is an inhibitor of the innate immune response. Overexpression of AXL is associated with numerous cancers (e.g., colon cancer, esophageal cancer, thyroid cancer, breast cancer, lung cancer, liver cancer, and astrocytoma-glioblastoma (Verma et al., Mol Cancer Ther, 10(10): 1763-1773, 2011).

AXL has been associated with regulation of cell invasion (Verma et al., Mol Cancer Ther, 10(10): 1763-1773, 2011); regulation of the JAK/STAT pathway, regulation of the RAS/RAF/MAPK/ERK1/2 pathway, and PI3K signaling pathways (Verma et al., Mol Cancer Ther, 10(10): 1763-1773, 2011); changes in cell motility and morphology, e.g., formation of filopodia (Verma et al., Mol Cancer Ther, 10(10): 1763-1773, 2011); and regulation of the epithelial-mesenchymal transition (EMT) (Gjerdrum et al., PNAS, 107(3): 1124-1129, 2010; Divine et al.; Oncotarget, 7: 77291-77305, 2016; Rankin et al., Cancer Res, 70(19), 7570-7579, 2010; Chichon et al., Oncogene, 33: 4185-4192, 2013).

In some aspects, the assay or assays described herein may identify interactions between AXL and IL1RL1 and VSIG10L.

ig. Proteins Interacting with LRRC4B

In some aspects, the assay or assays described herein may identify interactions between LRRC4B and BTN3A1 or BTN3A3.

ii. Interactions Between STM Receptors and PDPN

In some aspects, the STM proteins provided in Table 7 may be tested for interaction with PDPN in a high-throughput cell surface interaction screen, as described in Section IIA(iie) herein. PDPN may be constructed as a bait protein (as in Section IIB(iic)), and the STM proteins may be constructed as a prey library (as in Section IIB(iib)). In some aspects, selected interactions may be further tested with SPR assays (described in Section IIa(iii)).

Podoplanin (PDPN) is an STM receptor that acts as a regulator of actomyosin contractility in CAFs. In some aspects, the assays described herein may identify interactions between PDPN and cluster of differentiation 177 (CD177), a neutrophil receptor that has recently been identified as a marker of tumor-resident Tregs (Plitas et al., Immunity, 45: 1122-1134, 2016). In some aspects, the assay or assays may identify an interaction between PDPN and CD177, CLEC-2 (CLEC1B), and SIGLEC7.

In some aspects, the downstream effects of interaction with PDPN (e.g., interaction of CLEC-2 and/or CD177 with PDPN) may include increased fibroblast elongation (e.g., increased CAF elongation) and decreased fibroblast contractility (e.g., decreased CAF contractility) (Section IIIB(i)).

### III. Methods of Identifying a Modulator of a Protein-Protein Interaction

A. Assays for Modulation of Interaction

In some aspects, the invention features identifying a modulator of the interaction between a protein of Table 1 and a protein of Table 2, the method comprising: (a) providing a candidate modulator (e.g., a candidate modulator described in Section IV herein); (b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

In some aspects, the candidate modulator is provided to a cell (e.g., a mammalian cell), to cell culture media, to conditioned media, and/or to a purified form of a protein of Table 1 and/or a protein of Table 2. In some aspects, the candidate modulator is provided at a concentration of at least 0.1 nM, 0.5 nM, 1 nM, 10 nM, 50 nM, 100 nM, 250 nM, 500 nM, 750 nM, 1 μM, 2 μM, 3 μM, 5 μM, or 10 μM. In some aspects, the candidate modulator is provided at a concentration of between 0.1 nM and 10 μM. In some aspects, the candidate modulator is provided in a solution, e.g., in a soluble form.

In some aspects, the candidate modulator is identified as a modulator if the increase in binding is at least 70%. In some aspects, the increase in binding is at least 5%, at least 10%, at least 20%, at least 30%, at least 40%, at least 50%, at least 60%, at least 70%, at least 80%, at least 90%, at least 100%, or more than 100%. In some aspects, the increase in binding is at least 70%.

In some aspects, the candidate modulator is identified as a modulator if the decrease in binding is at least 70%. In some aspects, the decrease in binding is at least 5%, at least 10%, at least 20%, at least 30%, at least 40%, at least 50%, at least 60%, at least 70%, at least 80%, at least 90%, or 100%. In some aspects, the decrease in binding is at least 70%.

i. Assays for Modulation of Protein-Protein Interaction

In some aspects, the binding of the protein of Table 1 and the protein of Table 2 in the presence or absence of the candidate modulator is assessed in an assay for protein-protein interaction. Modulation of the interaction between the protein of Table 1 and the protein of Table 2 may be identified as an increase in protein-protein interaction in the presence of the modulator compared to protein-protein interaction in the absence of the modulator, e.g., an increase of 5%, 10%, 15%, 20%, 25%, 30%, 35%, 40%, 45%, 50%, 55%, 60%, 65%, 70%, 80%, 90%, 95%, 100%, or more than 100% in protein-protein interaction. Alternatively, modulation may be identified as a decrease in protein-protein interaction in the presence of the modulator compared to protein-protein interaction in the absence of the modulator, e.g., an decrease of 5%, 10%, 15%, 20%, 25%, 30%, 35%, 40%, 45%, 50%, 55%, 60%, 65%, 70%, 80%, 90%, 95%, or 100% in protein-protein interaction. The assay for protein-protein interaction may be, e.g., an SPR assay, a biolayer interferometry (BLI) assay, an enzyme-linked immunosorbent assay (ELISA), an extracellular interaction assay as described in Section IIAi, or a cell surface interaction assay as described in Section IIAii.

ia. SPR Assays for Modulation of Protein-Protein Interaction

In some aspects, the assay for protein-protein interaction is a surface plasmon resonance (SPR) assay, as described in Section IIAiii herein. In some aspects, modulation of the binding of the protein of Table 1 to the protein of Table 2 is measured as a difference in SPR signal response units (RU) in the presence compared to the absence of the modulator.

ib. BLI Assays for Modulation of Protein-Protein Interaction

In some aspects, the assay for protein-protein interaction is a BLI assay. In some aspects, the BLI assay is performed using isolated ECDs, e.g., isolated ECDs as described in Section IIB(i) herein. In some aspects, modulation of the binding of the protein of Table 1 to the protein of Table 2 is measured as a difference in wavelength shift (Δλ) measured at a biosensor tip in the presence compared to the absence of the modulator.

ic. ELISA for Modulation of Protein-Protein Interaction

In some aspects, the assay for protein-protein interaction is an ELISA. In some aspects, a first protein is bound to a plate (e.g., directly bound to a plate or bound to a plate via an affinity tag recognized by an antibody bound to a plate) and a second protein is provided in a soluble form, e.g., as an isolated ECD as described in Section IIB(i) herein. An interaction between the first protein and the second protein may be detected by providing an antibody that binds to the second protein or to an affinity tag thereof, wherein the antibody can be detected, e.g., visualized, in an assay for presence of the antibody.

id. Other Assays for Modulation of Protein-Protein Interaction

In some aspects, the assay is an extracellular interaction assay, as described in Section IIAi herein. In some aspects, the assay is a cell surface interaction assay, as described in Section IIAii herein. In some aspects, the assay is an isothermal titration calorimetry (ITC) assay, an assay comprising immunoprecipitation, or an assay comprising an ALPHASCREEN™ technology.

B. Assays for Changes in Downstream Activity

In some aspects, the invention features a method of identifying a modulator of a downstream activity of a protein of Table 1, the method comprising: (a) providing a candidate modulator (e.g., a candidate modulator described in Section IV herein); (b) contacting the protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and (c) measuring a downstream activity of the protein of Table 1 (e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia), wherein a change in the downstream activity in the presence of the candidate modulator relative to the downstream activity in the absence of the candidate modulator identifies the candidate modulator as a modulator of the downstream activity of the protein of Table 1.

In some aspects, the invention features a method of identifying a modulator of a downstream activity of a protein of Table 2, the method comprising: (a) providing a candidate modulator (e.g., a candidate modulator described in Section IV herein); (b) contacting the protein of Table 2 with a protein of Table 1 in the presence or absence of the candidate modulator under conditions permitting the binding of the protein of Table 2 to the protein of Table 1, wherein the protein of Table 1 and the protein of Table 2 are reported to interact in Table 3; and (c) measuring a downstream activity (e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia) of the protein of Table 2, wherein a change in the downstream activity in the presence of the candidate modulator relative to the downstream activity in the absence of the candidate modulator identifies the candidate modulator as a modulator of the downstream activity of the protein of Table 2.

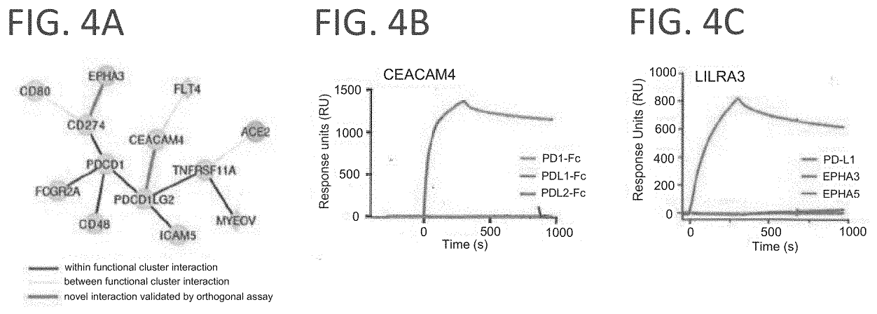

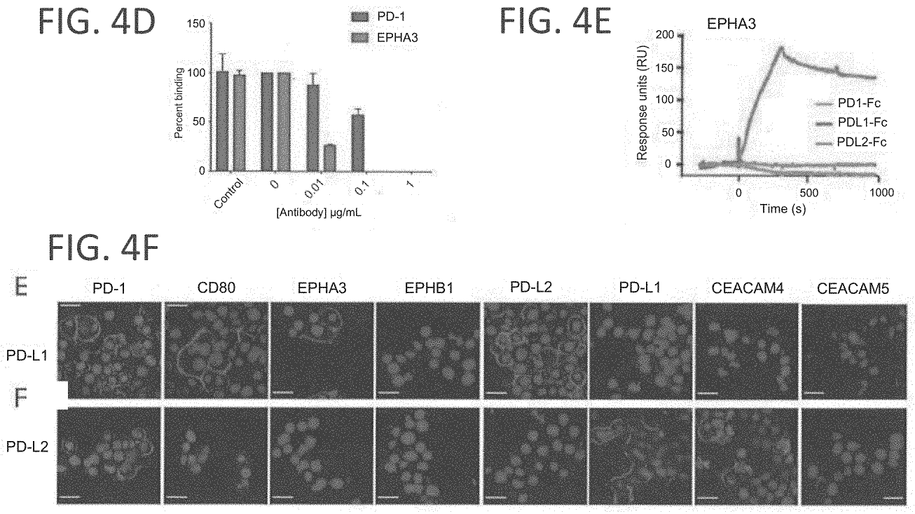

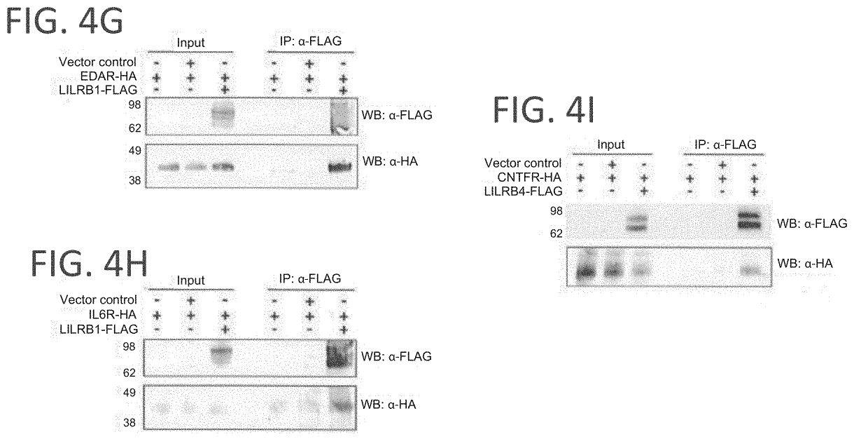

In some aspects, the candidate modulator is provided at a concentration of at least 0.1 nM, 0.5 nM, 1 nM, 10 nM, 50 nM, 100 nM, 250 nM, 500 nM, 750 nM, 1 μM, 2 μM, 3 μM, 5 μM, or 10 μM. In some aspects, the candidate modulator is provided at a concentration of between 0.1 nM and 10 μM. In some aspects, the candidate modulator is provided at a range of concentrations, e.g., as in FIG. 4F. In some aspects, the candidate modulator is provided is provided in a solution, e.g., in a soluble form. In some aspects, the candidate modulator is provided to an organism comprising the protein of Table 1 and the protein of Table 2, to a tissue comprising the protein of Table 1 and the protein of Table 2, to a cell (e.g., a mammalian cell), to cell culture media, to conditioned media, and/or to a purified form of a protein of Table 1 and/or a protein of Table 2.

In some aspects, the candidate modulator is identified as a modulator if the increase in the downstream activity of the protein of Table 1 or the protein of Table 2 is at least 30%. In some aspects, the increase in the downstream activity is at least 5%, at least 10%, at least 20%, at least 30%, at least 40%, at least 50%, at least 60%, at least 70%, at least 80%, at least 90%, at least 100%, or more than 100%. In some aspects, the increase in the downstream activity is at least 30%.

In some aspects, the candidate modulator is identified as a modulator if the decrease in the downstream activity of the protein of Table 1 or the protein of Table 2 is at least 30%. In some aspects, the decrease in the downstream activity is at least 5%, at least 10%, at least 20%, at least 30%, at least 40%, at least 50%, at least 60%, at least 70%, at least 80%, at least 90%, or 100%. In some aspects, the decrease in downstream activity is at least 30%.

In some aspects, the downstream activity of the protein of Table 1 or the protein of Table 2 is assessed in one or more assays, as described below.

In some aspects, the downstream activity is an activity relating to the development or progression of a disease, e.g., a cancer.

i. CAF Contractility

In some aspects, the downstream activity is cancer-associated fibroblast (CAF) actomyosin contractility (CAF contractility). In some aspects, the protein of Table 1 is PDPN and the downstream activity is CAF contractility. In some aspects, the protein of Table 1 is PDPN, the protein of Table 2 is CD177, and the downstream activity is CAF contractility.

In some aspects, the assay for CAF contractility is a gel contraction assay. In this assay, a population of fibroblast cells (e.g., cancer-associated fibroblast cells) (e.g., 100,000 cells) comprising at least one of the protein of Table 1 and the protein of Table 2 are mixed into a Matrigel-collagen mixture and placed into a well, e.g., a well in a 96-well plate. In aspects wherein the individual cell does not comprise both the protein of Table 1 and the protein of Table 2, the protein not comprised by the cell may be provided on another cell (e.g., a mammalian cell, e.g., a neutrophil or a T cell) or may be provided in or added to the Matrigel-collagen media or the cell culture media. The cell may additionally be treated with the modulator, e.g., by addition to the Matrigel-collagen media or the cell culture media. After the gels set for 20 minutes, the gel is detached from the sides of the well and cell culture media is added. Gels are incubated, e.g., for 72 hours, before imaging. Contractility of the population of cells is measured by comparing the well diameter and the final gel diameter. Increased or decreased contractility is calculated by comparing the gel contraction of cells to which the modulator is provided with that of cells to which the modulator has not been provided. In some instances, the population of cells that is treated with the modulator has decreased gel contraction relative to a population of cells that has not been treated with the modulator, e.g., CAF contractility is decreased in the presence of the modulator. In some aspects, CAF contractility is decreased by at least 10%, at least 20%, at least 30%, at least 40%, at least 50%, at least 60%, at least 70%, at least 80%, at least 90%, or 100% in the presence of the modulator as measured in a gel contraction assay. In some aspects, CAF contractility is decreased by at least 30% in the presence of the modulator as measured in a gel contraction assay. In some aspects, CAF contractility is decreased by at least 10%, at least 20%, at least 30%, at least 40%, at least 50%, at least 60%, at least 70%, or at least 80% as measured in a gel contraction assay.

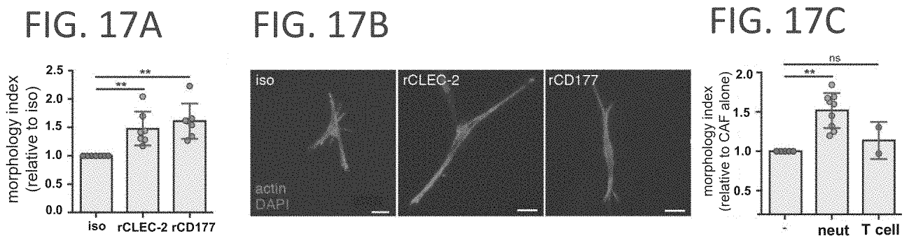

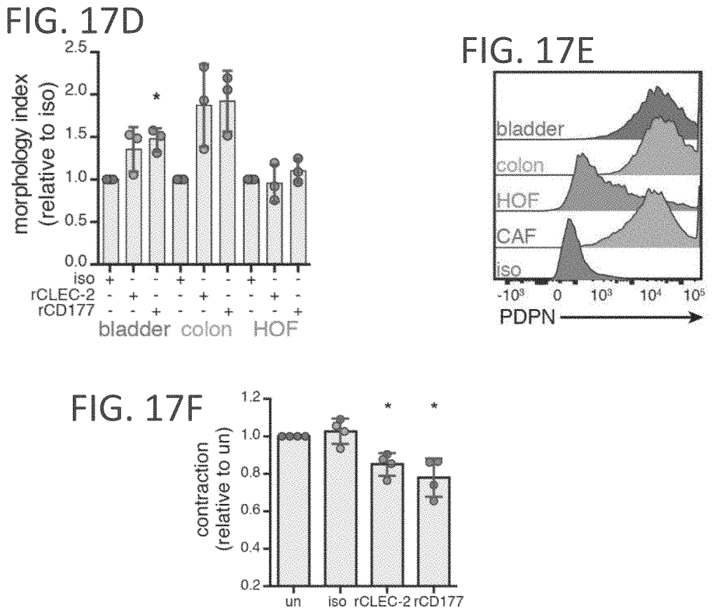

In other aspects, CAF contractility is measured using a 3D gel elongation assay, e.g., an assay as described in Example 8B. In this assay, a fibroblast cell, (e.g., a CAF) comprises at least one of the protein of Table 1 and the protein of Table 2. In aspects wherein the cell does not comprise both the protein of Table 1 and the protein of Table 2, the protein not comprised by the cell may be provided on another cell (e.g., a mammalian cell, e.g., a neutrophil or a T cell) or may be provided in or added to the cell culture media. The cell may additionally be treated with the modulator, e.g., by addition to the cell culture media. Decreased contractility of the fibroblast cell is indicated by increased elongation of the cell in the 3D gel relative to an isotype control (FIGS. 17A-17D). In some instances, the cell that is treated with the modulator is elongated relative to a control cell, e.g., CAF contractility is decreased in the presence of the modulator. In some aspects, CAF contractility is decreased by at least 10%, at least 20%, at least 30%, at least 40%, at least 50%, at least 60%, at least 70%, at least 80%, at least 90%, or 100% in the presence of the modulator as measured in a 3D gel elongation assay. In some aspects, CAF contractility is decreased by at least 30% in the presence of the modulator as measured in a 3D gel elongation assay. Morphology index may be calculated as in Astarita et al., Nat Immunol, 16: 75-84, 2015. In some aspects, the morphology index is calculated using the equation perimeter²/4π×area, wherein “perimeter” is the perimeter of the cell and “area” is the area of the cell. In some aspects, the morphology index of the cell is between 10 and 30, e.g., is 10, 11, 12, 13, 14, 15, 16, 17, 18, 19, 20, 21, 22, 23, 24, 25, 26, 27, 28, 29, or 30.

ii. Immune Checkpoint Inhibition

In some aspects, the downstream activity is immune checkpoint inhibition. In some aspects, the protein of Table 1 is PD-L1 and the downstream activity is immune checkpoint inhibition. In some aspects, the protein of Table 1 is PD-L1, the protein of Table 2 is EPHA3, and the downstream activity is immune checkpoint inhibition.

In other aspects, the protein of Table 1 is PD-L2 and the downstream activity is immune checkpoint inhibition. In some aspects, the protein of Table 1 is PD-L2, the protein of Table 2 is CEACAM4, ICAM5, NECTIN3, PSG9, or TNFRSF11A, and the downstream activity is immune checkpoint inhibition.

In some aspects, the assay for immune checkpoint inhibition is a cell-based assay, e.g., a cell-based assay as described in Skalniak et al., Oncotarget, 8: 72167-72181, 2017. In some aspects, the assay for immune checkpoint inhibition is an assay described in Mariathasan et al., Nature, 554: 544-548. In some aspects, the cells assayed are additionally treated with the modulator, e.g., by addition to the cell culture media. In some aspects, immune checkpoint inhibition is increased by at least 10%, at least 20%, at least 30%, at least 40%, at least 50%, at least 60%, at least 70%, at least 80%, at least 90%, or 100% in the presence of the modulator. In some aspects, immune checkpoint inhibition is increased by at least 30% in the presence of the modulator.

iii. Suppression of Cell Proliferation

In some aspects, the downstream activity is suppression of cell proliferation. In some aspects, the protein of Table 1 is PTPRD and the downstream activity is suppression of cell proliferation. In some aspects, the protein of Table 1 is PTPRD, the protein of Table 2 is BMP5, CEACAM3, IL1RAP, IL1RAPL2, LECT1, LRFN5, SIRPG, SLITRK3, SLITRK4, SLITRK6, or TGFA, and the downstream activity is suppression of cell proliferation. In some aspects, the PTPRD protein comprises a G203E and K204E; R232C and R233C; P249L; G285E; E406K; S431L; R561Q; P666S; E755K; V892I; S912F; R995C; or R1088C amino acid substitution mutation or a ΔG61 ΔE106 amino acid deletion mutation and the downstream activity is suppression of cell proliferation.

In other aspects, the protein of Table 1 is CNTN1 and the downstream activity is suppression of cell proliferation. In some aspects, the protein of Table 1 is CNTN1, the protein of Table 2 is CDH6, CHL1, FCGRT, PCDHB7, or SGCG, and the downstream activity is suppression of cell proliferation.

In yet other aspects, the protein of Table 1 is CHL1 and the downstream activity is suppression of cell proliferation. In some aspects, the protein of Table 1 is CHL1, the protein of Table 2 is CNTN1, CNTN5, SIRPA, L1CAM, or TMEM132A, and the downstream activity is suppression of cell proliferation.

In some aspects, the assay for suppression of cell proliferation is a colony formation assay, e.g., a colony formation assay as described in Yan et al., Cancer Res, 76(6): 1603-1614, 2016 or Ognibene et al., Oncotarget, 9(40): 25903-25921, 2018. In some aspects, the cells assayed in the colony formation assay are additionally treated with the modulator, e.g., by addition to the cell culture media. In some aspects, cell proliferation is decreased by at least 10%, at least 20%, at least 30%, at least 40%, at least 50%, at least 60%, at least 70%, at least 80%, at least 90%, or 100% in the presence of the modulator as measured in a colony formation assay. In some aspects, cell proliferation is decreased by at least 30% in the presence of the modulator as measured in a colony formation assay.

In some aspects, the assay for suppression of cell proliferation is a cell proliferation assay, e.g., a cell proliferation assay as described in Yan et al., Cancer Res, 76(6): 1603-1614, 2016. In some aspects, the cells assayed in the cell proliferation assay are additionally treated with the modulator, e.g., by addition to the cell culture media. In some aspects, cell proliferation is decreased by at least 10%, at least 20%, at least 30%, at least 40%, at least 50%, at least 60%, at least 70%, at least 80%, at least 90%, or 100% in the presence of the modulator as measured in a cell proliferation assay. In some aspects, cell proliferation is decreased by at least 30% in the presence of the modulator as measured in a cell proliferation assay.

iv. Modulation of Target Phosphorylation

In some aspects, the downstream activity is phosphorylation of or suppression of phosphorylation of a target protein, e.g., phosphorylation of EGFR or suppression of STAT3 phosphorylation. In some aspects, the protein of Table 1 is PTPRD and the downstream activity is suppression of STAT3 phosphorylation. In some aspects, the protein of Table 1 is PTPRD, the protein of Table 2 is BMP5, CEACAM3, IL1RAP, IL1RAPL2, LECT1, LRFN5, SIRPG, SLITRK3, SLITRK4, SLITRK6, or TGFA, and the downstream activity is suppression of STAT3 phosphorylation. In some aspects, the PTPRD protein comprises a G203E and K204E; R232C and R233C; P249L; G285E; E406K; S431L; R561Q; P666S; E755K; V892I; S912F; R995C; or R1088C amino acid substitution mutation or a ΔG61 ΔE106 amino acid deletion mutation and the downstream activity is suppression of STAT3 phosphorylation.

In some aspects, the assay for suppression of STAT3 phosphorylation is a Western blot for phosphorylated STAT3, e.g., a Western blot as described in Veeriah et al., PNAS, 106(23): 9435-9440, 2009 or Peyser et al., PLoS ONE, 10.1371/journal.pone.0135750, 2015. In some aspects, the cell assayed in the Western blot is additionally treated with the modulator, e.g., by addition to the cell culture media. In some aspects, suppression of STAT3 phosphorylation is increased by at least 10%, at least 20%, at least 30%, at least 40%, at least 50%, at least 60%, at least 70%, at least 80%, at least 90%, or 100% in the presence of the modulator as measured in a Western blot for phosphorylated STAT3. In some aspects, suppression of STAT3 phosphorylation is increased by at least 30% in the presence of the modulator as measured in a Western blot for phosphorylated STAT3.

In some aspects, suppression of STAT3 phosphorylation is decreased by at least 10%, at least 20%, at least 30%, at least 40%, at least 50%, at least 60%, at least 70%, at least 80%, at least 90%, or 100% in the presence of the modulator as measured in a Western blot for phosphorylated STAT3. In some aspects, suppression of STAT3 phosphorylation is decreased by at least 30% in the presence of the modulator as measured in a Western blot for phosphorylated STAT3.

In other aspects, the protein of Table 1 is PTPRF and the downstream activity is phosphorylation of EGFR. In some aspects, the protein of Table 1 is PTPRF, the protein of Table 2 is CD177, IL1RAP, or LRFN5, and the downstream activity is phosphorylation of EGFR.

In some aspects, the assay for phosphorylation of EGFR is a Western blot for phosphorylated EGFR, e.g., a Western blot as described in Du et al., J Cell Sci, 126: 1440-1453, 2013. In some aspects, the cell assayed in the Western blot is additionally treated with the modulator, e.g., by addition to the cell culture media. In some aspects, phosphorylation of EGFR is decreased by at least 10%, at least 20%, at least 30%, at least 40%, at least 50%, at least 60%, at least 70%, at least 80%, at least 90%, or 100% in the presence of the modulator as measured in a Western blot for phosphorylated EGFR. In some aspects, phosphorylation of EGFR is decreased by at least 30% in the presence of the modulator as measured in a Western blot for phosphorylated EGFR.

In some aspects, phosphorylation of EGFR is increased by at least 10%, at least 20%, at least 30%, at least 40%, at least 50%, at least 60%, at least 70%, at least 80%, at least 90%, or 100% in the presence of the modulator as measured in a Western blot for phosphorylated EGFR. In some aspects, phosphorylation of EGFR is increased by at least 30% in the presence of the modulator as measured in a Western blot for phosphorylated EGFR.

In other aspects, the protein of Table 1 is AXL and the downstream activity is phosphorylation of AXL. In some aspects, the protein of Table 1 is AXL, the protein of Table 2 is IL1RL1 or VSIG10L, and the downstream activity is phosphorylation of AXL.

In some aspects, the assay for phosphorylation of AXL is a Western blot for phosphorylated AXL. In some aspects, the cell assayed in the Western blot is additionally treated with the modulator, e.g., by addition to the cell culture media. In some aspects, phosphorylation of AXL is increased by at least 10%, at least 20%, at least 30%, at least 40%, at least 50%, at least 60%, at least 70%, at least 80%, at least 90%, or 100% in the presence of the modulator as measured in a Western blot for phosphorylated AXL. In some aspects, phosphorylation of AXL is decreased by at least 30% in the presence of the modulator as measured in a Western blot for phosphorylated AXL.

In some aspects, phosphorylation of AXL is increased by at least 10%, at least 20%, at least 30%, at least 40%, at least 50%, at least 60%, at least 70%, at least 80%, at least 90%, or 100% in the presence of the modulator as measured in a Western blot for phosphorylated AXL. In some aspects, phosphorylation of AXL is increased by at least 30% in the presence of the modulator as measured in a Western blot for phosphorylated AXL.

v. Inhibition of Cell Migration

In some aspects, the downstream activity is inhibition of cell migration. In some aspects, the protein of Table 1 is PTPRF and the downstream activity is inhibition of cell migration. In some aspects, the protein of Table 1 is PTPRF, the protein of Table 2 is CD177, IL1RAP, or LRFN5, and the downstream activity is inhibition of cell migration.

In some aspects, the downstream activity is inhibition of cell migration. In some aspects, the protein of Table 1 is PTPRS and the downstream activity is inhibition of cell migration. In some aspects, the protein of Table 1 is PTPRS, the protein of Table 2 is C6orf25, IL1RAP, IL1RAPL1, IL1RAPL2, LRFN1, LRFN5, LRRC4B, NCAM1, SLITRK1, SLITRK2, SLITRK3, SLITRK4, or SLITRK6, and the downstream activity is inhibition of cell migration.

In some aspects, the assay for inhibition of cell migration is a cell migration assay, e.g., a cell migration assay as described in Du et al., J Cell Sci, 126: 1440-1453, 2013. In some aspects, the cell assayed in the cell migration assay is additionally treated with the modulator, e.g., by addition to the cell culture media. In some aspects, inhibition of cell migration is increased by at least 10%, at least 20%, at least 30%, at least 40%, at least 50%, at least 60%, at least 70%, at least 80%, at least 90%, or 100% in the presence of the modulator as measured in a cell migration assay. In some aspects, inhibition of cell migration is decreased by at least 30% in the presence of the modulator as measured in a cell migration assay.

vi. Suppression of Tumor Formation

In some aspects, the downstream activity is suppression of tumor formation. In some aspects, the protein of Table 1 is CHL1 and the downstream activity is suppression of tumor formation. In some aspects, the protein of Table 1 is CHL1, the protein of Table 2 is CNTN1, CNTN5, SIRPA, L1CAM, or TMEM132A, and the downstream activity is suppression of tumor formation.

In some aspects, the assay for suppression of tumor formation is an in vitro tumorigenicity assay, e.g., a tumorigenicity assay as described in Tang et al., Oncogene, doi: 10.1038/s41388-018-0648-7, 2019, e.g., an XTT cell proliferation assay, a foci formation assay, a colony formation assay in soft agar, or a tumor formation assay in nude mice. In some aspects, the cell assayed in the tumorigenicity assay is additionally treated with the modulator, e.g., by addition to the cell culture media. In some aspects, suppression of tumor formation is increased by at least 10%, at least 20%, at least 30%, at least 40%, at least 50%, at least 60%, at least 70%, at least 80%, at least 90%, or 100% in the presence of the modulator as measured in a tumorigenicity assay. In some aspects, suppression of tumor formation is increased by at least 30% in the presence of the modulator as measured in a tumorigenicity assay.

vii. Suppression of Cell Invasion

In some aspects, the downstream activity is cell invasion. In some aspects, the protein of Table 1 is CNTN1 and the downstream activity is cell invasion. In some aspects, the protein of Table 1 is CNTN1, the protein of Table 2 is CDH6, CHL1, FCGRT, PCDHB7, or SGCG, and the downstream activity is cell invasion.

In other aspects, the protein of Table 1 is AXL and the downstream activity is cell invasion. In some aspects, the protein of Table 1 is AXL, the protein of Table 2 is IL1RL1 or VSIG10L, and the downstream activity is cell invasion.

In yet other aspects, the protein of Table 1 is CHL1 and the downstream activity is cell invasion. In some aspects, the protein of Table 1 is CHL1, the protein of Table 2 is CNTN1, CNTN5, SIRPA, L1CAM, or TMEM132A, and the downstream activity is cell invasion.

In some aspects, the assay for cell invasion is a gel invasion assay, e.g., a gel invasion assay as described in Yan et al., Cancer Res, 76(6): 1603-1614, 2016 or He et al., Biochem Biophys Res Commun, 438: 433-438, 2013. In some aspects, the cell assayed in the gel invasion assay is additionally treated with the modulator, e.g., by addition to the cell culture media. In some aspects, cell invasion is decreased by at least 10%, at least 20%, at least 30%, at least 40%, at least 50%, at least 60%, at least 70%, at least 80%, at least 90%, or 100% in the presence of the modulator as measured in a gel invasion assay. In some aspects, cell invasion is decreased by at least 30% in the presence of the modulator as measured in a gel invasion assay.

viii. Macrophage Polarization and Phagocytic Function

In some aspects, the downstream activity is suppression of the phagocytic function of phagocytes (e.g., macrophages), e.g., antibody-dependent cellular phagocytosis (suppression of phagocytosis). In some aspects, the protein of Table 1 is LILRB1 and the downstream activity is suppression of phagocytosis. In some aspects, the protein of Table 1 is LILRB1, the protein of Table 2 is CLEC6A, CXADR, EDAR, FLT4, IL6R, ILDR1, or LILRA5, and the downstream activity is suppression of phagocytosis.

Phagocytic function may be measured as, e.g., the proportion of macrophages comprising a fluorescent signal, wherein the presence of fluorescence indicates phagocytosis of a fluorescently labeled target cell. A representative assay for phagocytosis is described in Barkal et al., Nature Immunol, 19: 76-84, 2017. In some aspects, suppression of phagocytosis is decreased by at least 10%, at least 20%, at least 30%, at least 40%, at least 50%, at least 60%, at least 70%, at least 80%, at least 90%, or 100% in the presence of the modulator as measured in an assay for phagocytosis. In some aspects, suppression of phagocytosis is decreased by at least 30% in the presence of the modulator as measured in an assay for phagocytosis.

In some aspects, suppression of phagocytosis is increased by at least 10%, at least 20%, at least 30%, at least 40%, at least 50%, at least 60%, at least 70%, at least 80%, at least 90%, or 100% in the presence of the modulator as measured in an assay for phagocytosis. In some aspects, suppression of phagocytosis is increased by at least 30% in the presence of the modulator as measured in an assay for phagocytosis.

In some aspects, the downstream activity is promotion of M2 macrophage polarization. In some aspects, the protein of Table 1 is LILRB2 and the downstream activity is promotion of M2 macrophage polarization. In some aspects, the protein of Table 1 is LILRB2, the protein of Table 2 is IGSF8 or MOG, and the downstream activity is promotion of M2 macrophage polarization. M2 macrophage polarization may be assessed as in Chen et al., J Clin Invest, 128(12), 5647-5662. In some aspects, M2 macrophage polarization is decreased by at least 10%, at least 20%, at least 30%, at least 40%, at least 50%, at least 60%, at least 70%, at least 80%, at least 90%, or 100% in the presence of the modulator. In some aspects, M2 macrophage polarization is decreased by at least 30% in the presence of the modulator.

In some aspects, M2 macrophage polarization is increased by at least 10%, at least 20%, at least 30%, at least 40%, at least 50%, at least 60%, at least 70%, at least 80%, at least 90%, or 100% in the presence of the modulator. In some aspects, M2 macrophage polarization is increased by at least 30% in the presence of the modulator.

ix. Osteoclast Differentiation

In some aspects, the downstream activity is osteoclast differentiation.

In some aspects, the protein of Table 1 is LILRB1 and the downstream activity is osteoclast differentiation. In some aspects, the protein of Table 1 is LILRB1, the protein of Table 2 is CLEC6A, CXADR, EDAR, FLT4, IL6R, ILDR1, or LILRA5, and the downstream activity is osteoclast differentiation.

In some aspects, the protein of Table 1 is LILRB3 and the downstream activity is osteoclast differentiation. In some aspects, the protein of Table 1 is LILRB3, the protein of Table 2 is LRRC15 or LY6G6F, and the downstream activity is osteoclast differentiation.

In some aspects, the protein of Table 1 is LILRB4 and the downstream activity is osteoclast differentiation. In some aspects, the protein of Table 1 is LILRB4, the protein of Table 2 is CNTFR and the downstream activity is osteoclast differentiation.

In some aspects, the assay for osteoclast differentiation is an assay for multinucleated cells positive for tartrate-resistant acid phosphatase (TRAP) staining (TRAP+ multinucleated cells). TRAP+ status and multiple nuclei (e.g., three or more nuclei) are indicators that a cell is an osteoclast. A representative assay for TRAP+ multinucleated cells is provided in Mori et al., J Immunol, 181(7): 4742-4751, 2008. In some aspects, the cell assayed in the TRAP+ multinucleated cell assay is additionally treated with the modulator, e.g., by addition to the cell culture media. In some aspects, osteoclast differentiation is decreased by at least 10%, at least 20%, at least 30%, at least 40%, at least 50%, at least 60%, at least 70%, at least 80%, at least 90%, or 100% in the presence of the modulator as measured in an assay for TRAP+ multinucleated cells. In some aspects, osteoclast differentiation is decreased by at least 30% in the presence of the modulator as measured in an assay for TRAP+ multinucleated cells.

In other aspects, osteoclast differentiation is increased by at least 10%, at least 20%, at least 30%, at least 40%, at least 50%, at least 60%, at least 70%, at least 80%, at least 90%, or 100% in the presence of the modulator as measured in an assay for TRAP+ multinucleated cells. In some aspects, osteoclast differentiation is increased by at least 30% in the presence of the modulator as measured in an assay for TRAP+ multinucleated cells.

x. Activation of Signaling Pathways

In some aspects, the downstream activity is activation of a signaling pathway. In some aspects, the protein of Table 1 is AXL and the downstream activity is activation of the RAS/RAF/MAPK/ERK1/2 pathway, activation of the JAK/STAT pathway, or activation of the PI3K signaling pathway. In some aspects, the protein of Table 1 is AXL, the protein of Table 2 is IL1RL1 or VSIG10L, and the downstream activity is activation of the RAS/RAF/MAPK/ERK1/2 pathway, activation of the JAK/STAT pathway, or activation of the PI3K signaling pathway.

In some aspects, the protein of Table 1 is PTPRS and the downstream activity is activation of the PI3K signaling pathway. In some aspects, the protein of Table 1 is PTPRS, the protein of Table 2 is C6orf25, IL1RAP, IL1RAPL1, IL1RAPL2, LRFN1, LRFN5, LRRC4B, NCAM1, SLITRK1, SLITRK2, SLITRK3, SLITRK4, or SLITRK6, and the downstream activity is activation of the PI3K signaling pathway.

In other aspects, the protein of Table 1 is CNTN1 and the downstream activity is activation of the RhoA pathway or the Akt pathway. In some aspects, the protein of Table 1 is CNTN1, the protein of Table 2 is CDH6, CHL1, FCGRT, PCDHB7, or SGCG, and the downstream activity is activation of the RhoA pathway or the Akt pathway.

In some aspects, activation of the RAS/RAF/MAPK/ERK1/2 pathway, activation of the JAK/STAT pathway, activation of the RhoA pathway, activation of the Akt pathway, or activation of the PI3K signaling pathway is decreased by at least 10%, at least 20%, at least 30%, at least 40%, at least 50%, at least 60%, at least 70%, at least 80%, at least 90%, or 100% in the presence of the modulator. In some aspects, activation of the RAS/RAF/MAPK/ERK1/2 pathway, activation of the JAK/STAT pathway, activation of the RhoA pathway, activation of the Akt pathway, or activation of the PI3K signaling pathway is decreased by at least 30% in the presence of the modulator.

In some aspects, activation of the RAS/RAF/MAPK/ERK1/2 pathway, activation of the JAK/STAT pathway, activation of the RhoA pathway, activation of the Akt pathway, or activation of the PI3K signaling pathway is increased by at least 10%, at least 20%, at least 30%, at least 40%, at least 50%, at least 60%, at least 70%, at least 80%, at least 90%, or 100% in the presence of the modulator. In some aspects, activation of the RAS/RAF/MAPK/ERK1/2 pathway, activation of the JAK/STAT pathway, activation of the RhoA pathway, activation of the Akt pathway, or activation of the PI3K signaling pathway is increased by at least 30% in the presence of the modulator.

xi. Formation of Filopodia

In some aspects, the downstream activity is formation of filopodia. In some aspects, the protein of Table 1 is AXL and the downstream activity is formation of filopodia. In some aspects, the protein of Table 1 is AXL, the protein of Table 2 is IL1RL1 or VSIG10L, and the downstream activity is formation of filopodia. In some aspects, formation of filopodia is decreased by at least 10%, at least 20%, at least 30%, at least 40%, at least 50%, at least 60%, at least 70%, at least 80%, at least 90%, or 100% in the presence of the modulator. In some aspects, formation of filopodia is decreased by at least 30% in the presence of the modulator.

xii. Regulation of the EMT

In some aspects, the downstream activity is inhibition of the epithelial-mesenchymal transition (EMT). EMT increases the migratory and survival attributes of carcinoma cells, thus facilitating malignant progression (Gjerdrum et al., PNAS, 107(3): 1124-1129, 2010). In some aspects, the protein of Table 1 is AXL and the downstream activity is inhibition of the EMT. In some aspects, the protein of Table 1 is AXL, the protein of Table 2 is IL1RL1 or VSIG10L, and the downstream activity is inhibition of the EMT. EMT may be quantified as described in Gjerdrum et al., PNAS, 107(3): 1124-1129, 2010. In some aspects, inhibition of the EMT is increased by at least 10%, at least 20%, at least 30%, at least 40%, at least 50%, at least 60%, at least 70%, at least 80%, at least 90%, or 100% in the presence of the modulator. In some aspects, inhibition of the EMT is increased by at least 30% in the presence of the modulator.

xii. Tumor Growth

In some aspects, the downstream activity is tumor growth. In some aspects, the protein of Table 1 is PDPN, the protein of Table 2 is CD177, and the downstream activity is tumor growth. In some aspects, tumor growth is decreased by at least 10%, at least 20%, at least 30%, at least 40%, at least 50%, at least 60%, at least 70%, at least 80%, at least 90%, or 100% in the presence of the modulator. In some aspects, tumor growth is decreased by at least 30% in the presence of the modulator.

### IV. Modulators of Protein-Protein Interactions

In some aspects, the disclosure features an isolated modulator of the interaction between a protein of Table 1 and a protein of Table 2, wherein: (a) the protein of Table 1 and the protein of Table 2 are reported to interact in Table 3; and (b) the modulator causes an increase or decrease in the binding of the protein of Table 1 to the protein of Table 2 relative to binding in the absence of the modulator.

In some aspects, the disclosure features an isolated modulator of the downstream activity of a protein of Table 1 or a protein of Table 2, wherein (a) the protein of Table 1 and the protein of Table 2 are reported to interact in Table 3; and (b) the modulator causes a change in the downstream activity of the protein of Table 1 or the protein of Table 2 relative to downstream activity in the absence of the modulator.

In some aspects, the modulator is an inhibitor or an activator of the downstream activity of the protein of Table 1 or Table 2.

In some aspects, the change in the downstream activity is an increase or a decrease in the amount, strength, or duration of the downstream activity, e.g., a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia.

A. Small Molecules

In some aspects, the modulator or candidate modulator is a small molecule. Small molecules are molecules other than binding polypeptides or antibodies as defined herein that may bind, preferably specifically, to a protein of Table 1 and/or a protein of Table 2. Binding small molecules may be identified and chemically synthesized using known methodology (see, e.g., PCT Publication Nos. WO00/00823 and WO00/39585). Binding small molecules are usually less than about 2000 daltons in size (e.g., less than about 2000, 1500, 750, 500, 250 or 200 daltons in size), wherein such organic small molecules that are capable of binding, preferably specifically, to a polypeptide as described herein may be identified without undue experimentation using well known techniques. In this regard, it is noted that techniques for screening small molecule libraries for molecules that are capable of binding to a polypeptide target are well known in the art (see, e.g., PCT Publication Nos. WO00/00823 and WO00/39585). Binding small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some aspects, the binding of a protein of Table 1 and a protein of Table 2 is decreased (e.g., decreased by 5%, 10%, 20%, 30%, 40%, 50%, 60%, 70%, 80%, 90%, or 100%) in the presence of the small molecule. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100%) in the presence of the small molecule. In some aspects, a downstream activity (e.g., a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia) of the protein of Table 1 and/or the protein of Table 2 is decreased (e.g., decreased by 5%, 10%, 20%, 30%, 40%, 50%, 60%, 70%, 80%, 90%, or 100%) in the presence of the small molecule. In some aspects, a downstream activity (e.g., a downstream activity described in Section IIIB herein) of the protein of Table 1 and/or the protein of Table 2 is increased (e.g., increased by 5%, 10%, 20%, 30%, 40%, 50%, 60%, 70%, 80%, 90%, 100%, or more than 100%) in the presence of the small molecule.

B. Antibodies and Antigen-Binding Fragments

In some aspects, the modulator or candidate modulator is an antibody or an antigen-binding fragment thereof binding a protein of Table 1 and/or a protein of Table 2. In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

In some aspects, the binding of a protein of Table 1 and a protein of Table 2 is decreased (e.g., decreased by 5%, 10%, 20%, 30%, 40%, 50%, 60%, 70%, 80%, 90%, or 100%) in the presence of the antibody or antigen-binding fragment. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100%) in the presence of the antibody or antigen-binding fragment. In some aspects, a downstream activity (e.g., a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia) of the protein of Table 1 and/or the protein of Table 2 is decreased (e.g., decreased by 5%, 10%, 20%, 30%, 40%, 50%, 60%, 70%, 80%, 90%, or 100%) in the presence of the antibody or antigen-binding fragment. In some aspects, a downstream activity (e.g., a downstream activity described in Section IIIB herein) of the protein of Table 1 and/or the protein of Table 2 is increased (e.g., increased by 5%, 10%, 20%, 30%, 40%, 50%, 60%, 70%, 80%, 90%, 100%, or more than 100%) in the presence of the antibody or antigen-binding fragment.

C. Peptides

In some aspects, the modulator or candidate modulator is a peptide that binds to a protein of Table 1 and/or a protein of Table 2. The peptide may be the peptide may be naturally occurring or may be engineered. In some aspects, the peptide is a fragment of the protein of Table 1, the protein of Table 2, or another protein that binds to the protein of Table 1 or the protein of Table 2. The peptide may bind the binding partner with equal, less, or greater affinity than the full-length protein. In some aspects, the peptide performs all functions of the full-length protein. In other aspects, the peptide does not perform all functions of the full-length protein.

In some aspects, the binding of a protein of Table 1 and a protein of Table 2 is decreased (e.g., decreased by 5%, 10%, 20%, 30%, 40%, 50%, 60%, 70%, 80%, 90%, or 100%) in the presence of the peptide. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100%) in the presence of the peptide. In some aspects, a downstream activity (e.g., a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia) of the protein of Table 1 and/or the protein of Table 2 is decreased (e.g., decreased by 5%, 10%, 20%, 30%, 40%, 50%, 60%, 70%, 80%, 90%, or 100%) in the presence of the peptide. In some aspects, a downstream activity (e.g., a downstream activity described in Section IIIB herein) of the protein of Table 1 and/or the protein of Table 2 is increased (e.g., increased by 5%, 10%, 20%, 30%, 40%, 50%, 60%, 70%, 80%, 90%, 100%, or more than 100%) in the presence of the peptide.

D. Mimics

In some aspects, the modulator or candidate modulator is a mimic, e.g., a molecular mimic, that binds to a protein of Table 1 and/or a protein of Table 2. The mimic may be a molecular mimic of the protein of Table 1, the protein of Table 2, or another protein that binds to the protein of Table 1 or the protein of Table 2. In some aspects, the mimic may perform all functions of the mimicked polypeptide. In other aspects, the mimic does not perform all functions of the mimicked polypeptide.

In some aspects, the binding of a protein of Table 1 and a protein of Table 2 is decreased (e.g., decreased by 5%, 10%, 20%, 30%, 40%, 50%, 60%, 70%, 80%, 90%, or 100%) in the presence of the mimic. In some aspects, the binding of a protein of Table 1 and a protein of Table 2 is increased (e.g., increased by 5%, 10%, 20%, 30%, 40%, 50%, 60%, 70%, 80%, 90%, 100%, or more than 100%) in the presence of the mimic. In some aspects, a downstream activity (e.g., a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia) of the protein of Table 1 and/or the protein of Table 2 is decreased (e.g., decreased by 5%, 10%, 20%, 30%, 40%, 50%, 60%, 70%, 80%, 90%, or 100%) in the presence of the mimic. In some aspects, a downstream activity (e.g., a downstream activity described in Section IIIB herein) of the protein of Table 1 and/or the protein of Table 2 is increased (e.g., increased by 5%, 10%, 20%, 30%, 40%, 50%, 60%, 70%, 80%, 90%, 100%, or more than 100%) in the presence of the mimic.

### V. Methods of Treatment Comprising Modulators of Identified Protein-Protein Interactions

In some aspects, a modulator of a protein-protein interaction described in Section IIC herein is used to treat or delay progression of a pathological state, disease, disorder, or condition, e.g., a cancer.

In some aspects, the modulator increases or decreases the amount, strength, or duration of a downstream activity described in Section IIIB herein, e.g., CAF contractility, immune checkpoint inhibition, suppression of cell proliferation, modulation of target phosphorylation, inhibition of cell migration, suppression of tumor formation, suppression of cell invasion, macrophage polarization, regulation of phagocytosis, osteoclast differentiation, activation of a signaling pathway, or formation of filopodia, in an individual to whom the modulator has been administered.

A. Cancers

In some aspects, a modulator of a protein-protein interaction described in Section IIC herein (e.g., a small molecule, an antibody, an antigen-binding fragment, a peptide, a mimic, an antisense oligonucleotide, or an siRNA) is used to treat or delay progression of a cancer in a subject in need thereof. In some aspects, the subject is a human. The cancer may be a solid tumor cancer or a non-solid tumor cancer. Solid cancer tumors include, but are not limited to a bladder cancer, a melanoma, a breast cancer, a colorectal cancer, a lung cancer, a head and neck cancer, a kidney cancer, an ovarian cancer, a pancreatic cancer, or a prostate cancer, or metastatic forms thereof. In some aspects, the cancer is a bladder cancer. Further aspects of bladder cancer include urothelial carcinoma, muscle invasive bladder cancer (MIBC), or non-muscle invasive bladder cancer (NMIBC). In some aspects, the bladder cancer is a metastatic urothelial carcinoma (mUC). In some aspects, the cancer is a breast cancer. Further aspects of breast cancer include a hormone receptor-positive (HR+) breast cancer, e.g., an estrogen receptor-positive (ER+) breast cancer, a progesterone receptor-positive (PR+) breast cancer, or an ER+/PR+ breast cancer. Other aspects of breast cancer include a HER2-positive (HER2+) breast cancer. Yet other aspects of breast cancer include a triple-negative breast cancer (TNBC). In some aspects, the breast cancer is an early breast cancer. In some aspects, the cancer is a lung cancer. Further aspects of lung cancer include an epidermal growth factor receptor-positive (EGFR+) lung cancer. Other aspects of lung cancer include an epidermal growth factor receptor-negative (EGFR−) lung cancer. Yet other aspects of lung cancer include a non-small cell lung cancer, e.g., a squamous lung cancer or a non-squamous lung cancer. Other aspects of lung cancer include a small cell lung cancer. In some aspects, the cancer is a head and neck cancer. Further aspects of head and neck cancer include a squamous cell carcinoma of the head & neck (SCCHN). In some aspects, the cancer is a kidney cancer. Further aspects of kidney cancer include a renal cell carcinoma (RCC). In some aspects, the cancer is a liver cancer. Further aspects of liver cancer include a hepatocellular carcinoma. In some aspects, the cancer is a prostate cancer. Further aspects of prostate cancer include a castration-resistant prostate cancer (CRPC). In some aspects, the cancer is a metastatic form of a solid tumor. In some aspects, the metastatic form of a solid tumor is a metastatic form of a melanoma, a breast cancer, a colorectal cancer, a lung cancer, a head and neck cancer, a bladder cancer, a kidney cancer, an ovarian cancer, a pancreatic cancer, or a prostate cancer. In some aspects, the cancer is a metastatic urothelial carcinoma (mUC). In some aspects, the cancer is a non-solid tumor cancer. Non-solid tumor cancers include, but are not limited to, a B-cell lymphoma. Further aspects of B-cell lymphoma include, e.g., a chronic lymphocytic leukemia (CLL), a diffuse large B-cell lymphoma (DLBCL), a follicular lymphoma, myelodysplastic syndrome (MDS), a non-Hodgkin lymphoma (NHL), an acute lymphoblastic leukemia (ALL), a multiple myeloma, an acute myeloid leukemia (AML), or a mycosis fungoides (MF). In some aspects, the cancer is a colorectal cancer.

B. Methods of Delivery

The compositions utilized in the methods described herein (e.g., a modulator of a protein-protein interaction described in Section IIC, e.g., a small molecule, an antibody, an antigen-binding fragment, a peptide, a mimic, an antisense oligonucleotide, or an siRNA) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravascularly, mucosally, intrapericardially, intraumbilically, intraocularly, intraorbitally, orally, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some aspects, a modulator of a protein-protein interaction is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

A modulator of a protein-protein interaction described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The modulator need not be, but is optionally formulated with and/or administered concurrently with one or more agents currently used to prevent or treat the disorder in question. The effective amount of such other agents depends on the amount of the modulator present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### VI. Methods of Treatment Comprising PD-L1 Axis Binding Antagonists

A. Markers for Responsiveness to Atezolizumab

In some aspects, the invention comprises a method of identifying an individual having a cancer treatable with a PD-L1 axis binding antagonist (e.g., a cancer that is more likely to be treatable with a PD-L1 axis binding antagonist), the method comprising determining an expression level of a first member and a second member of at least one of the gene pairs of Table 15 in a sample from the individual, wherein an expression level of the first member of the gene pair that is above a first reference expression level and an expression level of the second member of the gene pair that is above a second reference expression level identifies the individual having a cancer treatable with a PD-L1 axis binding antagonist.

In some aspects, the invention comprises a method of identifying an individual having a cancer who may benefit from a treatment comprising a PD-L1 axis binding antagonist, the method comprising determining an expression level of a first member and a second member of at least one of the gene pairs of Table 15 in a sample from the individual, wherein an expression level of the first member of the gene pair that is above a first reference expression level and an expression level of the second member of the gene pair that is above a second reference expression level identifies the individual as one who may benefit from a treatment comprising a PD-L1 axis binding antagonist.

In some aspects, the invention comprises a method of selecting a therapy for an individual having a cancer, the method comprising determining an expression level of a first member and a second member at least one of the gene pairs of Table 15 in a sample from the individual, wherein an expression level of the first member of the gene pair that is above a first reference expression level and an expression level of the second member of the gene pair that is above a second reference expression level identifies the individual as one who may benefit from a treatment comprising a PD-L1 axis binding antagonist.

In some aspects, the individual has an expression level of the first member of the gene pair that is above a first reference expression level and an expression level of the second member of the gene pair that is above a second reference expression level and the method further comprises administering to the individual an effective amount of a PD-L1 axis binding antagonist.

In some aspects, the invention comprises a method of treating an individual having a cancer, the method comprising: (a) determining an expression level of a first member and a second member at least one of the gene pairs of Table 15 in a sample from the individual, wherein the expression level of the first member of the gene pair is above a first reference expression level and the expression level of the second member of the gene pair is above a second reference expression level; and (b) administering an effective amount of a PD-L1 axis binding antagonist to the individual.

In some aspects, the invention comprises a method of treating an individual having a cancer, the method comprising administering a PD-L1 axis binding antagonist to an individual who has been determined to have an expression level of a first member of a gene pair of Table 15 that is above a first reference expression level and an expression level of the second member of the gene pair that is above a second reference expression level.

In some aspects, the benefit comprises an extension in the individual's overall survival (OS) as compared to treatment without the PD-L1 axis binding antagonist. In other aspects, the benefit may comprise, e.g., an increase in time to recurrence or a reduced duration of treatment as compared to treatment without the PD-L1 axis binding antagonist.

In some aspects, the first member of the gene pair is SIGLEC6 and the second member of the gene pair is NCR1.

In some aspects, the first member of the gene pair is BTN3A1 and the second member of the gene pair is LRRC4B.

In some aspects, the first member of the gene pair is CD80 and the second member of the gene pair is CTLA4.

In some aspects, the first member of the gene pair is BTN3A3 and the second member of the gene pair is LRRC4B.

In some aspects, the first member of the gene pair is NCR1 and the second member of the gene pair is SIGLEC8.

In some aspects, the first member of the gene pair is CNTFR and the second member of the gene pair is LILRB4.

In some aspects, the first member of the gene pair is FGFR3 and the second member of the gene pair is LRRTM2.

In some aspects, the first member of the gene pair is FGFR4 and the second member of the gene pair is SIGLEC15.

In some aspects, the first member of the gene pair is FGFR1 and the second member of the gene pair is KL.

In some aspects, the first member of the gene pair is EFNB1 and the second member of the gene pair is TRHDE.

In some aspects, the first member of the gene pair is CTLA4 and the second member of the gene pair is PCDHGB4.

In some aspects, the first member of the gene pair is CTLA4 and the second member of the gene pair is FAM200A.

In some aspects, the first member of the gene pair is CA12 and the second member of the gene pair is SIGLEC6.

In some aspects, the first member of the gene pair is ILDR2 and the second member of the gene pair is CLEC12B.

In some aspects, the first member of the gene pair is EFNB1 and the second member of the gene pair is ITLN1.

In some aspects, the first member of the gene pair is CADM1 and the second member of the gene pair is CRTAM.

In some aspects, the first member of the gene pair is CD79B and the second member of the gene pair is CD244.

In some aspects, the first member of the gene pair is DAG1 and the second member of the gene pair is EFNB1.

B. Markers for Lack of Responsiveness to Atezolizumab

In some aspects, the invention comprises a method of identifying an individual having a cancer who may benefit from a treatment other than or in addition to a PD-L1 axis binding antagonist, the method comprising determining an expression level of a first member and a second member of at least one of the gene pairs of Table 16 in a sample from the individual, wherein an expression level of the first member of the gene pair that is above a first reference expression level and an expression level of the second member of the gene pair that is above a second reference expression level identifies the individual as one who may benefit from a treatment other than or in addition to a PD-L1 axis binding antagonist.

In some aspects, the invention comprises a method of selecting a therapy for an individual having a cancer, the method comprising determining an expression level of a first member and a second member at least one of the gene pairs of Table 16 in a sample from the individual, wherein an expression level of the first member of the gene pair that is above a first reference expression level and an expression level of the second member of the gene pair that is above a second reference expression level identifies the individual as one who may benefit from a treatment other than or in addition to a PD-L1 axis binding antagonist.

In some aspects, the invention comprises a method of identifying an individual having a cancer who may benefit from a treatment other than or in addition to a PD-L1 axis binding antagonist, the method comprising determining an expression level of a first member and a second member of at least one of the gene pairs of Table 16 in a sample from the individual, wherein an expression level of the first member of the gene pair that is above a first reference expression level and an expression level of the second member of the gene pair that is above a second reference expression level identifies an individual having a cancer resistant to treatment with a PD-L1 axis binding antagonist.

In some aspects, the invention comprises a method of selecting a therapy for an individual having a cancer, the method comprising determining an expression level of a first member and a second member at least one of the gene pairs of Table 16 in a sample from the individual, wherein an expression level of the first member of the gene pair that is above a first reference expression level and an expression level of the second member of the gene pair that is above a second reference expression level identifies an individual having a cancer resistant to treatment with a PD-L1 axis binding antagonist.

In some aspects, the individual has an expression level of the first member of the gene pair that is above a first reference expression level and an expression level of the second member of the gene pair that is above a second reference expression level and the method comprises administering to the individual an effective amount of a treatment other than or in addition to a PD-L1 axis binding antagonist.

In some aspects, the benefit comprises an extension in the individual's overall survival (OS) as compared to treatment without the treatment other than or in addition to a PD-L1 axis binding antagonist.

In some aspects, the sample from the individual is obtained from the individual prior to administration of an anti-cancer therapy. In some aspects, the sample from the individual is obtained from the individual after administration of an anti-cancer therapy.

In some aspects, the sample from the individual is a tumor tissue sample or a tumor fluid sample, e.g., a formalin-fixed and paraffin-embedded (FFPE) sample, an archival sample, a fresh sample, or a frozen sample.

In some aspects, (a) the expression level of the first member and the second member of the gene pair in the sample is a protein expression level; or (b) the expression level of the first member and the second member of the gene pair in the sample is an mRNA expression level.

In some aspects, the expression level of the first member and the second member of the gene pair in the sample is a mRNA expression level of the first member and the second member of the gene pair, respectively. In some aspects, the mRNA expression level of the first member and the second member of the gene pair is determined by in situ hybridization (ISH), RNA-seq, RT-qPCR, qPCR, multiplex qPCR or RT-qPCR, microarray analysis, SAGE, MassARRAY technique, FISH, or a combination thereof. In some aspects, the RNA-seq is TruSeq RNA Access technology (Illumina®).

In some aspects, the first member of the gene pair is EFNB1 and the second member of the gene pair is EVC2.

In some aspects, the first member of the gene pair is GPC4 and the second member of the gene pair is FGFRL1.

In some aspects, the first member of the gene pair is EFNB3 and the second member of the gene pair is EPHB4.

In some aspects, the first member of the gene pair is PTPRD and the second member of the gene pair is LRFN4.

In some aspects, the first member of the gene pair is EFNB1 and the second member of the gene pair is AQPEP.

In some aspects, the first member of the gene pair is EFNB1 and the second member of the gene pair is DSG4.

In some aspects, the first member of the gene pair is LDLR and the second member of the gene pair is LILRB5.

In some aspects, the first member of the gene pair is EFNB3 and the second member of the gene pair is EPHB3.

In some aspects, the first member of the gene pair is PLXNB3 and the second member of the gene pair is SEMA4G.

In some aspects, the first member of the gene pair is EFNB1 and the second member of the gene pair is EPHB6.

In some aspects, the first member of the gene pair is FLT4 and the second member of the gene pair is FLRT2.

In some aspects, the first member of the gene pair is FLT1 and the second member of the gene pair is ELFN1.

In some aspects, the first member of the gene pair is GPC4 and the second member of the gene pair is FGFR4.

In some aspects, the first member of the gene pair is GPC3 and the second member of the gene pair is TNFRSF11B.

In some aspects, the first member of the gene pair is FGFR4 and the second member of the gene pair is GPC6.

In some aspects, the first member of the gene pair is PLXNB1 and the second member of the gene pair is SEMA4B.

In some aspects, the first member of the gene pair is EDA and the second member of the gene pair is EDAR.

In some aspects, the first member of the gene pair is FGFR4 and the second member of the gene pair is NRXN2.

In some aspects, the first member of the gene pair is SEMA4D and the second member of the gene pair is PLXNB2.

In some aspects, the first member of the gene pair is FLT4 and the second member of the gene pair is NRP2.

In some aspects, the first member of the gene pair is FGFR4 and the second member of the gene pair is GPC3.

In some aspects, the first member of the gene pair is FGFR2 and the second member of the gene pair is RAMP1.

In some aspects, the first member of the gene pair is AXL1 and the second member of the gene pair is IL1RL1.

In some aspects, the first member of the gene pair is CD320 and the second member of the gene pair is IGSF5.

In some aspects, the first member of the gene pair is CD59 and the second member of the gene pair is STAB1.

In some aspects, the first member of the gene pair is CNTN3 and the second member of the gene pair is PTPRG.

In some aspects, the first member of the gene pair is EFNB1 and the second member of the gene pair is EPHA3.

In some aspects, the first member of the gene pair is EFNB3 and the second member of the gene pair is EPHB2.

In some aspects, the first member of the gene pair is EGF and the second member of the gene pair is TNFRSF11B.

In some aspects, the first member of the gene pair is ENPEP and the second member of the gene pair is SLITRK1.

In some aspects, the first member of the gene pair is FCGR3B and the second member of the gene pair is EDA2R.

In some aspects, the first member of the gene pair is IL20RA and the second member of the gene pair is CLEC14A.

In some aspects, the first member of the gene pair is IL6R and the second member of the gene pair is BTNL9.

In some aspects, the first member of the gene pair is IZUMO1 and the second member of the gene pair is LILRA5.

In some aspects, the first member of the gene pair is NGFR and the second member of the gene pair is LRRTM3.

In some aspects, the first member of the gene pair is NTM and the second member of the gene pair is AMIGO2.

In some aspects, the first member of the gene pair is PCDHB3 and the second member of the gene pair is IGSF11.

In some aspects, the first member of the gene pair is PTGFRN and the second member of the gene pair is TMEM59L.

In some aspects, the first member of the gene pair is TREM1 and the second member of the gene pair is VSIG8.

C. Reference Expression Levels

In some aspects, the reference expression level for the first member of the protein pair (i.e., the first reference expression level) is a pre-assigned expression level and the reference expression level for the first member of the protein pair (i.e., the second reference expression level) is a pre-assigned reference expression level.

In some aspects, the first reference expression level is between about 0.1 to about 0.5 counts per million (CPM), e.g, between about 0.15 to about 0.4 CPM, between about 0.2 to 0.3 CPM, or between about 0.225 to about 0.275 CPM.

In some aspects, the second reference expression level is between about 0.1 to about 0.5 counts per million (CPM), e.g, between about 0.15 to about 0.4 CPM, between about 0.2 to 0.3 CPM, or between about 0.225 to about 0.275 CPM.

In some aspects, the first reference expression level is between about 0.25 to about 0.5 counts per million (CPM) and the second reference expression level is between about 0.25 to about 0.5 CPM.

In some aspects, the first reference expression level is 0.25 CPM. In some aspects, the second reference expression level is 0.25 CPM. In some aspects, the first reference expression level is 0.25 CPM and the second reference expression level is 0.25 CPM.

In some aspects, the first reference expression level and the second reference expression level are expression levels of the first member and the second member of the gene pair, respectively, in a reference population of individuals having a cancer, e.g., a urinary tract cancer, e.g., a urinary tract carcinoma, e.g., a metastatic urothelial carcinoma (mUC).

D. Cancers

In some aspects, a PD-L1 axis binding antagonist is used to treat or delay progression of a cancer, e.g., a urinary tract cancer, in a subject in need thereof. In some aspects, the subject is a human. Urinary tract cancers include urothelial carcinomas (UC), non-urothelial carcinomas of the urinary tract, and carcinomas of the urinary tract having mixed histology. Non-urothelial carcinomas of the urinary tract include all subtypes listed in the World Health Organization classification, e.g., a squamous cell carcinoma, a verrucous carcinoma, an adenocarcinoma, a glandular carcinoma, a carcinoma of the Bellini collecting duct, a neuroendocrine carcinoma, or a small cell carcinoma. The adenocarcinoma may be an enteric adenocarcinoma, a mucinous adenocarcinoma, a signet-ring cell adenocarcinoma, or a clear cell adenocarcinoma. Urinary tract cancers may be located in the bladder, the renal pelvis, the ureter, or the urethra. In some aspects, the urinary tract cancer (e.g., urothelial carcinoma, non-urothelial carcinoma, or carcinoma of the urinary tract having mixed histology) is locally advanced, e.g., stage T4b N_{any} or T_{any} N2-3, according to the TNM classification, at the onset of treatment. In some aspects, the urinary tract cancer is a metastatic urothelial carcinoma (mUC), a metastatic form of a non-urothelial carcinoma of the urinary tract, or a metastatic form of a carcinoma of the urinary tract having mixed histology. In some aspects, the urinary tract cancer is TNM stage M1, according to the TNM classification, at the onset of treatment.

E. Immune Checkpoint Inhibitors

In some aspects, the methods of the invention include use of a PD-L1 axis binding antagonist, which may be a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some aspects, the PD-1 binding antagonist is a molecule that inhibits the binding of PD-1 to its ligand binding partners. In a specific aspect the PD-1 ligand binding partners are PD-L1 and/or PD-L2. In another instance, a PD-L1 binding antagonist is a molecule that inhibits the binding of PD-L1 to its binding ligands. In a specific aspect, PD-L1 binding partners are PD-1 and/or B7-1. In another instance, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide.

In some aspects, the PD-1 binding antagonist is an anti-PD-1 antibody (e.g., a human antibody, a humanized antibody, or a chimeric antibody), for example, as described below. In some aspects, the anti-PD-1 antibody is selected from the group consisting of MDX-1106 (nivolumab), MK-3475 (pembrolizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in WO2006/121168. MK-3475, also known as pembrolizumab or lambrolizumab, is an anti-PD-1 antibody described in WO 2009/114335. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some instances, the PD-1 binding antagonist is AMP-224. AMP-224, also known as B7-DCIg, is a PD-L2-Fc fusion soluble receptor described in WO 2010/027827 and WO 2011/066342.

In some aspects, the anti-PD-1 antibody is MDX-1106. Alternative names for “MDX-1106” include MDX-1106-04, ONO-4538, BMS-936558, and nivolumab. In some aspects, the anti-PD-1 antibody is nivolumab (CAS Registry Number: 946414-94-4). In a still further aspect, provided is an isolated anti-PD-1 antibody comprising a heavy chain variable region comprising the heavy chain variable region amino acid sequence from SEQ ID NO: 1 and/or a light chain variable region comprising the light chain variable region amino acid sequence from SEQ ID NO: 2. In a still further aspect, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 1)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,

and
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some aspects, the PD-L1 axis binding antagonist is a PD-L2 binding antagonist. In some aspects, the PD-L2 binding antagonist is an anti-PD-L2 antibody (e.g., a human antibody, a humanized antibody, or a chimeric antibody). In some aspects, the PD-L2 binding antagonist is an immunoadhesin.

In some aspects, the PD-L1 binding antagonist is an anti-PD-L1 antibody, for example, as described below. In some aspects, the anti-PD-L1 antibody is capable of inhibiting binding between PD-L1 and PD-1 and/or between PD-L1 and B7-1. In some aspects, the anti-PD-L1 antibody is a monoclonal antibody. In some aspects, the anti-PD-L1 antibody is an antibody fragment selected from the group consisting of Fab, Fab′-SH, Fv, scFv, and (Fab′)₂ fragments. In some aspects, the anti-PD-L1 antibody is a humanized antibody. In some aspects, the anti-PD-L1 antibody is a human antibody. In some aspects, the anti-PD-L1 antibody is selected from the group consisting of YW243.55.S70, MPDL3280A (atezolizumab), MDX-1105, and MEDI4736 (durvalumab), and MSB0010718C (avelumab). Antibody YW243.55.S70 is an anti-PD-L1 described in WO 2010/077634. MDX-1105, also known as BMS-936559, is an anti-PD-L1 antibody described in WO2007/005874. MEDI4736 (durvalumab) is an anti-PD-L1 monoclonal antibody described in WO2011/066389 and US2013/034559. Examples of anti-PD-L1 antibodies useful for the methods of this invention, and methods for making thereof are described in PCT patent application WO 2010/077634, WO 2007/005874, WO 2011/066389, U.S. Pat. No. 8,217,149, and US 2013/034559, which are incorporated herein by reference.

Anti-PD-L1 antibodies described in WO 2010/077634 A1 and U.S. Pat. No. 8,217,149 may be used in the methods described herein. In some aspects, the anti-PD-L1 antibody comprises a heavy chain variable region sequence of SEQ ID NO: 3 and/or a light chain variable region sequence of SEQ ID NO: 4. In a still further aspect, provided is an isolated anti-PD-L1 antibody comprising a heavy chain variable region and/or a light chain variable region sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 3)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

and
(b) the light chain sequence has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In one aspect, the anti-PD-L1 antibody comprises a heavy chain variable region comprising an HVR-H1, HVR-H2 and HVR-H3 sequence, wherein:

(SEQ ID NO: 5)
(a) the HVR-H1 sequence is GFTFSX₁SWIH;
(SEQ ID NO: 6)
(b) the HVR-H2 sequence is AWIX₂PYGGSX₃YYADSVKG;
(SEQ ID NO: 7)
(c) the HVR-H3 sequence is RHWPGGFDY;

further wherein: X₁ is D or G; X₂ is S or L; X₃ is T or S. In one specific aspect, X₁ is D; X₂ is S and X₃ is T. In another aspect, the polypeptide further comprises variable region heavy chain framework sequences juxtaposed between the HVRs according to the formula: (FR-H1)-(HVR-H1)-(FR-H2)-(HVR-H2)-(FR-H3)-(HVR-H3)-(FR-H4). In yet another aspect, the framework sequences are derived from human consensus framework sequences. In a further aspect, the framework sequences are VH subgroup III consensus framework. In a still further aspect, at least one of the framework sequences is the following:

(SEQ ID NO: 8)
FR-H1 is EVQLVESGGGLVQPGGSLRLSCAAS
(SEQ ID NO: 9)
FR-H2 is WVRQAPGKGLEWV
(SEQ ID NO: 10)
FR-H3 is RFTISADTSKNTAYLQMNSLRAEDTAVYYCAR
(SEQ ID NO: 11)
FR-H4 is WGQGTLVTVSS.

In a still further aspect, the heavy chain polypeptide is further combined with a variable region light chain comprising an HVR-L1, HVR-L2 and HVR-L3, wherein:
(a) the HVR-L1 sequence is RASQX₄X₅X₆TX₇X₈A (SEQ ID NO: 12);
(b) the HVR-L2 sequence is SASX₉LX₁₀S, (SEQ ID NO: 13);
(c) the HVR-L3 sequence is QQX₁₁X₁₂X₁₃X₁₄PX₁₅T (SEQ ID NO: 14);
   wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a still further aspect, X₄ is D; X₅ is V; X₆ is S; X₇ is A; X₈ is V; X₉ is F; X₁₀ is Y; X₁₁, is Y; X₁₂ is L; X₁₃ is Y; X₁₄ is H; X₁₅ is A.

In a still further aspect, the light chain further comprises variable region light chain framework sequences juxtaposed between the HVRs according to the formula: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the framework sequences are VL kappa I consensus framework. In a still further aspect, at least one of the framework sequence is the following:

(SEQ ID NO: 15)
FR-L1 is DIQMTQSPSSLSASVGDRVTITC
(SEQ ID NO: 16)
FR-L2 is WYQQKPGKAPKLLIY
(SEQ ID NO: 17)
FR-L3 is GVPSRFSGSGSGTDFTLTISSLQPEDFATYYC
(SEQ ID NO: 18)
FR-L4 is FGQGTKVEIKR.

In another aspect, provided is an isolated anti-PD-L1 antibody or antigen binding fragment comprising a heavy chain and a light chain variable region sequence, wherein:
(a) the heavy chain comprises an HVR-H1, HVR-H2 and HVR-H3, wherein further:

(SEQ ID NO: 5)
(i) the HVR-H1 sequence is GFTFSX₁SWIH;
(SEQ ID NO: 6)
(ii) the HVR-H2 sequence is AWIX₂PYGGSX₃YYADSVKG
(SEQ ID NO: 7)
(iii) the HVR-H3 sequence is RHWPGGFDY,
and

(b) the light chain comprises an HVR-L1, HVR-L2 and HVR-L3, wherein further:

(SEQ ID NO: 12)
(i) the HVR-L1 sequence is RASQX₄X₅X₆TX₇X₈A
(SEQ ID NO: 13)
(ii) the HVR-L2 sequence is SASX₉LX₁₀S;
and
(SEQ ID NO: 14)
(iii) the HVR-L3 sequence is QQX₁₁X₁₂X₁₃X₁₄PX₁₅T;

wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T. In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H and X₁₅ is A.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and 11. In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation mutation. In still a further aspect, the effector-less Fc mutation is an N297A or D265A/N297A substitution in the constant region.

In yet another aspect, provided is an anti-PD-L1 antibody comprising a heavy chain and a light chain variable region sequence, wherein:
(a) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, or
(b) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and 11. In a still further aspect, the light chain framework sequences are derived from a Kabat kappa I, II, II, or IV subgroup sequence. In a still further aspect, the light chain framework sequences are VL kappa I consensus framework. In a still further aspect, one or more of the light chain framework sequences are set forth as SEQ ID NOs: 15, 16, 17, and 18.

In a further aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

FR-H1
(SEQ ID NO: 27)
EVQLVESGGGLVQPGGSLRLSCAASGFTFS
FR-H2
(SEQ ID NO: 28)
WVRQAPGKGLEWVA
FR-H3
(SEQ ID NO: 10)
RFTISADTSKNTAYLQMNSLRAEDTAVYYCAR
FR-H4
(SEQ ID NO: 11)
WGQGTLVTVSS.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

FR-L1
(SEQ ID NO: 15)
DIQMTQSPSSLSASVGDRVTITC
FR-L2
(SEQ ID NO: 16)
WYQQKPGKAPKLLIY
FR-L3
(SEQ ID NO: 17)
GVPSRFSGSGSGTDFTLTISSLQPEDFATYYC
FR-L4
(SEQ ID NO: 26)
FGQGTKVEIK.

In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further aspect, the effector-less Fc mutation is an N297A or D265A/N297A substitution in the constant region.

In yet another aspect, provided is an anti-PD-L1 antibody comprising a heavy chain and a light chain variable region sequence, wherein:
(c) the heavy chain further comprises an HVR-H1, HVR-H2 and an HVR-H3 sequence having at least 85% sequence identity to GFTFSDSWIH (SEQ ID NO: 19), AWISPYGGSTYYADSVKG (SEQ ID NO: 20) and RHWPGGFDY (SEQ ID NO: 21), respectively, and/or
(d) the light chain further comprises an HVR-L1, HVR-L2 and an HVR-L3 sequence having at least 85% sequence identity to RASQDVSTAVA (SEQ ID NO: 22), SASFLYS (SEQ ID NO: 23) and QQYLYHPAT (SEQ ID NO: 24), respectively.

In a specific aspect, the sequence identity is 86%, 87%, 88%, 89%, 90%, 91%, 92%, 93%, 94%, 95%, 96%, 97%, 98%, 99% or 100%.

In another aspect, the heavy chain variable region comprises one or more framework sequences juxtaposed between the HVRs as: (FR-H1)-(HVR-H1)-(FR-H2)-(HVR-H2)-(FR-H3)-(HVR-H3)-(FR-H4), and the light chain variable regions comprises one or more framework sequences juxtaposed between the HVRs as: (FR-L1)-(HVR-L1)-(FR-L2)-(HVR-L2)-(FR-L3)-(HVR-L3)-(FR-L4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences are set forth as SEQ ID NOs: 8, 9, 10, and WGQGTLVTVSSASTK (SEQ ID NO: 29).

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences are set forth as SEQ ID NOs: 15, 16, 17, and 18. In a still further specific aspect, the antibody further comprises a human or murine constant region. In a still further aspect, the human constant region is selected from the group consisting of IgG1, IgG2, IgG2, IgG3, and IgG4. In a still further specific aspect, the human constant region is IgG1. In a still further aspect, the murine constant region is selected from the group consisting of IgG1, IgG2A, IgG2B, and IgG3. In a still further aspect, the murine constant region in IgG2A. In a still further specific aspect, the antibody has reduced or minimal effector function. In a still further specific aspect, the minimal effector function results from an “effector-less Fc mutation” or aglycosylation. In still a further aspect, the effector-less Fc mutation is an N297A or D265A/N297A substitution in the constant region.

In a still further aspect, provided is an isolated anti-PD-L1 antibody comprising a heavy chain and a light chain variable region sequence, wherein:
(a) the heavy chain variable region sequence has at least 85% sequence identity to the sequence:

(SEQ ID NO: 25)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTK,

(b) the light chain variable region sequence has at least 85% sequence identity to the sequence:

(SEQ ID NO: 4)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some aspects, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4. In some aspects, provided is an isolated anti-PD-L1 antibody comprising a heavy chain and a light chain variable region sequence, wherein the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 25. In some aspects, provided is an isolated anti-PD-L1 antibody comprising a heavy chain and a light chain variable region sequence, wherein the light chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4 and the heavy chain variable region sequence has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 25. In some aspects, one, two, three, four, or five amino acid residues at the N-terminal of the heavy and/or light chain may be deleted, substituted or modified.

In a still further aspect, provided is an isolated anti-PD-L1 antibody comprising a heavy chain and a light chain sequence, wherein:
(a) the heavy chain sequence has at least 85% sequence identity to the heavy chain sequence:

(SEQ ID NO: 30)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGOPENNYKTTPPVLD
SDGSFFLYSKLTVDKSRWQQGNVFSCSVMHEALHNHYTQKSLSLSPG,

and/or
(b) the light chain sequences has at least 85% sequence identity to the light chain sequence:

(SEQ ID NO: 31)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some aspects, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31. In some aspects, provided is an isolated anti-PD-L1 antibody comprising a heavy chain and a light chain sequence, wherein the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30. In some aspects, provided is an isolated anti-PD-L1 antibody comprising a heavy chain and a light chain sequence, wherein the light chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 31 and the heavy chain sequence has at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 30.

In some aspects, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In any of the aspects herein, the isolated anti-PD-L1 antibody can bind to a human PD-L1, for example a human PD-L1 as shown in UniProtKB/Swiss-Prot Accession No. Q9NZQ7.1, or a variant thereof.

In a still further aspect, provided is an isolated nucleic acid encoding any of the antibodies described herein. In some aspects, the nucleic acid further comprises a vector suitable for expression of the nucleic acid encoding any of the previously described anti-PD-L1 antibodies. In a still further specific aspect, the vector is in a host cell suitable for expression of the nucleic acid. In a still further specific aspect, the host cell is a eukaryotic cell or a prokaryotic cell. In a still further specific aspect, the eukaryotic cell is a mammalian cell, such as Chinese hamster ovary (CHO) cell.

The antibody or antigen binding fragment thereof, may be made using methods known in the art, for example, by a process comprising culturing a host cell containing nucleic acid encoding any of the previously described anti-PD-L1 antibodies or antigen-binding fragments in a form suitable for expression, under conditions suitable to produce such antibody or fragment, and recovering the antibody or fragment.

It is expressly contemplated that such PD-L1 axis binding antagonist antibodies (e.g., anti-PD-L1 antibodies, anti-PD-1 antibodies, and anti-PD-L2 antibodies), or other antibodies described herein for use in any of the aspects enumerated above may have any of the features, singly or in combination.

In some aspects, the immune checkpoint inhibitor is an antagonist directed against a co-inhibitory molecule (e.g., a CTLA-4 antagonist (e.g., an anti-CTLA-4 antibody), a TIM-3 antagonist (e.g., an anti-TIM-3 antibody), or a LAG-3 antagonist (e.g., an anti-LAG-3 antibody)), or any combination thereof.

In some aspects, the immune checkpoint inhibitor is an antagonist directed against TIGIT (e.g., an anti-TIGIT antibody). Exemplary anti-TIGIT antibodies are described in US Patent Application Publication No. 2018/0186875 and in International Patent Application Publication No. WO 2017/053748, which are incorporated herein by reference in their entirety.

F. Methods of Delivery

The compositions utilized in the methods described herein (e.g., PD-L1 axis binding antagonists) can be administered by any suitable method, e.g., as described in Section VB herein.

Immune checkpoint inhibitors (e.g., an immune checkpoint inhibitor described in Section VIE herein, e.g., an antibody, binding polypeptide, and/or small molecule) described herein (and any additional therapeutic agent) may be formulated, dosed, and administered in a fashion consistent with good medical practice. Factors for consideration in this context include the particular disorder being treated, the particular mammal being treated, the clinical condition of the individual, the cause of the disorder, the site of delivery of the agent, the method of administration, the scheduling of administration, and other factors known to medical practitioners. The immune checkpoint inhibitor need not be, but is optionally formulated with and/or administered concurrently with one or more agents currently used to prevent or treat the disorder in question. The effective amount of such other agents depends on the amount of the immune checkpoint inhibitor present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the treatment of a cancer, e.g., a cancer described in Section VID herein e.g., a urinary tract cancer, the appropriate dosage of an immune checkpoint inhibitor, e.g., a PD-L1 axis binding antagonist, an antagonist directed against a co-inhibitory molecule (e.g., a CTLA-4 antagonist (e.g., an anti-CTLA-4 antibody), a TIM-3 antagonist (e.g., an anti-TIM-3 antibody), or a LAG-3 antagonist (e.g., an anti-LAG-3 antibody)), or any combination thereof, described herein (when used alone or in combination with one or more other additional therapeutic agents) will depend on the type of disease to be treated, the severity and course of the disease, whether the PD-L1 axis binding antagonist is administered for preventive or therapeutic purposes, previous therapy, the patient's clinical history and response to the PD-L1 axis binding antagonist, and the discretion of the attending physician. The immune checkpoint inhibitor is suitably administered to the individual at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the individual receives, for example, from about two to about twenty, or e.g., about six doses of the immune checkpoint inhibitor). An initial higher loading dose, followed by one or more lower doses, may be administered. However, other dosage regimens may be useful. The progress of this therapy is easily monitored by conventional techniques and assays.

For example, as a general proposition, the therapeutically effective amount of an immune checkpoint inhibitor, for example, a PD-L1 axis binding antagonist antibody, an anti-CTLA-4 antibody, an anti-TIM-3 antibody, or an anti-LAG-3 antibody, administered to human will be in the range of about 0.01 to about 50 mg/kg of patient body weight, whether by one or more administrations. In some aspects, the antibody used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some aspects, the antibody is administered at 15 mg/kg. However, other dosage regimens may be useful. In one aspect, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w). In some aspects, anti-PD-L1 antibody MPDL3280A (atezolizumab) is administered at 1200 mg intravenously every three weeks (q3w). In some aspects, anti-PD-L1 antibody MPDL3280A (atezolizumab) is administered at 840 mg intravenously every two weeks (q2w). In some aspects, anti-PD-L1 antibody MPDL3280A (atezolizumab) is administered at 1680 mg intravenously every four weeks (q4w). The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some aspects, the individual has not been previously treated for the urinary tract cancer. In some aspects, the individual has previously been treated for the urinary tract cancer (e.g., a locally advanced or metastatic urinary tract carcinoma, e.g., a urothelial or non-urothelial carcinoma). In some aspects, the individual has previously been treated for the urinary tract cancer with a therapy comprising platinum (e.g., a therapy comprising gemcitabine and cisplatin; a therapy comprising gemcitabine and carboplatin; or a therapy comprising methotrexate, vinblastine, doxorubicin, and cisplatin (MVAC)). In some aspects, the individual has previously been treated for the urinary tract cancer with a therapy that does not comprise platinum. In some aspects, the individual has not been previously administered an immune checkpoint inhibitor.

G. Additional Therapeutic Agents

In some aspects, the PD-L1 axis binding antagonist is used with one or more additional therapeutic agents, e.g., a combination therapy. In some aspects, the composition comprising the PD-L1 axis binding antagonist further comprises the additional therapeutic agent. In another aspect, the additional therapeutic agent is delivered in a separate composition. In some aspects, the one or more additional therapeutic agents comprise an immunomodulatory agent, an anti-neoplastic agent, a chemotherapeutic agent, a growth inhibitory agent, an anti-angiogenic agent, a radiation therapy, a cytotoxic agent, a cell-based therapy, or a combination thereof.

Combination therapies as described above encompass combined administration (wherein two or more therapeutic agents are included in the same or separate formulations) and separate administration (wherein administration of a PD-L1 axis binding antagonist can occur prior to, simultaneously, and/or following, administration of the additional therapeutic agent or agents). In one aspect, administration of a PD-L1 axis binding antagonist and administration of an additional therapeutic agent occur within about one month, or within about one, two or three weeks, or within about one, two, three, four, five, or six days, of each other.

### VII. Methods of Treatment Comprising Agonists of CD177 Activity

A. Methods of Treatment

In some aspects, the invention comprises a method of treating an individual having a cancer comprising administering to the individual a treatment comprising an effective amount of an agonist of CD177 activity.

In some aspects, the invention comprises a method of identifying an individual having a cancer who may benefit from a treatment comprising an agonist of CD177 activity, the method comprising determining an expression level of podoplanin (PDPN) in a sample from the individual, wherein an expression level of PDPN in the sample that is above a reference PDPN expression level identifies the individual as one who may benefit from a treatment comprising an agonist of CD177 activity.

In some aspects, the invention comprises a method of selecting a therapy for an individual having a cancer, the method comprising determining an expression level of PDPN in a sample from the individual, wherein an expression level of PDPN in the sample that is above a reference PDPN expression level identifies the individual as one who may benefit from a treatment comprising an agonist of CD177 activity.

In some aspects, the individual has an expression level of PDPN in the sample that is above a reference PDPN expression level and the method further comprises administering to the individual an effective amount of an agonist of CD177 activity.

In some aspects, the invention comprises a method of treating an individual having a cancer, the method comprising (a) determining an expression level of PDPN in a sample from the individual, wherein the expression level of PDPN in the sample is above a reference PDPN expression level; and (b) administering to the individual an effective amount of an agonist of CD177 activity.

In some aspects, the invention comprises a method of treating an individual having a cancer, the method comprising administering to the individual an effective amount of an agonist of CD177 activity, wherein the expression level of PDPN in a sample from the individual has been determined to be above a reference PDPN expression level.

In some aspects, the CD177 activity is inhibition of PDPN.

In some aspects, the sample from the individual is a tumor tissue sample or a tumor fluid sample, e.g., a formalin-fixed and paraffin-embedded (FFPE) sample, an archival sample, a fresh sample, or a frozen sample.

In some aspects, the expression level of PDPN in the sample is a protein expression level of PDPN or an RNA expression level of PDPN. In some aspects, the expression level of PDPN in the sample is an RNA expression level of PDPN. In some aspects, the RNA expression level of PDPN is determined by in situ hybridization (ISH), RNA-seq, RT-qPCR, qPCR, multiplex qPCR or RT-qPCR, microarray analysis, SAGE, MassARRAY technique, FISH, or a combination thereof.

In some aspects, the benefit comprises an extension in the individual's recurrence-free survival (RFS) as compared to treatment without the agonist of CD177 activity. In other aspects, the benefit may comprise, e.g., an extension in the individual's overall survival (OS), an increase in time to recurrence, or a reduced duration of treatment as compared to treatment without the agonist of CD177 activity

B. Reference Expression Levels

In some aspects, the reference PDPN expression level is an expression level of PDPN in a population of individuals having a cancer, e.g., a population of individuals having a colorectal cancer (CRC).

In some aspects, the reference PDPN expression level is the 33^{rd} percentile, the 35^{th} percentile, the 40^{th} percentile, the 45^{th} percentile, the 50^{th} percentile, the 55^{th} percentile, the 60^{th} percentile, the 65^{th} percentile, the 66^{th} percentile, the 70^{th} percentile, the 75^{th} percentile, the 80^{th} percentile, the 85^{th} percentile, the 90^{th} percentile, the 95^{th} percentile, or the 99^{th} percentile of expression levels in the population of individuals having a cancer.

In some aspects, the reference PDPN expression level is the 50^{th} percentile of expression levels in the population of individuals having a cancer.

In some aspects, the reference PDPN expression level is the median of expression levels in the population of individuals having a cancer.

In some aspects, the reference PDPN expression level is the 33^{rd} percentile of expression levels in the population of individuals having a cancer.

In some aspects, the reference PDPN expression level is the 66^{th} percentile of expression levels in the population of individuals having a cancer.

In some aspects, the PDPN expression levels of the population of individuals are divided into tertiles, and the reference PDPN expression level is the lowest value in the second tertile.

In some aspects, the PDPN expression levels of the population of individuals are divided into tertiles, and the reference PDPN expression level is the lowest value in the third tertile.

In some aspects, the reference PDPN expression level is a pre-assigned PDPN expression level.

C. Cancers

In some aspects, the cancer is a CRC, a squamous cell carcinoma of the head and neck, or a glioma.

In some aspects, the individual has a colorectal cancer (CRC). In some aspects, the individual has had surgical resection of a CRC. In some aspects, the CRC of the individual is a stage I, stage II, or stage III, or stage IV CRC, e.g., a stage II CRC or a stage IV CRC, according to the TNM classification system at the onset of treatment. In some aspects, the CRC of the individual is a left-sided tumor, i.e., a tumor occurring in the distal colon (e.g., the distal third of the transverse colon, the splenic flexure the descending colon, the sigmoid colon, or the rectum) or a right-sided tumor, i.e., a tumor occurring in the proximal colon (e.g., the proximal two-thirds of the transverse colon, the ascending colon, and the cecum).

In some aspects, the agonist of CD177 activity results in an increase in the binding of PDPN and CD177 relative to binding of the two proteins in the absence of the agonist.

In some aspects, the agonist of CD177 activity results in a change in a downstream activity of PDPN relative to the downstream activity in the absence of the agonist of CD177 activity. In some aspects, the change in the downstream activity is a decrease in tumor growth or a decrease in cancer-associated fibroblast (CAF) contractility.

D. Agonists of CD177 Activity

In some aspects, the agonist of CD177 activity is a small molecule, an antibody or antigen-binding fragment thereof, a peptide, or a mimic.

In some aspects, the agonist of CD177 activity is a peptide, e.g., a CD177 peptide, e.g., an extracellular domain of CD177. The peptide may be multimerized, e.g., dimerized, trimerized, tetramerized, or pentamerized. In some aspects, the peptide is tetramerized, e.g., tetramerized using streptavidin.

In some aspects, the agonist of CD177 activity is an antibody or antigen-binding fragment thereof. In some aspects, the antibody or antigen-binding fragment thereof binds PDPN, e.g., is an antagonist antibody or antigen-binding fragment thereof that binds PDPN. In some aspects, the antibody or antigen-binding fragment thereof binds CD177, e.g., is an agonist antibody or antigen-binding fragment thereof that binds CD177.

In some aspects, the antigen-binding fragment is a bis-Fab, an Fv, a Fab, a Fab′-SH, a F(ab′)₂, a diabody, a linear antibody, an scFv, an ScFab, a VH domain, or a VHH domain.

Agonists of CD177 activity may be identified, e.g., using the methods for identifying modulation of an interaction or the methods for identifying a change in a downstream activity of a protein described in Sections IIIA and IIIB herein. For example, methods including surface plasmon resonance (SPR), biolayer interferometry (BLI), ELISA, or extracellular or cell surface interactions, as described herein, could be used to identify a modulator that increases the interaction between CD177 and PDPN, i.e., an agonist of CD177 activity.

In aspects in which the agonist of CD177 activity is an antibody (e.g., a CD177 agonist antibody or a PDPN antagonist antibody), the antibody may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

E. Methods of Delivery

The compositions utilized in the methods described herein (e.g., agonists of CD177 activity) can be administered by any suitable method, e.g., as described in Section VB herein.

F. Additional Therapeutic Agents

In some aspects, the agonist of CD177 activity is used with one or more additional therapeutic agents, e.g., a combination therapy, e.g., as described in Section VIF herein.

### VIII. Articles of Manufacture

In another aspect of the invention, an article of manufacture containing materials useful for the treatment, prevention, and/or diagnosis of the disorders described above is provided.

In some aspects, the invention comprises a solid surface or a set of solid surfaces (e.g., a multi-well plate or a set of multi-well plates) comprising a number of locations, each of the locations comprising a unique polypeptide from a collection of polypeptides, wherein each polypeptide comprises an extracellular domain, a tag, and an anchor, and wherein the collection of polypeptides comprises the extracellular domains of all or a subset of the proteins of Table 7. Exemplary collections of polypeptides are described in Section IIB.

In some aspects, the invention comprises a solid surface or a set of solid surfaces (e.g., a multi-well plate or a set of multi-well plates) comprising a number of locations, each of the locations comprising a plasmid encoding a unique polypeptide as described above. In some aspects, the solid surface or set of solid surfaces has been stamped with the polypeptide.

In some aspects, the invention comprises a solid surface or a set of solid surfaces (e.g., a multi-well plate or a set of multi-well plates), each of the locations comprising a unique polypeptide from a collection of polypeptides, wherein the collection of polypeptides comprises the extracellular domains of all or a subset of the proteins of Table 7, wherein said polypeptides are immobilized to one or more solid surfaces, wherein each of the one or more of said polypeptides is immobilized to a distinct location (e.g., a distinctly interrogatable location, e.g., a location that can be interrogated distinctly by the methods described herein) on said one or more solid surfaces. The distinct location may be an area on a surface where a cell line is plated, e.g., a well.

In some aspects, the solid surface or set of solid surfaces together comprise at least 965 locations, each of the locations comprising a unique polypeptide from a collection of polypeptides, wherein each polypeptide comprises an extracellular domain, a tag, and an anchor, and wherein the collection of polypeptides comprises the extracellular domains of at least 81% of the proteins of Table 7.

In some aspects, the solid surface or set of solid surfaces comprises at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, or 1195 locations, e.g., comprises 100-150, 150-200, 200-250, 250-300, 300-350, 350-400, 400-450, 450-500, 500-550, 550-600, 600-650, 650-700, 750-800, 800-850, 850-900, 900-950, 950-1000, 1000-1050, 1050-1100, 1100-1150, or 1195 locations, each comprising a unique polypeptide from the collection of polypeptides or a plasmid encoding such a polypeptide.

In some aspects, the collection of polypeptides comprises the extracellular domains of at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the proteins of Table 7.

In some aspects, the collection of polypeptides comprises the extracellular domains of at least 81% to 100% of the proteins of Table 7, e.g., comprises at least 85%, 90%, 95%, or 100% of (e.g., comprises all of) the proteins of Table 7, e.g., comprise the extracellular domains of 81%-85%, 83%-87%, 85%-89%, 87%-91%, 89%-93%, 91%-95%, 93%-97%, 95%-99%, or 100% of the proteins of Table 7.

In some aspects, the collection of polypeptides comprises the extracellular domains of at least 80% to 81% of the proteins of Table 7, e.g., comprises at least 80.1%, 80.2%, 80.3%, 80.4%, 80.5%, 80.6%, 80.7%, 80.75%, 80.8%, or 80.9% of the proteins of Table 7.

In some aspects, the collection of polypeptides comprises the extracellular domain of at least one of the proteins of Table 17, e.g., comprises the extracellular domains of at least 2, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or all 231 of the proteins of Table 17, e.g., comprise the extracellular domains of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100,101-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 214-220, 220-225, 225-230, or all 231 of the polypeptides of Table 17.

In some aspects, the invention comprises a set of containers (e.g., a set of vials), each vial comprising a plasmid encoding a unique polypeptide as described above.

### IX. Examples

The following are examples of methods and compositions of the invention. It is understood that various other aspects may be practiced, given the general description provided above, and the examples are not intended to limit the scope of the claims.

### Example 1. Assay for Interactions Between IgSF Proteins and a Human STM Receptor Library

Using a technology for unbiased detection of low-affinity interactions, a library containing most human single transmembrane (STM) receptors (1,226 receptors) was assayed for binding to 445 immunoglobulin superfamily (IgSF) proteins. IgSF proteins are known to function through formation of homo- and heterophilic complexes that mediate a wide array of functionalities, such as modulation of axon guidance or synaptic plasticity, control of cell migration and adhesion, and self vs. non-self recognition, and as such, these proteins constitute a major focus for drug development efforts.

A. IgSF Query Collection

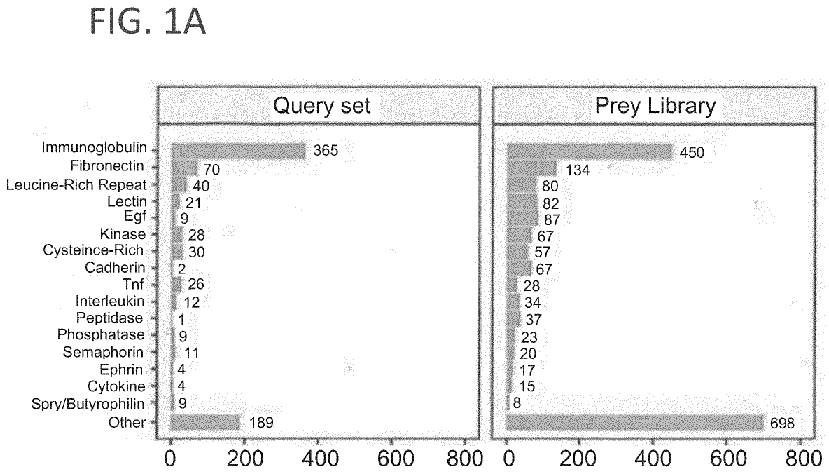

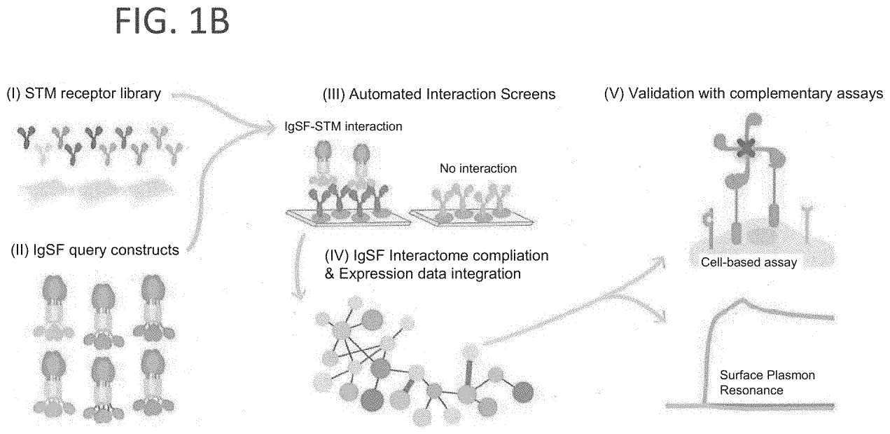

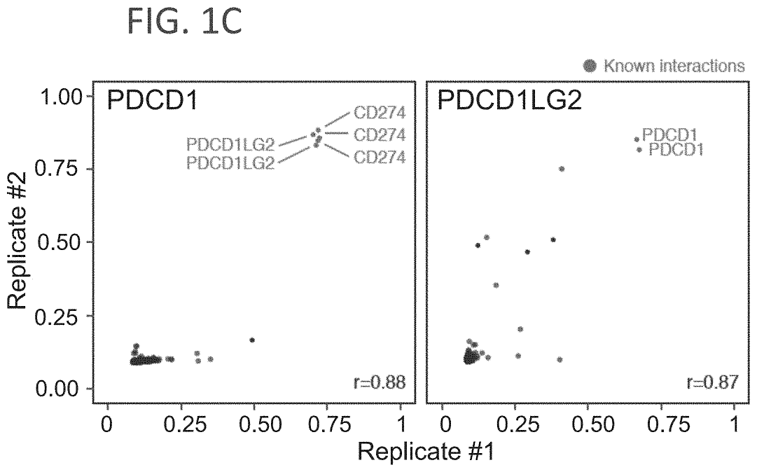

The immunoglobulin superfamily (IgSF) collection (query collection) was defined by integrating functional annotations and computational predictions from various computational algorithms for prediction of protein features, followed by careful manual curation and review. First, a list of proteins a predicted “Immunoglobulin-like domain superfamily,” according to InterPro (IPR036179), was downloaded from UniProt. This list was complemented with 98 selected proteins based on their participation in relevant biological functions, and was then further curated in light of evidence for extracellular domains and motifs relevant to receptor signaling functions, restricting it to a subset of 445 proteins (Clark et al., Genome Res, 13: 2265-2270, 2003; Daeron et al., Immunol Rev, 224:11-43, 2008; Yap et al., J Mol Biol, 426: 945-961, 2014). The final query set includes 365 human IgSF proteins (≈82% of the IgSF, according to InterPro annotations) and 98 additional proteins, some of which are also annotated with ‘Ig-like Fold’ in SwissProt (FIG. 1A; Table 4). IgSF proteins were cloned as previously described (Bushell et al., Genome Res, 18: 622-630, 2008; Martinez-Martin et al., Cell, 174(5): 1158-1171, 2018). In brief, the ECD of each IgSF query protein was fused to the pentameric helical region of rat cartilage oligomeric matrix protein (COMP) and β-lactamase, increasing avidity and allowing a colorimetric readout upon addition of the substrate nitrocefin (FIG. 1B). All clones were synthesized using sequences codon-optimized for mammalian cell expression.

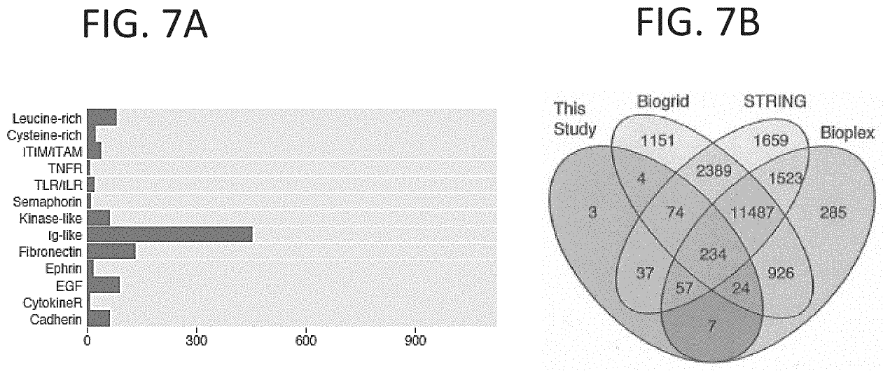

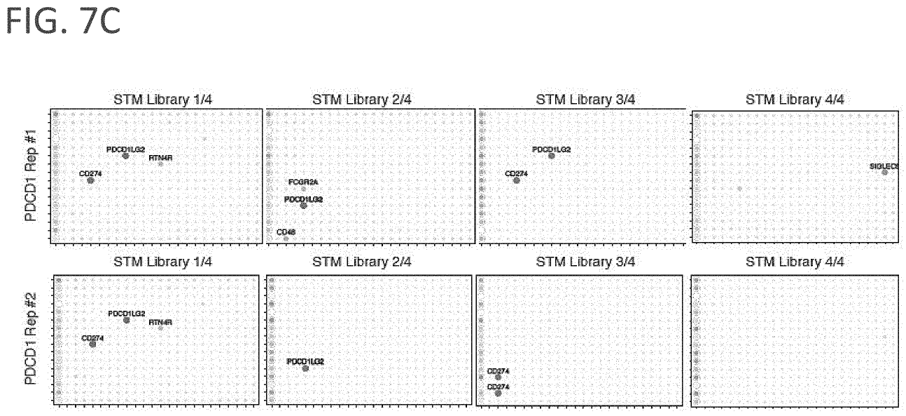

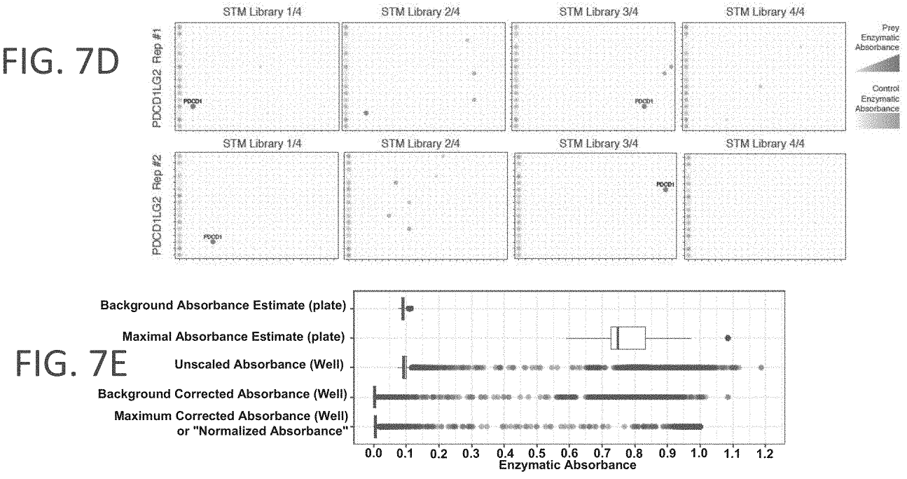

The power of this technology to detect receptor-ligand interactions characterized by a wide range of affinities, including transient interactions with micromolar K_{D}, has previously been demonstrated (Martinez-Martin et al., Cell, 174(5): 1158-1171, 2018). To further benchmark the sensitivity and reproducibility of this platform for detection of interactions between human receptors, PD-1/PDCD1 and PD-L2/PDCD1LG2 proteins were screened following the established procedure (FIG. 1B). Notably, all expected interactors were detected with high normalized signal (>0.75), high binding specificity and excellent reproducibility (Pearson's r>=0.87) (FIG. 1C and FIGS. 7C-7E), further demonstrating the robustness of the receptor interaction discovery platform.

B. Human STM Receptor Library

The list of single transmembrane (STM) receptors (prey library) was compiled by integrating functional annotations and computational predictions from various computational algorithms for prediction of protein features, followed by careful manual curation and review of published annotations (Clark et al., Genome Res, 13: 2265-2270, 2003; Martinez-Martin et al., Cell, 174(5): 1158-1171, 2018). The library consists of 1,266 unique human STM receptors (Table 5). STM receptors were expressed as extracellular domains (ECDs) fused to a human Fc tag (e.g., soluble ECDs). This facilitates protein expression, circumvents the need for solubilization in the presence of detergents, and allows robust capture on protein A-coated plates (FIG. 1B). In brief, the boundaries of the extracellular domain (ECD) were determined by predicting signal peptides and transmembrane helices or glycosylphosphatidylinositol (GPI)-linkage sites using publicly available servers for prediction of protein features (Phobius, TMHMM, SignalP3.0). The ECD of each receptor was synthesized and cloned into the pRK5 vector (Genentech) containing a C-terminal human Fc tag. For type II STM proteins, a HSV signal sequence was inserted upstream of an N-terminal Fc tag.

C. Cell Culture and Conditioned Media Generation

i. Cell Culture

Conditioned media for the STM receptor library was prepared using Expi293F cells (Thermo-Fisher), a suspension cell line adapted from HEK293 cells (epithelial human cells, embryonic kidney). Cells were cultivated under the following conditions: 37° C., 8% CO₂, 80% humidity and 150 rpm agitation speed. Expi293 Expression Medium (Life Technologies) was used as the seed train and production media. The same cell line and culture conditions were used for expression of the secreted, pentameric IgSF proteins. COS7 cells (fibroblast cell line derived from monkey kidney tissue, purchased from ATCC) or HEK-293 cells were used for transient expression of the relevant binding partners, expressed as full-length proteins (Genentech). Transfections were performed using Lipofectamine LTX with PLUS Reagent (Life Technologies) in Opti-MEM media (Life Technologies). Cells were cultured in DMEM media supplemented with 10% FBS, 2 mM L-glutamine, 100 U/mL penicillin and 100 μg/mL streptomycin in a 37° C. humidified, 5% CO₂ incubator.

ii. Conditioned Media Generation

The collection of STM receptors (prey library; cloned as the extracellular domain (ECD) fused to a human Fc tag (ECD-Fc)) was transiently transfected in human cells for expression as soluble proteins in the conditioned media. Cell culture and instrumentation for cell transfection automation have been described in detail (Bos et al., Biotechnol Bioeng, 112: 1832-1842, 2015). In brief, transfections were performed using Expi293 (Life Technologies), a suspension-adapted HEK293 line. Expi293 Expression Medium (Life Technologies) was used as the seed train and production medium. Cells were cultivated as a seed train in flasks and grown at 37° C., 5% CO₂, and 150 rpm agitation speed in a humidified incubator before transient transfection. A Tecan EVO liquid handling system (Tecan) and integrated MultiDrop Combi reagent dispenser (Thermo Fisher) was utilized for all automated cell culture operations. The prey library was prepared using micro-scale 1 mL transient transfections, using an automated platform that has been recently described (Bos et al., Biotechnol Bioeng, 112: 1832-1842, 2015; Martinez-Martin et al., Cell, 174(5): 1158-1171, 2018). In brief, DNA was purified at the miniprep scale using a high throughput plasmid purification system, and 1 μg of DNA was dispensed in each well. For generation of conditioned media enriched in oligomeric query proteins, 30 mL transient transfections using a total of 30 μg in each well were carried out in 50 ml tubespins processed in batches of 96 for efficiency using a Biomek FX liquid handling robot (Beckman Coulter), essentially as described (Bos et al., Biotechnol Bioeng, 112: 1832-1842, 2015). 25 KDa Linear polyethylenimine (PEI) was used for the transient transfection procedures, and conditioned media were harvested 7 days post-transfection. Cells were removed by spinning at 3,000 rpm for 30 minutes, and supernatants were stored at 4° C. until processed.

iii. Preparation of STM Library-Coated Plates for Protein-Protein Interaction Screens

The receptor-ligand screening technology utilized was based on the avidity-based extracellular interaction (AVEXIS) method (Bushell et al., Genome Res, 18: 622-630, 2008), further adapted for automated high throughput screening in 384 well plate format (Martinez-Martin et al., Cell, 174(5): 1158-1171, 2018). In brief, to prepare the conditioned media for the high-throughput extracellular interaction screens, the STM prey library and IgSF query proteins were produced in a human expression system as described above in order to maximize addition of the relevant post-translational modifications. Cell transfections were performed as described, and cell cultures were grown for 7 days before removing the cells by centrifugation at 3,000 g for 30 min. Protein A-coated plates (Thermo Scientific) were used to capture the prey library from conditioned media by overnight incubation followed by storage at 4° C. A similar procedure was used to prepare the IgSF query proteins, which were assayed directly in the conditioned media without any capturing step. Prior to screening, the concentration of each pentameric IgSF receptor was normalized using β-lactamase activity in the conditioned media as readout. Briefly, a dilution series of the supernatant was added to nitrocefin (0.125 mg/mL) and immediately transferred to a plate reader to record absorbance at 485 nm every minute for a total of 20 min. The expression levels for each query protein were normalized to threshold levels previously determined to identify interactions of ≤10 μM, as described (Bushell et al., Genome Res, 18: 622-630, 2008).

iv. Analysis of ECD-Fc Receptor Concentration in Conditioned Media

The concentration of each STM receptor (ECD-Fc) in the conditioned media utilized for the assays was measured using a human IgG, Fcγ time-resolved (TR)-FRET assay. AffiniPur F(ab′)₂ Goat anti-Human IgG, Fcγ (Jackson ImmunoResearch) conjugated with Europium Cryptate (Cisbio Bioassays), and AlexaFluor647R-AffiniPur F(ab′)₂ Donkey anti-Human IgG, Fcγ were used as donor and acceptor, respectively. Standards, controls and samples were diluted in assay diluent (PBS/0.5% BSA/0.05% Tween-20/15 ppm Proclin) and added to 1536-well MaKO white plates (Aurora Biotechnologies). A combined donor and acceptor reagent solution was then added to each well. Plates were allowed to incubate at ambient temperature for 1 hour, followed by reading using a PHERAstar FS (BMG Labtech) plate reader with an excitation wavelength of 320 nm and emission wavelengths of 665 nm and 620 nm. The TR-FRET signal was reported as the ratio of the two emission wavelengths (665 nm/620 nm) multiplied by 10,000. Sample quantification was obtained by interpolating the results from a 5 parameter fit of the standard. Data was processed using a custom software.

D. Automated Cell Surface Interaction Screen

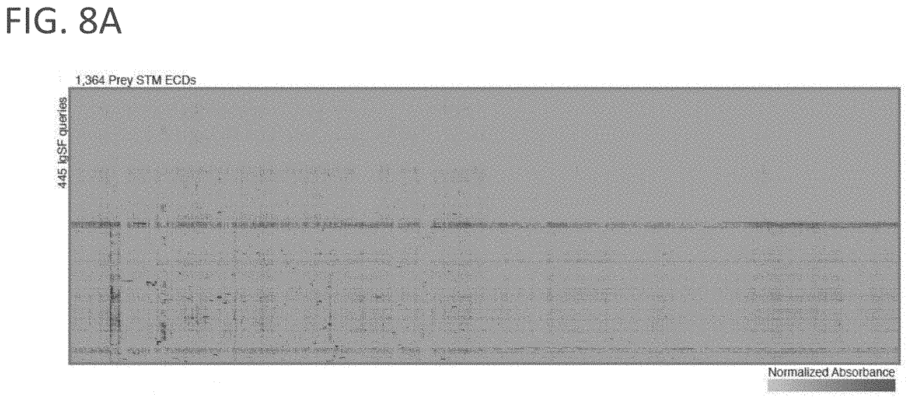

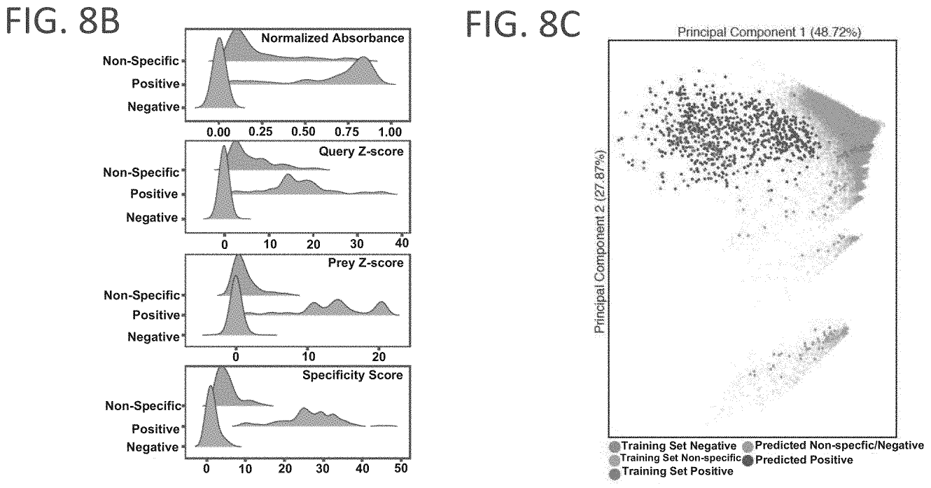

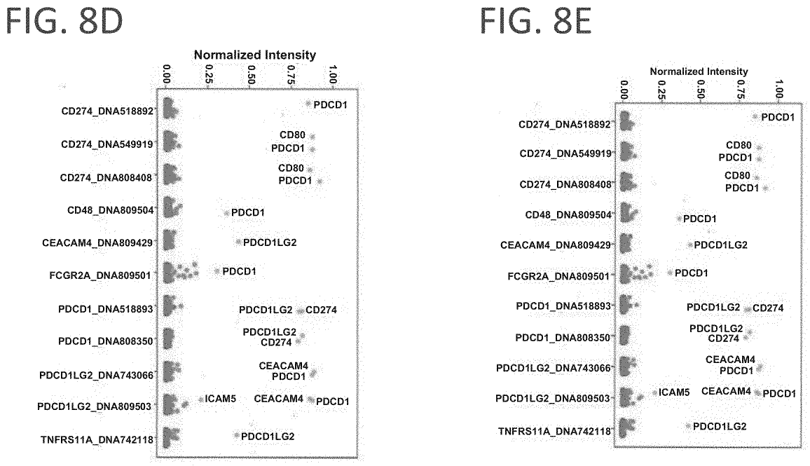

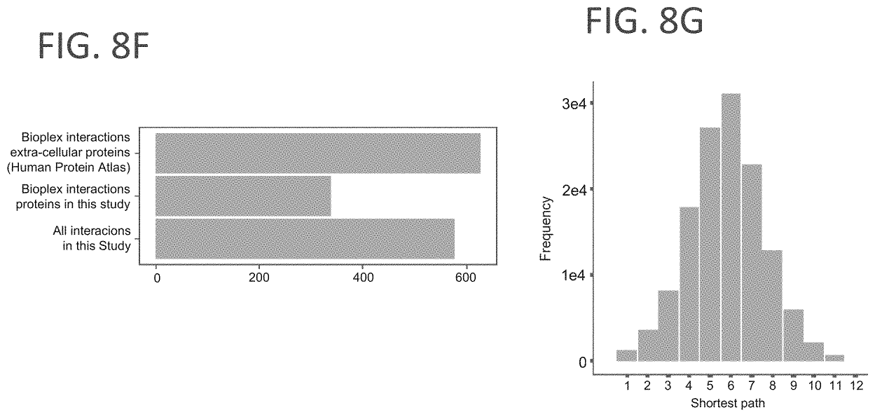

To systematically explore the interaction repertoire of the IgSF members, each query protein was individually screened against the entire library of STM receptors, which resulted in testing of ˜600,000 binary binding events from over 2,000 individual 384-well plates (FIG. 8A).

Preparation of STM receptor library-coated plates and screening of the oligomeric IgSF proteins against the STM receptor library were performed using an integrated robotic system consisting of automated liquid handling devices (plate dispensers and washers), to allow for high throughput analysis of protein-protein interactions and minimize manual operations to improve screening data quality (Martinez-Martin et al., Cell, 174(5): 1158-1171, 2018). The system was a fully automated microplate assay system that consists of several devices integrated with a robotic arm. This system was configured with a Biomek FX liquid handler (Beckman Coulter) with a 384-channel pipette head for plate-to-plate sample transfer, a Thermo Cytomat 9 hotel carousel (Thermo Fisher Scientific) for storing assay plates and tip boxes, a BioTek EL406 combination washer/dispenser (BioTek) for washing and dispensing reagents to plates, and a BioTek MultiFlo dispenser (BioTek) for dispensing additional reagents. A TECAN Infinite M1000 multimode microplate reader (Tecan) was used to record signal from screening plates. The method was developed using Beckman Coulter SAMI software, which schedules the methods according to the number of assay plates in a run and controls the execution of the entire automated processes.

Every 384 well screening plate used in this study was configured with 16 wells reporting the maximal enzymatic absorbance potential of the query construct (used as positive control for automation procedures), 16 blank wells to report plate background (negative control), and 346 randomly spotted prey proteins from the STM library. On average, this workflow allowed screening of up to 7 query proteins a day against a library consisting of 1,364 preys distributed over 4 screening plates (FIG. 7C). On the day of the screen, protein A-coated plates that had been incubated overnight with the conditioned media library were washed three times with PBS containing Ca²⁺ and Mg²⁺. Subsequently, plates were inoculated with the conditioned media containing the pentamerized IgSF query protein (50 μL/well) and incubated at room temperature for 1 h. Plates were then washed with PBS to remove any unbound IgSF pentamer. Nitrocefin (50 μL/well) was added (Calbiochem). Nitrocefin hydrolysis, indicated by a color change, is observed in the presence of β-lactamase activity and indicated that the IgSF query protein was bound to the individual STM receptor captured in the well (FIG. 1B). Plates were incubated at room temperature (RT) for 1 h, and IgSF-STM receptor interactions were assessed by measuring absorbance at 485 nm.

### Example 2. Analysis of IgSF Extracellular Interaction Screen Results and Prediction of the IgSF Interactome

To enable analysis of this large dataset and reliably identify non-specific (false positives) binders and compute a binding score in an automated manner, we developed a computational classification tool.

First, raw enzymatic absorbance values were corrected per screened plate by subtracting the plate's estimated background enzymatic absorbance (10%-ile) and subsequently scaling those values to the maximal enzymatic absorbance estimate (99.5%-ile) to derive a normalized absorbance value within the [0,1] range for each measured well. All absorbance controls and empty well values were filtered out and the normalized absorbance values were compiled in a query (row) by prey (column) data matrix. Using the data matrix, four predictive features were computed for each query-prey pair: 1) the normalized absorbance value 2) a query Z-score (Z-score across all preys on the 4 STM library plates for a single query); 3) a prey Z-score (Z-score per prey in the STM receptor library across all queries that were screened against it); and 4) a custom Specificity Score, computed as follows:

$${A}_{N}^{q , p} = Normalized query prey Absorbance ; {A}_{N}^{P} = {\sum }_{i = 1}^{q} {A}_{N}^{q , p} = Sum Prey Absorbance ;$$

$${A}_{N}^{S} = {\sum }_{i = 1}^{p} {A}_{N}^{P} = Sum Screen Absorbance ; {S}^{q , p} = \sqrt[2]{{A}_{N}^{q , p} * \frac{{A}_{N}^{S}}{{A}_{N}^{P}}} = Specificity Score$$

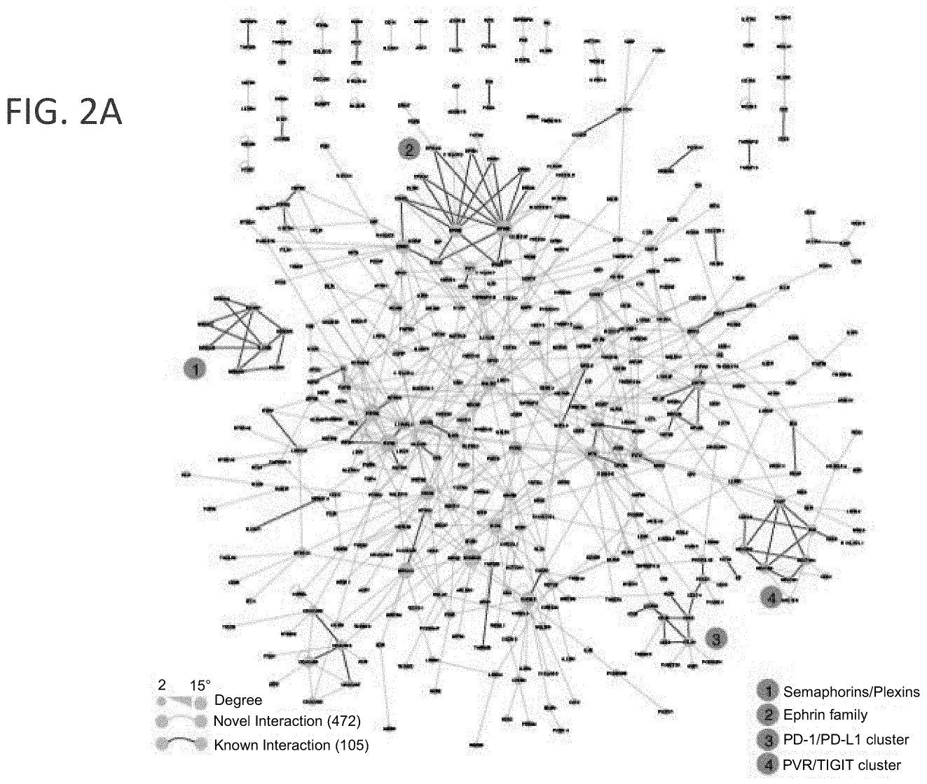

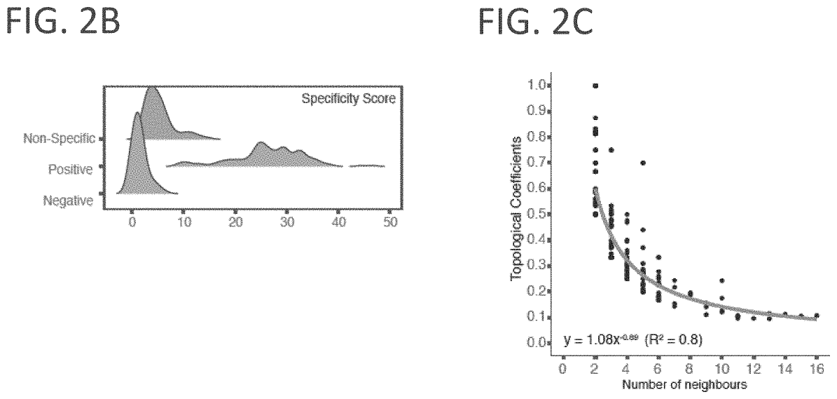

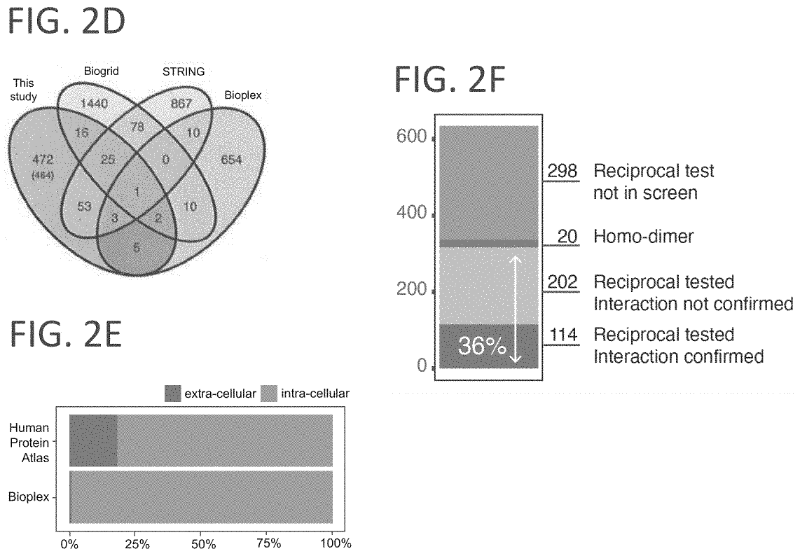

A supervised random forest classifier, implemented in the Caret R package, was trained using all four features on a benchmark data set of compiled true positive receptor interactions from literature, observed “nonspecific” preys from orthogonal library screens (Table 14) and true negative interactions, sampled at a 1:10 positive to negative ratio from the data points below the 99-%-ile (Normalized Absorbance <0.057) (FIGS. 8C-8E). All absorbance controls were removed prior to training and prediction. The training function in Caret was configured with the following parameters: training set size 75%, test set size 25%, repeated cross-validation (N=10, R=10), multi-class predictions (Positive, Negative, Non-specific), grid-search parameter optimization for Random Forest (mtry=20, .ntree=30) and Accuracy as performance metric. The predicted ‘highconfidence’ IgSF interactome was defined by additionally filtering by the following class probabilities: P(Positive) >=0.75, P(Non-specific)<=0.25 and P(Negative)<=0.05 (FIGS. 2B and 8B). Finally, for representation and data integration purposes, all interactions identified with multiple prey constructs for identical STM receptors and bi-directionally identified interactions were summarized into a non-directional, non-redundant list of unique binding partners.

This classification exercise resulted in 577 predicted high-confidence interactions between 440 unique IgSF and STM proteins, referred to as the “IgSF interactome” (FIG. 2A, Table 6). To facilitate data integration, analysis and visualization, the IgSF interactome was represented as a Cytoscape interaction network where “nodes” represent extracellular proteins and “edges” the interactions between them (FIG. 2A). The IgSF Interactome network is highly connected (FIG. 2A). Connectivity in biological networks has been the subject of many studies, which postulate that their scale-free properties contribute to robustness (Barabasi, Science, 325: 412-413, 2009). Indeed, a network analysis of the IgSF Interactome showed that each protein is connected on average to 2-3 neighboring proteins and that the topological coefficients follow a power-law distribution (FIG. 2C), a hallmark of scale-free networks. This suggests that the extracellular interactome is not made up of disconnected receptor-ligand pairs, but rather consists of modular components that have dense intra-connectivity and sparse, but relevant interconnectivity (Albert, J Cell Sci, 118: 4947-4957, 2005).

The IgSF interactome identified the remarkable number of 472 novel interactions, and recapitulated 105 previously documented interactions in the aggregate of Biogrid, Bioplex and STRING databases (FIG. 2D).

### Example 3. Integration of IgSF Interactome Data with Public Datasets

A. IgSF Interactome and Bioplex, Biogrid, and STRING Datasets

The IgSF interactome dataset was compared to the Bioplex, Biogrid, and STRING datasets (Chatr-Aryamontri et al., Nucleic Acids Res, 45: D369-D379, 2016; Huttlin et al., Nature, 545: 505-509, 2017; Szklarczyk et al., Nucleic Acids Res., 43: D447-452, 2015), currently the most systematically compiled resources for human protein interactions. The Bioplex dataset was downloaded from the Bioplex webpage (v2.0). The Biogrid dataset was downloaded from the Biogrid webpage (v3.4.160, physical interactions). The STRING dataset, including all STRING “evidence channels”, was downloaded from the STRING website (v.10.5) and filtered to retain only interactions with a STRING “combined score >0.7” based on the previously described weighted evidence channels (von Mering et al., Nucleic Acids Res, 33:D433-437, 2005). To compute the overlap percentages, all three protein interaction resources were restricted to the scope of binary interactions tested in this work, being the combinatorial set of all query by prey proteins. All network statistics were computed in Cytoscape (v.3.6.1) (Shannon et al., Genome Res, 13: 2498-2504, 2003).

In relative terms, our dataset showed the largest overlap with the STRING database (82/1,037) and the smallest overlap with the combined Bioplex in HEK293 and HCT116 cells (11/350), an important observation given the scale of the Bioplex effort (˜120,000 interactions among nearly 15,000 proteins), and the fact the nearly all proteins studied in our work are also part of the Bioplex and STRING databases (FIG. 7A). Integration of protein localization information from the Human Protein Atlas revealed that the number of interactions between two extracellular proteins found in Bioplex is disproportionally low, with only ˜1% of this dataset representing interactions between the set of extracellular proteins encoded by the human genome (FIG. 2E). The challenges of capturing plasma membrane-expressed proteins with a standard AP-MS workflow and the Bioplex projects' focus on intracellular tagged constructs offer logical explanations for this observation, and further highlight the relevance of the present study to address interactions that take place in the extracellular environment. Finally, given that 238 STM proteins represented on the network were also included as query proteins, it was possible to detect these as reciprocal interactions in our screen. Indeed, 114 interactions (out of 316, ˜36%) were observed both as query-prey and prey-query pair (FIG. 2F).

B. Network Clustering by Healthy Tissue Expression Correlation

The inherent challenges associated with the study of plasma membrane proteins have importantly hindered a systematic exploration of functional relationships between human receptors. To our knowledge, only a few studies have attempted to classify cell receptors into functional families through predicting shared ligand interactions from sequence and structure features, and virtually all predictions lack experimental validation. To facilitate a functional interpretation of the IgSF interactome, we integrated healthy tissue mRNA expression profiles for all proteins (network nodes) in the IgSF interactome from the Genotype-Tissue Expression (GTEx) project (Pierson et al., PLoS Comput Biol, 11, e1004220, 2015).

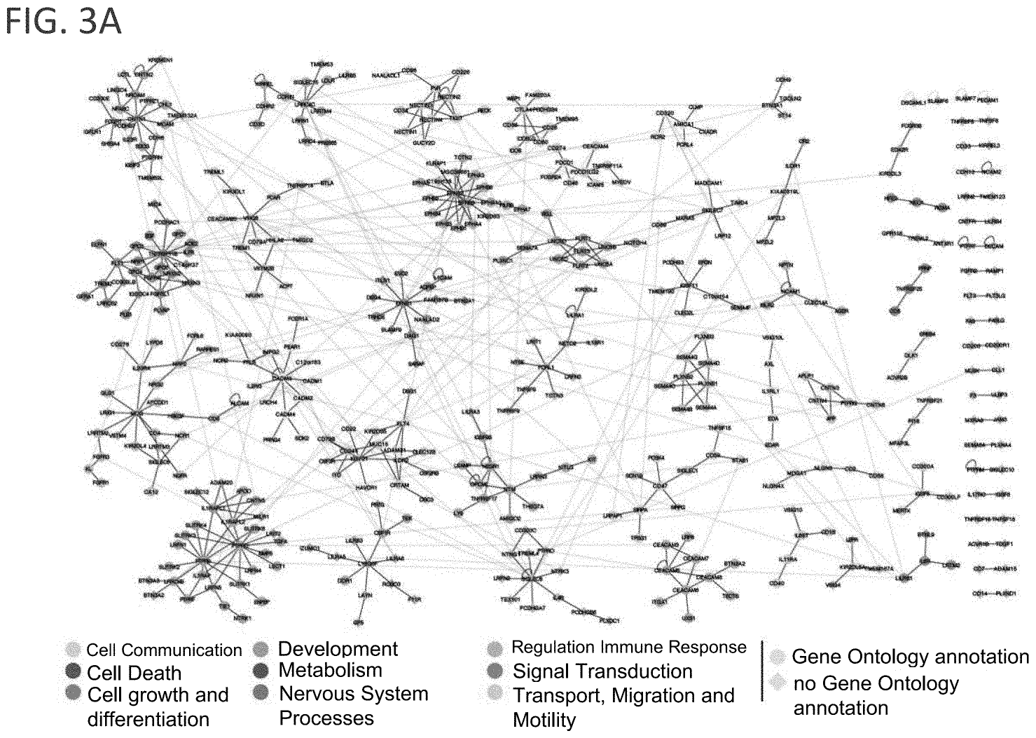

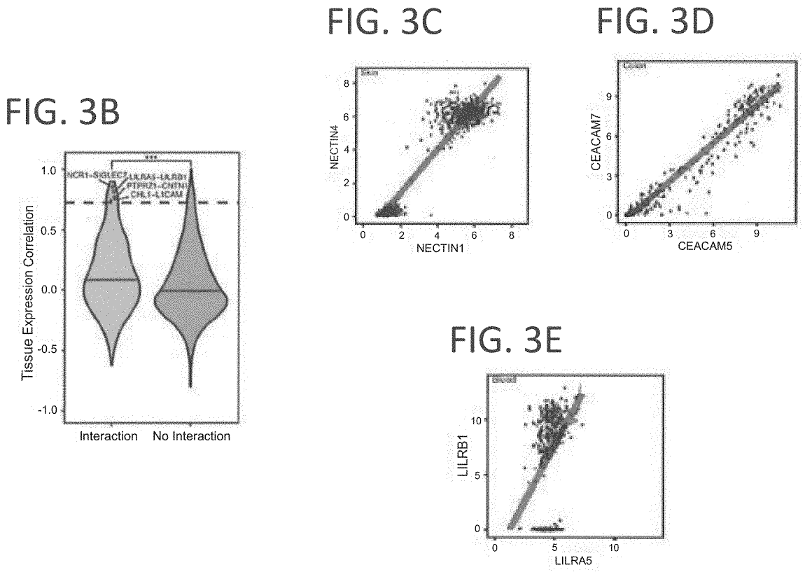

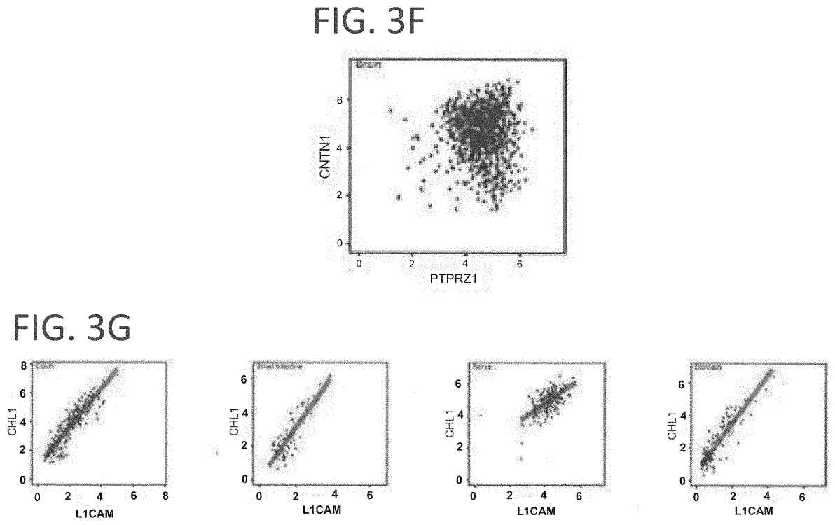

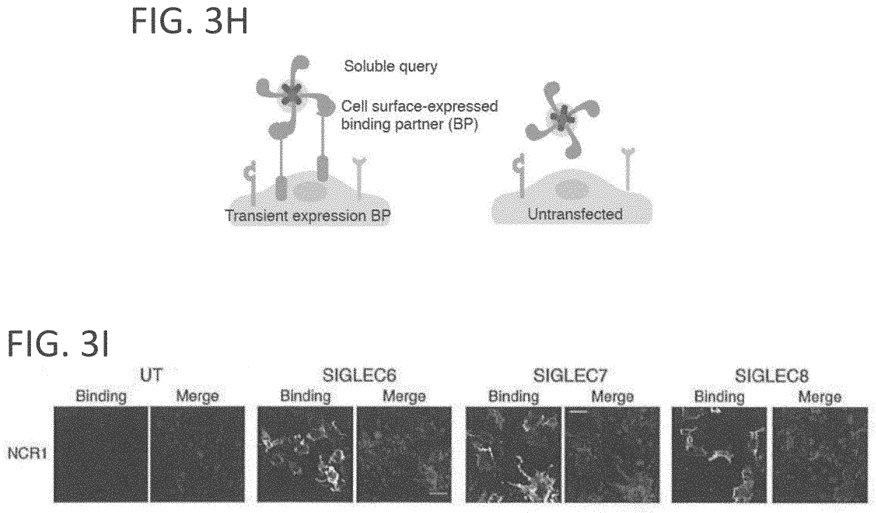

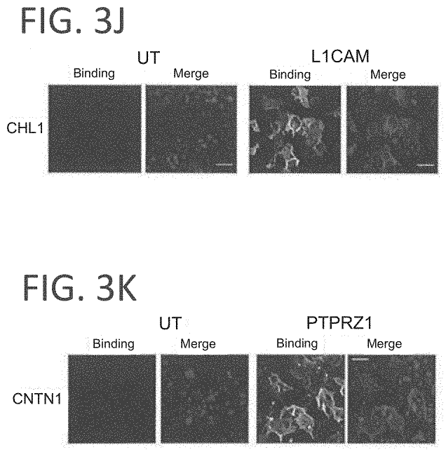

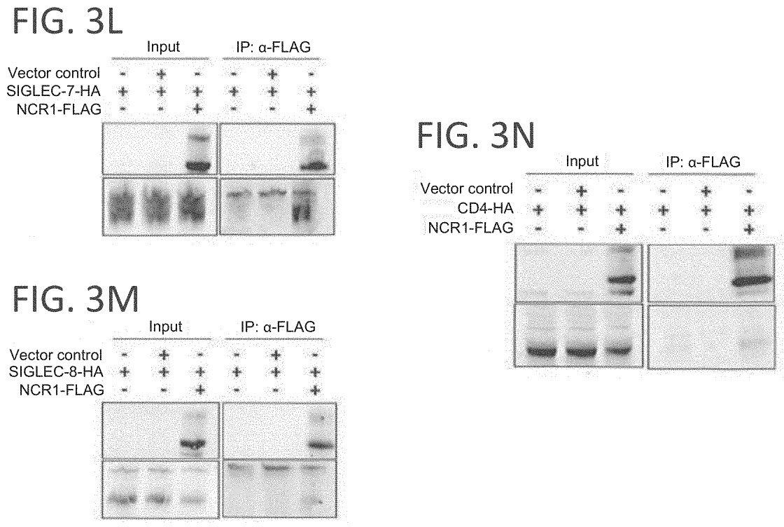

Tissue expression correlations between node pairs in GTEx were calculated as the Spearman coefficient on the log 2-transformed RPKM across all tissue detail categories. All test p-values were adjusted for multiple hypothesis testing using the Benjamini-Hochberg method. All heatmaps were plotted with the pheatmap R package. Unsupervised clustering was performed with the Euclidean distance and Ward linkage. All network statistics were computed in Cytoscape (v.3.6.1) (Shannon et al., Genome Res, 13: 2498-2504, 2003). Network clustering was also performed in Cytoscape using the Markov Clustering Algorithm (MCL), implemented in the ClusterMaker2 plugin, with the Spearman correlation coefficients as edge weights (inflation parameter=1.4, stop if residual increases=False, number of iterations=500). Gene Ontology (GO) enrichment statistics were computed per MCL cluster using the GOstats R package (Falcon and Gentleman, Bioinformatics, 23: 257-258, 2007). Significantly enriched GO Biological Process terms were determined by q-value <0.05. All significant terms were manually curated and aggregated in the overarching categories depicted in FIG. 3A.

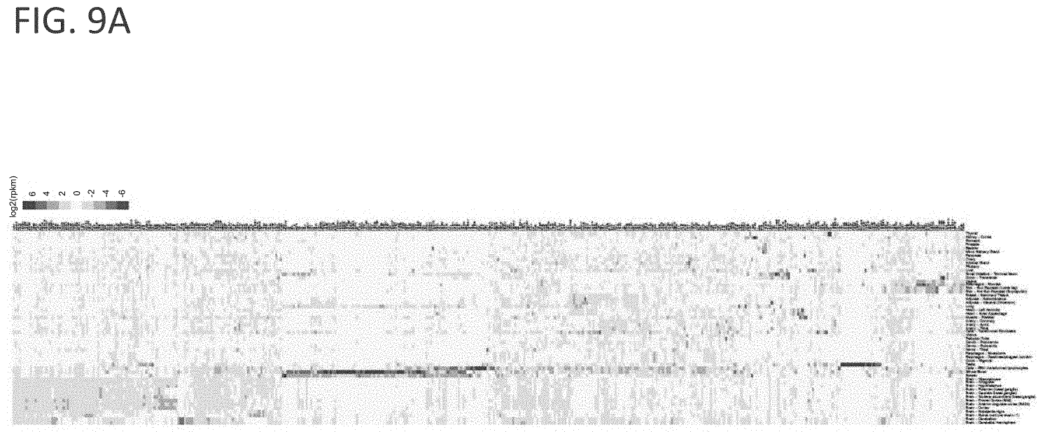

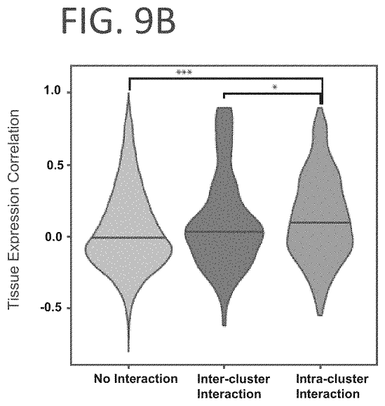

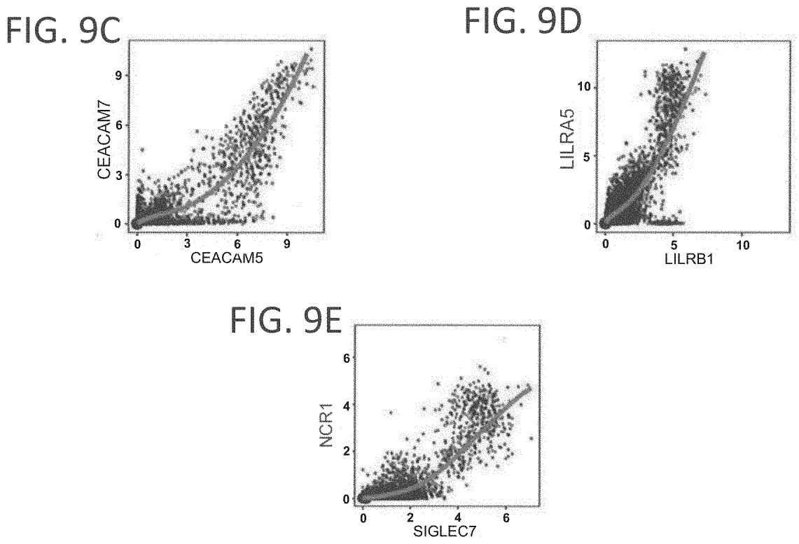

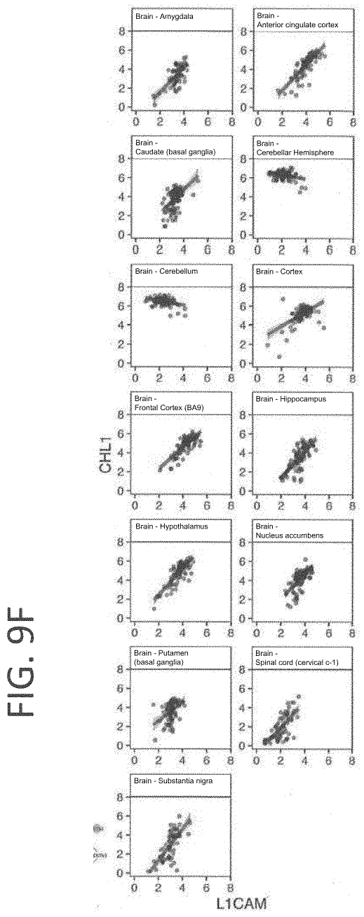

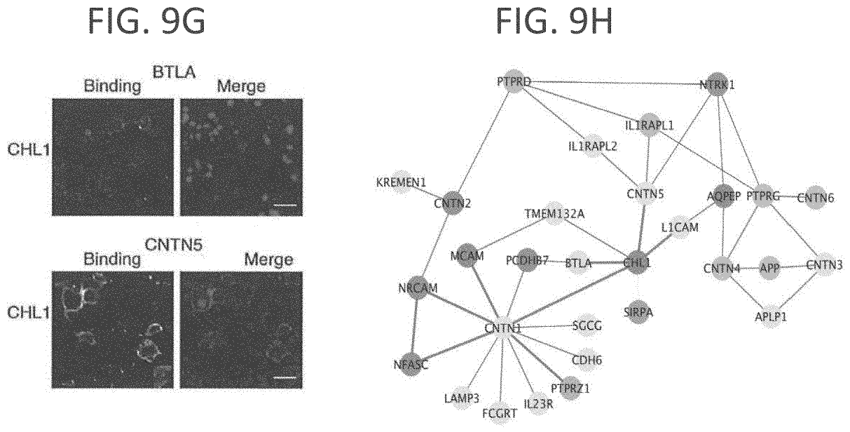

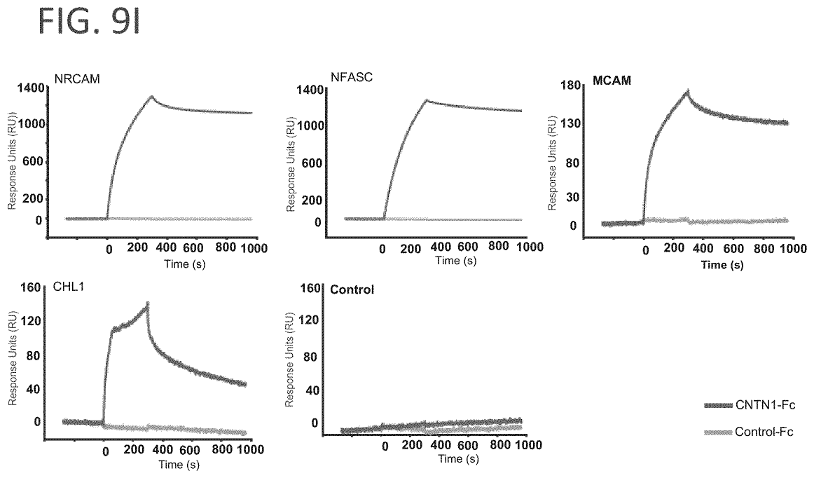

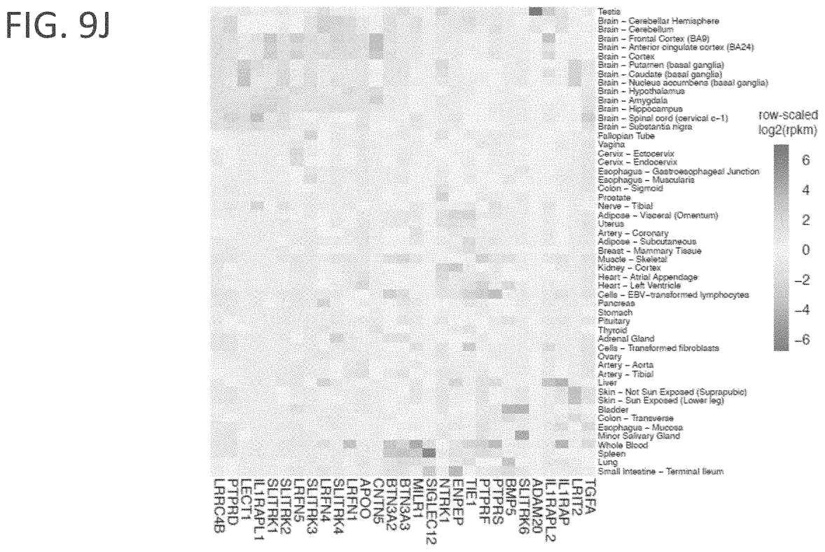

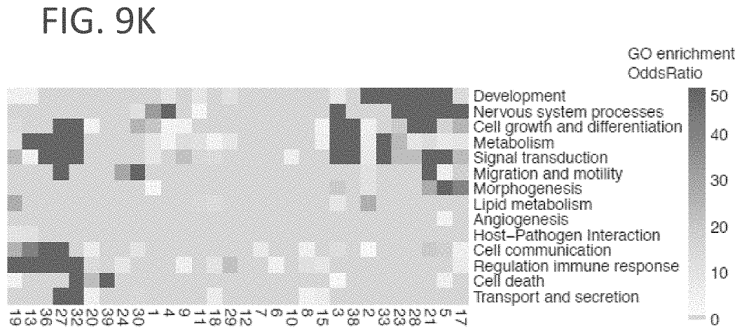

We hypothesized that modular components of connected IgSF and STM proteins are more likely to share a functional relationship and would be commonly expressed in the same tissue. In agreement with this hypothesis, the global tissue correlation coefficients for pairs of interacting proteins were indeed significantly higher compared to those of non-interacting pairs in our screen (p<1.2 10⁻²⁰, one-sided Wilcoxon rank-sum test) (FIGS. 3B and 9A). Given that there was a clear agreement between physical and co-expression associations, the expression correlation coefficients were used to inform clustering of the IgSF Interactome into modular components or ‘communities’ (FIG. 3A). As expected, co-expression coefficients were significantly higher within these communities, compared to all interactions between them (FIG. 9B).

The clustered Interactome displayed a number of tightly connected communities between closely related IgSF members, including the CEACAM proteins (FIG. 3A, cluster 18), the PVR/Nectin receptors (FIG. 3A, cluster 13), the Semaphorin/Plexin family (FIG. 3A, cluster 21) or the ephrin family of receptor tyrosine kinases (RTK) (FIG. 3A, cluster 5), among others. The co-expression and clustering results also revealed strong associations for many previously unknown protein pairs including MDGA1/neuroligins or the immune receptor AXL and its putative binding partners, suggesting a physiological role for these interactions in those tissues. Next, all network communities were interrogated with a custom Gene Ontology (GO) enrichment analysis to attempt functional classification of the proteins within the Interactome communities. This enrichment analysis showed that all sizeable communities (n>2) captured biological associations relevant to known IgSF functions such as immune response regulation, nervous system development, signal transduction and intercellular communication, and enabled assignment of putative functionalities for protein poorly characterized proteins, based on association with relatively well studied binding partners (FIG. 3A).

Although interacting proteins had significantly more correlated tissue expression patterns compared to non-interacting pairs, the modest mean shift between both distributions suggested that this difference was driven by selected interactions and/or interactions in a specific tissue context. Indeed, within the top 5% most correlated interacting pairs, many known protein pairs showed more prominent correlation patterns in discrete tissues, such as Nectin1-Nectin 4 (FIG. 3C) or CEACAM5-CEACAM7 (FIG. 3D and FIG. 9C), which were highly correlated in skin and colon, respectively.

Interestingly, many of the novel interactions found in the IgSF Interactome dataset also displayed a similarly strong tissue-dependent association. For example, LILRA5 and LILRB1 were highly associated in blood (FIG. 3E and FIG. 9D); PTPRZ1 and CNTN1, in brain (FIG. 3F); NCR1 and SIGLEC7 (FIG. 9E), in multiple tissues; or CHL1 and L1CAM (FIG. 3G and FIG. 9F), strongly associated across tissues, suggesting a relevant role for these interactions in certain biological scenarios. We next asked whether these interactions could take place “in-trans” on cells, using a tetramerization-based approach for high sensitivity detection of putative binders expressed on the cell surface (FIG. 3H). These results demonstrated binding of NCR1 to specific members of the SIGLEC family (FIG. 3I), the interaction between CHL1 and L1CAM (FIG. 3J and FIGS. 9G and 9H), as well as binding of CNTN1 to PTPRZ1 and the rest of putative binding partners (FIG. 3K and FIGS. 9H and 9I). Co-immunoprecipitation studies showed that NCR1 interacted with all the newly identified binding partners in the context of the plasma membrane, further supporting a role for these interactions (FIGS. 3L-3N). Together, these analyses enabled a description of possible functional associations between protein families that were previously undocumented, governed by co-expression of binding partners. Consequently, this effort empowers hypothesis driven studies of putative molecular functions for known and poorly characterized proteins, and provides a rationale for biologically relevant contexts where these proteins may function.

The GTEx dataset was downloaded from the GTEx Portal webpage (v7, reprocessed by our internal pipeline).

C. IgSF Interactome and TCGA Dataset

Perturbations in the extracellular interactomes are tightly associated with pathological processes including tumor growth and immunoevasion, often through uncharacterized mechanisms. Therefore, we sought to investigate which edges in our interactome connect nodes that are significantly deregulated (up- or downregulated) in comparison to their gene expression in tumor tissue to adjacent normal tissue as reported in The Cancer Genome Atlas (TCGA).

Tumor versus matched adjacent normal differences in TCGA were assessed for all indications that had at least 3 matched normal samples (N=20 out of 33 indications). Differential expression was tested for all TCGA indications with at least three matched Tumor and Adjacent Normal samples using a two-sided t-test. All test p-values were adjusted for multiple hypothesis testing using the Benjamini-Hochberg method. All heatmaps were plotted with the pheatmap R package. Unsupervised clustering was performed with the Euclidean distance and Ward linkage. All network statistics were computed in Cytoscape (v.3.6.1) (Shannon et al., Genome Res, 13: 2498-2504, 2003). Network clustering was also performed in Cytoscape using the Markov Clustering Algorithm (MCL), implemented in the ClusterMaker2 plugin, with the Spearman correlation coefficients as edge weights (inflation parameter=1.4, stop if residual increases=False, number of iterations=500).

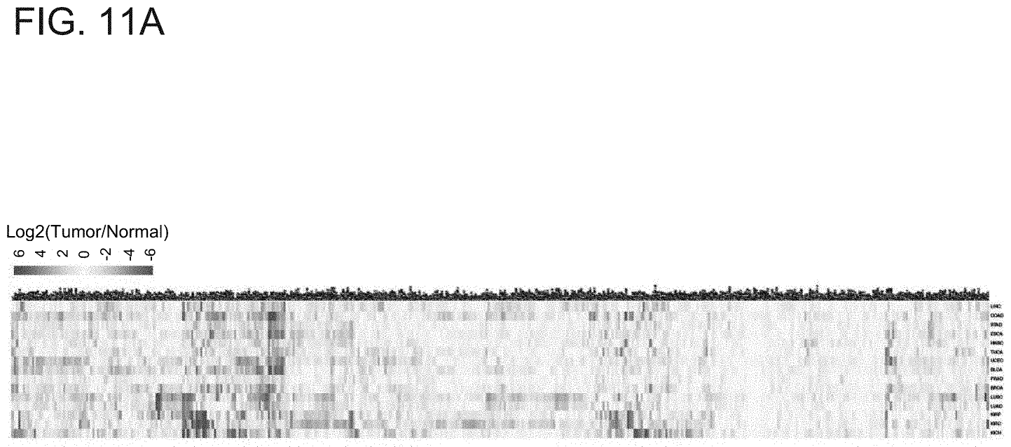

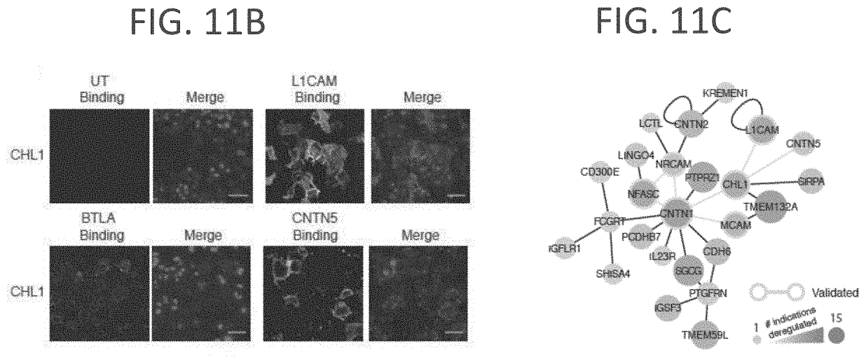

This analysis revealed that 398/485 nodes (˜82%) connected by 543/703 edges (˜75%) are jointly de-regulated in at least one TCGA indication (FIG. 6A), indicating that interactions involving IgSF and STM proteins are generally perturbed in tumors. We also observed that the number of deregulated nodes, as well as the number of edges connecting those, is highest in Lung Squamous Cell Carcinoma (LUSC), Colorectal Adeno Carcinoma (COAD) and Kidney Renal Carcinoma (KIRC) indications, where around 150 network genes are significantly overexpressed (FIGS. 6B, and 11A). This analysis confirmed and further validated that many relatively well-characterized interaction pairs are up-regulated in multiple shared TCGA indications, such as the inhibitory receptor CTLA4 and CD80 (5/19 indications), the PVR/nectin family (4/19 indications) or several members of the semaphorin and ephrin receptor families, among others (FIG. 6A). Conversely, these results also revealed that functionally related interaction pairs such as CADM2 and CADM3, FLRT2 and UNCC (9/19 indications each) or some members of the PTPR family and their interacting partners, are jointly downregulated across multiple shared tumor types, consistently with their proposed functions as tumor suppressors. Interestingly, our analyses also highlighted such striking synonymous co-modulation patterns for many of the newly reported interactions in this study. Of note, a number of interacting pairs, such as the inhibitory receptor pairs CD300LF-CD300LG and LILRB4-CNTFR, or the co-stimulatory molecules ICOS-ICOSLG are negatively synchronized in (up- and down-regulated, respectively), suggesting that absence of these interactions may play a role in tumor progression. Finally, this analysis revealed that most proteins identified as hubs (nodes connected by 5 or more edges), including PTPRD, PTPRS, NTRK2, CNTN1 or CD300LG, are significantly downregulated across multiple indications (FIGS. 6A-6D), suggesting that disruption of interactions mediated by these proteins may play a role in cancer progression, and potential roles for poorly characterized proteins such as the CD300 family of immune receptors.

The TCGA RNA-Seq data was downloaded from the NCI Genomic Data Commons PanCanAtlas Publications webpage.

### Example 4. Methods for Validation of Binding Partners

Selected interactions identified in Examples 1 and 2 were validated using one or more orthogonal techniques, e.g., surface plasmon resonance (SPR), biolayer interferometry, and co-immunoprecipitation (co-IP).

A. Surface Plasmon Resonance

Putative receptor-ligand interactions were analyzed by SPR using a Biacore 8K (GE Healthcare) or Proteon instrument (Biorad). Indicated proteins were immobilized on CM5 (GE Healthcare) or GLC (Biorad) sensor chips, respectively, using a standard amino coupling method. Analytes were run at the concentrations indicated in each case, in HBS-P buffer (0.01 M Hepes, 0.15 M NaCl and 0.005% surfactant P20, pH7.4) or in PBS-0.01 TWEEN® 20 when the Proteon instrument was used. For kinetic calculations, ligands were immobilized at low resonance units, and K_{D} values were calculated in equilibrium. For kinetics experiments, his-tagged recombinant proteins were used as analytes. In all cases, bulk refractive index changes were removed by subtracting the reference flow responses, and sensorgrams were analyzed using the Proteon BiaEvaluation software version 4.1 (Biorad).

B. Biolayer Interferometry

Interactions between AXL and the binding partners IL1RL1 and Gas6 were assayed by biolayer interferometry using an Octet Red system, as previously described (Husain et al., Mol Cell Proteomics, 18: 2310-2323, 2019). Briefly, recombinant AXL, MERTK or Tyro3 ectodomains were biotinylated in vitro using the EZ-LINK™ Sulfo-NHS Biotinylation Kit (Thermo Scientific), and captured on streptavidin-coated sensors. These receptors were tested for binding to Gas6 or IL1RL1, expressed as recombinant ectodomains assayed in PBS buffer. Data were analyzed using Forte Pall (Port Washington, N.Y.) software 9.0.

C. Recombinant Proteins

The following proteins were purchased from Sino Biologicals: CNTFR, LDLR, SLITRK4, and BTN3A3. The following proteins were obtained from R&D Biosystems: CHL1, MCAM, SIRPA, CNTN1, SLITRK2, SLITRK3, LRFN5, EDAR, FLT4, PD-L1, PD-1, EPHA3, EPHA4, BTNA2A, BTN3A1, AXL, IL1RL1, VSIG10L, FLRT1, FLRT2, FLRT3, NCR1, MDGA1, TREML2, IGSF9B, LILRA3, Gas6, MERTK, and Tyro3. CEACAM4, expressed as ECD-Fc, was expressed in mammalian cells and purified in house using standard affinity chromatography techniques (Ramani et al., 2012).

D. Co-Immunoprecipitation of Novel Binding Partners

The following STM proteins were expressed as full-length proteins fused to a C-terminal HA tag: FAM187B, LRRC4B, LRRC4C, VSIG8, CDH9, ST14, TGOLN2, IGSF5, IL1RL1, VSIG10L, PD-L2, PD-L1, LILRA3, LILRB4, LILRB5, and NCR1. The following STM proteins were expressed as full-length proteins fused a C-terminal Flag tag: BTN2A1, BTN3A1, BTN3A2, BTN3A3, AXL, CD300A, CD300C, CD300LF, CEACAM4, EPHA3, IL6R, EDAR, ILDR1, CNTFR, LDLR, SIGLEC7, SIGLEC8, and CD4. HEK293 cells were co-transfected with the relevant protein pairs (expressed as HA- and Flag-tagged fusions, respectively) using LIPOFECTAMINE™ LTX Reagent. Relatively low plasmid concentrations were used to avoid high overexpression of the proteins. Typically, 6×10⁵ cells were seeded in M6 well plates and reverse transfected with 1 μg of a 1:1 DNA mixture. Cell lysates were washed with PBS and lysed ˜18 hours post-transfection using RIPA buffer (50 mM Tris HCL pH 7.4, 150 mM NaCl, 2 mM EDTA, 0.5 (w/v) Na-deoxycholate, 0.1% (w/v) SDS, 1% (v/v) NP40). Equal amounts of the lysates were incubated with EZVIEW™ Red ANTI-FLAG® M2 Affinity Gel (Millipore Sigma) at 4° C. overnight, following the manufacturer's instructions. The beads were extensively washed with lysis buffer and proteins were eluted using loading buffer SDS sample buffer (Thermo Fisher Scientific) using denaturing conditions. Immuno-precipitates were analyzed by western blotting using anti-Flag (Cell Signaling Technologies) and anti-HA antibodies (Abcam), using a LICOR instrument.

### Example 5. Validation of EPHA3 and CEACAM4 Binding Partners

The functional interaction map (FIG. 3A) showed that related receptors within a protein family showed distinct binders or differential tissue expression relative to other members within the protein community. Two notable examples of such divergent behavior were the ephrin receptor EPHA3 and the cell surface protein CEACAM4, identified as binding partners for PD-L1 and PD-L2, respectively, within the immune regulatory cluster (FIG. 4A).

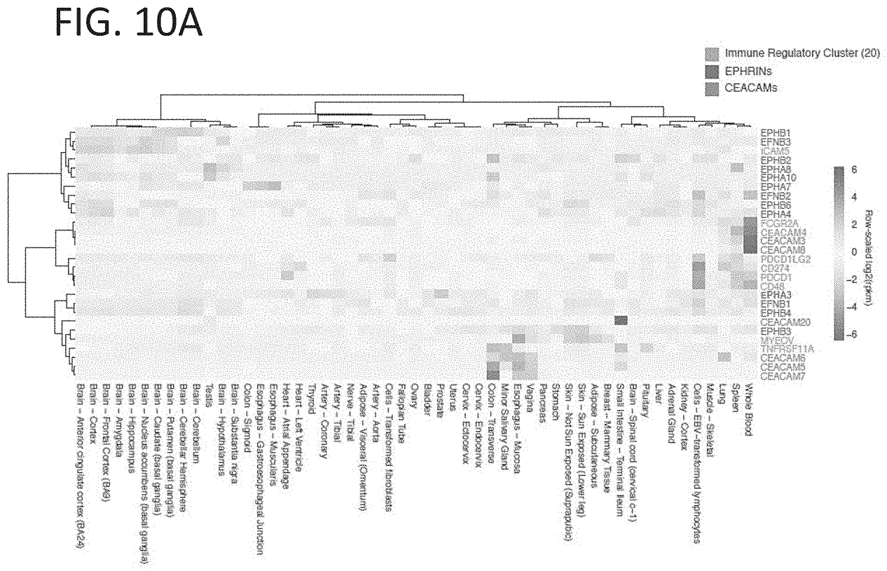

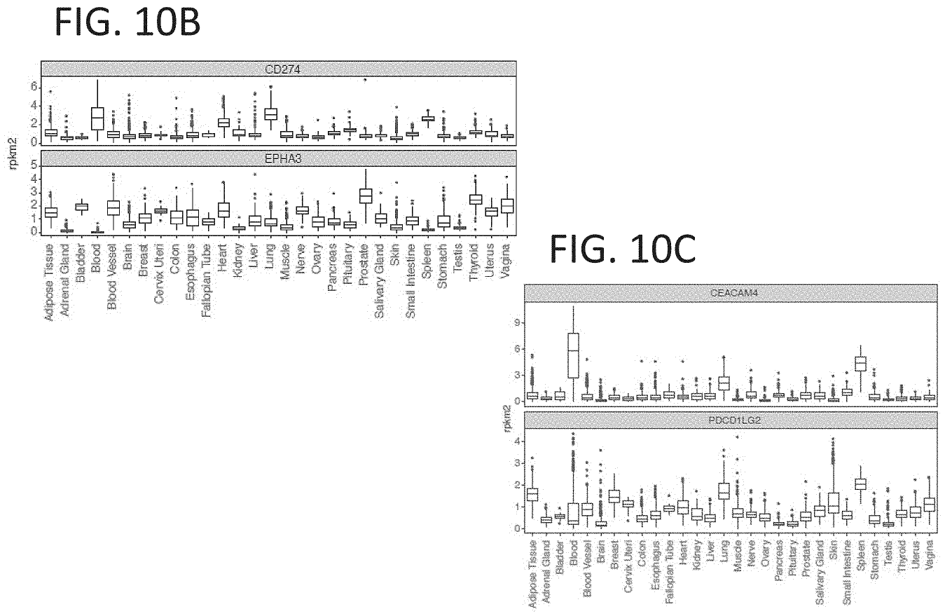

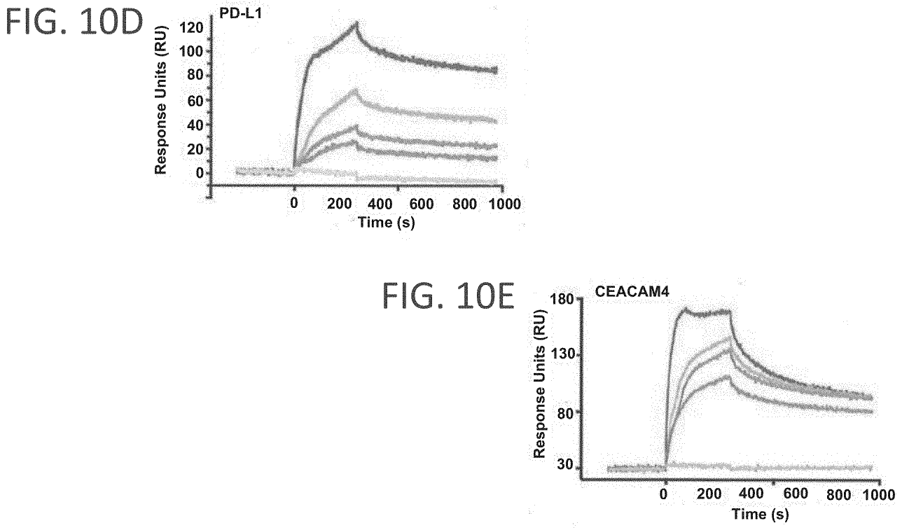

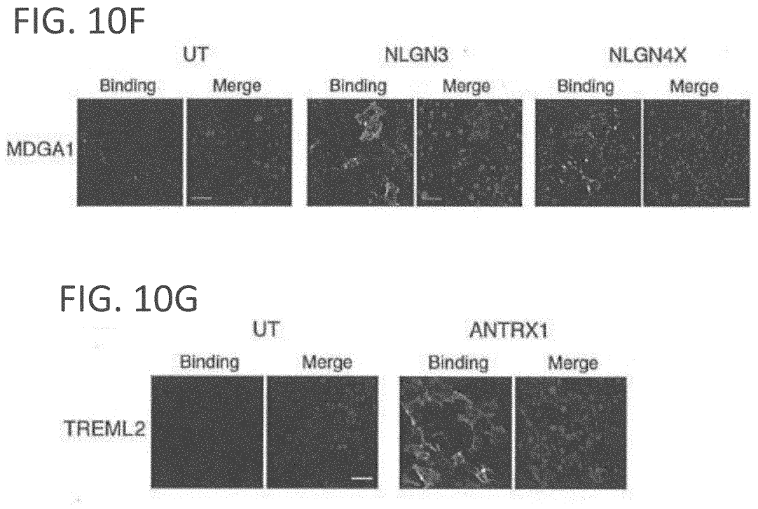

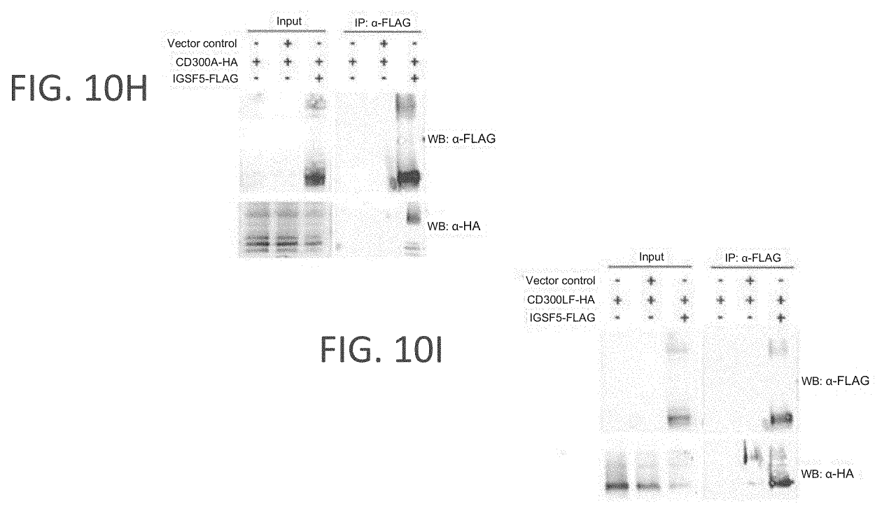

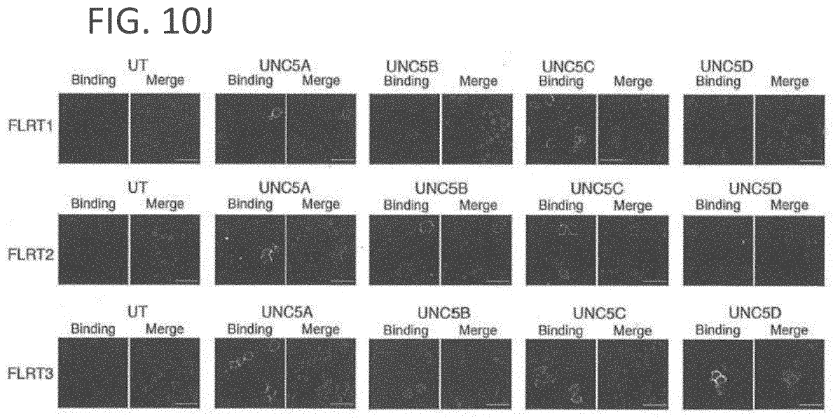

Interestingly, certain ephrin family members (EPHA3, EFNB1, EPHB4) displayed a more promiscuous expression pattern relative to the rest of the family (FIGS. 10A and 10B). Similarly, the family of Carcinoembryonic antigen-related cell adhesion molecules (CEACAM) was distinctly divided into two tissue clusters, where the group consisting of CEACAM4, CEACAM3 and CEACAM8 showed significant overlap in tissue expression patterns with the immune regulatory cluster, including shared tissue co expression for CEACAM4 and PD-L2 (FIGS. 10A and 10C).

To further investigate these putative immunoregulators, both interactions were studied by surface plasmon resonance (SPR) using purified recombinant proteins. Specific binding of EPHA3 to PD-L1 (FIG. 4E) and CEACAM4 to PD-L2 (FIG. 4B) with moderate binding affinities was confirmed (PD-L1/EPHA3: K_{D}: 2.8×10₋₈±2.7, PD-L2/CEACAM4, K_{D}: 8.4×10₋₈±69) (FIGS. 10D and 10E), illustrating the capacity for robust and sensitive detection of receptor-ligand interactions through the discovery pipeline.

SPR analysis also confirmed the newly identified interaction between the LILR family member LILRA3 and EPHA3 (FIG. 4C). PD-L1 interacted with its known binding partners PD-1 and PD-L2 expressed on cells, as well as EPHA3, whereas no interaction with a related member of the ephrin family or the CEACAM proteins tested was observed, further demonstrating the specificity of the PD-L1/EPHA3 interaction (FIG. 4F). In turn, PD-L2 bound to PD-L1 and PD-1 expressed on the cell surface, as well as with the new interactor CEACAM4. No binding to CEACAM5, a protein related to CEACAM4, or the PD-L1 binder EPHA3 was observed, corroborating a specific interaction between PD-L2 and CEACAM4 (FIG. 4F). Interestingly, the therapeutic antibody atezolizumab, known to block the PD-1/PD-L1 axis and thus currently used as immunotherapy for the treatment of solid tumors (Shah et al., Hum Vaccin Immunother, 14: 269-276, 2018), also competed the interaction with EPHA3 (FIG. 4D).

### Example 6. Validation of PTPR Binding Partners

Functional clustering of the IgSF interactome network (Example 2) revealed a remarkable association between members of the receptor-type protein tyrosine phosphatase (PTPR) family and nervous system-related proteins, including the neurotrophin receptors (NTRK), the interleukin-1-receptor accessory proteins (ILRAP), the Slit and NTRK like family (Slitrk), and the Leucine Rich and Fibronectin Type III domain (LRFN) proteins. Interestingly, the netrin-G ligand-3 (NGL-3/LRRC4B) was also found to interact with PTPRs proteins as well as with specific members of the butyrophilin (BTN) family (FIG. 3A).

A. Cell Surface Interaction Assays

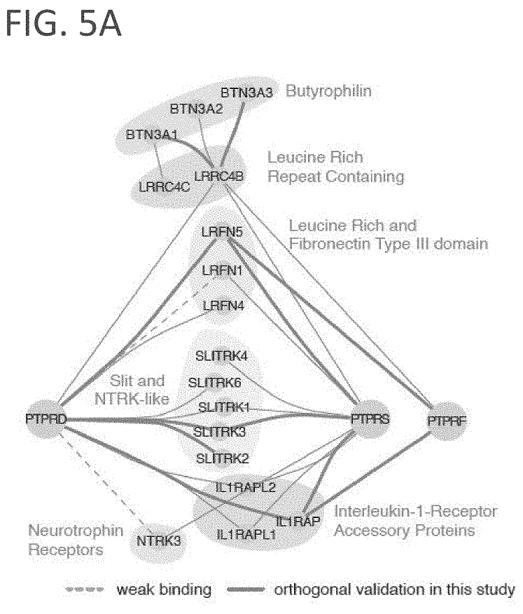

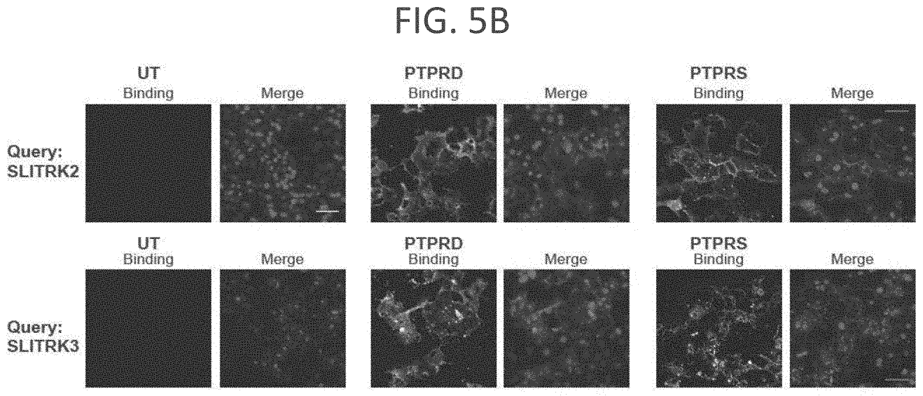

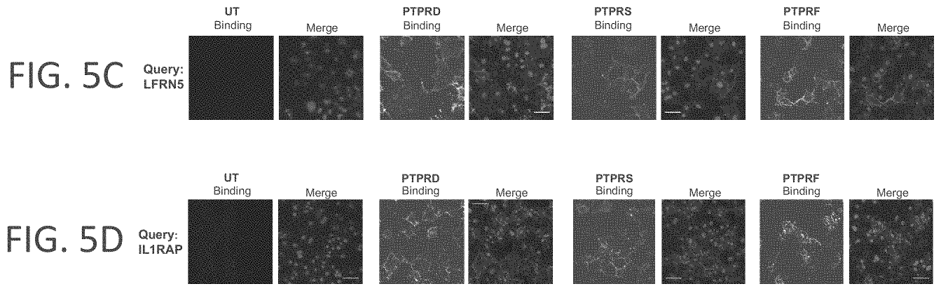

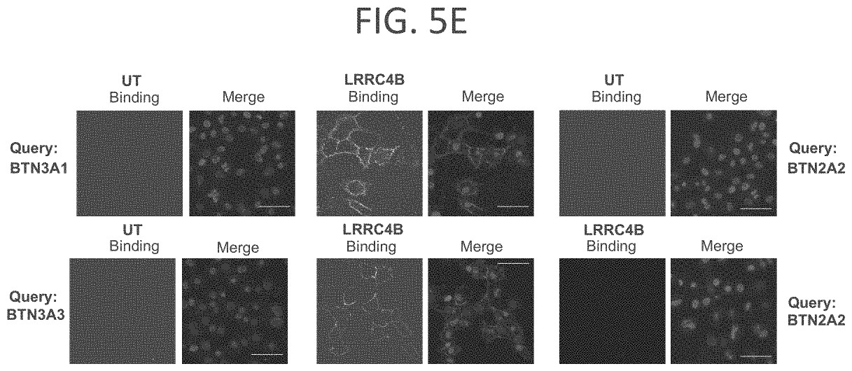

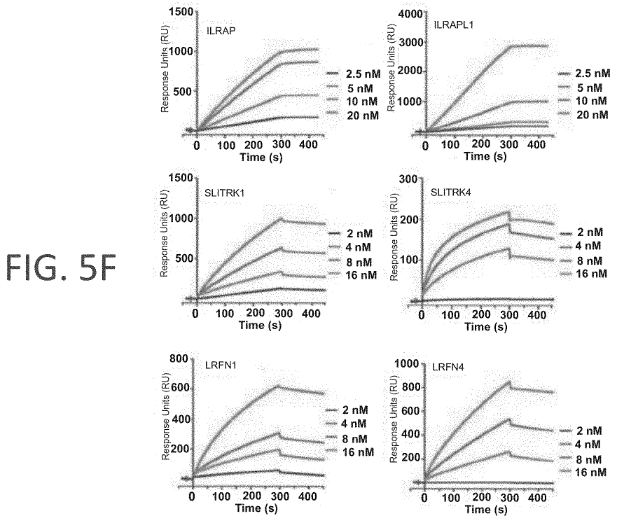

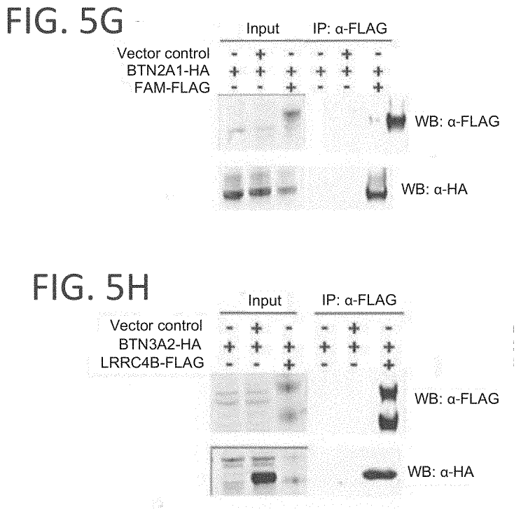

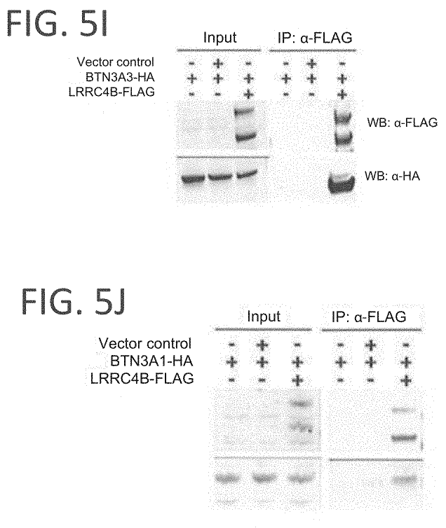

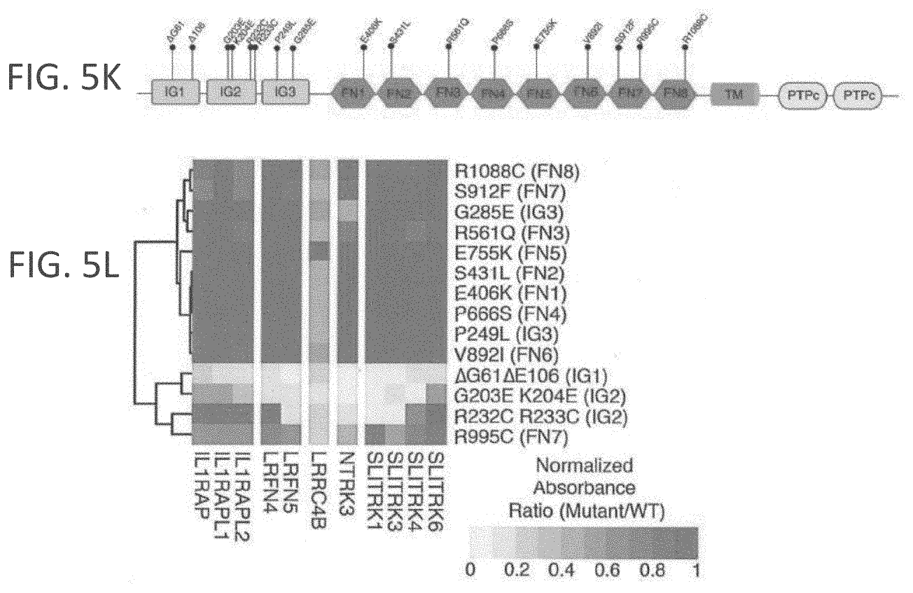

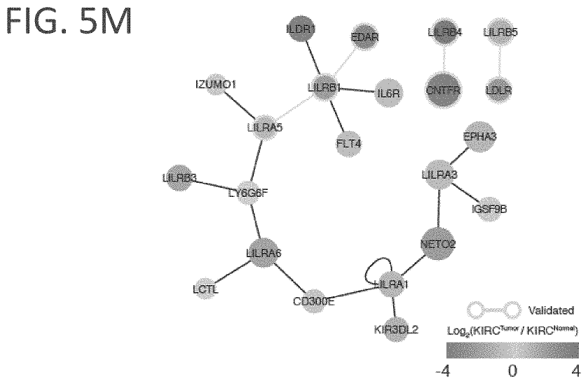

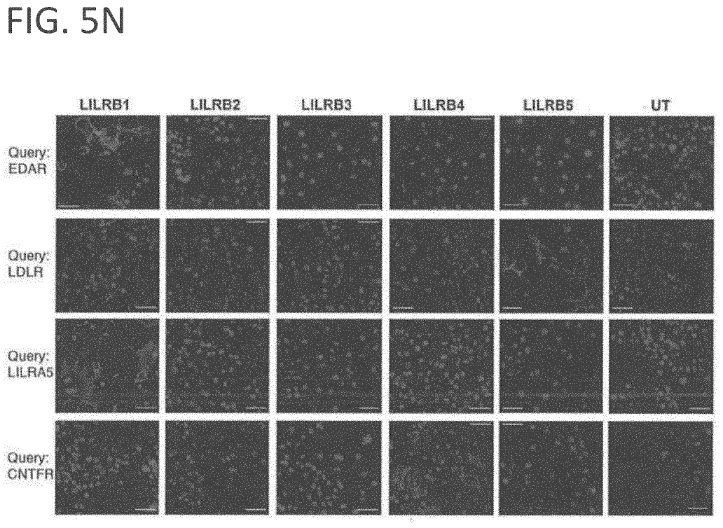

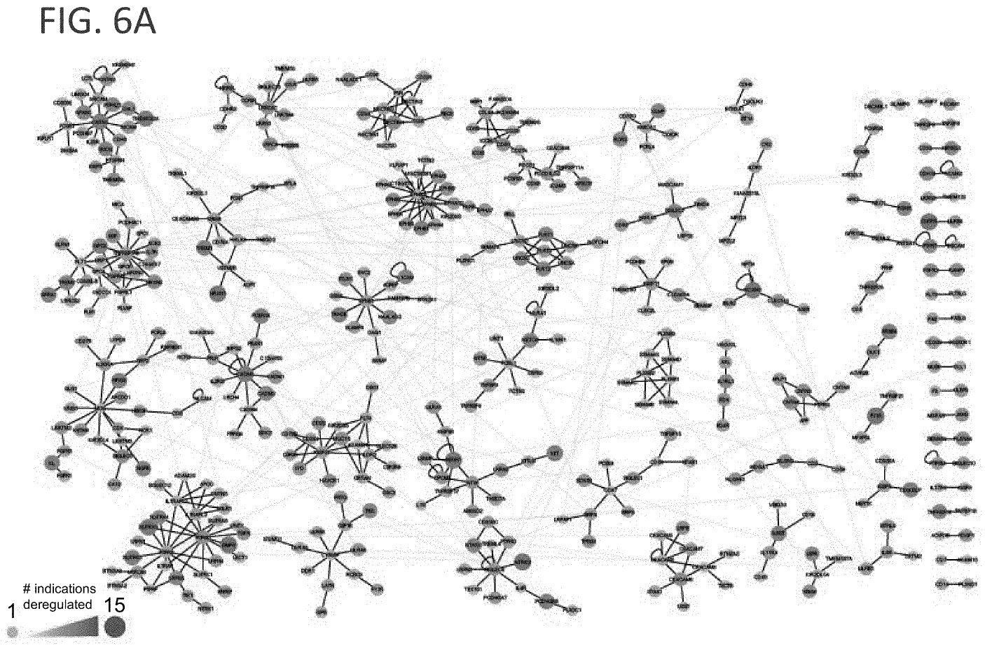

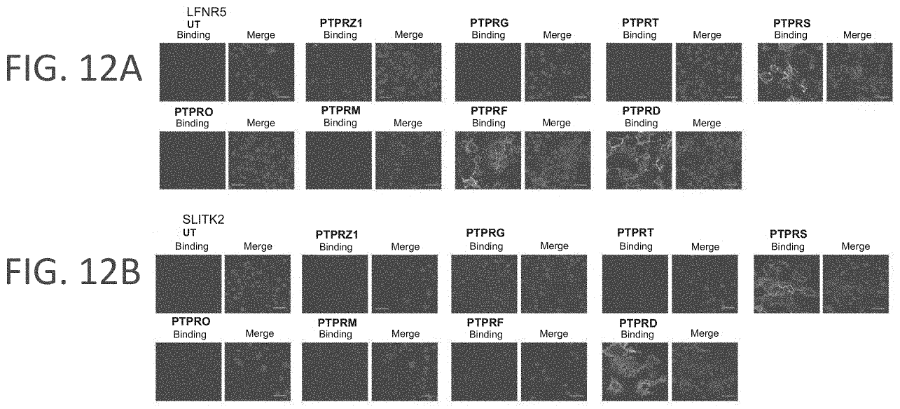

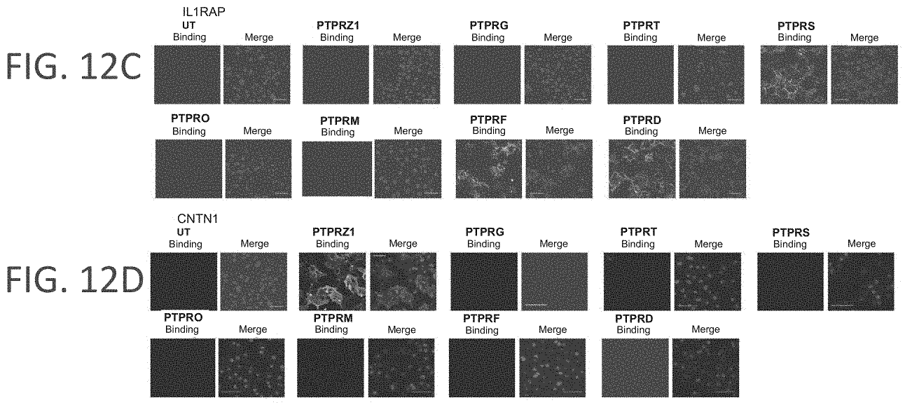

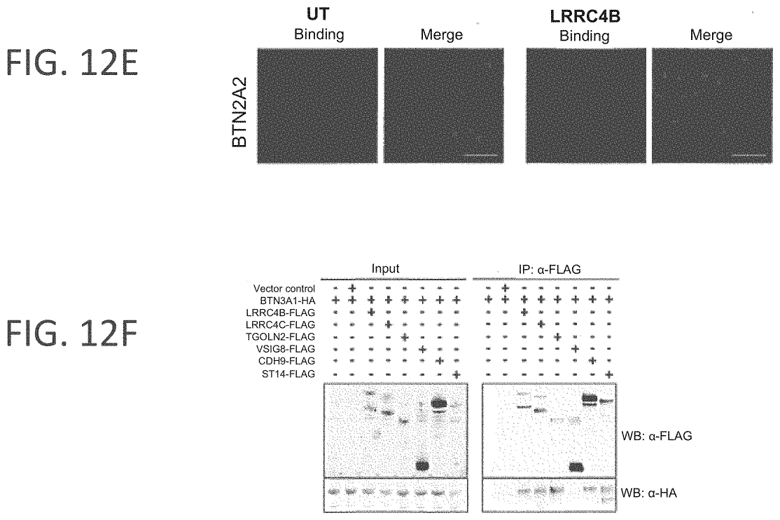

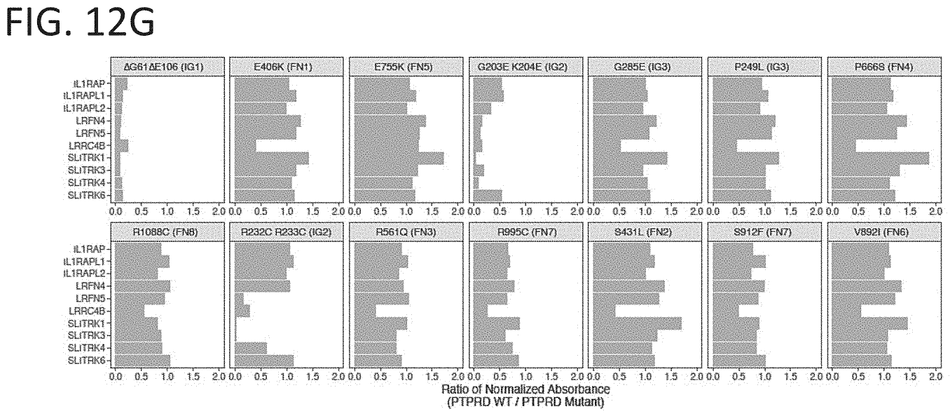

The PTPR family is the largest family of plasma membrane-expressed receptor protein tyrosine phosphatases (Du and Grandis, Chin J Cancer, 34: 61-69, 2015). This community revealed complex association patterns between members of the PTPR family and four different families of nervous system-related proteins, including the Neurotrophin receptors (NTRK), the interleukin-1-receptor accessory proteins (ILRAP), the Slit and NTRK-like family (SLITRK), and the Leucine Rich and Fibronectin Type III domain (LRFN) proteins (FIG. 3A, cluster 1; FIG. 5A). In addition, the netrin-G ligand-3 (NGL-3/LRRC4B), known to interact with PTPRs proteins, was found to bind specific members of the butyrophilin family, an emerging family of therapeutically relevant immune receptors (Arnett and Viney, Nat Rev Immunol, 14: 559-569, 2014). To confirm and further study the complex connections between these protein families, PTPRD, PTPRS and PTPRF were expressed as cell surface proteins and tested for binding to their identified interaction partners, using the cell-based methodology described. These results confirmed the specific interaction between the SLITRK2 and SLITRK3 proteins and both PTPRD and PTPRS respectively (FIG. 5B). Additionally, direct binding of LFRN5 (FIG. 5C) and IL1RAP (FIG. 5D) proteins to all three family members PTPRD, PTPRS and PTPRF was observed. In line with these results, SPR analysis further confirmed PTPRD binding to SLITRK1, SLITRK4, LFRN1, LFRN4, IL1RAP and IL1RAPL1 proteins (FIG. 5F). Finally, to investigate whether SLITRK, IL1RAP and LRFN family members specifically recognize select PTP receptors or rather maintain the capacity for promiscuous crosstalk, we tested binding to eight members of the PTPR family. Interestingly, these assays confirmed selective PTPR recognition of its cognate ligands in the respective families (FIGS. 12A-12D).

In addition, we confirmed binding of cell surface expressed LRRC4B to the butyrophilin proteins BTN3A1 and BTN3A3 (FIGS. 5E, 5G and 5H), but not the homologous family member BTN2A2 (FIG. 12E), as predicted by the Interactome results. Of note, these new interactors were confirmed in co-immunoprecipitation studies (FIGS. 5G and 5I), which also demonstrated the interaction between LRRC4B and BTN3A2 (FIG. 5H). Further analysis of other members of the butyrophilin family, including BTN2A1 and BTN3A1 (FIGS. 5G, 5J, and 12F) demonstrated specific binding to the newly identified interactors, indicating that these proteins can form complexes in the cell.

Taken together, this comprehensive validation effort corroborated interacting partners previously described for the PTPR family, identified new interactions between individual family members and to our knowledge, assessed for the first time between-family selectivity. Furthermore, receptor-specific interactors for a number of butyrophilin proteins are uncovered, shedding light on an otherwise poorly characterized and yet highly relevant immunoreceptor family.

B. Disease-Relevant PTPRD Variants

Although a few genes have been exhaustively characterized, most disease-associated mutations are poorly studied and the alterations in protein interactomes underlying disease remain mostly unknown, in part owing to very limited understanding of interacting networks. Emerging data indicate that PTPRD functions as a tumor suppressor and that genetic and/or epigenetic deregulation causing a loss-of-function phenotype may result in altered signaling transduction and increased tumor growth. In fact, PTPRD is frequently mutated in glioblastoma, malignant melanoma and lung adenocarcinoma among other tumors (Peyser et al., PLoS One, 10: e0135750, 2015; Veeriah et al., Proc Natl Acad Sci USA, 9435-9440, 2009). We therefore utilized the receptor interaction discovery pipeline, described previously in this study, to examine a collection of cancer-relevant PTPRD variants. We queried the Catalogue Of Somatic Mutations In Cancer (COSMIC) and the TCGA data portal as well as individually published studies to identify non-synonymous mutations relatively prevalent across tumor types (Table 9).

Interestingly, in addition to alterations in the protein tyrosine phosphatase domain, frequent mutations were located throughout the immunoglobulin (IG) and fibronectin (FN) domains that compose the ECD, suggesting an impact on the landscape of PTPRD interactions that mediate cell communication (FIG. 5K). To examine whether these cancer-relevant mutations would interfere with the interacting network described for PTPRD, a collection of 14 mutants representing all domains in the ECD were screened with the interaction discovery pipeline (FIGS. 5K and 5L and Table 9). Overall, mutations in the FN domains did not substantially modify the PTPRD interaction repertoire, whereas mutations concentrated in the IG domain had a significant effect (FIG. 5L), showing a significant reduction in binding to most binding partners, in agreement with the reports so far and existing structures (Mohebiany et al., FEBS J, 280: 388-400, 2012; Veeriah et al., Proc Natl Acad Sci USA, 9435-9440, 2009).

Interestingly, most of the mutations studied significantly impact the interactions to LRRC4B and NTRK3, with mutations in IG1-3 and FN domains 1-3 completely abrogating binding, suggesting that selected interactions are generally perturbed in the context of the tumor. Conversely, binding to ILRAP family members IL1RAP, IL1RAPL1, and ILRAPL2 was not impaired or was only marginally impaired, with the exception of mutants in the IG1 and IG2 domains (FIGS. 5K and 5L). Interestingly, we also observed that selected mutations selectively impact binding to specific family members. Within the SLITRK family, the R232C/R233C double mutation impaired binding to SLITRK1/3 proteins, whereas binding to SLITRK6 was generally comparable to wt PTRPD. Similarly, within the LRFN family, the G203E/K204E double mutant showed absence of binding to both LRFN4 and LRFN5, while the R232C/R233C double mutant showed a more significant reduction in binding to LRFN5 but not LRFN4 (FIG. 5L). These results suggest that the highly specific interaction network rewiring events identified by the PTPRD mutant screen may play a role in disease and suggest a broader scope of biological functions and signaling properties that had remained uncharacterized for PTPRD.

### Example 7. Validation of CHL1 and CNTN1 Binding Partners

The IgSF interactome network (Example 2) revealed an association between the neural cell adhesion molecule CHL1 and the contactin family member CNTN1, two cell surface proteins that play roles in extracellular matrix, cell adhesion and synapse formation in the nervous system (FIG. 3A). The interactions between CHL1 and CNTN1 and other interacting proteins were confirmed using cell surface interaction assays and further assayed using SPR.

A. Cell Surface Interaction Assays

Cell surface interaction assays as described in Example 5A were conducted for CNTN1 and CHL1. CHL1 expressed on the cell surface was confirmed to interact with CNTN5 and L1CAM and to interact more weakly with the immune receptor BTLA (FIG. 11C).

CNTN1 expressed on the cell surface bound specifically to the soluble query PTPRZ1, but to no other related members of this receptor family, including PTPRD or PTPRS (FIG. 12D), which were found to associate to the LFRN, SLITRK, and IL1RAP proteins (FIGS. 5A and 12A-12D). This result further highlights the accuracy of the binding profiles identified in the IgSF interactome screens and suggests that these receptors exert specific functions through selective recognition of binding partners.

B. SPR Assays

Selected CNTN1 binders were assayed by SPR as described in Examples 4C and 4D. CNTN1 was expressed as an extracellular domain (ECD) fused to a human Fc tag and was immobilized on CM5 sensor chips. The analytes NRCAM, NFASC, MCAM, and CHL1 and a control were formulated as described in Example 4B and were injected at a concentration of 250 nM. These experiments demonstrated a direct interaction between CNTN1 and the nervous system proteins NRCAM, NFASC, MCAM, and CHL1 (FIG. 9I).

### Example 8. Validation of LILR Family Binding Partners

The LILR proteins are a family of emerging, yet poorly characterized immune receptors, some of which are known to bind to MHC class I. We focused on LILRB1, LILRB3, LILRB4 and LILRB5, receptors characterized by the presence of immunoreceptor tyrosine-based inhibitory motifs (ITIM) and thus having putative immunosuppressive functions (Brown et al., Tissue Antigens, 64: 215-225, 2004; van der Touw et al., Cancer Immunol Immunother, 66:1079-1087, 2017).

Interestingly, our study identified LILR receptor-specific interactions, including binding of EDAR and LILRA5 to LILRB1, expressed on the plasma membrane; and specific interactions between CNTFR and LDLR with LILRB4 and LILRB5 receptors, respectively (FIG. 5N). Furthermore, EDAR (FIG. 4G), IL6R (FIG. 4H) and CNTFR (FIG. 4I) efficiently co-immunoprecipitated with LILRB1 and LILRB4, respectively, indicating that these new identified binding partners can take place in the context of the plasma membrane.

A. Cell Surface Interaction Assays

Cell surface interaction assays as described in Example 5A were conducted for LILR family proteins and relevant binding partners. FLT4, EDAR, IL6R, CNTFR, and LDLR were expressed on the cell surface, and LILRB1, LILRB4, and LILRB5 were expressed as soluble multimerized proteins. These assays confirmed the interaction between LILRB1 and the receptors EDAR, IL6R and the vascular endothelial growth factor receptor 3 VEGFR3 (FLT4), the interaction between LILRB4 and CNTFR, and the interaction between LILRB5 and LDLR (FIG. 11B).

B. Analysis of LILRB1 in Network Integrations

Integration of the IgSF network with TCGA (Example 3C) revealed a synchronic upregulation of both LILRB1 and the lymphangiogenesis receptor FLT4/VEGFR3, but not other LILRB1 interacting partners, across multiple tumor indications, a pattern that was particularly significant in renal cancer (FIG. 5M). These observations are also in agreement with annotations in the Protein Cell Atlas and with independent efforts that suggest that FLT4 is a negative prognostic in certain tumors. In addition, increasing evidence indicates that LILRB1 plays a critical role in immune responses. We and others have identified LILRB1 as a prominent viral target (Chan et al., Proc Natl Acad Sci USA, 111: 2722-2727, 2014; Chapman et al., Immunity, 11: 603-613, 1999; Hirayasu et al., Nat Microbiol, 1: 16054, 2016; Martinez-Martin et al., Nat Commun, 7: 11473, 2016), and a recent study has demonstrated a central role for LILRB1 in controlling the phagocytic function of macrophages through recognition of MHC-I (Barkal et al., Nat Immunol, 19: 76-84, 2018), altogether indicating that LILRB1 represents a promising therapeutic target.

### Example 9. IgSF Interactome and Cancer

The IgSF Interactome was evaluated in the context of transcriptionally profiled samples from a large phase 2 trial (IMvigor210) on metastatic urothelial cancer patients treated with the anti-PD-L1 therapeutic antibody atezolizumab. These findings revealed interacting protein communities associated with CD8+ T-effector (Teff) cell function and distinct tumor immunological phenotypes. Further investigation uncovered protein interaction signatures highly correlated with lack of response to treatment and survival, with improved predictive power for clinical outcome.

A. Differential Gene Expression in Tumors

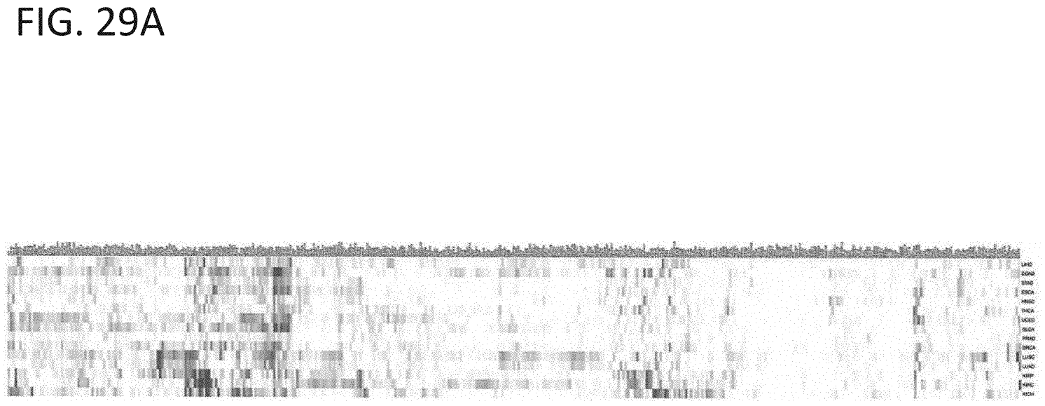

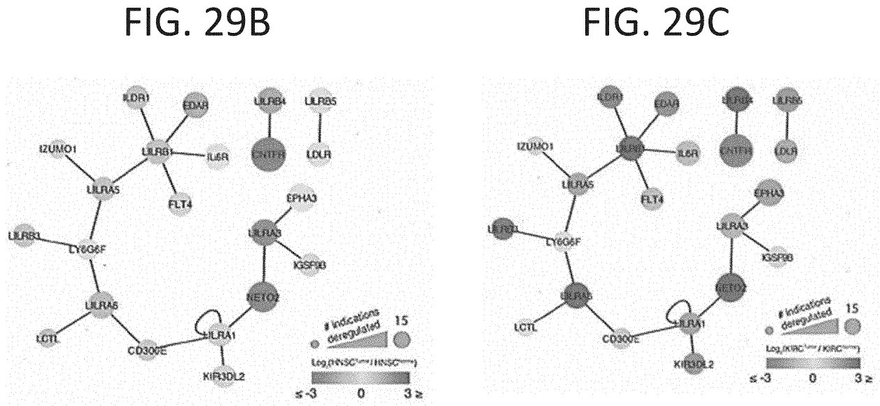

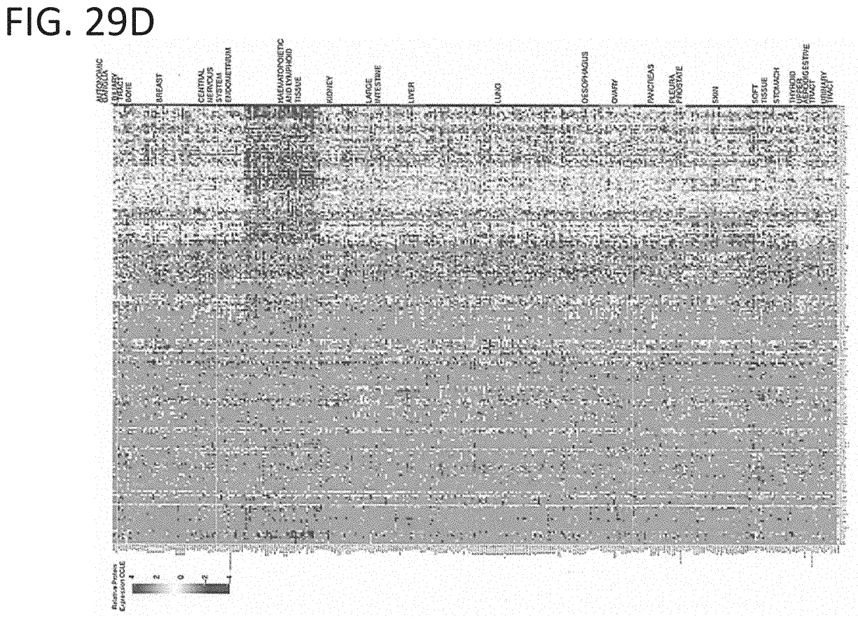

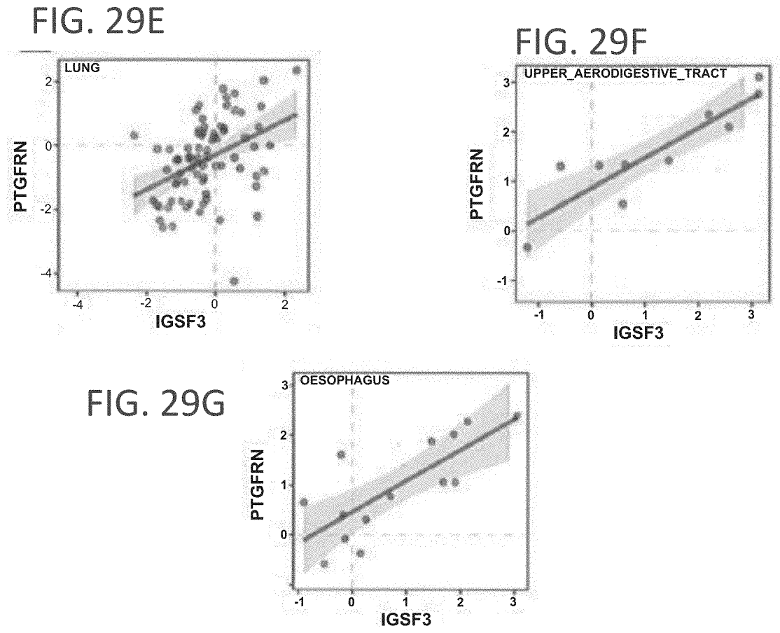

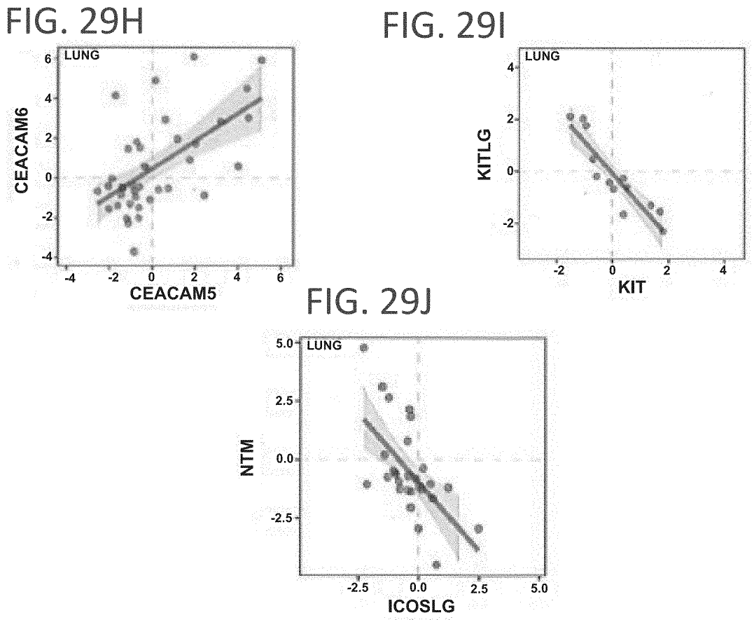

Tissue integrity during homeostasis is primarily dictated by the interactions between cell surface proteins and, consequently, perturbations of the extracellular interactome are tightly associated with pathological processes such as tumor growth and immune evasion. Understanding of how protein interaction networks are perturbed or rewired in disease significantly limited, fundamentally due to the poor coverage of extracellular interactomes. Thus, to connect genomic information and functional significance, differential mRNA expression data between tumor and adjacent normal tissue, as reported in TCGA, was incorporated into the IgSF interactome (FIG. 6A and FIGS. 29A-29J). Remarkably, we observed that over 90% of the IgSF genes studied were significantly differentially expressed (two-sided t-test, |Log Fold-Change|>1, q<0.05) in at least one tumor indication (FIG. 6A). In fact, a systematic comparison between pairs of unrelated genes and the interacting pairs in the IgSF interactome reported in this study showed that the number of jointly dysregulated interactions was consistently higher within the IgSF across all TCGA indications (FIG. 6B). Notably, integration of the IgSF Interactome and TCGA highlighted interacting proteins that were jointly down-regulated in most tumors, including CADM3-CADM2, UNC5C-FLRT2, and some members of the PTPR family and their interacting partners, observations that are consistent with tumor suppressor functions described for these genes (Bae et al., Sci Rep, 7: 272, 2017; Chang et al., Clin Cancer Res, 16: 5390-5401, 2010). Conversely, this analysis also confirmed and further supported the observation that well-characterized interacting pairs are commonly jointly up-regulated in multiple shared TCGA indications, including well-described immune receptor pairs such as CTLA4/CD80 or PD-1/PD-L1 (FIGS. 6C and 6D). In addition, as illustrated by certain members of the LILR family, a number of interacting proteins were found to be differentially expressed in opposing directions (up- and downregulated, respectively) in multiple tumor indications (FIGS. 29B and 29C), suggesting that disruption of these interactions may influence cell communication in the tumor microenvironment.

Next, we sought to investigate which interactions were correlated at the protein level, which may have been missed at the RNA level. To do so, the recently published proteomics resource for 375 cancer cell lines from the Cancer Cell Line Encyclopedia (CCLE) (Nusinow et al., Cell, 180: 387-402 e316, 2020) was integrated with the IgSF interactome. Similar to the observations made using GTEX, the Pearson correlations for the subset of interacting pairs was significantly higher compared to the complement of non-interacting pairs, albeit less outspoken (one-sided Wilcoxon rank-sum text, p<0.05) and with overall lower correlation scores to their GTEX counterparts, likely due to the larger number of patients in GTEX than cell lines in the CCLE, and the typically larger amount of missing values in proteomics datasets. Nevertheless, comparison of the IgSF Interactome with protein expression also revealed many interactions that were strikingly correlated, such as CEACAM5 and CEACAM6 in large intestine lineages (FIG. 6F) and lung cancer cell lines (FIG. 29H); the newly validated protein pairs CNTN1-NRCAM in lung cell lines (FIGS. 6G, 9H, and 9I); LRRC4B and BTN3A1 (FIGS. 6H, 5H, and 5J); interactions between the UNC and FLRT families (FIGS. 6I and 10A-10J), or the CNTN1-PTPRZ1 pair, which interestingly, showed a strong correlation in healthy brain tissue (FIG. 3A) as well as a strong association in hematopoietic and lymph cell lines (FIG. 6J), suggesting a yet uncharacterized role for these interactions in immune cells. Similarly, the IGSF3-PTGFRN pair, for the first time reported to interact in this study and largely of unknown function, showed prominent correlation across cancer cell line lineages (FIGS. 6K, 6L, and 29E-29G). We also observed strong correlation for the TAM receptor AXL (FIG. 6M), a receptor tyrosine kinase and important regulator of innate immunity increasingly appreciated as a determinant of resistance in tumors (Zhu et al., Mol Cancer, 18: 153, 2020). Interestingly, AXL protein levels were significantly correlated with the new binding partner VSIG10L, capable of interacting in cis in the plasma membrane (FIG. 6N). Although there was no protein expression data available for the second putative interactor identified, IL1RL1, we were also able to confirm a specific interaction between AXL and IL1RL1 using multiple methods, suggesting new modulators for this important RTK (FIGS. 6O-6Q).

Finally, tightly negatively correlated interactions were observed for protein pairs such as KIT-KITLG or ICOSLG-NTM in lung (FIGS. 29I and 29J), patterns that might suggest an important suppressive role for these interactions (Chung et al., mSystems, 4, 2019).

Taken together, these findings indicate that extracellular protein interactions between IgSF and STM proteins are commonly perturbed in tumors, possibly reflecting differences in the immune cell infiltrate in the tumor, or an immune-evasive process by the tumor cells. Evaluation of the IgSF Interactome in the context of the TCGA enables elucidation of interacting protein networks impacted in specific tumor types, offering molecular insights into specific pathways dysregulated in cancer and thus suggesting opportunities for therapeutic development.

B. Interaction Signatures Associated with T Cell Function and Clinical Outcome.

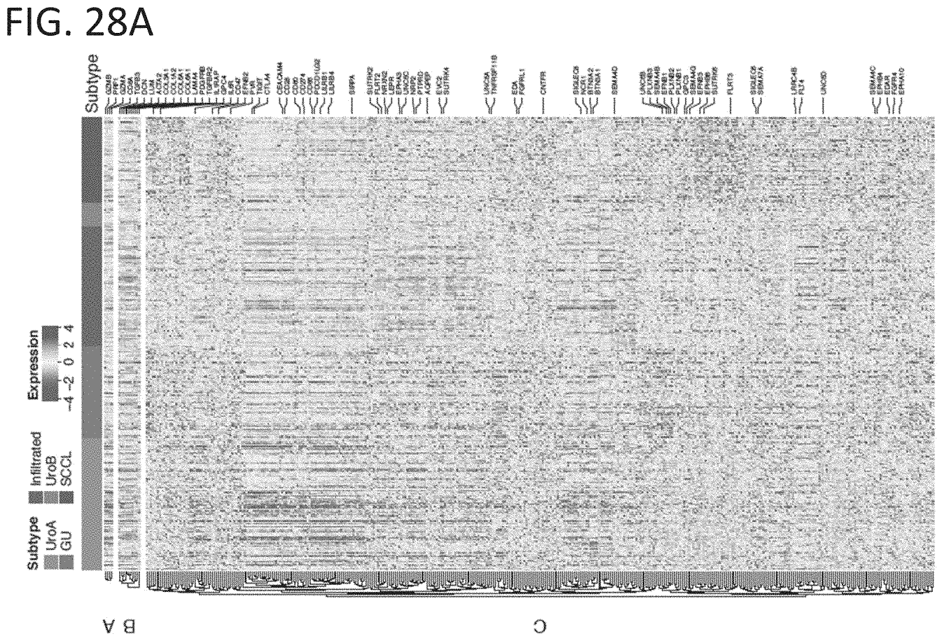

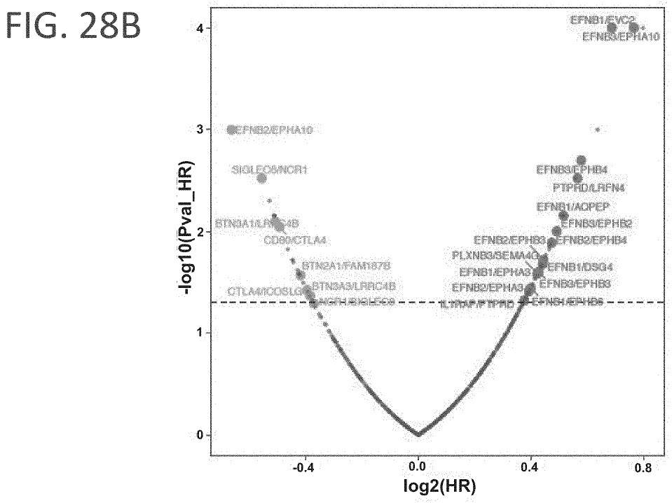

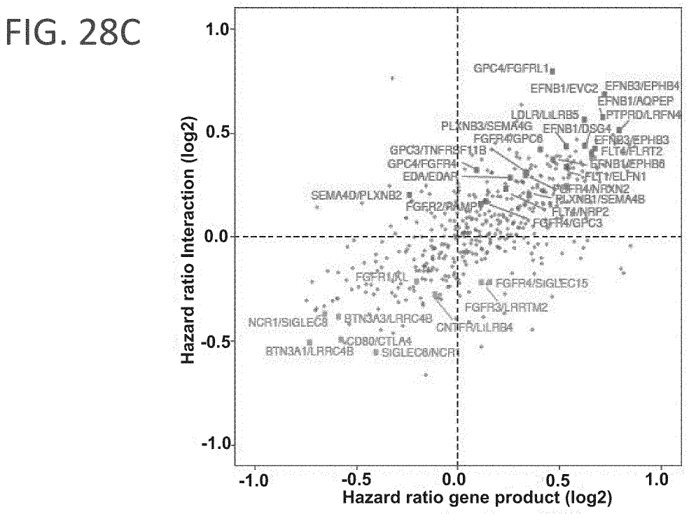

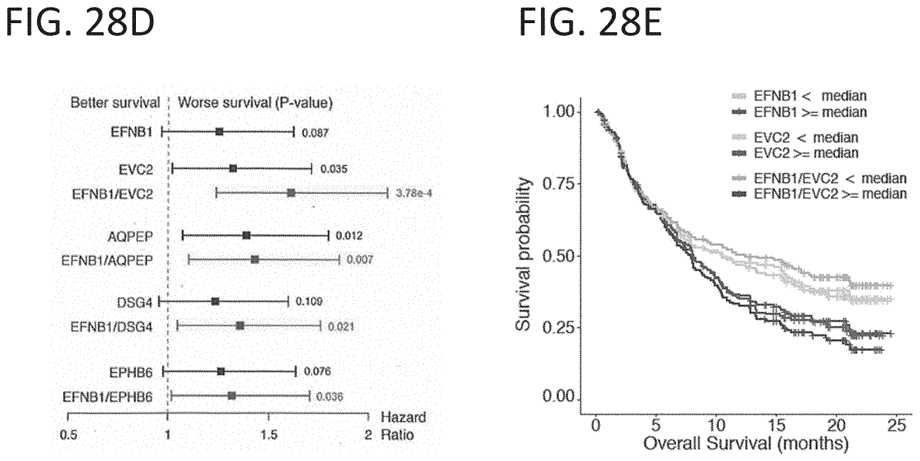

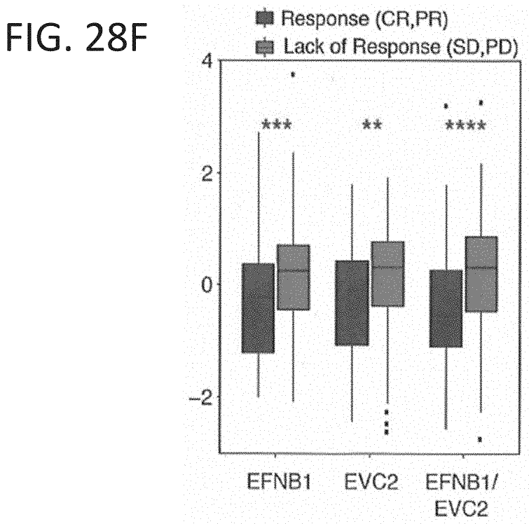

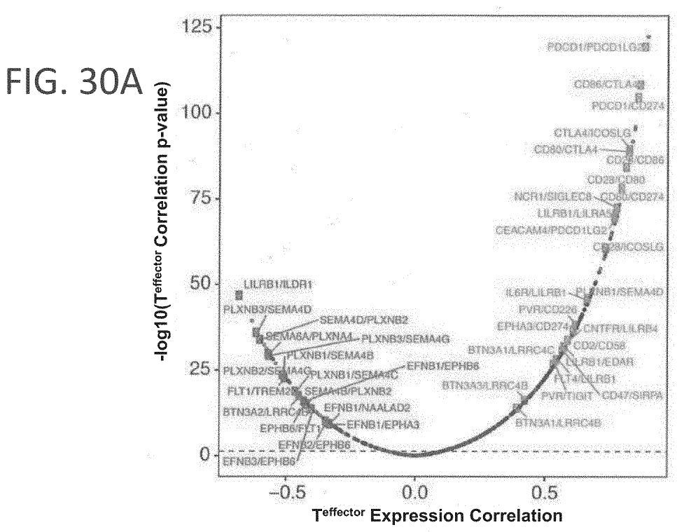

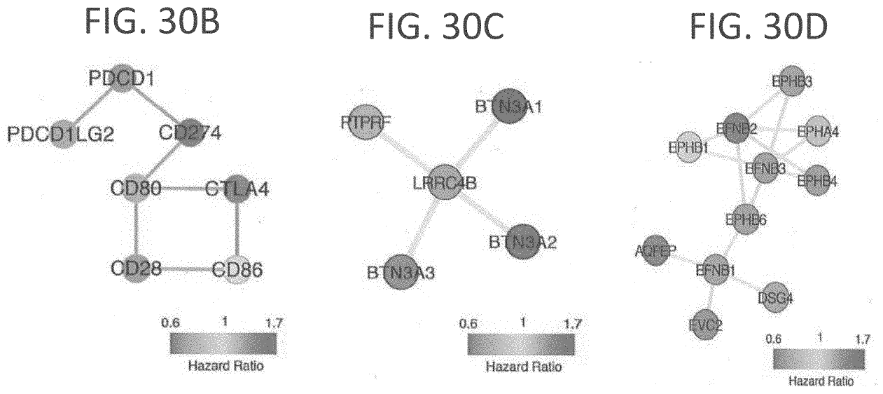

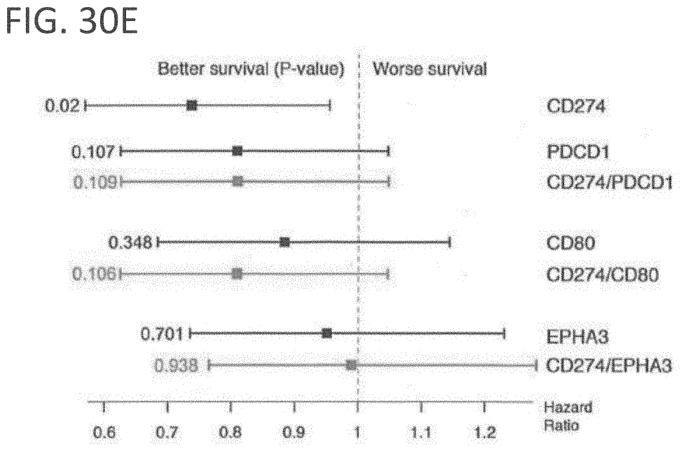

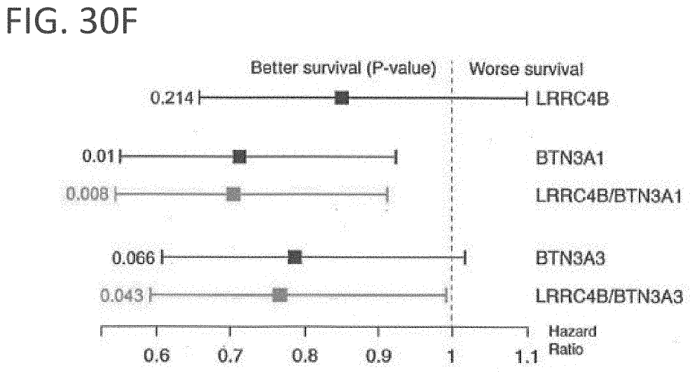

The development of cancer immunotherapies to reinvigorate pre-existing anti-tumoral responses signifies an unparalleled leap forward in the treatment of cancer. In particular, therapeutic antibodies that block inhibitory checkpoints, such as PD-1-PD-L1, have been shown to induce durable responses in patients with multiple cancers. Notwithstanding the measurable success of the checkpoint therapies, these responses only occur in a subset of the patients, suggesting the existence of unknown non-redundant pathways that influence tumorigenesis (Sharma et al., Cell, 168: 707-723, 2017). Thus, to comprehensively elucidate the receptor-ligand networks associated with clinical outcome and identify new determinants of resistance and response to therapy, the IgSF Interactome was studied in the context of a large cohort from a phase 2 trial (IMvigor210) which included 298 transcriptome samples from metastatic urothelial cancer patients treated with the PD-L1 blocking antibody atezolizumab (Mariathasan et al., Nature, 554: 544-548, 2018). Interestingly, subsets of IgSF Interactome proteins exhibited strong correlations with immune cold (or non-inflamed) or hot (inflamed) tumors, defined by a gene set associated with CD8+ T_{eff} cells (Mariathasan et al., 2018). Immune hot tumors were further stratified using a pan-fibroblast TGFb signature associated with stroma-high tumors. correlated with an immune excluded phenotype (Mariathasan et al., Nature, 554: 544-548, 2018). Notably, we observed that newly identified interacting pairs such as PD-L2/CEACAM4, LILRB1/IL6R, and LILRB4/CNTFR were strongly correlated with hot tumors, including interactions between NCR1 and the SIGLEC6 or SIGLEC8 proteins, or members of the SLITRK family that bound to PTPRD, which were predominantly associated with stroma-rich tumors (FIG. 28A). These immune receptor interactions were largely absent from immune cold tumors, in line with the notion that these tumors lack an active immune response (Chen and Mellman, Nature, 541: 321-330, 2017). Conversely, cell adhesion families including the semaphorin and the plexin receptors or UNC and FLTR proteins, alongside newly identified interactors for these families such as UNCSD or glypican-3 (GPC3), were prominently associated immune cold tumors (FIG. 28A). Such a T_{eff} cell signature has been shown to correlate with PD-L1 expression in immune cells and better response to treatment (Mariathasan et al., Nature, 554: 544-548, 2018). In line with this observation, our analysis highlighted interacting proteins that showed a clear association with the CD8+ T_{eff} gene set, including the PD-1/PD-L1 family, well-characterized immune receptors pairs such as CTLA4/CD80, CD28/CD86 and CD47/SIRPA, as well as newly identified proteins pairs such as PD-L1/EPHA3 or the LILRB1 binders EDAR and IL6R (FIG. 30A). On the contrary, certain protein communities were negatively correlated with the CD8+ T_{eff} genes, including pairs within the semaphorin/plexin family and the putative FLT1 interactors TREM2 and EPHB6 (FIG. 30A).

We then sought to investigate IgSF Interactome features beyond T_{eff} cell function, by first evaluating the association between the interacting protein pairs and clinical outcome. Patients were categorized in responders and non-responders to Atezolizumab, as previously described (Mariathasan et al., Nature, 554: 544-548, 2018). Approximately 80 protein interactions (˜15% of the Interactome network) were significantly associated with either an improved or worsened clinical outcome for this cohort (FIG. 28B and Tables 15 and 16). As expected, interactions within the CD80 and PD-1 families of immunoreceptors were strongly correlated with response to atezolizumab. In addition, new putative protein pairs such as NCR1/SIGLEC6, or the interactions between LRRC4B and specific butyrophylin members, were strongly correlated with response (FIG. 28B). Conversely, interactions within the semaphorin/plexin family, as well as specific PTPRD regulators such as LFRN4 and IL1RAP were significantly associated with lack of response to treatment (FIG. 28B). Next, we investigated whether interacting gene pairs showed a synergistic effect on the predicted clinical outcome by comparing the ‘protein interaction’ hazard ratio (HR), derived from their joint expression, to the hazard ratio of their individual gene expression. Notably, 137 protein pairs, representing ˜25% of the IgSF Interactome network, showed a significantly exaggerated hazard ratio over single proteins (FIG. 28C). Protein pairs for which this synergistic effect was observed included SIGLEC6 and NCR1; BTN3A1 and LRRC4B; CD80 and CTLA4; BTN3A3 and LRRC4B; EFNB1 and TRHDE; CTLA4 and PCDHGB4; CTLA4 and FAM200A; CA12 and SIGLEC6; ILDR2 and CLEC12B; EFNB1 and ITLN1; CADM1 and CRTAM; CD79B and CD244; and DAG1 and EFNB1, associated with responsiveness to atezolizumab therapy, and EFNB1 and EVC2; GPC4 and FGFRL1; EFNB3 and EPHB4; PTPRD and LRFN4; EFNB1 and AQPEP; EFNB1 and DSG4; LDLR and LILRB5; EFNB3 and EPHB3; PLXNB3 and SEMA4G; EFNB1 and EPHB6; FLT4 and FLRT2; AXL1 and IL1RL1; CD320 and IGSF5; CD59 and STAB1; CNTN3 and PTPRG; EFNB1 and EPHA3; EFNB3 and EPHB2; EGF and TNFRSF11B; ENPEP and SLITRK1; FCGR3B and EDA2R; IL20RA and CLEC14A; IL6R and BTNL9; IZUMO1 and LILRA5; NGFR and LRRTM3; NTM and AMIGO2; PCDHB3 and IGSF11; PTGFRN and TMEM59L; and TREM1 and VSIG8, associated with lack of responsiveness to atezolizumab therapy.

Among those, we observed new interactions for proteins with well characterized roles in tumors, such as the vascular endothelial growth factor receptors FLT1 and FLT4, or the regulators of cell death and immune responses EDAR, TNFRSF11B and CD47. As expected, interactions within the PD-L1 family were associated with response to atezolizumab and overall survival (FIGS. 30B and 30E). In addition, a number of the novel receptor interactions were also strongly correlated with response to treatment, illustrated by the LRRC4B/BTN3A1/BTN3A3 or NCR1/SIGLEC6 protein pairs (FIGS. 30C and 30F and Tables 15 and 16).

Conversely, interactions within protein families such as the fibroblast growth factor receptor (FGFR) tyrosine kinases or members of the LILR family were identified as interactions significantly associated with lack of response (FIG. 28C and Tables 15 and 16). A prominent example of such synergistic interactions was ephrinB1 (EFNB1), for which we confirmed binding members within the Ephrin family, together with newly identified previously unrelated interactors (FIG. 30D). Multiple EFNB1 interactions, including EVC2, AQPEP and EPHB6, showed a significantly higher association with non-responders when both genes in the pair were expressed relative to each of the genes in isolation (FIG. 28D). Consistently, expression of the interacting protein pair was significantly associated with lack of response to treatment and poor prognosis, illustrated by synergistic predictive effect between EFNB1 and the Hedgehog signaling machinery protein EVC2 (FIGS. 28E and 28F).

Collectively, the study of the IgSF Interactome in the context of a large cohort of patients defines a gene signature map associated with CD8+ T_{eff} function as well as specific tumor immunophenotypes, providing unique insights into receptor function, crosstalk and association with cancer progression. Further, these results highlight protein communities associated with response to PD-L1 blockade, and identify interacting gene signatures with improved predictive power for clinical outcome.

### Example 10. Correlation of CAF-Expressed PDPN with Poor Outcomes in CRC Patients

The tumor microenvironment (TME) consists of a mixture of tumor and nonmalignant cells, including non-hematopoietic stromal cell types such as blood endothelial cells (BECs), lymphatic endothelial cells (LECs), and cancer-associated fibroblasts (CAFs), as well as hematopoietic immune cells. The tumor cells and the stroma establish a dynamic crosstalk that plays a critical role in regulating tumor growth and progression (Turley et al., Nat Rev Immunol, 15: 669-682, 2015; Peranzoni et al., Cell Mol Life Sci, 70: 4431-4448, 2013). Although the relationship between the stroma and tumor-infiltrating immune cells remains far less understood, compelling evidence indicates that the stroma importantly influences anti-tumor immune responses and responsiveness to immunotherapies (Turley et al., Nat Rev Immunol, 15: 669-682, 2015). Fibroblasts, which constitute the majority of stromal cells within the TME, regulate the structure and function of the tissue via extracellular matrix (ECM) remodeling and secretion of a plethora of inflammatory factors. In addition, increasing evidence suggests that CAFs impair anti-tumor immunity, supporting tumor cell growth and dissemination (Turley et al., Nat Rev Immunol, 15: 669-682, 2015; Chen et al., Nat Rev Drug Discov, 18: 99-115). In line with this notion, a recent report demonstrated that CAFs are major drivers of increased metastasis in response to TGF-b and poor outcomes in colorectal cancer (CRC) (Calon et al., Nat Genet, 47: 320-329, 2015).

Notwithstanding the emerging role for CAFs and other stromal cells in immunosuppression and resistance to immunotherapy, the downstream molecular circuitry responsible for crosstalk between CAFs and tumor-infiltrating immune cells remains poorly understood. Numerous reports have shown that podoplanin (PDPN, gp38, Aggrus, or T1α), a single transmembrane (STM)-containing receptor predominantly expressed by CAFs and LECs, is over-expressed in tumors and is associated with poor prognosis (Astarita et al., Nat Immunol, 16: 75-84, 2015). However, the function of PDPN in the tumor unclear, and fundamental aspects of PDPN biology have remained largely uncharacterized. We have shown that in mouse lymph node fibroblasts, PDPN is a master regulator of actomyosin contractility and is inhibited by the C-type lectin receptor CLEC-2 (Clec1b), which is predominantly expressed in platelets and dendritic cells (Astarita et al., Nat Immunol, 16: 75-84, 2015; Acton et al., Nature, 514: 498-502, 2014). Upon inflammation, CLEC-2 attenuates PDPN-mediated contractility, resulting in reduced lymph node stiffness and enhanced immunity (Astarita et al., Nat Immunol, 16: 75-84, 2015). It is unknown whether PDPN functions similarly in human fibroblasts to confer contractility and mediate interactions with immune cells, particularly in the unique environment that surrounds solid tumors.

Here, a gene signature representing the presence of PDPN⁺ CAFs was found to correlate with a significant reduction in patient survival, indicating that expression of PDPN in human fibroblasts may importantly influence tumor progression.

A. PDPN Expressed by CAFs Correlates with Poor Outcome in Human CRC

PDPN expression is highly elevated in several cancers, including squamous cell carcinoma of the head and neck, gliomas, and colon cancers, and is associated with poor survival outcomes. These studies are largely based on immunohistochemistry analysis and do not always define the cellular source of PDPN. This distinction is important when considering the function of this protein as it can be expressed by tumor cells, LECs, CAFs, and even some immune cells, all of which exert different effects in the TME (Astarita et al., Front Immunol, 3: 283, 2012; Turley et al., Nat Rev Immunol, 15: 669-682, 2015). A recent report identified TGFb signaling in PDPN+FAP+ CAFs as a major driver of metastasis in CRC patients (Calon et al., Nat Genet, 47: 320-329, 2015).

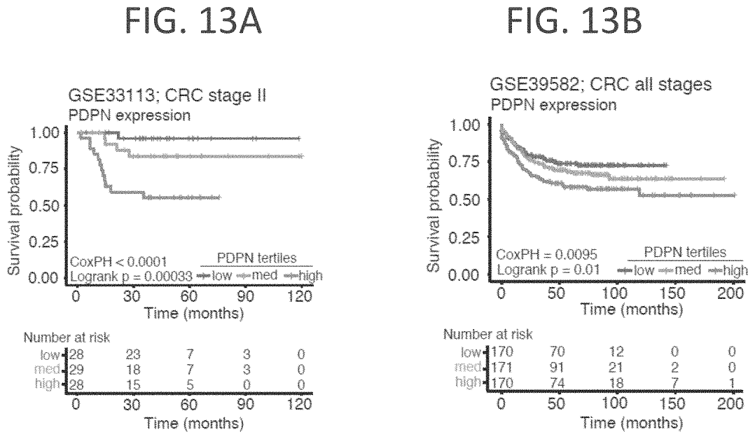

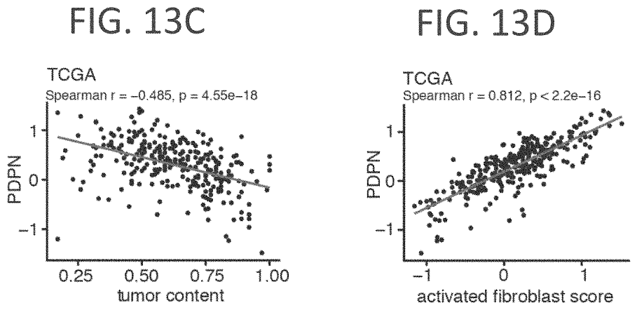

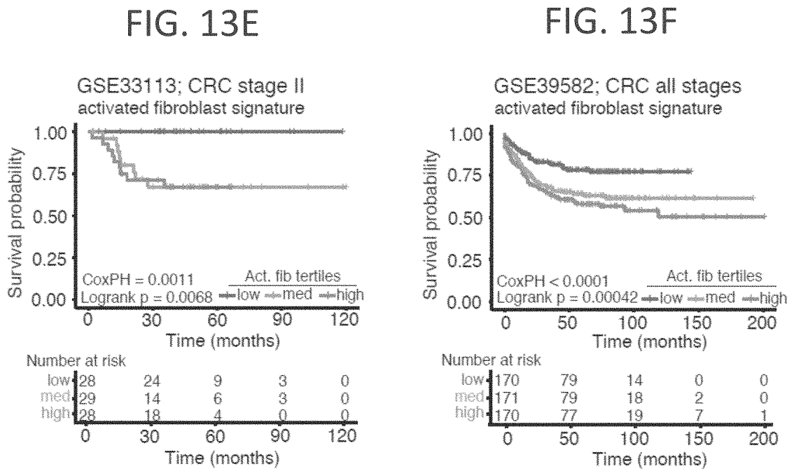

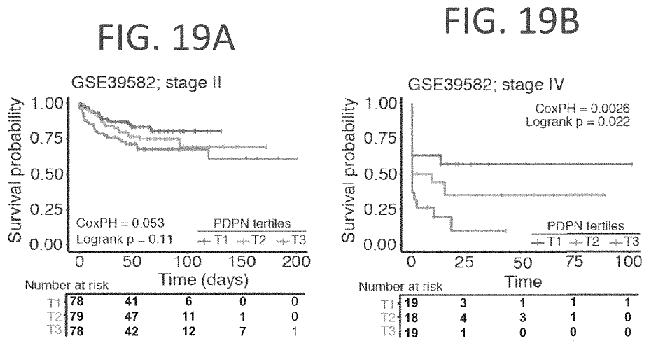

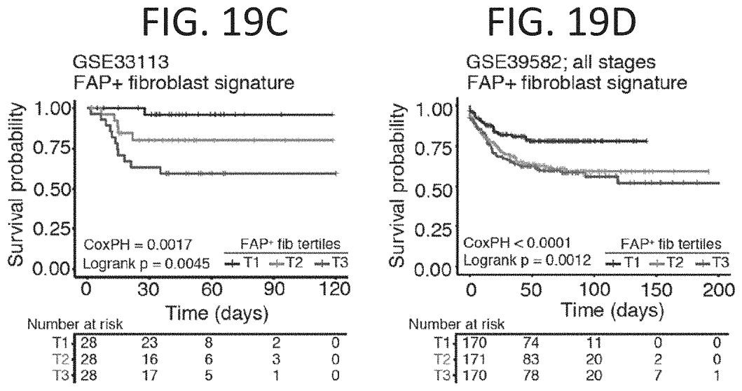

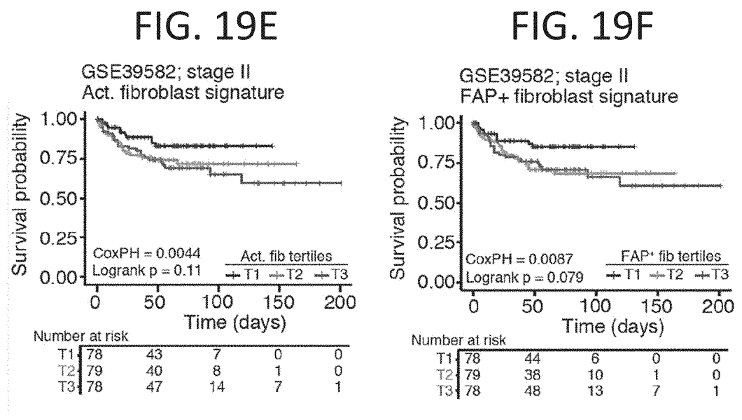

Thus, to determine whether PDPN expression at the RNA level was a significant prognostic factor in CRC, bulk tumor gene expression and recurrence-free survival (RFS) data from two previously published human CRC studies were queried. In a cohort consisting of stage II patients (de Sousa et al., Cell Stem Cell, 9: 476-485, 2011) (GSE33113, n=85), we found that PDPN expression was significantly associated with reduced survival (FIG. 13A). Similarly, in a larger CRC cohort that included patients from all stages (Marisa et al., PLoS Med, 10: e1001453, 2013) (GSE39582, n=511), PDPN expression was again significantly associated with reduced RFS, both in the entire cohort and in stage II patients (FIGS. 13B and 19A). In late stage disease, PDPN expression was a highly significant risk factor for stage IV (FIG. 19B), but not for stage III CRC patients (Table 10).

B. PDPN Functions as a Regulator of Cell Tension/Contraction in Primary Human Fibroblasts

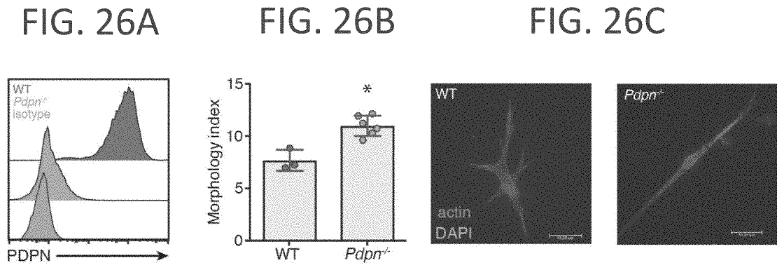

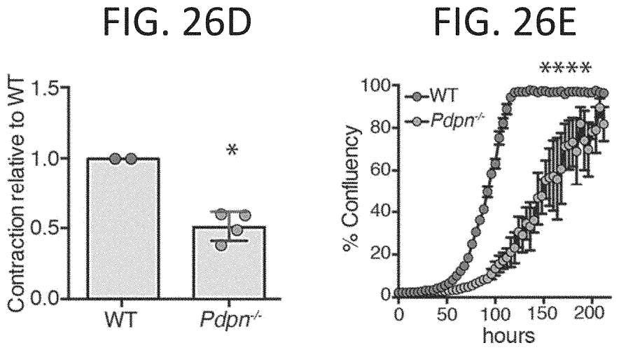

In light of the significant association between PDPN expression and reduced survival in CRC, we next sought to determine the function of PDPN in primary fibroblasts isolated from CRC lesions. Pdpn−/− cells were generated using a Cas9-RNP-CRISPR system in primary human CAFs grown from cancerous colorectal tissue, which express high endogenous levels of PDPN (FIG. 26A). Upon seeding these cells into 3D collagen matrices, we observed that Pdpn−/− CAFs exhibited a markedly polarized morphology compared to WT CAFs, with an approximately 70% more elongated phenotype (FIGS. 26B-26C). In addition, to test whether the PDPN deletion had other functional consequences, we performed a variety of assays to measure growth rate, the ability of cells to generate tension, and the ability of cells to adhere to surfaces. To test contraction, the CAFs were seeded into 3D matrices and were allowed contract the gels over a period of three days. Consistent with our hypothesis that PDPN is a master regulator of fibroblast contractility, the PDPN-deficient cells were significantly impaired in their ability to contract the gels (FIG. 26D). Finally, we examined whether a lack of PDPN affects the growth of these cells, given that fibroblast contractility can control cell growth and survival. Notably, Pdpn−/− fibroblasts grew significantly more slowly compared with WT cells (FIG. 26E). Altogether, these results indicate that PDPN plays a major role in actomyosin contractility in human cells and promotes fibroblast growth in a cell-autonomous manner.

C. Activated Fibroblast Signature

We next asked whether PDPN expression in the tumor microenvironment (TME) was associated primarily with fibroblasts or with tumor cells. We first curated a list of activated fibroblast genes (activated fibroblast signature) from the literature and our own data (Table 11) and used it to infer fibroblast levels in bulk tumor RNA-seq data. We then utilized published DNA copy number-based tumor content data (Taylor et al., Cancer Cell, 33: 676-689, 2018) and investigated the association between PDPN expression, inferred fibroblast levels, and tumor content. We found that PDPN mRNA levels had a strong positive correlation with inferred activated fibroblast levels (FIG. 13D), but had a negative correlation with tumor content (FIG. 13C), indicating that PDPN expression in the TME comes largely from fibroblasts.

D. FAP⁺ Fibroblast Signature

We next sought to capture the presence of cancer-associated fibroblasts (CAFs) at a finer level in RNA-seq datasets. We utilized data from a recent study that defined a role for FAP⁺ stromal cells in driving CRC (Calon et al., Nat Genet, 47: 320-329, 2015). We performed differential expression tests among the four sorted cell populations of the study (FAP⁺ fibroblasts, CD31⁺ endothelial cells, immune cells (CD45⁺ cells), and tumor cells (EpCAM⁺ cells) and defined a FAP⁺ fibroblast signature from the top 35 genes expressed only by fibroblasts and not by other cells (Table 12).

E. Association of Activated Fibroblast Signature and FAP⁺ Fibroblast Signature with Survival

We then investigated whether the activated fibroblast signature and the FAP⁺ fibroblast signature had prognostic value in human CRC. Both signatures were significantly associated with poor relapse-free survival (RFS) in both GSE33113 and GSE39582 (FIGS. 13E-13F, 19C-19D). Because GSE33113 included only stage II patients, we then asked whether the stage II cohort in GSE39582 confirmed the signatures as negative prognostics. We found that both signatures were also associated with poor RFS in the GSE39582 stage II cohort (FIGS. 19E-19F). Both fibroblast signatures remained significant predictors of RFS when included in multivariate models fit to all patients while controlling for stage (Table 10). These data indicate that PDPN is an indicator of poor outcomes in CRC and that PDPN-expressing fibroblasts in the TME contribute to tumor growth.

### Example 11. Characterization of the Extracellular Interactome

In an effort to better understand PDPN function in CAFs and to overcome the limitations of most commonly used receptor-ligand screening approaches, we built a library representing most STM human receptors engineered for controlled display on the cell surface. Additionally, we developed a tetramer-based screening method to detect transient, low affinity interactions. Here, we utilized this new platform to study the STM interactome of PDPN in humans, and identified the neutrophil marker CD177 as a novel binding partner.

A. A New Platform for Controlled Protein Display on the Plasma Membrane and Enhanced Detection of Transient Receptor-Ligand Interactions

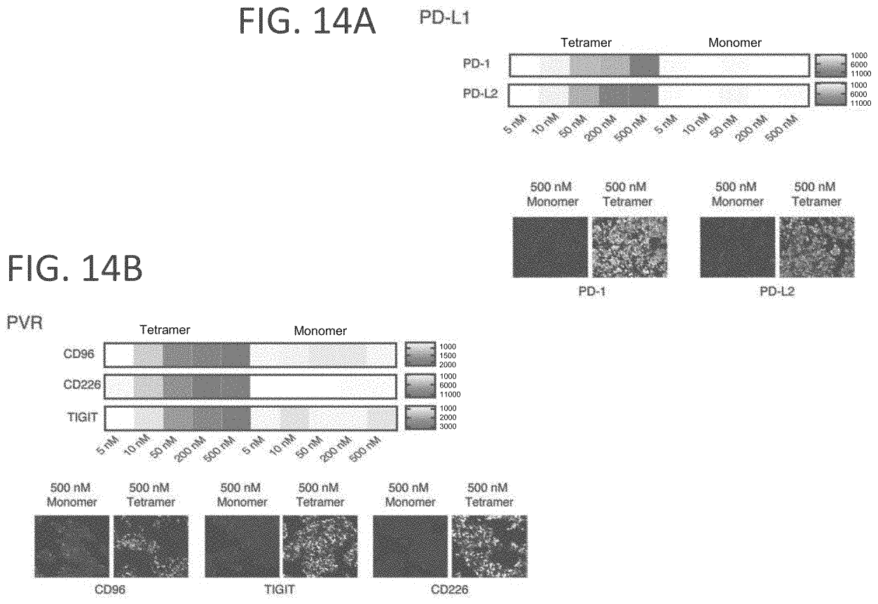

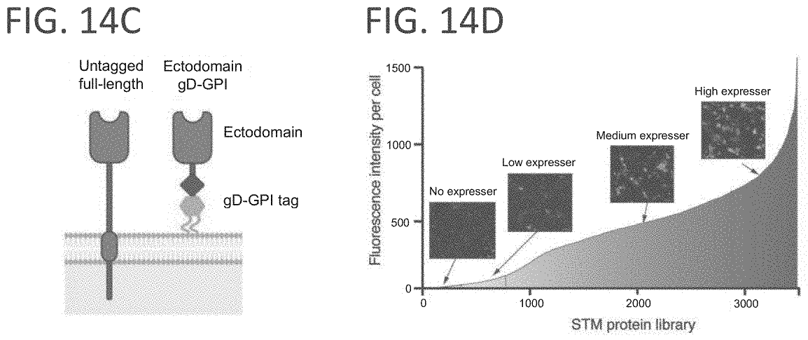

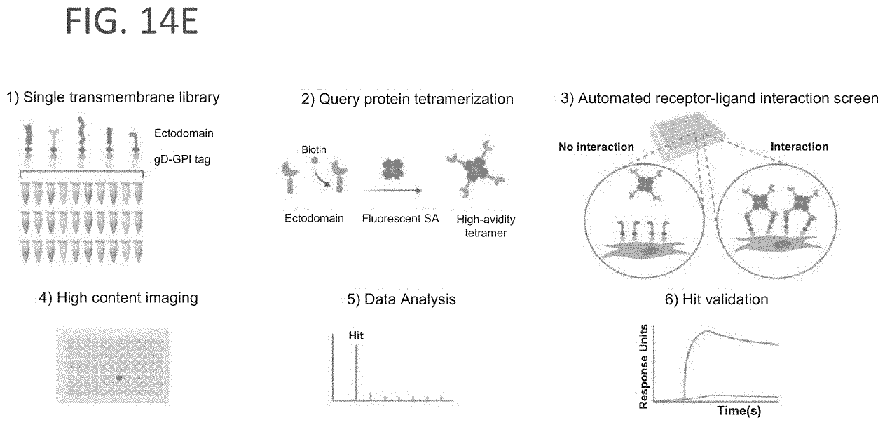

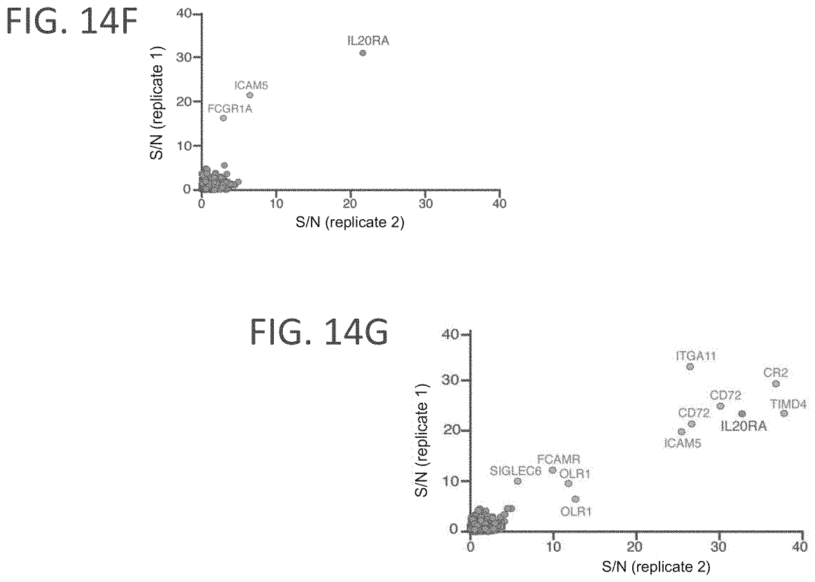

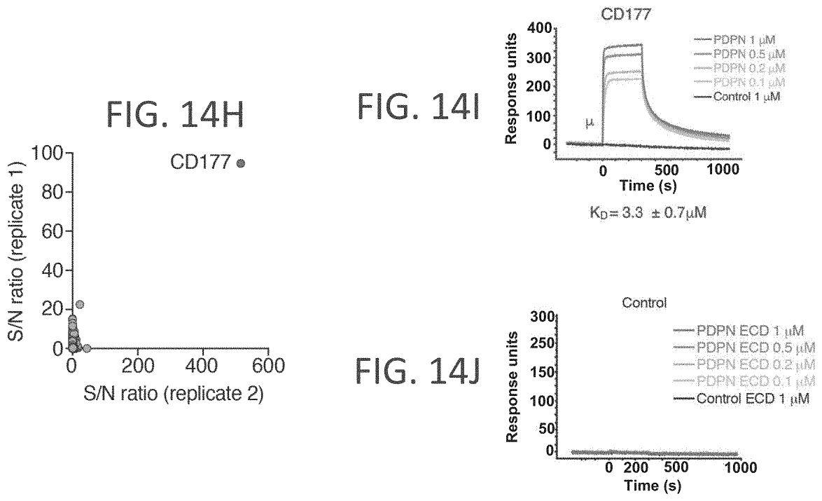

Protein interactions in the extracellular milieu are often characterized by transient, low affinity binding (K_{D} in the micromolar to millimolar range) that challenge identification by most commonly used approaches (Martinez-Martin, 2017). To facilitate detection of such interactions, we developed a tetramer-based method for enhanced protein multimerization and binding avidity. First, this approach was used to test interactions between the immune receptors PD-L1/CD274 and the poliovirus receptor PVR and their respective ligands. Briefly, PD-L1 or PVR, the query proteins, were expressed as recombinant biotinylated ectodomains, and then tetramerized using fluorescent streptavidin to enable quantification of receptor-ligand interactions. Next, the monomeric or tetrameric PD-L1 or PVR were tested for binding to cells transiently expressing the relevant binding partners. Tetramerization of the query protein significantly enhanced detection of receptor-ligand interactions over the monomeric ectodomain, including micromolar affinity interactions such as PD-1-PD-L1 (FIGS. 14A and 14B).

We first tested this protein library for expression on transiently transfected cells, using a semi-automated transfection procedure. Notably, from over 3,500 STM proteins analyzed for cell surface expression, medium to high cell surface expression levels were achieved for over 75% of the STM proteins, whereas only ˜10% of the proteins did not show detectable expression on the plasma membrane, indicating that most of the receptors in the library are displayed on the cell surface and available for interaction with the relevant binding partners (FIG. 14D).

Next, we sought to utilize the newly developed ectodomain-gD-GPI STM protein collection (FIG. 14C) in combination with the tetramer-based screening to enhance discovery of receptor-ligand interactions in high throughput. To do so, a method for automated cell transfection and screening was implemented, followed by high content imaging for detection of fluorescent tetramer binding to the cell surface (FIG. 14E). We first used this platform to study the immune receptor B7-H3/CD276 for cell surface interactors (FIG. 14F). The interleukin-20 receptor subunit alpha (IL20-RA) was detected as an interactor, in agreement with recent findings (Husain et al., Mol Cell Proteomics, 18: 2310-2323, 2019). To demonstrate that the gD tag and GPI anchor did not impact detection of protein interactions at the cell surface, B7-H3 was also screened against a library of STM proteins that were expressed as full-length proteins in the absence of any tags (FIG. 14G). In line with the previous results, this assay identified IL20-RA as a high scoring hit, which demonstrates that the ectodomain-gD-GPI library is amenable for detection of protein interactions on the cell surface.

B. gD-GPI-Tagged Human STM Receptor Library

A list of STM receptors was compiled using bioinformatic analysis using various algorithms for prediction of protein features, such as protein domains and subcellular localizations, followed by manual curation and review of published annotations. This list was used to create a library consisting of most human STM receptor proteins (Table 7). STM receptors were expressed as extracellular domains (ECDs) fused to a glycosylphosphatidyl-inositol (GPI)-glycoprotein D (gD) tag (gD-GPI) to enhance expression at the cell surface (FIG. 14E). The boundaries of the extracellular domain (ECD) were annotated as in Example 1B. The ECD of each receptor, containing its native signal sequence, was synthesized and cloned into a pRK5 vector (Genentech) in frame with a gD-GPI tag.

C. Tetramerized Query Protein

The query protein PDPN was expressed as the PDPN extracellular domain (ECD) fused to an avi tag allowing site-directed biotinylation, was biotinylated, and was tetramerized for increased binding avidity using fluorescent streptavidin (FIG. 14E). To tetramerize ECDs, allophycocyanin (APC)-conjugated streptavidin was purchased from Prozyme as custom vials, typically at a concentration of 2 mg/mL. Avi-tagged, biotinylated PDPN ECDs were tetramerized following the protocol provided by the NIH Tetramer Core Facility. In brief, APC-conjugated streptavidin was added over 10 time intervals to maximize tetramer formation versus dimeric or trimeric species. The amount of streptavidin added was calculated based on the molecular weight of the PDPN and the streptavidin reagent, assuming 100% biotinylation. Streptavidin was added at room temperature with the samples protected from light, and tetramers were subsequently stored on ice until the assay was performed. Tetramers were prepared fresh on the day of the assay.

D. Automated Single-Clone, Cell-Based Receptor Discovery Platform

The library of human STM receptors was screened for interaction with PDPN using a cell surface receptor interaction screen (FIG. 14E), as described in Section 2A (ii) herein. The tetramerized PDPN construct of Example 8B was used as a query protein. The gD-GPI-tagged human STM receptor library of Example 8A was used as a prey library.

i. Transfection of Cells

The library of STM human receptors was expressed on COS-7 cells. Cells were transiently transfected with individual receptor clones following a reverse transfection protocol using a semi-automated procedure. Lipofectamine LTX-Plus reagent (Life Technologies) was used for transfection, following manufacturer's instruction with minor modifications. Briefly, 25 μL of Lipofectamine LTX-Plus mixture in Opti-MEM medium (Thermo) was dispensed to 384 well microtiter plates containing 6 ng of DNA per well. The DNA-lipofectamine complexes were incubated for 30 min at 37° C., and subsequently the cells (resuspended in DMEM media at 0.125 million cells/mL) were aliquoted in the assay plates using an automatic cell dispenser.

ii. Cell Surface Interaction Screen

A cell surface interaction screen for PDPN binding partners was performed 48 h after transfection. A number of GFP-tagged clones were included to control for cell transfection efficiency. Analysis of PDPN ECD binding to the cell surface was performed using an integrated robotic system consisting of automated liquid handling devices (plate dispensers and washers), to allow for high throughput analysis of PPIs while minimizing manual operations to increase data quality. Growth media was removed from cell cultures and cells were washed an incubated with 1% bovine serum albumin (BSA) for 30 min. Following washing, the cells were incubated with the PDPN ECD tetramer for 45 min at 4° C. Cell surface binding was assayed in PBS containing 0.1% BSA supplemented with calcium and magnesium. Following incubation with PDPN, the cells were washed and fixed with 4% PFA and stored at 4° C. protected from light. Images were acquired from individual wells using a high content microscope (In Cell 6000, GE Healthcare) assisted by a robotic arm. Cell surface tetramer staining was represented as fluorescent signal intensities (FIG. 14E).

iii. Image Processing

Image data were exported as tiff files and processed using the Developer Toolbox version X software. Images were analyzed using a custom analysis module, and segmentations were performed based on positive cell surface staining. Typically, events in the range of 10-500 signal/noise ratio may be considered “hits” in the analysis module. Minimal post-processing analysis and exclusion parameters were set up to obtain optimal outline of desired objects and minimize any background signals due to screening artifacts. PDPN binding to the cell surface was represented as signal/noise ratio (FIG. 14E).

A single novel binding PDPN binding partner, CD177 (NB1, PRV1), was identified (FIG. 14H). CD177 is a GPI-linked cell surface glycoprotein expressed in neutrophils and a minor fraction of intratumoral regulatory T cells (Tregs) (Plitas et al., Immunity, 45: 1122-1134, 2016).

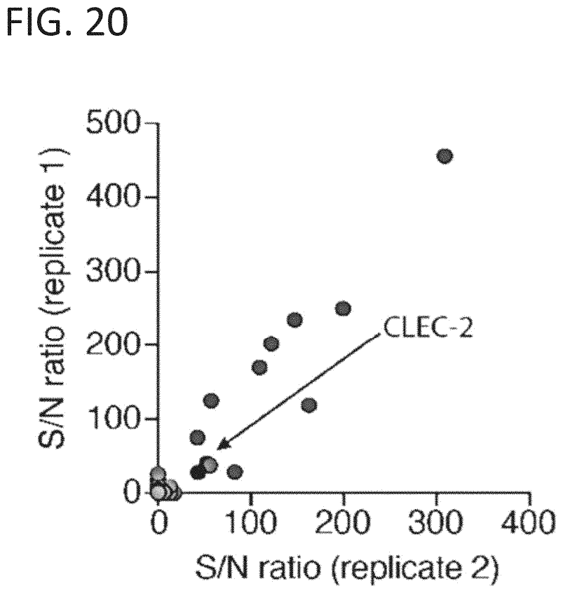

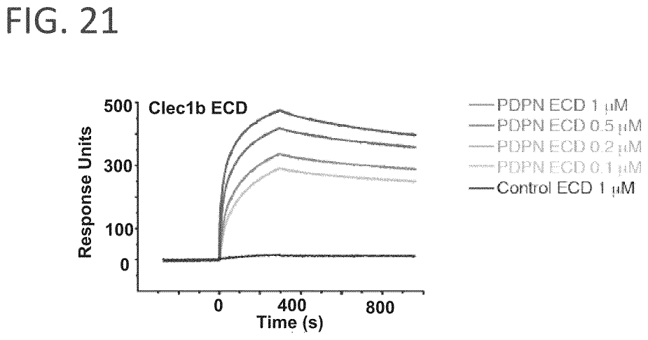

Similar screens performed against a library consisting of most STM-like receptors expressed as full-length proteins confirmed interaction between PDPN and human CLEC-2 (FIG. 20A); CLEC-2 is a known PDPN binding partner in murine models (Astarita et al., Nat Immunol, 16: 75-84, 2015).

iv. Validation of Interactions Between PDPN and CD177 and CLEC-2

To further validate the interactions between PDPN and CD177 and CLEC-2, we assayed binding using surface plasmon resonance (SPR). SPR was measured using a Proteon XPR36 Instrument (Biorad) or a Biacore 3000 (GE Healthcare). The purified proteins (CD177 or CLEC-2, expressed as recombinant ECDs) were immobilized on GLC or CM5 sensor chips, respectively, using the amine coupling method. Avidity AVITAG™ (Avi)-tagged PDPN was run as a soluble analyte at the indicated concentrations in PBS containing 0.01% Tween-20 (when the Proteon Instrument was used), or HBS-P buffer (0.01M Hepes, 0.15 M NaCl, 0.005% surfactant P20, pH 7.4), for Biacore assays. Bulk refractive index changes were removed by substrating a reference flow response. For K_{D} calculations, 300-400 resonance units were immobilized, and kinetic data were fit to equilibrium or a Langmuir model to calculate kinetic parameters for CD177 and CLEC-2 binding, respectively. For kinetics calculations, Avi-tagged monomeric PDPN ectodomain was used as the analyte. All sensorgrams were analyzed with BiaEvaluation 4.1 (Biacore) or Proteon Manager 3.1.0.6 (Proteon) software.

These studies corroborated the interaction between PDPN and CD177 (FIGS. 14I-14J) and between PDPN and CLEC-2 (FIG. 20B). Interestingly, PDPN interacted with CD177 with micromolar binding affinity (K_{D}=3.3±0.7 μM), one order of magnitude lower than the affinity detected for CLEC-2 (K_{D}=210±0.3 nM) (FIG. 14I), illustrating the sensitivity of our approach to detect protein-protein interactions (PPIs) of a range of affinities.

v. Assessment of Interactions Between Mouse PDPN, CD177, and CLEC-2

We also used SPR to test whether mouse CD177 and PDPN would interact. We were able to recapitulate mouse CLEC-2 binding to PDPN, but were unable to detect any binding of mouse CD177 to mouse PDPN in a variety of formats (not shown), possibly due to the low sequence conservation in mouse and human CD177, suggesting that this interaction plays a relevant function specific to the human immune system.

### Example 12. PDPN, CLEC-2, and CD177-Expressing Cells in the Tumor Microenvironment

Primary human blood and colon tissues were analyzed to determine which cell types express PDPN and CD177.

A. Blood Samples

Blood samples were obtained from healthy donors under the Genentech blood donation program. Neutrophils were isolated from blood using the MACSExpress Neutrophil isolation kit (Miltenyi). Peripheral blood mononuclear cells (PBMCs) were purified from heparinized blood by Ficoll (Lymphoprep) gradient centrifugation, followed by isolation of T cells using the Pan T Cell isolation kit (Miltenyi).

B. CAFs

Primary human colorectal cancer-associated fibroblasts (CAFs) were purchased from Vitro Biopharma and grown in MSC-Gro low serum growth media (Vitro Biopharma) for a maximum of 5-6 passages. The HT-29 cells were purchased from ATCC and grown in McCoy's 5a media with 10% FBS. All cells were cultured at 37° C. and 5% CO₂.

C. CD177 in Blood and Colon Cells

i. CD177 Expression in Blood Neutrophils

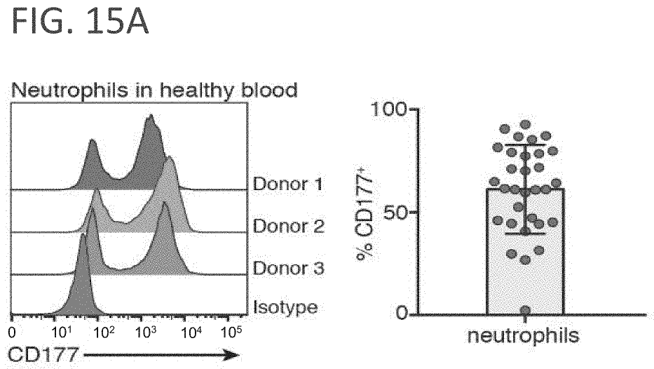

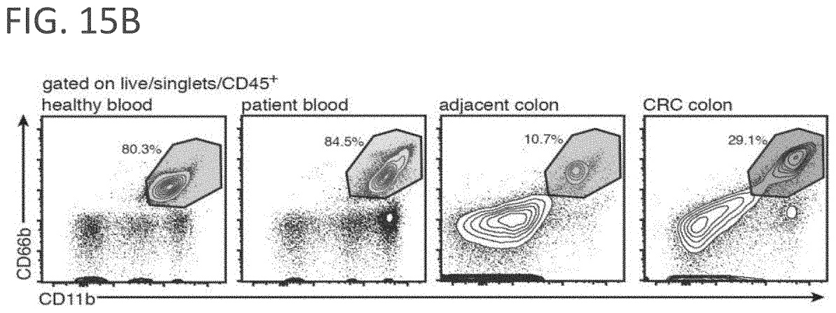

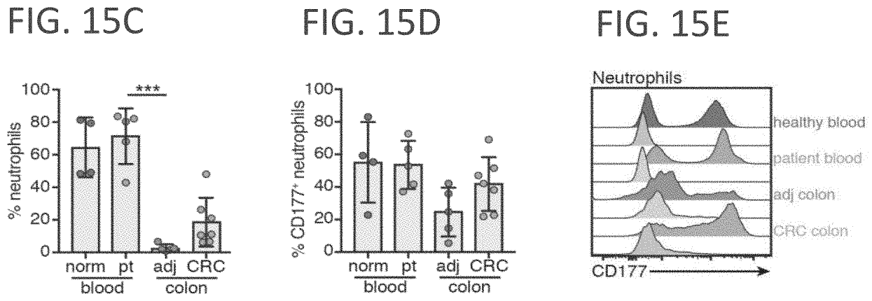

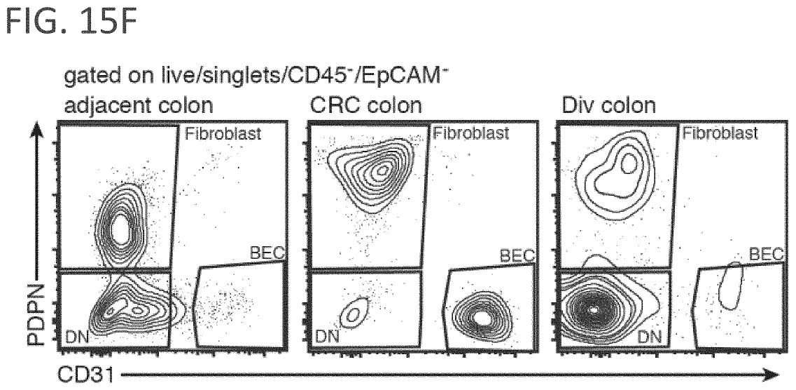

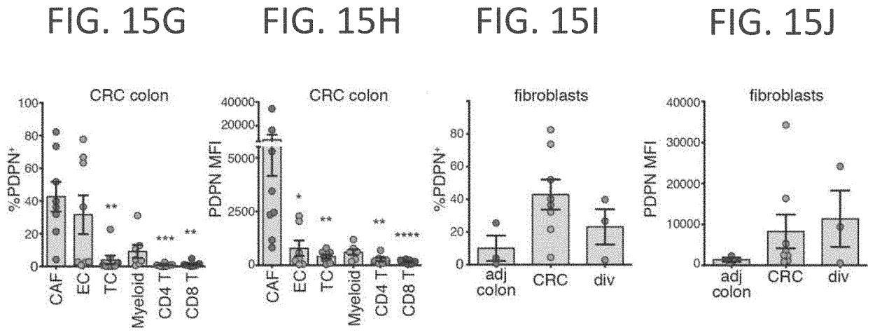

We first confirmed the reported expression of CD177 on neutrophils in the blood. As previously reported (Bai et al., Blood, 130: 2092-2100, 2017), we found that the percentage of neutrophils expressing CD177 varied widely between individuals, with an average of about 60% of neutrophils being CD177⁺ (FIG. 15A).

ii. Expression of PDPN and CD177 in Microarray Data

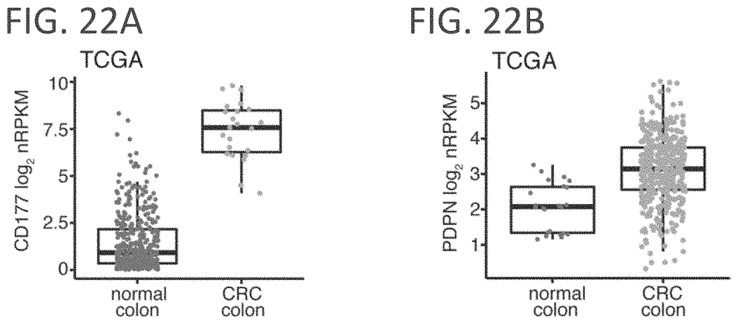

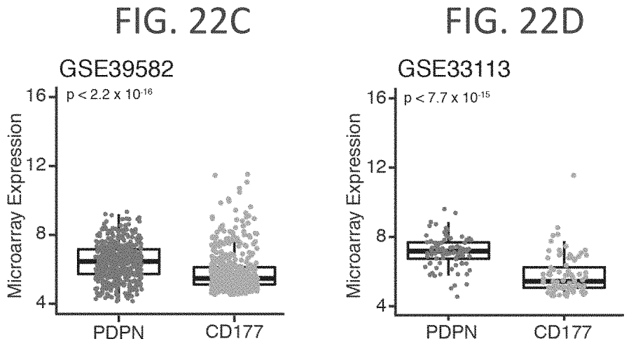

We next investigated RNA expression of PDPN and CD177 in the GSE33113 and GSE39582 microarray datasets. Expression of PDPN was significantly higher in tumor tissue compared to normal tissue, and expression of CD177 was much lower than expression of PDPN in tumors (FIGS. 22A-22B).

iii. Expression of PDPN and CD177 in Cell Compartments

We next examined the expression patterns of PDPN and CD177 in human blood and colon tissues from healthy individuals and colorectal cancer (CRC) patients. RNA expression of PDPN and CD177 was significantly higher in tumors compared with normal tissues in the TCGA data set (FIGS. 22A and 22B). In the two data sets examined in FIGS. 13A-13F, CD177 and PDPN were expressed, but it is impossible to discern the cell source of RNA in bulk tumor RNA data (FIGS. 22C and 22D). Thus, to study expression and cellular distribution at the protein level, fresh human tissues were digested into single cell suspensions using a digestion protocol that allowed for good recovery of all cell types, including stromal cells and immune cells, with good viability. Fluorescence-activated cell sorting (FACS) was performed, and samples were gated as in FIG. 15B.

We found that normal adjacent colon tissue contained relatively few neutrophils, whereas tumors had a large influx of neutrophils (FIGS. 15B-15C). As in the blood, tumor neutrophils expressed varying amounts of CD177, but the ratio of CD177⁺ neutrophils was similar between the blood and tumor for an individual (FIGS. 15D-15E). Interestingly, CD177⁺ neutrophils were slightly enriched in the tumor relative to the adjacent normal tissue (FIGS. 15D-15E).

As was previously reported (Plitas et al., Immunity, 45: 1122-1134, 2016), we also observed that a small proportion of tumor Tregs expressed CD177, whereas CD177 was rarely observed on Tregs in non-tumor tissue, blood, or tonsil (data not shown). These CD177⁺ Tregs were much less abundant than CD177⁺ neutrophils.

We then examined the non-hematopoietic stromal compartment. Gates were chosen to include events that were live (as indicated by absence of 7-AAD staining), were singlets, were CD45⁻ (i.e., were not immune cells, and were EpCAM⁻) (i.e., were not tumor cells) (FIG. 15F). We found that approximately 40% of cancer-associated fibroblasts (CAFs), defined as total CD31⁻ stroma, were PDPN⁺ (FIG. 15G). While PDPN was also expressed by some lymphatic endothelial cells (LECs) and a small percentage of macrophages, the levels of PDPN on a per-cell basis were significantly higher on CAFs (FIG. 15H), indicating that they are the major source of PDPN in the tumor microenvironment (TME). This finding confirms the conclusions drawn from our bioinformatics analysis (FIG. 13D). Additionally, PDPN staining on CAFs was 6-fold higher than staining on fibroblasts from adjacent normal tissue, and a higher percentage of cells were PDPN⁺ (FIGS. 15I-15J).

We also examined fibroblasts in colon samples from patients with diverticulitis, an inflammatory condition, using the methods describe above. Interestingly, the fibroblasts in these tissues also upregulated PDPN relative to the normal tumor-adjacent tissue (FIGS. 15I-15J). This finding is in line with previous observations that PDPN can be upregulated by a variety of inflammatory stimuli, such as TGF-β and IL-6 (Astarita et al., Front Immunol, 3: 283, 2012).

Overall, these data confirm that CAFs are the major source of PDPN in tumor tissue and indicate that both PDPN⁺ CAFs and CD177⁺ neutrophils are enriched in colon tumor tissues.

D. IHC and Immunofluorescence Localization of PDPN and CD177

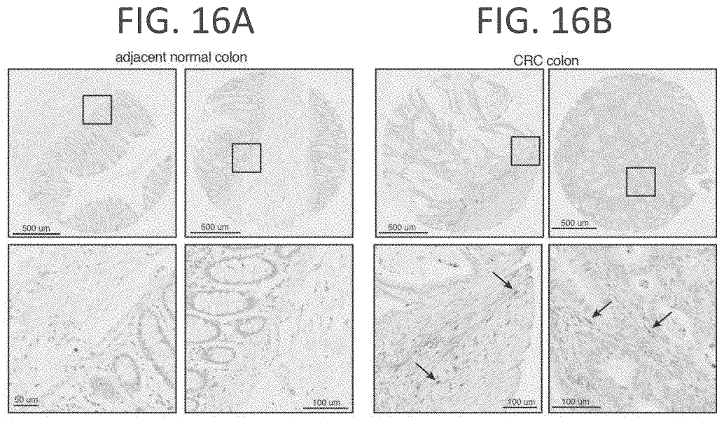

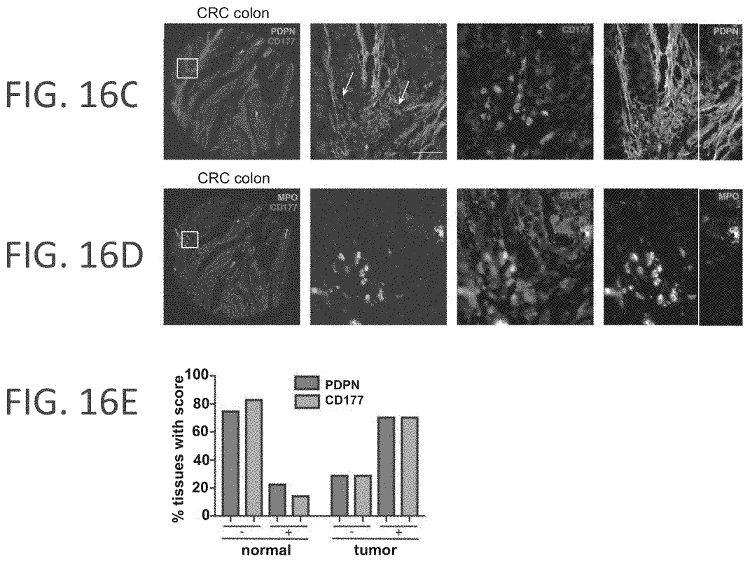

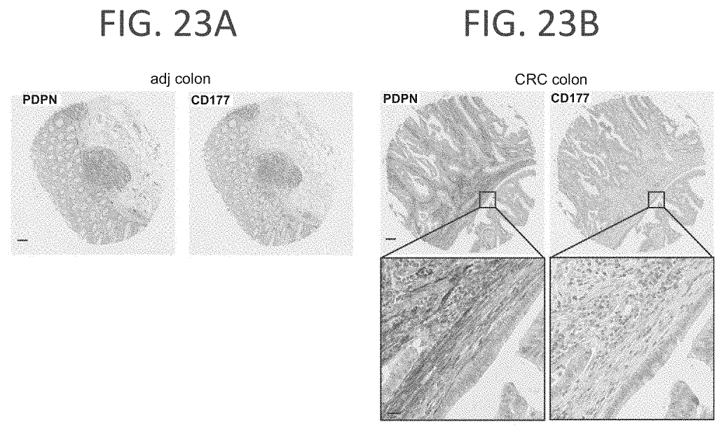

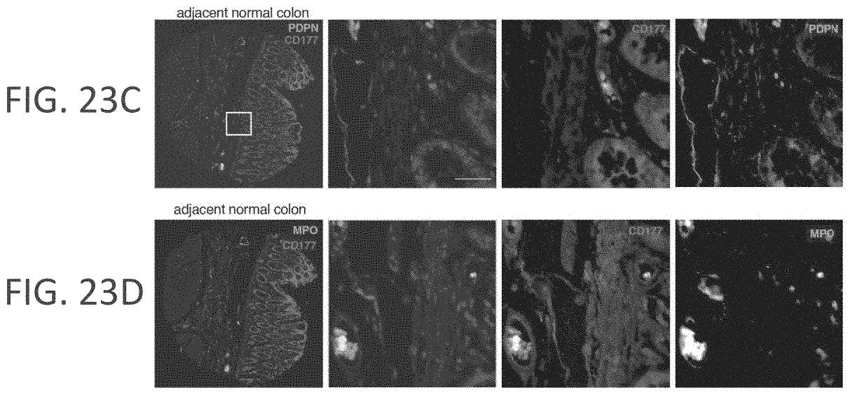

To examine the localization of PDPN⁺ and CD177⁺ cells within the TME, we performed dual immunohistochemistry (IHC) and immunofluorescence staining for PDPN and CD177 in human colon tissues from CRC tumors (CRC colon) and normal adjacent tissue (adj colon). In normal tissues, PDPN staining was largely restricted to lymphatic vessels, and few neutrophils were observed (FIGS. 16A, 23A, and 23C). In contrast, in tumor samples, PDPN was strongly upregulated in CAFs in the surrounding tumor stroma (FIGS. 16B-16C and 23B), whereas it was rarely observed on the tumor cells. The PDPN⁺ fibroblasts were generally aligned, creating large swaths of stroma running between tumor beds (FIGS. 16B-16C). These fibers tended to run parallel to the tumor beds. Overall, while only about 20% of normal colon cells displayed some PDPN staining, greater than 60% of the tumor samples did (FIG. 16E). Co-staining with myeloperoxidase (MPO) confirmed that the majority of the CD177⁺ cells were neutrophils (FIGS. 16D, and 23D); this data, in combination with the FACS analysis, revealed that most CD177 signal would come from neutrophils. Similarly to PDPN⁺ cells and in agreement with our tissue analyses (FIG. 15D), the amount of CD177⁺ neutrophils was also greatly increased in the tumor samples, with over 60% of tumor samples exhibiting CD177+ staining (FIG. 16E). Some CD177+ cells were identified in peritumoral stroma, where they closely interacted with PDPN⁺ cells (FIGS. 16B-16C); however, most PDPN+ cells were not in contact with a CD177+ cell, as they were located sparsely throughout the tumor microenvironment. Overall, these results indicate that CD177-expressing neutrophils can interact with CAFs expressing PDPN in CRC tissue, suggesting that this newly discovered molecular interaction may influence CAF functions.

### Example 13. Functional Effects of Interactions Between PDPN, CLEC-2, and CD177

We next investigated the functional effects of the interactions between PDPN and CD177 and between PDPN and CLEC-2 on actomyosin contractility in CAFs. We performed 3D elongation experiments as described in Example 10B. 100 nM of recombinant CLEC-2 or CD177 was added to the media. These experiments showed that both CLEC-2 and CD177 caused elongation of CAFs (FIGS. 17A-17B).

To test whether the observed effects occurred when CD177 and CLEC-2 were expressed endogenously on a cell, we performed similar assays in which CAFs were co-cultured with primary neutrophils (CD177⁺ CLEC-2⁺) or with T cells (CD177− CLEC2−). CAFs were seeded with a 5:1 ratio of neutrophils (neut) or T cells isolated from blood. We observed that the neutrophils caused CAF elongation to a similar extent as the recombinant proteins, whereas the T cells did not (FIG. 17C).

To confirm whether this phenotype was true across multiple types of fibroblasts, we repeated this experiment with primary human fibroblasts from healthy colon, bladder, and ovarian tissues. We observed increased elongation only in the two fibroblasts that expressed PDPN, the colon and bladder fibroblasts (FIGS. 17D-17E). These results indicate that CLEC-2 and CD177 are acting through PDPN on the plasma membrane to modulate fibroblast elongation.

We next tested whether CLEC-2 and CD177 also inhibit CAF contractility. Both proteins inhibited contractility by about 50% (FIG. 17F).

### Example 14. Phosphoproteomics Analysis

Next, we sought to study the mechanism underlying CD177 modulation of PDPN functions in primary human CAFs isolated from CRC tissue, which express high endogenous levels of PDPN. First, we analyzed changes in the transcriptional profile of CAFs upon stimulation with CLEC-2 and CD177. No significant changes in the CAF transcriptome were observed (data not shown), suggesting that PDPN targeting may have a more rapid, transient impact on the CAF proteome. Thus, to comprehensively survey signaling pathways and effector proteins modulated by CD177 and CLEC-2, we performed global quantitation of the proteome and phospho-proteome using a deep, multiplexed proteomics approach.

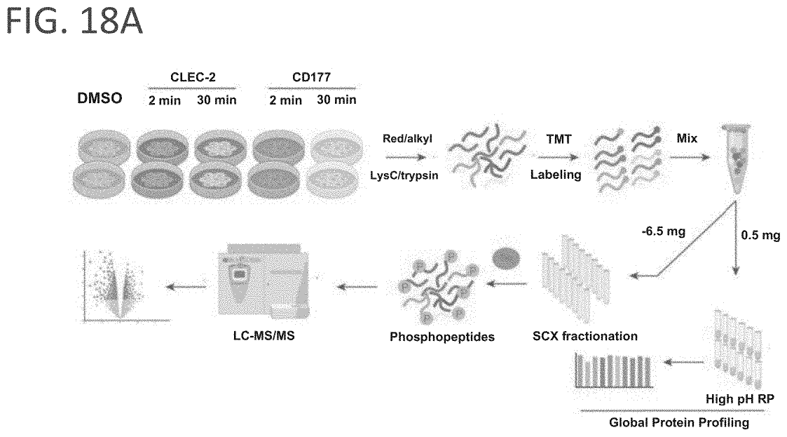

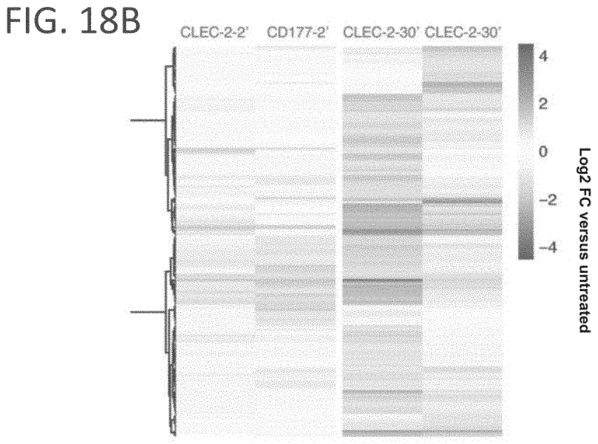

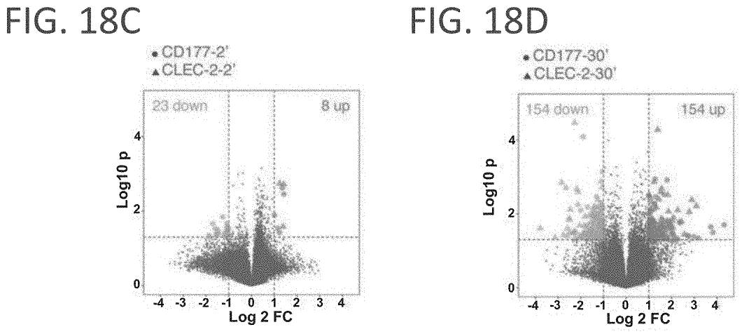

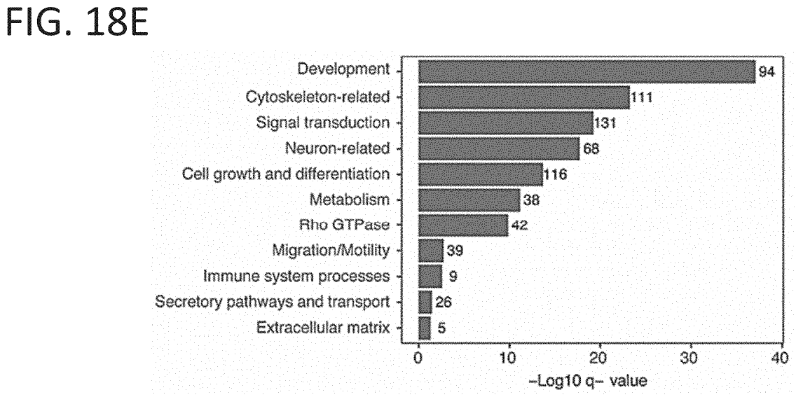

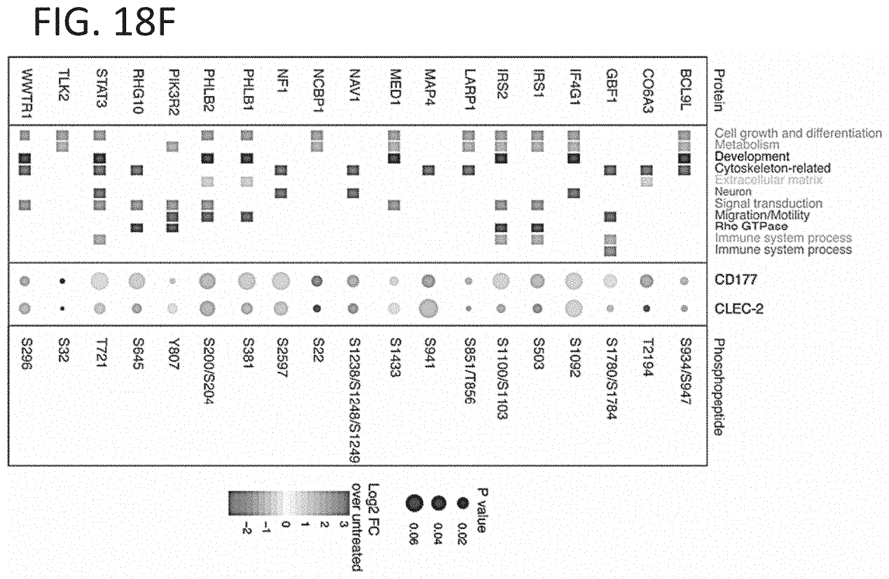

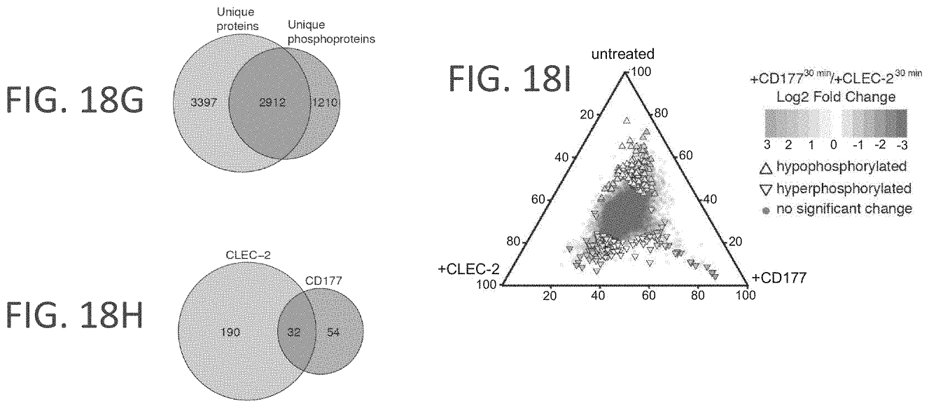

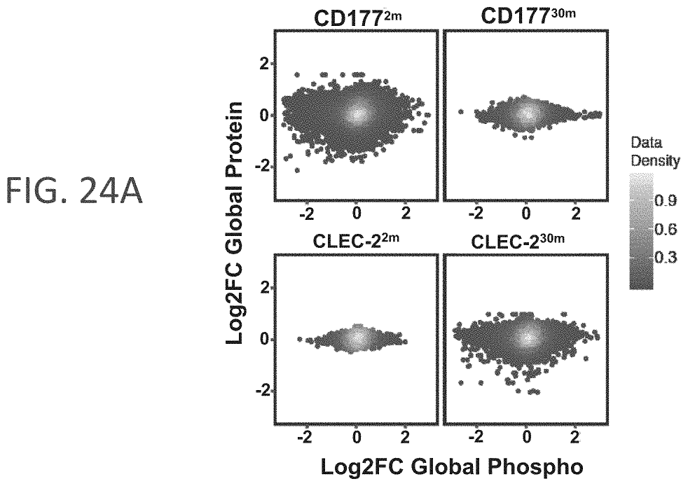

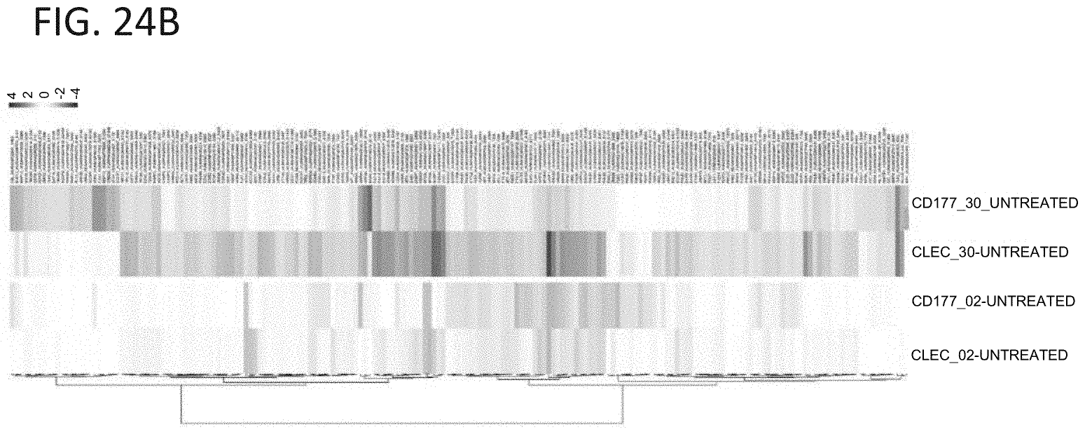

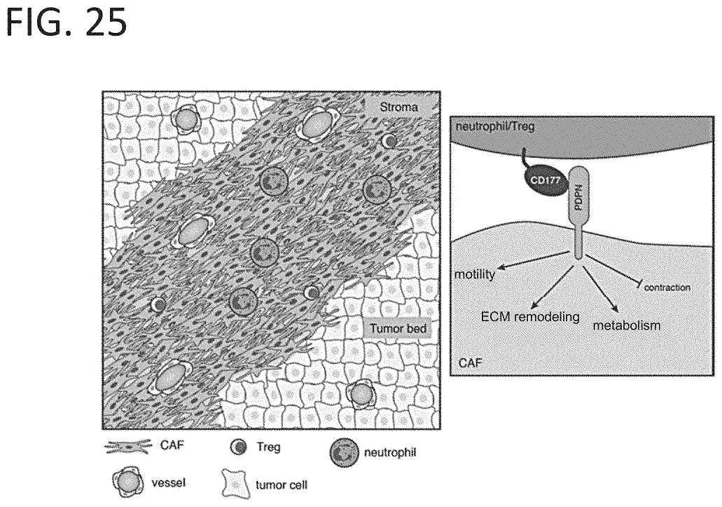

Briefly, CAFs were stimulated with recombinant CD177 or CLEC-2 proteins for 2 min and 30 min, and the resulting whole cell extracts were subjected to global phosphorylation analysis using liquid chromatography coupled to tandem mass spectrometry (LC-MS/MS) (FIG. 18A). We utilized a proteomics profiling method that combines 10-plex tandem mass tag (TMT) labeling of proteome fractions for relative quantitation of phosphopeptides across all conditions, with strong cation exchange fractionation and TiO2 phosphopeptide enrichment to allow for deep phosphoproteomics profiling. Global proteome profiling was also performed to assess protein abundance, followed by computational data processing for total protein and phosphosite quantitation, and finally statistical testing for relative differences between treatment groups (Beausoleil et al., Nat Biotechnol, 24: 1285-1292, 2006). Importantly, this method demonstrated that CD177 or CLEC-2 stimulation did not significantly alter the levels of total protein at 2 minutes or 30 minutes, and thus allowed us to conclude that the observed differential phosphorylation events could be attributed specifically to signaling changes, altogether enabling assessment of global protein levels for almost 3,000 (˜70%) of the quantified phospho-sites (FIGS. 18G and 24A). Changes in the global phosphoproteome were more prominent after 30 min stimulation compared with 2 min (FIGS. 18B and 24F). At 2 min, 30 proteins were significantly modified by either CD177 or CLEC-2 (FIG. 18C), whereas about 300 phosphosites, corresponding to 226 proteins, were significantly regulated after 30 min stimulation with the PDPN binders (FIG. 18D).

To functionally categorize the pathways modulated by PDPN engagement, the phosphoproteins significantly regulated by CD177 or CLEC-2 were analyzed for enrichment in Gene Ontology (GO) terms. These results highlighted major roles for PDPN in cytoskeleton rearrangement, cell motility, extracellular matrix deposition, and secretory pathways, in line with previous data from our laboratory and others (Astarita et al., Nat Immunol, 16: 75-84, 2015; Acton et al., Nature, 514: 498-502, 2014; Martinez et al., Cell Rep, 29: 2810-2822 e2815, 2019) (FIGS. 18E and 24A). Important modulators of microtubule function, cytoskeleton organization, and transport processes were substantially modulated upon PDPN targeting, including plectin (PLEC), the microtubule-associated proteins MAP1S or MAP4, the guanine-nucleotide exchange factors GBF1 and RGH10, or the pleckstrin homology-like domain family B members 1 and 2 (PHLB1 and PHLB2), among others (FIG. 18F). Moreover, and interestingly, these data also suggested prominent alterations in cell growth and differentiation, as well as metabolic processes related to cellular stress and response to exogenous stimuli, biological processes that are relevant to CAF biology that have not been previously linked with PDPN (FIG. 18E). Several key modulators of these processes were significantly post-translationally modified upon CLEC-2 and CD177 stimulation, including a number of transcriptional regulators and RNA binding molecules such as MED1, WWTR1/TAZ, LARP1, and NCBP1 (FIG. 18F).

This global assessment of the phosphoproteome supports a critical role of PDPN signaling in altering CAF cell morphology and cytoskeleton rearrangement, as well as previously unappreciated processes such as cell growth. Furthermore, these data indicate that the binding of either CLEC-2 or CD177 can profoundly alter PDPN signaling into CAFs, indicating that the interactions of immune cells with CAFs could change the TME in previously unappreciated ways.

A. Phosphoproteomics Methods

i. Proteomic Sample Preparation

CAFs were grown to ˜70% confluency on 15 cm dishes. The day of the assay, the cells were starved for 2 hours, followed by stimulation with recombinant CLEC-2 or CD177 for 2 or 30 minutes (Table 13). Stimulations were performed at 37° C. in serum free media. After stimulation, the cells were washed with ice-cold PBS and subsequently harvested and lysed in 20 mM HEPES pH 8.0, 9 M urea containing 1 mM sodium orthovanadate, 2.5 mM sodium pyrophosphate, and 1 mM β-glycerophosphate. Lysates from 5 conditions were analyzed. These were: untreated, CLEC-2 treated for 2 min & 30 min, and CD177 treated for 2 min & 30 min. Two bioreplicates of each 5-plex experiment was performed and combined to make a 10-plex experiment. Samples were sonicated using a Misonix Microson XL sonicator followed by centrifugation at 20,000 g for 20 min at 15° C. Protein concentration was measured using Bradford assay (BioRad). Proteins (1 mg/condition) were reduced with 5 mM dithiothreitol (DTT) at 37° C. for 1 h followed by alkylation with 15 mM iodoacetamide (IAA) at room temperature for 20 min in the dark. Samples were diluted to a final concentration of 2M urea prior to digestion with Lys-C (Wako, Japan) at an enzyme:substrate ratio (E:S) of 1:50 at 37° C. for 4 h followed by tryptic (Promega) digestion at an E:S ratio of 1:50 at 37° C. overnight. The peptide solution was acidified with 20% trifluoroacetic acid (TFA) prior to solid-phase extraction using C18 cartridge (50 mg absorbent) from Waters. Peptides were eluted with 2×0.5 mL of 40% acetonitrile (ACN)/0.1% TFA followed by peptide concentration measurement using a quantitative colorimetric peptide assay kit (Thermo). Equal amounts per condition were subjected to lyophilization overnight.

ii. TMT Labeling

The peptide mixture was reconstituted in 1000 μL of HEPES (200 mM, pH 8.5)+300 μL of ACN, and 100 μL of TMT reagent was added to each of the ten samples (each vial of TMT reagent (Thermo) was dissolved in 40 μL of ACN). Labeling was performed at RT for 1.5 h. A small portion (2 μL) from each condition was mixed, desalted, and analyzed to determine labeling efficiency as well as the relative protein abundance in each sample. The reaction was quenched with 100 μL of 5% hydroxylamine once labeling efficiency was determined to be at least 95%. Samples were mixed at equal amounts followed by acidification using 20% TFA and lyophilized overnight. The 10-plex TMT labeled peptide mixture was desalted using C18 cartridge (200 mg absorbent) from Waters. Sample was eluted with 3×1 mL of 60% ACN/0.1% TFA. A small amount (˜0.5 mg) was saved for total protein profiling and ˜6.5 mg was subjected to global phosphorylation analysis.

iii. Global Phosphorylation & Global Protein Analyses

Strong cation exchange (SCX) fractionation was performed using a PolySulfoethyl 4.6 mm ID×200 mm, 5 μm, 200 Å column (The Nest Group) at a flow rate of 1 mL/min on the HP1100 HPLC system (Agilent Technologies). Sixteen fractions were collected and then desalted followed by phosphopeptide enrichment using the TiO2 enrichment Phos-TiO kit (GL Sciences). For protein profiling, the sample was subjected to high pH reverse phase fractionation, in which 96 fractions were collected and pooled into 24 fractions. Samples were desalted with C18 stagetip prior to mass spectrometry analysis.

iv. Mass Spectrometry Analysis

Desalted peptides were reconstituted in 2% ACN/0.1% formic acid (FA)/water and loaded onto a C18 column (1.7 μm BEH, 130 Å, 0.1×250 mm, New Objective) using a NanoAcquity UPLC system (Waters) at a flow rate of 0.7 μL/min. A gradient of 2% to 30% solvent B (0.1% FA/2% water/ACN) at 0.5 μL/min was applied over 155 min with a total analysis time of 180 min to separate the peptides. Peptides were analyzed using an Orbitrap Fusion Lumos instrument (Thermo). Precursor ions (MS1) were analyzed in the Orbitrap (AGC target 1E6, 120,000 mass resolution, 50 ms maximum injection time) and 10 most abundant species were selected for fragmentation (MS2). Ions were filtered based on charge state ≥2 (z=2, 3 & 4-6) and monoisotopic peak assignment and dynamic exclusion (45 s±10 ppm) was enabled. Each precursor was isolated at a mass width of 0.5 Th followed by fragmentation using collision-induced dissociation (CID at 35 NCE), MS2 AGC target was set at 2.0E4 with a maximum injection time of 200 ms. Multiple fragment ions were isolated using synchronous precursor selection (SPS) prior to HCD (55 NCE, SPS notches=8, AGC target=2.0E5, maximum injection time of 150 ms) MS3 fragmentation and Orbitrap analysis at 50,000 resolution. For global phosphorylation analysis, a neutral loss of 79.97 Da was specified to activate a multistage activation allowing for better identification of phosphorylated species. In addition, MS3 maximum injection time was set to 350 ms.

v. Mass Spectrometry Data Analysis

MS/MS data was searched using the Mascot search algorithm (Matrix Sciences) against a concatenated forward-reverse target-decoy database (downloaded from UniProt in June 2016) consisting of Homo sapiens proteins and common contaminant sequences. Spectra were assigned using a precursor mass tolerance of 50 ppm and fragment ion tolerance of 0.8 Da. Static modifications included carbamidomethyl cysteine (+57.0215 Da), TMT (229.1629 Da) on both the N-terminus of the peptides and lysine residues. Variable modification included oxidized methionine (+15.994 Da) and phosphorylation on serine, threonine and tyrosine residues (+79.9663 Da) for phosphorylation analysis. Trypsin specificity with up to 3 missed cleavages was specified. Peptide spectral matches were filtered at 5% false discovery rate (FDR) followed by protein filtering at 2% FDR. For phosphorylated species, the AScore algorithm was applied to determine the exact phosphorylation site localization16. MS3 TMT quantification was performed using Mojave module, filtering out TMT peaks in MS3 scans that summed to less than 30,000 across all 10 channels. Each peptide was quantified by summing the TMT signal for each sample from all PSMs. Finally, peptides shorter than 7 residues were filtered out.

vi. Mass Spectrometry Statistical Analysis

For the global phospho-proteomics assay, all tryptic phospho-peptides covering identical phospho-site(s) were grouped under a single phospho-site group identifier, including groups that cover more than a single phospho-site. Then, for each phospho-site group (or protein in the global proteome assay) a model was fitted in MSstats v3.7.137 using a Tukey Median Polish summary on all quantified peptides across replicates with imputation of missing values below a censoring threshold of 28. Within MSstats, the model estimated fold change and statistical significance was computed for all compared treatment groups. Significantly altered phospho-site groups were determined by setting a threshold of |Log 2(Fold-Change)|>1 and p-value <0.05. All subsets of significantly altered phospho-site groups were then annotated and tested for over-represented biological annotations (Gene Ontology, PFAM, and KEGG), within each group comparison, with the GoStats package (Falcon and Gentleman, 2007). Significant annotations were defined by a q-value <0.05, group size >2 and genome occurrence <1000 threshold. The significant terms were then further manually grouped into simplified, non-redundant categories for biological process terminology and tested for over-representation with a Fisher's exact test.

### Example 15. Enhancement of Tumor Growth by PDPN⁺ CAFs is Restrained by CD177

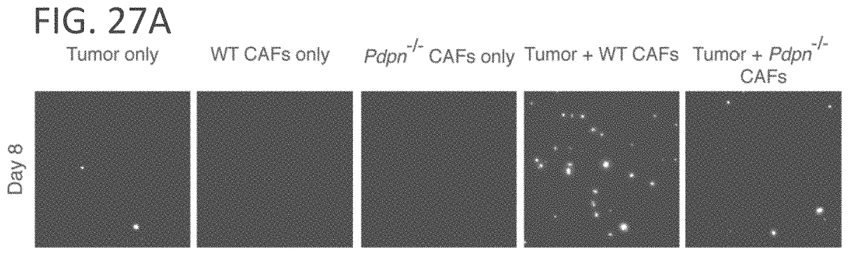

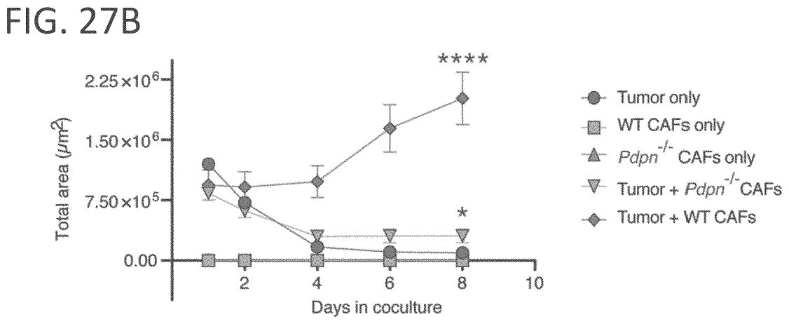

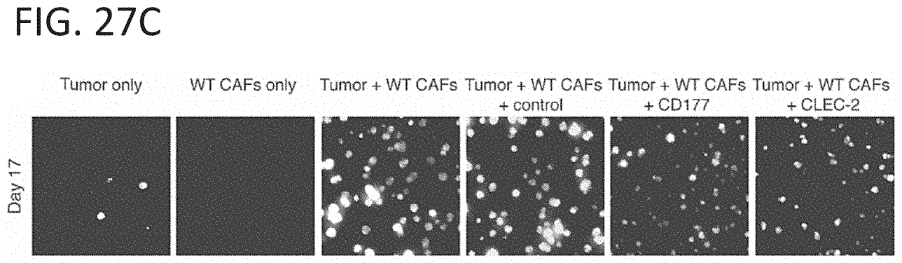

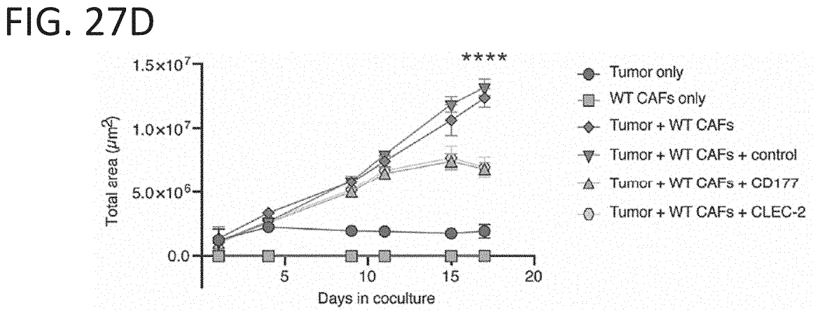

In light of our results showing that PDPN controls CAF growth and actomyosin contractility, and the prognostic significance of PDPN in CRC patients, we next decided to assess whether CRC CAFs could directly support tumor growth. A SW480 tumor cell line expressing red fluorescent protein was generated to enable quantification of tumor organoid growth. Tumor cells were grown alone or in co-culture with WT or PDPN-deficient CAFs, and the CAFs were assessed for their ability to promote tumor organoid growth. WT CAFs significantly enhanced tumor growth compared to tumor cells grown alone. Notably, the Pdpn−/− CAFs were significantly worse at supporting organoid growth, which indicates a central role for PDPN signaling in maintaining the tumor-supporting functions of CAFs (FIGS. 27A and 27B). Next, given that CLEC-2 and CD177 significantly affected the morphology and contractility of CAFs, we sought to evaluate whether CD177 or CLEC-2 would impact the ability of the CAFs to support tumor organoid growth. To this end, tumor cells and CAF co-cultures were stimulated with CD177 and CLEC-2 to mimic interactions between CAFs and immune cells. Tumor organoid growth was evaluated in cultures for 17 days. Remarkably, the increased growth caused by PDPN+ CAFs was significantly decreased upon CD177 or CLEC-2 stimulation, indicating that these molecules inhibit PDPN functions and thereby limit the pro-tumorigenic potential of the CAFs (FIGS. 27C and 27D).

A. Tumor Organoid Culture Methods

To generate the 3D organoid cultures, 2,000 SW-480 tumor cells stably expressing red fluorescent protein (RFP) were seeded in 100 μL of MATRIGELI® Matrix (CORNING®, 356231) with or without 100,000 WT or Pdpn−/− CAFs. Tumor organoids were prepared as described previously (Dominguez et al., Cancer Discov, 10: 232-253, 2019). Briefly, the mixtures were plated in 24-well glass-bottom plates coated with 100 μL of the Matrigel® to prevent attachment of the cells to the coverslip. Next, 2 mL of the tumor cell growth media was added on top of the cultures following gel polymerization, and cultures were monitored for 12-17 days. For CLEC-2 and CD177 treatments, 150 nM of tetramerized CLEC-2 or CD177, provided as tetramers comprising biotinylated extracellular domains of CD177 or CLEC-2 conjugated to streptavidin, were added directly to the MATRIGEL®-cell mixture prior to plating. Additionally, 150 nM of each tetramer was supplemented in the media after plating the organoids, and tetramer additions were repeated every 72 h until day 15. The plates were imaged every 4 days on a NIKON® Ti-E inverted microscope with an automated stage (Applied Scientific Instrumentation), using a NIKON® 4× Plan Fluor objective (NA: 0.13). Images were recorded in tetramethylrhodamine (TRITC) and brightfield channels, and spheres representing tumor organoids were stitched and focused on a single maximum image projection with an extended depth of focus module (EDF, Nikon®). The total area of tumor organoids for each image was analyzed in MATLAB® (vR2018a, MATHWORKS®).

Lengthy table referenced here
US20220119490A1-20220421-T00001
Please refer to the end of the specification for access instructions.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

LENGTHY TABLES
The patent application contains a lengthy table section. A copy of the table is available in electronic form from the USPTO web site (https://seqdata.uspto.gov/?pageRequest=docDetail&DocID=US20220119490A1). An electronic copy of the table will also be available from the USPTO upon request and payment of the fee set forth in 37 CFR 1.19(b)(3).

1-261. (canceled)

262. A method of treating an individual having a cancer comprising administering to the individual an effective amount of an agonist of CD177 activity.

263. The method of claim 262, wherein the expression level of PDPN in a sample from the individual has been determined to be above a reference PDPN expression level.

264. The method of claim 262, wherein the CD177 activity is inhibition of PDPN.

265. The method of claim 262, wherein the sample from the individual is a tumor tissue sample, a tumor fluid sample, a formalin-fixed and paraffin-embedded (FFPE) sample, an archival sample, a fresh sample, or a frozen sample, and wherein: (a) the expression level of PDPN in the sample is a protein expression level of PDPN or an RNA expression level of PDPN; or (b) the expression level of PDPN in the sample is an RNA expression level of PDPN.

266. The method of claim 262, wherein the reference PDPN expression level is an expression level of PDPN in a population of individuals having a cancer, and wherein the reference PDPN expression level is the 50^{th} percentile of expression levels in the population or the 66^{th} percentile of expression levels in the population.

267. The method of claim 262, wherein the cancer is a CRC, a stage II CRC, a stage IV CRC, a squamous cell carcinoma of the head and neck, or a glioma.

268. The method of claim 262, wherein:
(a) the agonist of CD177 activity results in an increase in the binding of PDPN and CD177 relative to binding of the two proteins in the absence of the agonist; or
(b) the agonist of CD177 activity results in a change in a downstream activity of PDPN relative to the downstream activity in the absence of the agonist of CD177 activity.

269. The method of claim 268, wherein the change in the downstream activity is a decrease in tumor growth or a decrease in cancer-associated fibroblast (CAF) contractility.

270. The method of claim 262, wherein the agonist of CD177 activity is a small molecule, an antibody or antigen-binding fragment thereof, a peptide, or a mimic.

271. The method of claim 270, wherein:
(a) the peptide is a CD177 peptide or an extracellular domain of CD177;
(b) the antibody or antigen-binding fragment thereof binds PDPN and is an antagonist antibody or antigen-binding fragment thereof; or
(c) the antibody or antigen-binding fragment thereof binds CD177 and is an agonist antibody or antigen-binding fragment thereof.

272. The method of claim 271, wherein the peptide is multimerized or tetramerized.

273. A collection of polypeptides, wherein each polypeptide comprises an extracellular domain, a tag, and an anchor, and wherein the collection of polypeptides comprises the extracellular domains of at least 81% of the proteins of Table 7.

274. The collection of polypeptides of claim 273, wherein the collection of polypeptides comprises the extracellular domains of all of the proteins of Table 7.

275. The collection of polypeptides of claim 273, wherein:
(a) the anchor is capable of tethering the extracellular domain to the surface of a plasma membrane of a cell, and/or
(b) the tag can be directly or indirectly visualized.

276. The collection of polypeptides of claim 273, wherein (a) the anchor is a glycosylphosphatidyl-inositol (GPI) polypeptide and/or (b) the tag is a glycoprotein D (gD) polypeptide.

277. A collection of vectors encoding the collection of polypeptides of claim 273.

278. A collection of cells comprising the collection of vectors of claim 277.

279. The collection of cells of claim 278, wherein a plurality of the cells are capable of expressing at least one polypeptide of claim 273, optionally wherein different cells express different polypeptides.

280. The collection of polypeptides of claim 273, wherein each of the one or more of said polypeptides is immobilized to a distinct location on one or more solid surfaces.

281. A method for identifying a protein-protein interaction, the method comprising:
(a) providing the collection of polypeptides of claim 273, optionally wherein said polypeptides are immobilized on one or more solid surfaces;
(b) contacting the collection of step (a) with a multimerized query protein under conditions permitting the binding of the multimerized query protein and at least one of the extracellular domains of the polypeptides; and
(c) detecting an interaction between the multimerized query protein and the at least one extracellular domain, thereby identifying a protein-protein interaction.

282. The method of claim 281, wherein one or more of said polypeptides each is immobilized to a distinct location on said one or more solid surfaces, and wherein the distinct location comprises a cell that displays the polypeptide.

283. The method of claim 282, wherein the cells are mammalian cells.

284. The method of claim 281, wherein the interaction is a transient interaction; a low-affinity interaction; or a micromolar-affinity interaction.

285. The method of claim 281, wherein:
(a) the multimerized query protein is a dimerized, trimerized, tetramerized, or pentamerized query protein; and/or
(b) the multimerized query protein comprises an isolated extracellular domain of the query protein.

286. A method of identifying a modulator of the interaction between a protein of Table 1 and a protein of Table 2, the method comprising:
(a) providing a candidate modulator;
(b) contacting a protein of Table 1 with a protein of Table 2 in the presence or absence of the candidate modulator under conditions permitting the binding of the protein of Table 1 to the protein of Table 2, wherein the protein of Table 1 and the protein of Table 2 are reported to interact in Table 3; and
(c) measuring the binding of the protein of Table 1 to the protein of Table 2, wherein an increase or decrease in binding in the presence of the candidate modulator relative to binding in the absence of the candidate modulator identifies the candidate modulator as a modulator of the interaction between the protein of Table 1 and the protein of Table 2.

287. A method of identifying a modulator of a downstream activity of a protein of Table 1, the method comprising:
(a) providing a candidate modulator;
(b)
(i) contacting the protein of Table 1 with a protein of Table 2 in the presence or absence of the candidate modulator under conditions permitting the binding of the protein of Table 1 to the protein of Table 2; or
(ii) contacting the protein of Table 2 with a protein of Table 1 in the presence or absence of the candidate modulator under conditions permitting the binding of the protein of Table 2 to the protein of Table 1,
wherein the protein of Table 1 and the protein of Table 2 are reported to interact in Table 3; and
(c)
(i) measuring a downstream activity of the protein of Table 1, wherein a change in the downstream activity in the presence of the candidate modulator relative to the downstream activity in the absence of the candidate modulator identifies the candidate modulator as a modulator of the downstream activity of the protein of Table 1; or
(ii) measuring a downstream activity of the protein of Table 2, wherein a change in the downstream activity in the presence of the candidate modulator relative to the downstream activity in the absence of the candidate modulator identifies the candidate modulator as a modulator of the downstream activity of the protein of Table 2.

288. The method of claim 286, wherein the increase or decrease in binding is at least 70%, as measured by surface plasmon resonance, biolayer interferometry, or an enzyme-linked immunosorbent assay (ELISA).

289. The method of claim 287, wherein:
(a) the modulator is an inhibitor of the downstream activity of the protein of Table 1 or Table 2 and/or the change in the downstream activity is a decrease in the amount, strength, or duration of the downstream activity; or
(b) the modulator is an activator of the downstream activity of the protein of Table 1 or Table 2 and/or the change in the downstream activity is an increase in the amount, strength, or duration of the downstream activity.

290. The method of claim 286, wherein the modulator is a small molecule, an antibody or antigen-binding fragment thereof, a peptide, a mimic, an antisense oligonucleotide, or a small interfering RNA (siRNA).

291. The method of claim 290, wherein the antibody or antigen-binding fragment thereof binds the protein of Table 1 or the protein of Table 2.

292. The method of claim 286, wherein:
(a) the protein of Table 1 is podoplanin (PDPN) and the protein of Table 2 is CD177;
(b) the protein of Table 1 is PD-L1 (CD274) and the protein of Table 2 is EPHA3;
(c) the protein of Table 1 is PD-L2 (PDCD1LG2) and the protein of Table 2 is CEACAM4, ICAM5, NECTIN3, PSG9, or TNFRSF11A;
(d) the protein of Table 1 is PTPRD and the protein of Table 2 is BMP5, CEACAM3, IL1RAP, IL1RAPL2, LECT1, LRFN5, SIRPG, SLITRK3, SLITRK4, SLITRK6, or TGFA;
(e) the protein of Table 1 is PTPRS and the protein of Table 2 is C6orf25, IL1RAP, IL1RAPL1, IL1RAPL2, LRFN1, LRFN5, LRRC4B, NCAM1, SLITRK1, SLITRK2, SLITRK3, SLITRK4, or SLITRK6;
(f) the protein of Table 1 is PTPRF and the protein of Table 2 is CD177, IL1RAP, or LRFN5;
(g) the protein of Table 1 is CHL1 and the protein of Table 2 is CNTN1, CNTN5, SIRPA, L1CAM, or TMEM132A;
(h) the protein of Table 1 is CNTN1 and the protein of Table 2 is CDH6, CHL1, FCGRT, PCDHB7, or SGCG;
(i) the protein of Table 1 is LILRB1 and the protein of Table 2 is CLEC6A, CXADR, EDAR, FLT4, IL6R, ILDR1, or LILRA5;
(j) the protein of Table 1 is LILRB2 and the protein of Table 2 is IGSF8 or MOG;
(k) the protein of Table 1 is LILRB3 and the protein of Table 2 is LRRC15 or LY6G6F;
(l) the protein of Table 1 is LILRB4 and the protein of Table 2 is CNTFR;
(m) the protein of Table 1 is LILRB5 and the protein of Table 2 is APLP2, CD177, CLEC10A, CLECSF13, LDLR, PILRA, or UNC5C;
(n) the protein of Table 1 is AXL and the protein of Table 2 is IL1RL1 or VSIG10L; or
(o) the protein of Table 1 is LRRC4B and the protein of Table 2 is BTN3A1 or BTN3A3.

293. An isolated modulator of the interaction between a protein of Table 1 and a protein of Table 2, wherein:
(a) the protein of Table 1 and the protein of Table 2 are reported to interact in Table 3; and
(b) the modulator causes an increase or decrease in the binding of the protein of Table 1 to the protein of Table 2 relative to binding in the absence of the modulator; or
(c) the modulator causes a change in the downstream activity of the protein of Table 1 or the protein of Table 2 relative to downstream activity in the absence of the modulator.

294. The modulator of claim 293, wherein the increase or decrease in binding is at least 70%, as measured by surface plasmon resonance, biolayer interferometry, or an enzyme-linked immunosorbent assay (ELISA).

295. The modulator of claim 293, wherein:
(a) the modulator is an inhibitor of the downstream activity of the protein of Table 1 or Table 2 and the change in the downstream activity is a decrease in the amount, strength, or duration of the downstream activity; or
(b) the modulator is an activator of the downstream activity of the protein of Table 1 or Table 2 and the change in the downstream activity is an increase in the amount, strength, or duration of the downstream activity.

296. The modulator of claim 293, wherein the modulator is a small molecule, an antibody or antigen-binding fragment thereof, a peptide, or a mimic.

297. The modulator of claim 296, wherein the antibody or antigen-binding fragment thereof binds the protein of Table 1 or the protein of Table 2.

298. A method of treating an individual having a cancer, the method comprising administering a PD-L1 axis binding antagonist to an individual who has been determined to have an expression level of a first member of a gene pair of Table 15 that is above a first reference expression level and an expression level of the second member of the gene pair that is above a second reference expression level.


SEQUENCE LISTING (33 sequences)

SEQ ID NO: 1 - protein, 440 aa, Artificial Sequence
  Synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence
  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, Artificial Sequence [sequence omitted]

SEQ ID NO: 4 - protein, 108 aa, Artificial Sequence
  Synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 5 - protein, 10 aa, Artificial Sequence
  Synthetic construct
  MISC_FEATURE (6)..(6): Xaa is Asp or Gly
GFTFSXSWIH

SEQ ID NO: 6 - protein, 18 aa, Artificial Sequence
  Synthetic construct
  MISC_FEATURE (4)..(4): Xaa is Ser or Leu
  MISC_FEATURE (10)..(10): Xaa is Thr or Ser
AWIXPYGGSXYYADSVKG

SEQ ID NO: 7 - protein, 9 aa, Artificial Sequence
  Synthetic construct
RHWPGGFDY

SEQ ID NO: 8 - protein, 25 aa, Artificial Sequence [sequence omitted]

SEQ ID NO: 9 - protein, 13 aa, Artificial Sequence
  Synthetic construct
WVRQAPGKGLEWV

SEQ ID NO: 10 - protein, 32 aa, Artificial Sequence
  Synthetic construct
RFTISADTSKNTAYLQMNSLRAEDTAVYYCAR

SEQ ID NO: 11 - protein, 11 aa, Artificial Sequence
  Synthetic construct
WGQGTLVTVSS

SEQ ID NO: 12 - protein, 11 aa, Artificial Sequence
  Synthetic construct
  MISC_FEATURE (5)..(5): Xaa is Asp or Val
  MISC_FEATURE (6)..(6): Xaa is Val or Ile
  MISC_FEATURE (7)..(7): Xaa is Ser or Asn
  MISC_FEATURE (9)..(9): Xaa is Ala or Phe
  MISC_FEATURE (10)..(10): Xaa is Val or Leu
RASQXXXTXXA

SEQ ID NO: 13 - protein, 7 aa, Artificial Sequence
  Synthetic construct
  MISC_FEATURE (4)..(4): Xaa is Phe or Thr
  MISC_FEATURE (6)..(6): Xaa is Tyr or Ala
SASXLXS

SEQ ID NO: 14 - protein, 9 aa, Artificial Sequence
  Synthetic construct
  MISC_FEATURE (3)..(3): Xaa is Tyr, Gly, Phe, or Ser
  MISC_FEATURE (4)..(4): Xaa is Leu, Tyr, Phe, or Trp
  MISC_FEATURE (5)..(5): Xaa is Tyr, Asn, Ala, Thr, Gly, Phe, or Ile
  MISC_FEATURE (6)..(6): Xaa is His, Val, Pro, Thr, or Ile
  MISC_FEATURE (8)..(8): Xa is Ala, Trp, Arg, Pro, or Thr
QQXXXXPXT

SEQ ID NO: 15 - protein, 23 aa, Artificial Sequence
  Synthetic construct
DIQMTQSPSSLSASVGDRVTITC

SEQ ID NO: 16 - protein, 15 aa, Artificial Sequence
  Synthetic construct
WYQQKPGKAPKLLIY

SEQ ID NO: 17 - protein, 32 aa, Artificial Sequence
  Synthetic construct
GVPSRFSGSGSGTDFTLTISSLQPEDFATYYC

SEQ ID NO: 18 - protein, 11 aa, Artificial Sequence
  Synthetic construct
FGQGTKVEIKR

SEQ ID NO: 19 - protein, 10 aa, Artificial Sequence
  Synthetic construct
GFTFSDSWIH

SEQ ID NO: 20 - protein, 18 aa, Artificial Sequence
  Synthetic construct
AWISPYGGSTYYADSVKG

SEQ ID NO: 21 - protein, 9 aa, Artificial Sequence
  Synthetic construct
RHWPGGFDY

SEQ ID NO: 22 - protein, 11 aa, Artificial Sequence
  Synthetic construct
RASQDVSTAVA

SEQ ID NO: 23 - protein, 7 aa, Artificial Sequence
  Synthetic construct
SASFLYS

SEQ ID NO: 24 - protein, 9 aa, Artificial Sequence
  Synthetic construct
QQYLYHPAT

SEQ ID NO: 25 - protein, 122 aa, Artificial Sequence
  Synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 26 - protein, 10 aa, Artificial Sequence
  Synthetic construct
FGQGTKVEIK

SEQ ID NO: 27 - protein, 30 aa, Artificial Sequence
  Synthetic construct
EVQLVESGGGLVQPGGSLRLSCAASGFTFS

SEQ ID NO: 28 - protein, 14 aa, Artificial Sequence
  Synthetic construct
WVRQAPGKGLEWVA

SEQ ID NO: 29 - protein, 15 aa, Artificial Sequence
  Synthetic construct
WGQGTLVTVSSASTK

SEQ ID NO: 30 - protein, 447 aa, Artificial Sequence
  Synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, Artificial Sequence
  Synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 115 aa, Artificial Sequence
  Synthetic Construct
QSLEESGGRLVKPDETLTITCTVSGIDLSSNGLTWVRQAPGEGLEWIGTINKDASAYYASWAKGRLTISKPSSTKVDLKITSPTTEDTATYFCGRIAFKTGTSIWGPGTLVTVSS

SEQ ID NO: 33 - protein, 112 aa, Artificial Sequence
  Synthetic Construct
AIVMTQTPSPVSAAVGGTVTINCQASESVYSNNYLSWFQQKPGQPPKLLIYLASTLASGVPSRFKGSGSGTQFTLTISGVQCDDAATYYCIGGKSSSTDGNAFGGGTEVVVR